SEQUENCE LISTING (345 sequences)

SEQ ID NO: 1 - DNA, 3504 bp, Klebsiella pneumoniae
  misc_feature 1..3504: note = wild type Gene J, nifJ
  source 1..3504: mol_type = unassigned DNA
atgaaaacaatggatggcaacgccgcggcggcctggatctcttatgcctttaccgaggtcgcggcgatttaccccataaccccctccacgccgatggcggaaaacgtcgacgagtgggcggcgcaggggaaaaagaacctttttggccagccggtgcgcttaatggagatgcagtcggaggccggcgcggcaggcgcggtccacggcgcgctgcaggccggggcgctcaccaccacctatacggcctcccaggggctgctgctgatgatccccaacatgtacaaaatcgccggtgaactgctgccgggcgtctttcacgtcagcgcccgggcgctggcgaccaattcgctgaatatttttggcgatcaccaggatgtgatggcggtccgccagaccggctgcgcgatgctggcggagaacaacgtgcagcaggtgatggatttgtcggcggtggcgcatctggcggcgattaagggacgcatcccgtttgttaacttcttcgacggttttcgcacctcgcacgaaattcaaaaaatcgaggtgttggaatatgagcagctggcgacgctgctggaccggcccgcgctcgacagcttccgccgtaacgcgctgcatccggatcatccggtcatccgcggaacggcgcagaacccggatatctacttccaggagcgggaggcgggcaaccgtttttatcaggcgctgcccgacattgtcgaaagctatatgacgcaaatcagcgcgctcaccggccgggagtatcatctgtttaactataccggcgcggcggatgccgaacgggtgattatcgcaatgggatcggtctgcgataccgttcaggaagtggtggatacgctgaacgcggcgggagagaaggtcgggctgctcagcgtgcatctgtttcgccctttttcgctggcccacttcttcgcccagctgccgaaaaccgtgcagcggattgccgtccttgaccgtaccaaagagcccggcgctcaggctgaaccgctgtgcctggatgtgaaaaacgccttctaccaccacgacgatgcgccgctaatcgtcggcggccgctatgcgcttggcgggaaggatgttctgcccaacgatatcgcggccgtctttgataacctcaataaaccgctgccgatggacgggtttaccctcgggattgtcgatgatgtaacctttacttcgttaccgccggcccagcagacgctggcggtctcgcatgacggcatcaccgcctgtaaattttggggcatgggctcggacggcaccgtcggggccaataaaagcgcgatcaaaattatcggcgataaaacgccgctctacgcgcaggcctacttttcttatgactcgaaaaaatccggcggcattaccgtttcacatttacgcttcggcgaccggccgatcaattcgccctacctgatccatcgggccgattttatctcctgttcgcagcagtcctacgttgagcgctacgatctgctggacggattaaagccgggcgggacctttttactcaactgcagctggtccgatgcggagctggagcagcatctgccggtcggctttaaacgctatctggcgcgggaaaatatccatttttataccctgaacgccgtggatatcgcccgcgagctcgggctgggcgggcgctttaatatgctgatgcaggcggcgttctttaagctggcggcgattatcgacccgcagaccgcggcggattacctcaagcaggcggttgaaaaaagctacggcagcaaaggggcggcggtgattgagatgaaccagcgggcgatcgagctgggcatggcctcgctgcatcaggtgacgattccggcgcactgggcgacgctggatgaacccgcggcgcaagcatcagccatgatgccggatttcatccgcgatattctgcagccgatgaaccgccagtgcggcgaccagctgccggtgagcgcgttcgtcggtatggaggacgggacctttccttcgggcaccgccgcgtgggagaaacgcgggatcgcgctggaagtgccggtctggcagccggagggctgcacgcagtgtaaccagtgcgcctttatctgcccgcatgcggcgatccgcccggcgctgctcaacggcgaagagcatgacgccgcgccggttgggctgctgagcaaacccgcgcagggagcgaaggagtatcactaccatctggctatctcgccgctggattgttccggctgcggcaactgcgtggatatctgtcccgcgcgcggcaaggcgttaaaaatgcagtctctcgatagccagcgtcagatggcgccggtctgggactatgcgctggcgctgacgccgaagagcaatccgtttcgtaagacgacggtcaaaggcagccagtttgaaaccccgctgctggagttttccggcgcctgcgcgggatgcggtgaaacgccttatgcccgcctgataacccagctgtttggcgaccggatgctgatcgctaacgccaccggctgctcttctatctggggagccagcgcgccgtcgattccctataccaccaaccaccgcggccatggcccggcatgggcgaactcgctgtttgaggataatgcggagtttggcctcggcatgatgctcggcggccaggcggtgcgtcaacaaatcgccgatgatatgaccgccgcgctggcgctaccggtcagcgacgaacttagcgacgcaatgcgccagtggctggcgaagcaggatgaaggcgagggcacccgcgagcgcgcggaccggctcagcgaacggctggcggcggaaaaagagggcgtgccgctgttggagcagctgtggcagaaccgcgactattttgttcgtcgttcgcagtggattttcggcggcgacggctgggcctacgatatcggcttcggcggtctcgatcacgtgctggcgagcggggaagacgtcaatattctggtgtttgacaccgaggtttactccaataccggcggccagtcgtctaaatcgaccccggtggcggccatcgcgaagtttgccgcgcagggcaaacgcacgcggaaaaaagatctcggcatgatggcgatgagctacggcaatgtgtacgtggcccaggtcgcgatgggcgctgataaagatcagaccctacgggcgatcgccgaggccgaagcctggccgggaccgtcgctggtgattgcctacgccgcctgcattaaccacgggctgaaagccggtatgcgctgcagccagcgcgaggcgaaacgggcggttgaggcgggatactggcacctgtggcgctatcatccgcagcgggaagcggaaggtaagacgccgtttatgctcgattccgaagagccggaggagagcttccgcgactttttgcttggcgaagtgcgctacgcctcgctgcacaaaacgacgccgcatctggcggatgcgctctttagccgaaccgaggaggacgcgcgggcccgctttgcccagtatcggcggctggccggcgaggagtag

SEQ ID NO: 2 - DNA, 3504 bp, synthetic construct
  misc_feature 1..3504: note = synthetic Gene J
  source 1..3504: mol_type = other DNA
atgaaaactatggacggtaacgctgcggctgcatggattagctacgcctttaccgaagtggctgcgatctacccgattacgccgagcaccccgatggcggaaaatgtggacgaatgggctgcgcagggcaagaagaacctcttcggccagccggtgcgcctgatggagatgcagtcggaagcgggtgcagcaggtgctgtgcatggcgccttgcaagctggcgcactgacgaccacctacaccgcgtcgcagggcctgttgctgatgatcccaaacatgtacaaaatcgcgggtgaactgctgccgggtgtctttcatgtttcggcacgcgcactggccaccaatagcctcaacatctttggcgatcatcaggatgtaatggcggtgcgccaaacgggctgcgcgatgttggccgagaataacgtccagcaagttatggatttgtccgcggtagcccacttggcagcgatcaaaggtcgcattccgttcgtgaacttcttcgatggctttcgcaccagccacgaaatccagaagatcgaggttctggaatatgaacagctggccaccttgttggatcgtccggccctggacagcttccgccgtaacgcccttcacccggaccacccggtcatccgtggcaccgcccagaacccggacatctacttccaggaacgtgaggccggtaaccgtttctatcaggcgctcccggatattgtggaatcttacatgacccagatttctgccctgactggtcgcgagtatcacctgtttaactacactggtgctgcggatgcggagcgcgtgatcatcgcgatgggctctgtctgtgacaccgtccaagaggtggttgacacgctgaatgcagcgggtgagaaagttggtctgctctccgttcatcttttccgcccgttttcgttagcgcacttcttcgcccaactgccgaaaactgtacagcgtatcgcagtattggaccgtacgaaagagccaggtgctcaagcagagccgctgtgcctcgatgtgaagaatgccttttaccaccatgacgatgccccgttgattgtgggtggtcgctatgccttgggcggtaaggacgtgttgccgaacgatattgcggccgtgtttgataacctgaacaaaccgctgccgatggacggcttcacgctgggtatcgtggacgatgttaccttcacctctctcccgccagcgcagcagaccctggcggtttctcacgacggcatcacggcatgtaagttttggggcatgggctccgacggcacggttggtgcgaacaagtccgcgatcaagattatcggcgacaaaacgccactgtatgcgcaagcgtacttttcctacgactcgaagaagagcggtggtattaccgtcagccatctgcgttttggtgatcgcccgatcaactccccgtatttgatccatcgcgcggatttcatctcgtgcagccagcaaagctatgttgaacgctacgatctgctggatggccttaaaccgggtggcacctttctgctgaactgctcctggagcgatgccgaactggagcaacatctgccggtcggtttcaaacgttatctggcacgcgagaatatccacttctacactctcaacgctgtggacatcgcccgtgagcttggtttgggtggccgtttcaacatgctgatgcaggctgccttcttcaaactggccgcgatcattgacccgcagactgctgcggactatctgaagcaggctgttgagaaaagctatggcagcaaaggtgcggcggtcatcgagatgaaccagcgtgccatcgagcttggcatggccagcctgcaccaggtgacgatcccggcacattgggccaccctggatgagccagcggcgcaggcgtccgcgatgatgccggactttatccgcgacatcctgcaaccgatgaaccgtcagtgcggcgaccagcttccggtgtcggcttttgtcggcatggaagatggcaccttcccgtccggcacggccgcatgggagaaacgtggcatcgcccttgaggtgccagtctggcagccggaaggctgcacgcagtgcaaccagtgcgccttcatttgtccgcacgccgcgattcgtccggcgttgttgaatggcgaagagcatgatgctgccccggttggcctgctgagcaaaccggcacaaggcgctaaagaatatcactatcatctggcgattagcccgctggactgctccggctgtggcaactgcgttgacatttgtccagctcgtggcaaagcgttgaagatgcagtctctggatagccaacgccagatggctccggtgtgggattatgcgctggcgctgaccccgaagtctaacccgtttcgtaaaaccaccgtcaaaggctcgcagttcgaaaccccgctgctggagtttagcggtgcgtgcgctggttgtggcgaaacgccgtatgcgcgcctcattacccagctgtttggcgaccgcatgctgattgccaatgccaccggctgttccagcatctggggcgcatctgcgccgagcatcccgtataccaccaatcatcgtggtcatggtccggcctgggcgaatagcctgtttgaggacaatgccgaatttggtttaggtatgatgctgggcggtcaagctgtgcgtcaacagatcgcggacgatatgacggctgcgttagcgctcccggtttccgatgagctgagcgacgcgatgcgccagtggttggcgaaacaggacgagggtgaaggcacgcgtgagcgtgcggaccgtctgagcgagcgcttagccgcggagaaagagggcgttccgctgttagagcagctgtggcaaaatcgtgattactttgtgcgtcgcagccagtggattttcggcggtgacggctgggcctatgatattggcttcggtggcctggaccacgtcctcgccagcggtgaggatgtgaacattctggtatttgacaccgaagtctactcgaacaccggcggtcaaagcagcaaatcgaccccggtcgcggccatcgccaagttcgcggctcagggcaagcgcacccgcaagaaagacctgggtatgatggcgatgagctacggcaacgtctatgtagcccaggtggcgatgggtgcggataaagatcaaactctgcgcgccattgcggaagctgaagcgtggccaggcccgtcgctggtgattgcgtatgcggcctgcatcaatcatggcctgaaggccggtatgcgttgcagccaacgtgaggcgaagcgcgctgttgaggcgggctactggcacctgtggcgttatcacccgcagcgcgaagcggaaggcaagacgccgtttatgttagatagcgaagaaccggaagagtcgttccgtgactttctgttgggtgaggtgcgctacgcatccctgcacaagaccaccccgcacctcgccgatgcccttttcagccgtaccgaagaagatgcgcgtgcgcgctttgcgcaataccgtcgcctggctggcgaagagtaa

SEQ ID NO: 3 - DNA, 882 bp, Klebsiella pneumoniae
  misc_feature 1..882: note = wild type Gene H, nifH
  source 1..882: mol_type = unassigned DNA
atgaccatgcgtcaatgcgctatttacggtaaaggcggtatcggtaaatccaccaccacgcagaacctcgtcgccgcgctggcggagatgggtaagaaagtgatgatcgtcggctgcgatccgaaggcggactccacccgtctgattctgcacgccaaagcacagaacaccattatggagatggccgcggaagtcggctcggtcgaggacctcgaactcgaagacgtgctgcaaattggctacggcgatgtgcgctgcgcggaatccggcggcccggagccaggcgtcggctgcgcgggacgcggcgtgatcacggcgatcaactttcttgaagaagaaggcgcctacgaggacgatctcgatttcgtgttctatgacgtgctcggcgacgtggtctgcggcggcttcgccatgccgatccgcgaaaacaaagcccaggagatctacatcgtctgctccggcgaaatgatggcgatgtacgcggccaacaatatctccaaagggatcgttaaatacgccaaatccggcaaggtgcgcctcggcggcctgatctgtaactcacgtcagaccgaccgtgaagacgaactgattattgccctggcggaaaagctcggtacccagatgatccactttgtgccccgcgacaacatcgtgcagcgcgcggagatccgccgcatgacggttatcgagtacgaccccgcctgtaaacaggccaacgaataccgcaccctggcgcagaagatcgtcaacaacaccatgaaagtggtgccgacgccctgcaccatggatgagctggaatcgctgctgatggagttcggcatcatggaagaggaagacaccagcatcattggcaaaaccgccgccgaagaaaacgcggcctga

SEQ ID NO: 4 - DNA, 882 bp, synthetic construct
  misc_feature 1..882: note = synthetic Gene H
  source 1..882: mol_type = other DNA
atgaccatgcgtcagtgcgcgatttatggcaaaggtggtattggcaaaagcacgacgacccagaacttggtggcggccctggccgagatgggtaaaaaggttatgattgtgggttgcgacccgaaggccgacagcacgcgcctgattctgcacgcgaaagcacaaaacacgattatggagatggctgccgaggttggtagcgtggaggatctggagctggaggacgttctgcaaattggttacggtgatgttcgttgcgcagagagcggtggtccggaaccaggtgtcggctgtgcgggtcgtggtgtgattaccgctatcaatttcctggaagaagagggtgcgtacgaagatgatctggatttcgttttctacgatgtgctgggtgatgtcgtgtgcggtggttttgcaatgccgattcgcgagaataaggcacaagaaatttacattgtctgtagcggcgagatgatggcaatgtacgctgctaacaacatcagcaagggtattgttaaatacgcaaaaagcggtaaggttcgcttgggtggtttgatttgcaacagccgtcagaccgaccgtgaggacgaactgatcatcgccctggctgagaaactgggcacccaaatgatccacttcgtgccacgcgataatattgttcaacgtgcagaaatccgccgtatgaccgtcattgagtatgacccggcatgcaagcaagcgaacgagtaccgcaccttggcacagaaaatcgtgaacaacaccatgaaggttgttccgacgccgtgtacgatggacgagctggagagcctgctgatggagttcggcattatggaggaggaggacaccagcattatcggtaagaccgcagcggaggagaatgcggcataa

SEQ ID NO: 5 - DNA, 1452 bp, Klebsiella pneumoniae
  misc_feature 1..1452: note = wild type Gene D, nifD
  source 1..1452: mol_type = unassigned DNA
atgatgaccaacgcaacgggcgaacgtaatctggcgctgatccaggaagtcctggaggtgttcccggaaaccgcgcgaaaagagcgcagaaagcacatgatggtcagcgatccgaaaatgaagagcgtcggcaagtgcattatctctaaccgcaaatcacaacccggcgtaatgaccgtacgcggctgcgcctacgccggttccaaaggggtggtatttgggccgattaaggatatggcccatatttcgcacggaccggctggctgcggccagtattcccgcgccgaacgacgcaactactacaccggagtcagcggcgtcgatagcttcggcacgctgaacttcacctctgattttcaggagcgcgacatcgtcttcggcggcgataaaaagctcagcaagctgattgaagagatggagttgctgttcccgctcaccaaagggatcaccattcagtcggaatgcccggtggggctgatcggtgatgatatcagcgcggtggccaacgccagcagcaaggcgctggataaaccggtgatcccggtacgctgcgaaggctttcgcggcgtgtcgcagtctctggggcaccatatcgccaacgacgtggtgcgcgactggatcctgaacaatcgcgaaggacagccgtttgaaaccaccccttacgatgtggcgatcatcggcgactacaacatcggcggcgacgcctgggcctcgcgcattctgctggaagagatggggctacgggtagtcgcgcagtggtccggcgacggcacgctggtggagatggagaataccccattcgtcaagctgaacctggttcactgctaccgttcgatgaactatatcgcccgccatatggaggagaaacatcagattccgtggatggagtacaacttcttcgggccgaccaaaatcgccgaatcgctgcgcaaaatcgccgaccagttcgacgataccattcgcgcgaacgccgaagcggtgatcgcccggtatgaggggcagatggcggcgattatcgccaaatatcgcccgcgcctggaggggcgtaaggtgctgctctatatcggaggcctgcggccgcgccacgttattggcgcctatgaggatctcgggatggagatcatcgccgccggctacgagtttgcccataacgatgattacgaccgcaccctgccggatctgaaagagggcacgctgctgttcgatgacgccagcagctacgagctggaagcgttcgtcaaggcgctgaagcccgaccttatcggctccggcatcaaggaaaaatatatcttccagaaaatgggcgtgccgttccgccagatgcactcgtgggactattccggcccgtaccacggctacgatggtttcgccattttcgcccgcgatatggatatgaccctgaacaacccggcgtggaacgaactgaccgctccgtggctgaagtctgcgtga

SEQ ID NO: 6 - DNA, 1452 bp, synthetic construct
  misc_feature 1..1452: note = synthetic Gene D
  source 1..1452: mol_type = other DNA
atgatgactaatgctactggcgaacgtaacctggcactgattcaagaagtactggaagtgttcccggaaaccgcgcgcaaagagcgccgtaaacacatgatggtttctgacccgaaaatgaaatctgtgggtaaatgcatcatctctaatcgcaaatctcagccgggtgtcatgactgttcgtggctgtgcgtacgcaggttctaaaggtgtcgtattcggcccgatcaaagatatggcgcatatctctcatggcccggcaggctgtggccagtactctcgcgcggaacgtcgtaactactacacgggcgtttctggcgttgactctttcggcacgctgaacttcacctctgacttccaggaacgtgacatcgttttcggtggcgataaaaagctgtccaaactgatcgaagaaatggaactgctgttcccgctgactaaaggcattactatccaaagcgaatgtccggtgggtctgatcggtgatgacatcagcgcggtcgcaaacgcatcttccaaagccctggataagccggtgatcccggttcgttgcgagggcttccgcggcgtttctcagtctctgggtcatcacatcgcaaacgatgttgtgcgtgactggattctgaacaaccgtgaaggtcagccttttgaaaccaccccttatgacgttgcgattattggcgactataacatcggcggcgacgcctgggcatcccgcatcctgctggaggagatgggtctgcgtgttgtcgcacagtggtctggcgatggcaccctggttgaaatggaaaacaccccgtttgttaaactgaacctggttcactgctaccgctccatgaactacattgcccgtcacatggaagaaaaacatcagatcccttggatggaatacaacttcttcggtccgactaaaatcgcagaatccctgcgtaaaatcgccgatcagtttgatgataccattcgcgcgaacgctgaagcagtaattgcgcgctacgaaggccagatggcagcaatcattgctaagtaccgtccgcgcctggaaggtcgtaaagtgctgctgtacatgggtggtctgcgtccacgtcatgtgatcggtgcctacgaggacctgggcatggagatcatcgcagcgggttacgaatttgcacacaacgacgactatgatcgtacgctgccagacctgaaagaaggtacgctgctgtttgacgacgccagctcttatgaactggaagccttcgtgaaagcgctgaaaccagacctgatcggctccggcatcaaggaaaaatacattttccagaaaatgggcgtgccgttccgccagatgcactcctgggactactccggtccgtaccacggctacgacggtttcgctatcttcgctcgtgacatggatatgaccctgaataacccagcgtggaatgaactgaccgcaccgtggctgaaatctgcataa

SEQ ID NO: 7 - DNA, 1563 bp, Klebsiella pneumoniae
  misc_feature 1..1563: note = wild type Gene K, nifK
  source 1..1563: mol_type = unassigned DNA
atgagccaaacgattgataaaattaatagctgttatccgctattcgaacaggatgaataccaggagctgttccgcaataagcggcagctggaagaggcgcacgatgcgcagcgcgtgcaggaggtctttgcctggaccaccaccgccgagtatgaagcgctgaatttccgacgcgaggcgctgaccgttgacccggcgaaagcctgccagccgcttggcgcggtgctttgctcgctgggatttgccaacaccctgccgtatgtgcacggctctcaggggtgcgtggcctactttcgcacctattttaaccgccatttcaaagagccgatcgcctgcgtctccgactcgatgaccgaagacgcggcggtcttcggcggcaacaacaatatgaacctgggcctgcagaacgccagcgcgctgtacaaaccggagatcattgcggtgtccaccacctgcatggcggaagttatcggcgatgacctgcaggcgtttatcgccaacgctaaaaaagatggcttcgtcgacagcagcatcgccgtgccccacgcccatacgccaagctttatcggcagccacgtcaccggctgggataacatgtttgaaggcttcgccaaaaccttcactgcggactaccaggggcagccgggcaaattgccgaagctcaatctggtgaccggctttgaaacctatctcggcaacttccgcgtattaaagcggatgatggaacagatggcggtgccgtgcagcctgctctccgatccgtcggaagttctcgacacgcccgccgacggtcactatcggatgtattccggcggcaccacgcagcaggagatgaaagaggcccctgacgccatcgatacgctgctcctgcagccgtggcagctgctgaagagcaaaaaagtggtgcaggagatgtggaaccagcccgccaccgaggtcgccattccgctggggctggccgccaccgatgaactgctgatgaccgtcagccagcttagcggcaagccgattgccgacgccctcacccttgagcgcggccggctggttgacatgatgctcgactcccacacctggctgcacggcaagaagtttggcctgtacggcgatccggacttcgtgatgggcctcacccgcttcctgctggagctgggctgcgagccaacggtgatcctgagccataacgccaacaaacgctggcaaaaagcgatgaacaaaatgctcgatgcctcgccgtacgggcgcgatagcgaagtgtttatcaactgcgatttgtggcacttccgttcgctgatgttcacccgtcagccggactttatgatcggcaactcctacggcaagtttatccagcgcgataccctggcgaagggtaaagcctttgaagtgccgcttatccgcctcggctttccgctgttcgaccgccaccatctgcaccgccagacaacctggggttatgaaggggcgatgaacattgtgacgacgctggtgaacgccgtgctggagaaactggatagcgataccagccagctgggcaaaaccgattacagcttcgatctcgtccgttaa

SEQ ID NO: 8 - DNA, 1563 bp, synthetic construct
  misc_feature 1..1563: note = synthetic Gene K
  source 1..1563: mol_type = other DNA
atgtctcaaactatcgataaaatcaactcttgttacccgctgttcgagcaggacgaatatcaggaactgttccgtaacaaacgtcagctggaagaagcgcacgacgcacagcgcgtgcaggaagtgttcgcatggaccaccaccgcggaatacgaagctctgaacttccgtcgcgaagccctgacggttgatccggcgaaagcgtgccagcctctgggtgcggttctgtgcagcctgggttttgccaacaccctgccgtatgtccacggttcccagggctgcgtagcctacttccgtacctatttcaaccgccactttaaagaaccaatcgcgtgcgtgtccgacagcatgacggaggacgcggcagttttcggtggtaacaacaacatgaacctgggcctgcaaaatgcttccgcactgtacaaaccggaaatcatcgcagtgtctaccacctgcatggcagaggttattggtgatgatctgcaagcatttattgccaacgcaaagaaagacggtttcgttgacagctctatcgcggttccgcacgctcataccccgtccttcatcggttctcacgtaactggttgggacaacatgttcgaaggcttcgcaaaaacttttaccgcagactatcaaggccaaccgggtaaactgccgaagctgaacctggtgaccggctttgaaacctacctgggcaactttcgtgtcctgaagcgcatgatggagcagatggcggttccgtgttctctgctgtctgacccgtctgaggttctggacactccagcggacggccactatcgcatgtattctggtggcaccactcagcaggaaatgaaagaggccccagacgcgattgacaccctgctgctgcaaccgtggcagctgctgaaaagcaagaaagttgttcaggaaatgtggaaccagccggcaacggaagttgcaatcccgctgggtctggcagctactgacgaactgctgatgaccgtgtcccaactgagcggcaaaccaatcgcggatgctctgaccctggaacgcggtcgcctggtggacatgatgctggacagccacacgtggctgcatggcaagaaatttggcctgtacggtgacccggacttcgtaatgggcctgacccgtttcctgctggaactgggctgcgagccgactgttatcctgtctcacaacgctaacaaacgttggcagaaggccatgaacaaaatgctggatgcgagcccatacggccgtgatagcgaagtgttcatcaactgcgacctgtggcatttccgctctctgatgtttacgcgtcagccggatttcatgatcggtaactcttacggcaaattcatccagcgtgacactctggccaaaggcaaagcgtttgaagtgccgctgattcgtctgggctttccgctgttcgaccgtcaccacctgcaccgccagaccacctggggttacgaaggcgcgatgaacatcgtaactactctggtaaacgcagtactggaaaagctggacagcgatacttcccagctgggcaaaaccgactattctttcgatctggttcgttaa

SEQ ID NO: 9 - DNA, 663 bp, Klebsiella pneumoniae
  misc_feature 1..663: note = wild type Gene Y, nifY
  source 1..663: mol_type = unassigned DNA
atgtccgacaacgataccctattctggcgtatgctggcgctgtttcagtctctgccggacctacagccggcgcaaatcgtcgactggctggcgcaggagagcggcgagacgctgacgccagagcgtctggcgaccctgacccagccgcagctggccgccagctttccctccgcgacggcggtgatgtcccccgctcgctggtcgcgggtgatggcgagcctgcagggcgcgctgcccgcccatttacgcatcgttcgccctgcccagcgcacgccgcagctgctggcggcattttgctcccaggatgggctggtgattaacggccatttcggccagggacgactgttttttatctacgcgttcgatgaacaaggcggctggttgtacgatctgcgccgctatccctccgccccccaccagcaggaggccaacgaagtgcgcgcccggcttattgaggactgtcagctgctgttttgccaggagataggcgggcccgccgccgcgcggctgatccgccatcgcatccacccgatgaaagcgcagcccgggacgacgattcaggcacagtgcgaggcgatcaatacgctgctggccggccgtttgccgccgtggctggcgaagcggcttaacagggataaccctctggaagaacgcgttttttaa

SEQ ID NO: 10 - DNA, 663 bp, synthetic construct
  misc_feature 1..663: note = synthetic Gene Y
  source 1..663: mol_type = other DNA
atgtctgacaatgataccctgttttggcgcatgctggcgctgtttcagtcgctgccggatttgcagccggctcaaatcgtcgattggctggcgcaggaatccggcgaaaccctgacgccggagcgccttgccaccctgacccaaccgcaactcgcggcgtcgttcccatccgcgacggcagtgatgagcccggctcgctggagccgcgttatggcttctctgcaaggcgccctcccagcccacttgcgcatcgtacgtccggcgcagcgtaccccgcaactgctcgccgcgttttgcagccaagacggccttgttatcaatggtcatttcggccagggtcgtctgttcttcatttacgcctttgacgagcagggcggctggctgtatgacttgcgccgctatccgagcgcaccgcaccagcaggaagcgaatgaggtgcgtgctcgtctgattgaagattgccagctgctgttctgccaggagattggcggtccggcagcagcgcgtccgatccgccaccgcatccatccgatgaaggcgcagccgggtactacgattcaggcgcagtgtgaagctatcaacaccctgctggccggtcgcctgccgccgtggctcgccaaacgtttgaaccgtgataacccgctggaagagcgtgtgttttaa

SEQ ID NO: 11 - DNA, 1374 bp, Klebsiella pneumoniae
  misc_feature 1..1374: note = wild type Gene E, nifE
  source 1..1374: mol_type = unassigned DNA
atgaagggaaatgaaattctggcgctgctggatgaaccggcctgtgaacacaaccataaacaaaaatccggctgcagcgcgcccaaacccggcgccaccgccgcgggctgcgcgttcgacggcgcgcagataaccctgctgcccatcgccgacgtggcgcatctggtccacggccccatcggctgcgccggaagctcatgggataaccgcggcagcgccagctccggccccacccttaatcggctcgggttcaccaccgatctcaacgaacaggacgtgattatgggccgcggcgaacgccgactgtttcacgccgtgcgccatatcgtcacccgctatcatccggcggcggtctttatctacaacacctgcgtaccggccatggagggcgatgacctggaagcggtatgccaggccgcgcagaccgccaccggcgtaccggttatcgctattgacgccgccggtttctacggcagtaaaaatctcggtaaccggccggcgggcgacgtcatggtcaaacgggtcatcggccagcgcgagcccgccccctggccggagagcacgctctttgccccggagcagcgtcacgatattggcctgattggcgaattcaatattgccggcgagttctggcatattcagccgctgctcgacgaactggggatccgcgtgctcggcagcctctccggtgatggccgcttcgccgagatccagaccatgcaccgggcgcaggccaatatgctggtctgctcgcgggcgttaattaacgtcgccagagccctggagcagcgctacggcacgccgtggttcgaaggcagcttttacgggatccgcgccacctctgacgccctgcgccagctggcggcgctgctgggcgacgacgaccttcgccagcgcaccgaagcgctgattgcgcgggaggaacaggcggcggaactggcgctacagccgtggcgcgaacagctgcgcggccgcaaagcgctgctctataccggcggggtgaaatcctggtcggtggtatcggcgctgcaggatttgggcatgaccgtggtggcaaccggcacgcgtaaatccaccgaagaggataaacagcggatccgcgagctgatgggcgaagaggcggtaatgctggaagagggcaacgcccgcacgctgctggatgtggtctatcgctatcaggccgacctgatgattgccggcggacgcaatatgtacaccgcctataaagccaggctgccgtttctcgatatcaatcaggagcgcgaacacgccttcgctggctatcaggggatcgtcaccctcgcccgccagctgtgtcagaccatcaacagccccatctggccgcaaacccattctcgcgccccgtggcgctaa

SEQ ID NO: 12 - DNA, 1374 bp, synthetic construct
  misc_feature 1..1374: note = synthetic Gene E
  source 1..1374: mol_type = other DNA
atgaagggtaacgagattcttgctctgctggacgaaccggcctgtgaacacaaccataaacagaaatccggctgtagcgccccaaagccgggtgcgacggcggctggctgcgctttcgatggtgcgcagatcaccctgctcccgattgcggacgttgcccacctcgtgcatggcccaatcggttgcgcaggtagctcttgggacaaccgtggcagcgcctccagcggtccgaccctgaatcgtttgggctttaccactgacttgaatgaacaagatgtgatcatgggtcgcggcgagcgtcgcctgttccacgctgtgcgccatattgtcacccgttaccacccagcggcagtattcatctacaatacgtgcgtgccggctatggaaggcgatgacctggaggccgtgtgtcaggcagcccagactgcgaccggcgtcccggtaatcgcaattgatgcggctggcttctacggttcgaagaacctgggcaaccgtccggcaggcgatgtcatggttaaacgcgtcattggccaacgtgagccagcgccgtggccggagagcaccctgtttgccccggagcaacgtcatgacattggcttgatcggtgagttcaacattgcgggcgagttttggcacattcagccgctgcttgatgagctgggtatccgcgttttgggttcgctcagcggcgatggtcgtttcgccgagattcaaaccatgcaccgtgcccaggcgaacatgctggtgtgcagccgtgctctgatcaatgttgcgcgtgctctggaacagcgctatggcaccccgtggtttgaaggctcgttctatggtatccgcgcgaccagcgacgccctgcgccagttagcggcgctgctgggcgatgacgacctccgtcagcgcaccgaggcgctgatcgcgcgtgaagaacaggcggctgagctggccctgcaaccgtggcgtgaacagctgcgtggccgcaaggccctgctctacacgggtggtgtcaaaagctggtctgtggtgtccgcgcttcaggatctgggtatgaccgtggttgccacgggcacgcgtaagagcacggaagaggataaacagcgcatccgcgaattgatgggcgaagaggccgtgatgcttgaagaaggcaacgcacgtaccttattggatgtagtttatcgctatcaagcagacctgatgattgccggtggccgcaacatgtataccgcctacaaagcgcgcttgccgttcctggacatcaaccaggaacgcgagcacgcgtttgcgggctaccaaggcatcgtgaccttagcgcgccagctgtgccaaacgattaacagcccgatctggccgcagactcattcccgcgcaccgtggcgctaa

SEQ ID NO: 13 - DNA, 1386 bp, Klebsiella pneumoniae
  misc_feature 1..1386: note = wild type Gene N, nifN
  source 1..1386: mol_type = unassigned DNA
atggcagacattttccgcaccgataagccgctggcggtcagccccatcaaaaccggccagccgctcggcgcaatcctcgccagcctcgggatcgaacacagcatccctctggtccacggcgcgcaggggtgcagcgccttcgccaaagtcttttttattcaacatttccacgacccggttcccctgcagtcgacggcgatggaccccacgtcgacgattatgggcgcggacggcaatatttttaccgccctggataccctctgccagcgcaacaatccgcaggctatcgtactgctcagcaccgggctgtcggaggcccagggcagcgatatttcccgcgtggttcgccagtttcgcgaagagtatccccggcataagggggtggcgatattgacggttaacacgccggatttttatggctccatggagaacggcttcagcgcggtgttagagagcgtcattgagcagtgggtgccgccggcgccgcgcccggctcagcgcaatcgccgggtcaatctgctggtcagccatctctgttcgccgggcgatatcgagtggctgcgccgatgcgtcgaagcctttggtctgcagccgataatcctgccggacctggcgcaatcgatggacggccacctggcgcagggcgatttctcgccgctgacccagggcgggacgccgctgcgccagatagagcagatggggcaaagcctgtgcagcttcgccattggcgtctcccttcatcgcgcctcatcgctgctggccccgcgctgccgcggcgaggttatcgccctgccgcacctgatgaccctcgaacgctgcgacgcctttattcatcaactggcgaaaatttccggacgcgccgttcccgagtggctggaacgccagcgcggccagctacaggatgcgatgatcgactgccatatgtggctccagggccagcgcatggcgatagcggcggaaggcgatttgctggcggcgtggtgtgatttcgccaacagccaggggatgcagcccggcccgctggtggcccctaccggtcatcccagcctgcgccagctgccggtggaacgggtggtgccgggggatctggaggatctgcaaaccctgctgtgcgcgcatcccgccgacctgctggtggcgaactcgcacgcccgcgacctggcggagcagtttgcgctgccgctggtgcgcgcgggttttccgctctttgacaagctcggcgaattccgccgggtgcgacaggggtatagcgggatgcgcgatacgctgtttgagctggcaaacctgatacgcgagcgtcaccaccacctcgcccactaccgatcgccgctgcgccagaaccccgaatcgtcactctccacaggaggcgcttatgccgccgattaa

SEQ ID NO: 14 - DNA, 1386 bp, synthetic construct
  misc_feature 1..1386: note = synthetic Gene N
  source 1..1386: mol_type = other DNA
atggcagacattttccgcactgataagccgttggctgtgtcgccgatcaagaccggccagccgctgggtgcgatcctggcgtccctgggtatcgagcactcgattccgctggtacatggcgcgcagggctgttcggcttttgccaaggttttctttatccagcacttccacgatccggtcccgctgcaaagcacggcaatggacccgaccagcaccatcatgggcgctgatggtaacatcttcaccgcgctggacactctctgccaacgcaataacccgcaagcaattgtgctgctgagcaccggcctctccgaggcgcagggcagcgacatttcccgtgtagtgcgtcagttccgtgaagaatatccgcgtcataaaggcgtggcgattctgactgttaacaccccggacttttacggtagcatggagaacggcttttccgctgtcctggagtctgtgattgaacagtgggttccgccagccccacgtccggcgcagcgcaatcgtcgcgtcaatcttttggtgagccatctctgtagcccaggcgatattgagtggctgcgccgttgcgtcgaggccttcggtctgcaaccgatcattctgccggatctggctcagagcatggacggccaccttgctcagggtgacttttcgccgctgacgcagggcggcacgccgttgcgccaaatcgagcagatgggccagagcctttgctcttttgcgattggcgtcagcctgcaccgtgcgagcagcctgctggctccgcgttgtcgtggcgaagtcatcgccttgccgcacctcatgaccttggaacgctgcgacgcctttatccatcagttggcgaaaatcagcggtcgcgccgttccggagtggctggaacgccagcgcggtcagctgcaagacgccatgatcgattgccacatgtggctgcaaggccagcgcatggcgattgccgccgaaggcgacctgctggcagcgtggtgcgatttcgcgaactctcaaggtatgcagccgggtccactggttgctccgacgggtcatccgagcctgcgtcagttgccggtggagcgcgtggtgccgggtgatctggaggatcttcagaccctcttatgcgcacatccggccgacttactggtggcgaactcccacgcccgtgatttagcagagcaattcgccctgccgctggtgcgcgcaggcttcccgctgtttgacaaactgggcgaatttcgtcgtgttcgccagggttatagcggtatgcgtgataccctgttcgagttggcgaacctgatccgtgaacgccatcatcatctggctcattatcgcagcccgctgcgccagaacccagaatcctcgttgtctacgggtggcgcgtacgcagcggattaa

SEQ ID NO: 15 - DNA, 825 bp, Klebsiella pneumoniae
  misc_feature 1..825: note = wild type Gene U, nifU
  source 1..825: mol_type = unassigned DNA
atgtggaattactccgagaaagtgaaagaccatttttttaacccccgcaatgcgcgcgtggtggacaacgccaacgcggtaggcgacgtcggttcgttaagctgcggcgacgccctgcgcctgatgctgcgcgtcgacccgcaaagcgaaatcattgaggaggcgggcttccagaccttcggctgcggcagcgccatcgcctcctcctccgcgctgacggagctgattatcggccataccctcgccgaagccgggcagataaccaatcagcagattgccgattatctcgacggactgccgccggagaaaatgcactgctcggtgatgggccaggaggccctgcgcgcggccatcgccaactttcgcggcgaaagccttgaagaggagcacgacgagggcaagctgatctgcaaatgcttcggcgtcgatgaagggcatattcgccgcgcggtacagaacaacgggctgaccacccttgccgaggtgatcaactacaccaaagcgggcggcggctgcacctcttgccacgaaaaaatcgagctggccctggcggagatcctcgcccagcagccgcagacgacgccagccgtggccagcggcaaagatccgcactggcagagcgtcgtcgataccatcgcagaactgcggccgcatattcaggccgacggcggcgatatggcgctactcagcgtcaccaaccaccaggtgaccgtcagcctctccggcagctgtagcggctgcatgatgaccgatatgaccctggcctggctgcagcaaaaactgatggaacgtaccggctgttatatggaagtggtggcggcctga

SEQ ID NO: 16 - DNA, 825 bp, synthetic construct
  misc_feature 1..825: note = synthetic Gene U
  source 1..825: mol_type = other DNA
atgtggaactacagcgagaaagtcaaggaccatttcttcaatccgcgcaacgcgcgtgttgtggataacgcaaatgcggtgggcgacgtcggcagcttatcttgtggcgatgctctccgcttgatgctgcgcgtggacccgcagagcgaaatcatcgaagaagcgggctttcagaccttcggctgcggcagcgcgattgcgtcgtccagcgcactgacggagctgatcatcggtcacaccctggcggaagcgggtcagatcaccaaccagcagatcgccgactatctggacggcttaccgccggaaaagatgcactgctctgtaatgggccaggaagctcttcgtgcggccattgctaactttcgcggtgaatcgctggaagaggagcatgacgagggtaagctgatctgcaagtgcttcggcgtcgatgaaggccatattcgccgtgctgtccagaacaacggtcttacgactctggccgaggtgatcaattacaccaaggcaggtggcggttgtaccagctgccatgagaaaatcgagctggccctggccgagattctcgcccaacagccgcaaaccaccccggcagttgcgtccggtaaagatccgcactggcagagcgtcgtggataccatcgctgaactgcgtccacatatccaagcggacggtggtgacatggcgctgttgtccgtgacgaaccaccaagtgactgtttcgctgtcgggcagctgttctggctgcatgatgaccgacatgaccctggcgtggctgcaacagaaattgatggagcgtaccggctgctatatggaagttgttgccgcctaa

SEQ ID NO: 17 - DNA, 1203 bp, Klebsiella pneumoniae
  misc_feature 1..1203: note = wild type Gene S, nifS
  source 1..1203: mol_type = unassigned DNA
atgaaacaggtttatctcgataacaacgccaccacccgtctggacccgatggtcctggaagcgatgatgccctttttgaccgatttttacggcaacccctcgtcgatacacgattttggcattccggcccaggcggctctggaacgcgcgcatcagcaggctgcggcgctgctgggcgcggagtatcccagcgagatcatctttacctcctgcgccaccgaagccaccgccaccgccatcgcctcggcgatcgccctgctgcctgagcgtcgcgaaatcatcaccagcgtggtcgaacatccggcgacgctggcggcctgcgagcacatggagcgcgagggctaccggattcatcgcatcgcggtagatggcgagggggcgctggacatggcgcagttccgcgcggcgctcagcccgcgcgtcgcgttggtcagcgtgatgtgggcgaataacgaaaccggggtgcttttcccgatcggcgaaatggcggagctggcccatgaacaaggggcgctgtttcactgcgatgcggtgcaggtggtcgggaaaataccgatcgccgtgggccagacccgcatcgatatgctctcctgctcggcgcataagttccacgggccaaaaggcgtaggctgtctttatctgcggcggggaacgcgctttcgcccgctgctgcgcggcggtcaccaggagtacggtcggcgagccgggacagaaaatatctgcggaatcgtcggcatgggcgcggcctgcgagctggcgaatattcatctgccgggaatgacgcatatcggccaattgcgcaacaggctggagcatcgcctgctggccagcgtgccgtcggtcatggtgatgggcggcggccagccggcggtgcccggcacggtgaatctggcctttgagtttattgaaggtgaagccattctgctgctgttaaaccaggccgggatcgccgcctccagcggcagcgcctgcacctcaggctcgctggaaccctcccacgtgatgcgggcgatgaatatcccctacaccgccgcccacggcaccatccgcttttctctctcgcgctacacccgggagaaagagatcgattacgtcgtcgccacgctgccgccgattatcgaccggctgcgcgcgctgtcgccctactggcagaacggcaagccgcgcccggcggacgccgtattcacgccggtttacggctaa

SEQ ID NO: 18 - DNA, 1203 bp, synthetic construct
  misc_feature 1..1203: note = synthetic Gene S
  source 1..1203: mol_type = other DNA
atgaaacaagtgtacctggacaacaacgcgaccacccgcctggacccgatggttctggaagcgatgatgccgtttctcacggatttctatggcaatccgtccagcatccatgacttcggcatcccggcacaagcggcgctggaacgtgcgcaccagcaagctgcggcactgctgggcgcagagtacccgtctgaaatcattttcacgagctgtgcgaccgaggccactgcaaccgccattgcgtcggccatcgcgttattgccggaacgccgcgaaatcatcacctcggtagtggagcacccggctacgctggcggcgtgcgagcacatggaacgcgaaggctatcgcatccatcgcattgcggtggatggcgaaggtgcgctggacatggcccagttccgtgcagcgctctcgccgcgtgtcgcgttggtgagcgtgatgtgggccaacaacgaaaccggcgtgctgttcccgattggcgaaatggccgagcttgcccacgagcagggcgctctgttccactgcgatgccgttcaggtcgttggcaaaatcccaattgctgttggccagacgcgcatcgacatgctgtcttgctccgcgcacaagtttcatggtccgaagggtgttggttgcttgtacttacgtcgtggcacgcgctttcgtccgctgcttcgcggtggccatcaagaatatggtcgccgtgccggcactgagaatatctgtggcatcgtcggcatgggcgctgcgtgcgaactggcgaacatccatctgccgggtatgacccatattggccagttacgcaatcgcctggagcaccgtctgctcgccagcgtgccgtccgtgatggttatgggcggtggtcagccggctgtaccgggtactgtcaacctggcgttcgagtttatcgaaggtgaagcgatcctgctcttgctgaaccaggctggcattgccgcaagctccggctccgcgtgtacctctggcagcttggagccgagccatgtgatgcgcgccatgaacattccatacaccgcggctcacggcaccattcgttttagcctgagccgttatacgcgcgagaaagagatcgactacgtcgttgcgaccctcccgccaatcattgatcgtctgcgtgccttgtccccgtattggcagaatggtaagccgcgtccggcagatgcagtctttaccccggtttacggttaa

SEQ ID NO: 19 - DNA, 1143 bp, Klebsiella pneumoniae
  misc_feature 1..1143: note = wild type Gene V, nifV
  source 1..1143: mol_type = unassigned DNA
atggaacgcgtgctgattaacgataccaccctgcgcgacggcgagcagagccccggcgtcgcctttcgcaccagcgaaaaggtcgccattgccgaggcgctttacgccgcaggaataacggcgatggaggtcggcaccccggcgatgggcgacgaggagatcgcgcggatccagctggtgcgtcgccagctgcccgacgcgaccctgatgacctggtgtcggatgaacgcgctggagatccgccagagcgccgatctgggcatcgactgggtggatatctcgattccggcttcggataagctgcggcagtacaaactgcgcgagccgctggcggtgctgctggagcggctggcgatgtttatccatcttgcgcataccctcggcctgaaggtatgcatcggctgcgaggacgcctcgcgggccagcggccagaccctgcgcgctatcgccgaggtcgcgcagcaatgcgccgccgcccgcctgcgctatgccgatacggtcggcctgctcgacccttttaccaccgcggcgcaaatctcggccctgcgcgacgtctggtccggcgaaatcgaaatgcatgcccataacgatctgggtatggcgaccgccaatacgctggcggcggtaagcgccggggccaccagcgtgaatacgacggtcctcggtctcggcgagcgggcgggcaacgcggcgctggaaaccgtcgcgctgggccttgaacgctgcctgggcgtggagaccggcgtgcatttttcggcgctgcccgcgtcctgtcagagggtcgcggaagccgcgcagcgcgccatcgacccgcagcagccgctggtcggcgagctggtgtttacccatgagtcaggtgtccacgtggcggcgctgctgcggcacagcgagagctaccagtccatcgccccttccctgatgggccgcagctaccggctggtgctgggcaaacactccgggcgtcaggcggtcaacggcgtttttgaccagatgggctatcacctcaacgccgcgcagattaaccagctgctgcccgccatccgccgcttcgccgagaactggaagcgcagcccgaaagattacgagctggtggctatctacgacgagctgtgcggtgaatccgctctgcgggcgagggggtaa

SEQ ID NO: 20 - DNA, 1143 bp, synthetic construct
  misc_feature 1..1143: note = synthetic Gene V
  source 1..1143: mol_type = other DNA
atggagcgcgtcttgatcaacgatactaccctgcgtgatggcgaacaatctccgggcgtagcgtttcgtacctccgagaaagttgccatcgcggaggcactgtacgctgcgggtatcaccgcgatggaagtcggcactccggcgatgggtgatgaagagatcgcccgcattcagctggtgcgtcgtcaactgccggacgcgacgcttatgacctggtgccgtatgaacgctctggaaatccgtcagagcgcggatctgggtattgactgggtggatatctcgatcccagcatccgacaagctgcgtcagtacaagctgcgtgagccgctggccgtgctgctggagcgccttgcgatgtttatccatctggcccacacgttaggcctcaaagtatgtattggttgcgaggatgcgagccgtgcgtctggtcagaccctgcgcgccattgccgaggtggcccagcaatgcgcggctgcgcgcttgcgttacgctgacaccgtgggcctgctggacccgttcaccaccgcagcccagatcagcgccctgcgtgacgtttggtcgggcgagatcgagatgcatgctcacaatgatctgggcatggctaccgcgaacacgctggcggcagtttcggctggcgccacgtcggtgaacactaccgtcctcggtctgggtgaacgtgcaggcaacgcagccctggaaaccgttgcgctgggcctggaacgctgcctgggcgtggaaaccggcgtccatttcagcgcgctcccagcgagctgtcagcgcgtcgcggaggctgcacagcgcgcaatcgacccgcaacagccgctggtgggtgaattggttttcacccacgagtctggtgttcacgttgcggcgctgctgcgccacagcgaatcctatcaatctattgccccaagcctcatgggccgtagctaccgtctggtgctcggcaagcattcgggtcgtcaggctgtcaacggtgttttcgaccagatgggttaccacctgaatgcggcgcagatcaatcagttgctgccggccattcgccgcttcgccgagaattggaaacgctctccgaaagactacgaactggttgcgatctatgacgaattgtgcggtgaatccgcccttcgtgctcgcggctaa

SEQ ID NO: 21 - DNA, 258 bp, Klebsiella pneumoniae
  misc_feature 1..258: note = wild type Gene W, nifW
  source 1..258: mol_type = unassigned DNA
atggagtggttttatcaaattcccggcgtggacgaacttcgctccgccgaatctttttttcagtttttcgccgtcccctatcagcccgagctgcttggccgctgcagcctgccggtgctggcaacgtttcatcgcaaactccgcgcggaggtgccgctgcaaaaccggctcgaggataacgaccgcgcgccctggctgctggcgcgaagactgctcgcggagagctatcagcaacagtttcaggagagcggaacatga

SEQ ID NO: 22 - DNA, 258 bp, synthetic construct
  misc_feature 1..258: note = synthetic Gene W
  source 1..258: mol_type = other DNA
atggagtggttttaccagattccgggtgtagacgaattgcgcagcgctgaatccttctttcagttcttcgcggttccataccagccggaactgctgggccgctgctcgcttccggtgttagcgacgttccaccgtaaactgcgtgcggaggtcccgctgcaaaaccgtctggaggacaatgatcgtgcgccgtggctcttggcgcgccgcctcctggccgaatcttatcagcagcaatttcaggagagcggcacctaa

SEQ ID NO: 23 - DNA, 447 bp, Klebsiella pneumoniae
  misc_feature 1..447: note = wild type Gene Z, nifZ
  source 1..447: mol_type = unassigned DNA
atgagaccgaaattcacctttagcgaagaggtccgcgtcgtacgcgcgattcgtaacgacggcaccgtggcgggcttcgcgcccggcgcgctgctggtcaggcgcggcagcaccggctttgtgcgcgactggggcgtttttttgcaagatcagattatctaccagatccactttccggaaaccgatcggatcatcggctgccgcgagcaggagctgatccccatcacccagccgtggctggccggaaatttgcaatacagggatagcgtgacctgccagatggcgctcgcggtcaacggcgatgtggtcgtgagcgccggccagcggggacgcgttgaggctaccgatcggggagagctcggcgacagctacaccgtcgactttagcggccgctggttcagggtcccggtgcaggccatcgcccttatagaggaaagagaagaatga

SEQ ID NO: 24 - DNA, 447 bp, synthetic construct
  misc_feature 1..447: note = synthetic Gene Z
  source 1..447: mol_type = other DNA
atgcgcccgaaattcaccttctctgaagaggtccgcgtagttcgcgcgattcgtaatgatggcaccgtggcgggttttgcgccaggtgcgctgctggttcgtcgcggttcgacgggctttgtgcgtgactggggtgtgttcctgcaagaccagatcatctatcaaatccactttccggaaaccgaccgcattatcggctgtcgcgagcaggagttaatcccgattacccagccgtggttggctggtaacctccagtatcgtgacagcgtcacgtgccaaatggcactggctgtcaacggtgacgtggttgtgagcgccggtcaacgtggccgtgtggaggccactgatcgtggcgaacttggcgattcctacaccgtggacttcagcggccgttggttccgcgttccggtccaggccatcgcgctgattgaagagcgcgaagaataa

SEQ ID NO: 25 - DNA, 801 bp, Klebsiella pneumoniae
  misc_feature 1..801: note = wild type Gene M, nifM
  source 1..801: mol_type = unassigned DNA
atgaacccgtggcaacgttttgcccggcagcggctggcgcgcagccgctggaatcgcgatccggcggccctggatccggccgacacgccggcttttgaacaggcctggcaacgccagtgccatatggagcagacgatcgtcgcgcgggtccctgaaggcgatattccggcggcgttgctggagaatatcgctgcctcccttgccatctggctcgacgagggggattttgcgccgcccgagcgcgctgccatcgtgcgccatcacgcccggctggaactcgccttcgccgatatcgcccgccaggcgccgcagccggatctctccacggtacaggcatggtatctgcgccaccagacgcagtttatgcgcccggaacagcgtctgacccgccatttactgctgacggtcgataacgaccgcgaagccgtgcaccagcggatcctcggcctgtatcggcaaatcaacgcctcgcgggacgctttcgcgccgctggcccagcgccattcccactgcccgagcgcgctggaagagggtcgtttaggctggattagccgtggcctgctctatccgcagctcgagaccgcgctgttttcactggcggaaaacgcgctaagccttcccatcgccagcgaactgggctggcatcttttatggtgcgaagcgattcgccccgccgcgcccatggagccgcagcaggcgctggagagcgcgcgcgattatctttggcagcagagccagcagcgccatcagcgccagtggctggaacagatgatttcccgtcagccgggactgtgcgggtag

SEQ ID NO: 26 - DNA, 801 bp, synthetic construct
  misc_feature 1..801: note = synthetic Gene M
  source 1..801: mol_type = other DNA
atgaatccgtggcagcgctttgcccgtcaacgccttgctcgcagccgctggaaccgtgatccggctgctctcgacccagccgataccccagcgttcgagcaggcgtggcagcgtcaatgccatatggaacaaaccatcgtagcgcgtgtcccggaaggcgatattccggctgccttactggaaaacatcgcggccagcctggcgatctggctggacgagggtgacttcgctccgccggagcgcgctgcgattgtgcgtcatcatgcacgtctggagctggcgtttgccgacattgcccgccaggcaccgcaaccggatctgagcacggttcaagcgtggtatctgcgtcaccagactcaattcatgcgtccggagcagcgtctgacccgtcacctgctcctgacggtcgataatgatcgcgaggcggtgcatcaacgcatccttggcctgtatcgtcagatcaacgcgagccgtgacgccttcgccccactggcacagcgccactctcattgcccgtccgccttggaagaaggccgtctgggctggatctcccgtggtctgctgtacccgcagctcgaaaccgcgttgtttagcctggcggaaaacgcactgtcgctgccgattgcgtcggaattgggttggcacctgttatggtgcgaggccattcgtccggcagccccgatggagccgcaacaggcccttgaatctgcgcgcgactacttgtggcagcagagccagcagcgccaccagcgtcaatggctggagcagatgatttcccgccaaccgggcctgtgtggttaa

SEQ ID NO: 27 - DNA, 531 bp, Klebsiella pneumoniae
  misc_feature 1..531: note = wild type Gene F, nifF
  source 1..531: mol_type = unassigned DNA
atggcgaacattggtattttctttggcaccgataccggtaaaacccgcaaaatcgcgaaaatgatccataagcaactgggcgagctggcggatgccccggtcaacattaaccgcacgacgctggacgactttatggcctatccggtgctgctgctgggcacgccgacgctcggcgacggccagctgccggggctggaggccggatgcgaaagcgagtcatggagcgaatttatcagcggcctcgacgacgccagcctgaaagggaaaaccgtggcgctgttcggcctcggcgatcagagaggctatccggacaacttcgtcagcgggatgcgcccgctgttcgacgccctgagcgcgcgcggcgcgcagatgattggcagctggccaaatgagggttatgaattcagcgcgtcctcggcgctggaaggcgaccgctttgttgggctggtgctggatcaggataaccagttcgaccagaccgaagcgcgtctggcgagctggcttgaggagattaaacgcaccgtgctgtag

SEQ ID NO: 28 - DNA, 531 bp, synthetic construct
  misc_feature 1..531: note = synthetic Gene F
  source 1..531: mol_type = other DNA
atggcgaacatcggcatcttctttggtacggataccggcaaaacccgcaagattgcgaagatgattcacaaacagctgggcgagctggccgatgccccggttaacatcaatcgtaccactttggatgactttatggcttacccagtcctgttgctcggcacgccgacgcttggtgatggtcaactgccgggcttagaggcgggctgcgagagcgaaagctggtctgagtttatctccggtctggatgacgcttccctgaagggcaaaaccgtggcgctgtttggcctgggcgaccagcgtggttacccggacaacttcgtgtcgggtatgcgtccgctgttcgacgcgctgagcgcccgtggcgcccagatgattggtagctggccgaacgaaggttatgagtttagcgcatcgtccgcgctggaaggcgaccgcttcgtcggcttggtgctggatcaagacaatcagttcgaccagaccgaagcgcgcctggcgtcttggcttgaagagatcaaacgcaccgttctgtaa

SEQ ID NO: 29 - DNA, 1407 bp, Klebsiella pneumoniae
  misc_feature 1..1407: note = wild type Gene B, nifB
  source 1..1407: mol_type = unassigned DNA
atgacttcctgctcctctttttctggcggcaaagcctgccgcccggcggatgacagcgcattgacgccgcttgtggccgataaagctgccgcgcacccctgctactctcgccatgggcatcaccgtttcgcgcggatgcatctgcccgtcgcgcccgcctgcaatttgcagtgcaactactgtaatcgcaaattcgattgcagcaacgagtcccgccccggggtatcgtcaacgctgctgacgcctgaacaggcggtcgtgaaagtgcgtcaggtcgcgcaggcgatcccgcagctttcggtggtgggcatcgccgggcccggcgatccgctcgccaatatcgcccgcacctttcgcaccctggagctgatccgcgaacagctgccggacctgaaattatgcctgtcgaccaacggactggtgctgcctgacgcggtggaccgcctgctggatgtcggcgttgaccacgtcacggtcaccattaacaccctcgacgcggagattgccgcgcaaatctacgcctggctatggctggacggcgaacgctacagcgggcgcgaagcgggagagatcctgattgcccgtcagcttgagggcgtacgcaggctgaccgccaaaggcgtgctggtgaaaataaattcggtgctgatccccggtatcaacgatagcggcatggccggcgtgagccgcgcgctgcgggccagcggcgcgtttatccataatattatgccgctgatcgccaggccggagcacggcacggtgtttggcctcaacggccagccggagccggacgccgagacgctcgccgccacccgcagccggtgcggcgaagtgatgccgcagatgacccactgccaccagtgtcgcgccgacgccattgggatgctcggcgaagaccgcagccagcagtttacccagcttccggcgccagagagtctcccggcctggctgccgatcctccaccagcgcgcgcagctgcacgccagcattgcgacccgcggcgaatctgaagccgatgacgcctgcctggtcgccgtggcgtcaagccgcggggacgtcattgattgtcactttggtcacgccgaccggttctacatttacagcctctcggccgccggtatggtgctggtcaacgagcgctttacgcccaaatattgtcaggggcgcgatgactgcgagccgcaggataacgcagcccggtttgcggcgatcctcgaactgctggcggacgttaaagccgtattctgcgtgcgtatcggccatacgccgtggcaacagctggaacaggaaggcattgaaccctgcgttgacggcgcgtggcggccggtctccgaagtgctgcccgcgtggtggcaacagcgtcgggggagctggcctgccgcgttgccgcataagggggtcgcctga

SEQ ID NO: 30 - DNA, 1407 bp, synthetic construct
  misc_feature 1..1407: note = synthetic Gene B
  source 1..1407: mol_type = other DNA
atgacctcttgttcgtcgttttctggcggtaaagcgtgccgtccggccgatgactccgcgctgactccgctggtggccgacaaggcagctgcgcacccgtgctatagccgccacggccatcaccgcttcgcgcgtatgcacctgccagtcgctccggcctgcaacttacaatgcaactactgcaaccgcaagttcgattgcagcaatgaaagccgtccgggcgtgtcctctaccctgctgacgccggaacaggctgtggtgaaggtgcgccaggtcgcccaagctatcccgcagctgtcggtggtcggtattgctggtccgggcgatccgcttgcgaatatcgcccgcaccttccgtaccttggagcttattcgcgaacagttgccggacctgaaactgtgcctgagcaccaacggcttggtgctgccagatgccgttgatcgtctgctcgatgtgggcgtggatcacgttaccgtcaccattaacaccctggacgcagaaatcgcagcgcaaatctacgcgtggttgtggctggatggcgaacgctactccggtcgcgaagccggcgaaattctcattgcccgccagctggaaggcgtacgtcgcctgaccgcgaaaggtgtgctcgtcaagatcaacagcgtattgattccgggcatcaatgacagcggcatggcgggtgttagccgtgcgctgcgcgcgtctggtgcgttcatccacaacatcatgccactgattgcgcgtccggagcatggcactgttttcggtctgaacggccagccggaaccggacgcggaaaccctggcggcgacgcgctcccgctgcggcgaggttatgccacaaatgacccactgccaccagtgccgtgccgacgcgattggcatgcttggtgaggatcgctcgcaacagtttacgcaattaccggctccggagtccctcccggcctggctgccgatcctgcatcagcgtgctcagttgcatgcgagcatcgccacgcgcggtgagagcgaagccgatgacgcctgcctggtggccgttgcgtcgagccgtggcgatgtaattgactgccatttcggccatgccgaccgtttctatatctatagcctgtctgcggctggtatggttctggttaacgaacgtttcaccccgaaatactgccagggtcgcgatgactgcgagccgcaggacaatgccgcacgctttgctgccatccttgagttgctggcggacgtcaaagcggtgttttgtgtgcgtatcggccataccccgtggcaacagctggagcaggaaggcatcgaaccgtgcgtggatggcgcctggcgtccggtatccgaggtcctgccggcatggtggcagcagcgccgtggtagctggccggctgcattgccgcacaaaggcgttgcgtaa

SEQ ID NO: 31 - DNA, 504 bp, Klebsiella pneumoniae
  misc_feature 1..504: note = wild type Gene Q, nifQ
  source 1..504: mol_type = unassigned DNA
atgccgccgctcgactggttgcggcgcttatggctgctgtaccacgcggggaaaggcagctttccgctgcgcatggggcttagcccgcgcgattggcaggcgctgcggcggcgcctgggcgaggtggaaacgccgctcgacggcgagacgctcacccgtcgccgcctgatggcggagctcaacgccacccgcgaagaggagcgccagcagctgggcgcctggctggcgggctggatgcagcaggatgccgggccgatggcgcagattatcgccgaggtttcgctggcgtttaaccatctctggcaggatcttggtctggcatcgcgcgccgaattgcgcctgctgatgagcgactgctttccacagctggtggtgatgaacgaacacaatatgcgctggaaaaagttcttttatcgtcagcgctgtttgctgcaacagggggaagttatctgccgttcgccaagctgcgacgagtgctgggaacgcagcgcctgttttgagtag

SEQ ID NO: 32 - DNA, 504 bp, synthetic construct
  misc_feature 1..504: note = synthetic Gene Q
  source 1..504: mol_type = other DNA
atgccgccattggactggttgcgtcgtttgtggttactctatcacgccggcaaaggcagctttccgcttcgtatgggcttgtcgccgcgtgactggcaagctctgcgccgtcgcctgggcgaggtggaaacgccgctggatggcgaaaccctgacccgtcgccgtctgatggcggagctgaatgcgacccgcgaagaagaacgccagcagctgggtgcctggctggccggttggatgcaacaggatgccggtccgatggcgcagattatcgcagaggtgagcctggcgttcaaccatctctggcaggaccttggcctcgcgagccgcgctgaactgcgtctgctgatgtctgactgcttcccgcagctggttgttatgaacgagcacaacatgcgctggaagaaattcttttaccgccagcgttgcctgctgcaacagggcgaagtcatctgtcgcagcccgtcttgcgatgaatgctgggaacgttctgcgtgctttgagtaa

SEQ ID NO: 33 - DNA, 1117 bp, synthetic construct
  misc_feature 1..1117: note = synthetic lacI sequence of Ptac-T7 synthetic controller
  source 1..1117: mol_type = other DNA
caattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcac

SEQ ID NO: 34 - DNA, 589 bp, synthetic construct
  misc_feature 1..589: note = synthetic lacI promoter sequence of Ptac-T7 synthetic controller
  source 1..589: mol_type = other DNA
attcaccaccctgaattgactctcttccgggcgctatcatgccataccgcgaaaggttttgcaccattcgatggtgtcaacgtaaatgcatgccgcttcgccttcgcgcgcgaattggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggcggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctg

SEQ ID NO: 35 - DNA, 55 bp, synthetic construct
  misc_feature 1..55: note = synthetic tac promoter sequence of Ptac-T7 synthetic controller
  source 1..55: mol_type = other DNA
ttgacaattaatcatcggctcgtataatgtgtggaattgtgagcggataacaatt

SEQ ID NO: 36 - DNA, 40 bp, synthetic construct
  misc_feature 1..40: note = synthetic insulator 1 sequence of Ptac-T7 synthetic controller
  source 1..40: mol_type = other DNA
tgcagttttattctctcgccagcactgtaataggcactaa

SEQ ID NO: 37 - DNA, 34 bp, synthetic construct
  misc_feature 1..34: note = synthetic Enterobacteria phage T7 RNA polymerase (RNAP) ribosome binding site (rbs, RBS) sequence of Ptac-T7 synthetic controller
  source 1..34: mol_type = other DNA
tatccaaaccagtagctcaattggagtcgtctat

SEQ ID NO: 38 - DNA, 120 bp, synthetic construct
  misc_feature 1..120: note = synthetic umuD degradatiobn tag sequence of Ptac-T7 synthetic controller
  source 1..120: mol_type = other DNA
gtgttgtttatcaagcctgcggatctccgcgaaattgtgacttttccgctatttagcgatcttgttcagtgtggctttccttcaccggcagcagattacgttgaacagcgcatcgatctg

SEQ ID NO: 39 - DNA, 2568 bp, synthetic construct
  misc_feature 1..2568: note = synthetic Enterobacteria phage T7 RNA polymerase (RNAP) scaffold sequence of Ptac-T7 synthetic controller
  source 1..2568: mol_type = other DNA
ggtggcaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgacaaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagagttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtacgattccggctgacgctgcgaacctgttcaaagcagtgcgcgaaactatggttgacacatatgagtcttgtgatgtactggctgatttctacgaccagttcgctgaccagttgcacgag

SEQ ID NO: 40 - DNA, 21 bp, synthetic construct
  misc_feature 1..21: note = synthetic Biobrick suffix (BBa_G00001) sequence of Ptac-T7 synthetic controller
  source 1..21: mol_type = other DNA
tactagtagcggccgctgcag

SEQ ID NO: 41 - DNA, 12 bp, synthetic construct
  misc_feature 1..12: note = synthetic translational stop (BBa_B0042) sequence of Ptac-T7 synthetic controller
  source 1..12: mol_type = other DNA
ttagttagttag

SEQ ID NO: 42 - DNA, 69 bp, synthetic construct
  misc_feature 1..69: note = synthetic terminator (BBa_B0054) sequence of Ptac-T7 synthetic controller
  source 1..69: mol_type = other DNA
attagcagaaagtcaaaagcctccgaccggaggcttttgactaaaacttcccttggggttatcattggg

SEQ ID NO: 43 - DNA, 20 bp, synthetic construct
  misc_feature 1..20: note = synthetic VR primer (BBa_G00102) sequence of Ptac-T7 synthetic controller
  source 1..20: mol_type = other DNA
gctcactcaaaggcggtaat

SEQ ID NO: 44 - DNA, 41 bp, synthetic construct
  misc_feature 1..41: note = synthetic terminator (BBa_B0062) sequence of Ptac-T7 synthetic controller
  source 1..41: mol_type = other DNA
cagataaaaaaaatccttagctttcgctaaggatgatttct

SEQ ID NO: 45 - DNA, 3671 bp, synthetic construct
  misc_feature 1..3671: note = synthetic pSa origin sequence of Ptac-T7 synthetic controller
  source 1..3671: mol_type = other DNA
gctagagctgtcagaccagagctccaaaccaacgttttatctataccgctacagggtatttaattcctatttaatctgcgctagaatgaggcatgtttaaccgaatctgacgttttccctgcaaatgccaaaatactatgcctatctccgggtttcgcgtgacggccaagacccggaaaaccaaaaatacggtttgctcgaatacgcgaacgccaaaggcttcgcgccgctacagatcgaggaagaaattgccagcagagcaaaggactggcgcaagcgcaagctcggagcaatcatcgaaaaggccgagcgtggcgacgtgctactgacgccggagattacgcgcattgccggttccgccctcgccgccttggaaattctcaaagcggcgagcgagcgcggcctaatcgtccatgtgaccaaacagaagatcatcatggacggcagcctacaaagcgacatcatggcaaccgtgcttggcttggctgcacagatcgagcggcatttcattcaggcacgtaccaccgaggcgctacaagtcgccagagagcgcggcaagacgctcgggcgacccaagggcagcaaatcgagcgccttgaagctggacagccgtattgatgaagtacaggcatacgtgaaccttggcttgccgcaaagtcgcgcagccgagttgttaggcgtcagccctcacaccttgcgcctgttcatcaaacgccggaacatcaaacccacaaacactagaccaaccatcaccatgccggggagggaacaacatgcctaagaacaacaaagcccccggccatcgtatcaacgagatcatcaagacgagcctcgcgctcgaaatggaggatgcccgcgaagctggcttagtcggctacatggcccgttgccttgtgcaagcgaccatgccccacaccgaccccaagaccagctactttgagcgcaccaatggcatcgtcaccttgtcgatcatgggcaagccgagcatcggcctgccctacggttctatgccgcgcaccttgcttgcttggatatgcaccgaggccgtgcgaacgaaagaccccgtgttgaaccttggccggtcgcaatcggaatttctacaaaggctcggaatgcacaccgatggccgttacacggccacccttcgcaatcaggcgcaacgcctgttttcatccatgatttcgcttgccggcgagcaaggcaatgacttcggcattgagaacgtcgtcattgccaagcgcgcttttctattctggaatcccaagcggccagaagatcgggcgctatgggatagcaccctcaccctcacaggcgatttcttcgaggaagtcacccgctcaccggttcctatccgaatcgactacctgcatgccttgcggcagtctccgcttgcgatggacatttacacgtggctgacctatcgcgtgttcctgttgcgggccaagggccgccccttcgtgcaaatcccttgggtcgccctgcaagcgcaattcggctcatcctatggcagccgcgcacgcaactcgcccgaactggacgataaggcccgagagcgggcagagcgggcagcactcgccagcttcaaatacaacttcaaaaagcgcctacgcgaagtgttgattgtctatcccgaggcaagcgactgcatcgaagatgacggcgaatgcctgcgcatcaaatccacacgcctgcatgtcacccgcgcacccggcaagggcgctcgcatcggcccccctccgacttgaccaggccaacgctacgcttggcttggtcaagccttcccatccaacagcccgccgtcgagcgggcttttttatccccggaagcctgtggatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccgcaacagatcgttttataaatttttttttttgaaaaagaaaaagcccgaaaggcggcaacctctcgggcttctggatttccgatcaacgcaggagtcgttcggaaagtagctgttccagaattataggcgcagagacaccagattccaagatggctctgttaaattgttgtagtatgtagtatcatacaacatactacagtacagaggcccgcaagaatggcaatcactaaacaagacatttggcgagcagccgacgaactggacgccgaaggcatccggcccactttggccgccgtgcgcaagaaactcggaagcggtagcttcacaaccatttccgatgcaatggctgaatggaaaaaccgcaagaccgccaccctgccctcatcagacccattgccggttgcagtcaacgagcatcttgccgagcttggcaatgcgctatgggctatcgccctggcgcacgccaacgcccggtttgacgaagatcggaaacagatcgaggccgacaaagcggccatcagccagcagcttgccgaagcaatcgaactagccgacaccttcacccgcgaaaacgaccagctccgcgaacgagtgaatcagctcgaacctatggaacgcgagcgcgacaagctggccgaccagcttgccgaagtgaaacgccgcagcggcgaagaactaaaccgctgcatggaaaagctcacccaacgcgataacgaggctatcgaggcccgcaaacaggccaaggaggccatcgagcgcgccgccagtctgcaaggtcaggtggaagccctcaaagagcaggtcgccaatctcacagccgtcttgaaaacaggaggcaaacaatgaaaagcgcccttgccgcccttcgcgcggtcgcggccgctgtcgtcctaatcgtcagtgtgcccgcttgggccgacttccggggtgaagtcgtccgaatccttgacggtgacactatcgacgttttggtgaaccgtcagaccatccgcgtgagattggccgatattgacgcaccggaaagcggccaagccttcggctcccgtgctcgccaacggctcgccgacttgacctttcgccaagaggttcaagtgaccgaaaaagaggttgatcggtatggccgcactcttggggtcgtttacgcgccgttgcaataccccggcggccaaacacaactcaccaacatcaatgcgatcatggttcaagaaggcatggcctgggcttaccgttattacggcaaaccaaccgacgcgcagatgtacgagtatgaaaaagaggcccgccgccaacggctcggcctttggtcagacccgaatgctcaggagccttggaaatggcgtcgcgcctcgaaaaatgccacgaactgacaccgggcacgccccttgttcgacgcgccgcaggcacgtcgaatttaccgccgggacg

SEQ ID NO: 46 - DNA, 1175 bp, synthetic construct
  misc_feature 1..1175: note = synthetic spectinomycin resistance sequence of Ptac-T7 synthetic controller
  source 1..1175: mol_type = other DNA
cccctcgtcccgacacttccagatcgccatagcgcacagcgcctcgagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaatggtaccc

SEQ ID NO: 47 - DNA, 72 bp, synthetic construct
  misc_feature 1..72: note = synthetic terminator (BBa_B0053) sequence of Ptac-T7 synthetic controller
  source 1..72: mol_type = other DNA
tccggcaaaaaaacgggcaaggtgtcaccaccctgccctttttctttaaaaccgaaaagattacttcgcgtt

SEQ ID NO: 48 - DNA, 20 bp, synthetic construct
  misc_feature 1..20: note = synthetic VF2 primer (BBa_G000101) sequence of Ptac-T7 synthetic controller
  source 1..20: mol_type = other DNA
tgccacctgacgtctaagaa

SEQ ID NO: 49 - DNA, 78 bp, synthetic construct
  misc_feature 1..78: note = synthetic terminator (BBa_B0055) sequence of Ptac-T7 synthetic controller
  source 1..78: mol_type = other DNA
aaggaatattcagcaatttgcccgtgccgaagaaaggcccacccgtgaaggtgagccagtgagttgattgctacgtaa

SEQ ID NO: 50 - DNA, 12 bp, synthetic construct
  misc_feature 1..12: note = synthetic translational stop (BBa_B0042) sequence of Ptac-T7 synthetic controller
  source 1..12: mol_type = other DNA
ttagttagttag

SEQ ID NO: 51 - DNA, 22 bp, synthetic construct
  misc_feature 1..22: note = synthetic Biobrick prefix (BBa_G00000) sequence of Ptac-T7 synthetic controller
  source 1..22: mol_type = other DNA
gaattcgcggccgcttctagag

SEQ ID NO: 52 - DNA, 23 bp, synthetic construct
  misc_feature 1..23: note = synthetic Enterobacteria phage T7 wild type (WT) promoter of full synthetic cluster
  source 1..23: mol_type = other DNA
taatacgactcactatagggaga

SEQ ID NO: 53 - DNA, 28 bp, synthetic construct
  misc_feature 1..28: note = synthetic nifH ribosome binding site (rbs, RBS) of full synthetic cluster
  source 1..28: mol_type = other DNA
acaataaactaacataaggaggataaat

SEQ ID NO: 54 - DNA, 882 bp, synthetic construct; identical to SEQ ID NO: 4
  misc_feature 1..882: note = synthetic nifH of full synthetic cluster
  source 1..882: mol_type = other DNA

SEQ ID NO: 55 - DNA, 50 bp, synthetic construct
  misc_feature 1..50: note = synthetic insulator 1 of full synthetic cluster
  source 1..50: mol_type = other DNA
gcgtgcgtacaccttaatcaccgcttcatgctaaggtcctggctgcatgc

SEQ ID NO: 56 - DNA, 34 bp, synthetic construct
  misc_feature 1..34: note = synthetic nifD ribosome binding site (rbs, RBS) of full synthetic cluster
  source 1..34: mol_type = other DNA
aaaaattcacatccctatctagcggaggagccgg

SEQ ID NO: 57 - DNA, 1452 bp, synthetic construct; identical to SEQ ID NO: 6
  misc_feature 1..1452: note = synthetic nifD of full synthetic cluster
  source 1..1452: mol_type = other DNA

SEQ ID NO: 58 - DNA, 50 bp, synthetic construct
  misc_feature 1..50: note = synthetic insulator 2 of full synthetic cluster
  source 1..50: mol_type = other DNA
caaacaccccatgtcgatactgaacgaatcgacgcacactcccttccttg

SEQ ID NO: 59 - DNA, 34 bp, synthetic construct
  misc_feature 1..34: note = synthetic nifK ribosome binding site (rbs, RBS) of full synthetic cluster
  source 1..34: mol_type = other DNA
caatctcatactctcaaaaattaggcgaggtaac

SEQ ID NO: 60 - DNA, 1563 bp, synthetic construct; identical to SEQ ID NO: 8
  misc_feature 1..1563: note = synthetic nifK of full synthetic cluster
  source 1..1563: mol_type = other DNA

SEQ ID NO: 61 - DNA, 40 bp, synthetic construct
  misc_feature 1..40: note = synthetic insulator 3 of full synthetic cluster
  source 1..40: mol_type = other DNA
cctgattgtatccgcatctgatgctaccgtggttgagtta

SEQ ID NO: 62 - DNA, 26 bp, synthetic construct
  misc_feature 1..26: note = synthetic nifY ribosome binding site (rbs, RBS) of full synthetic cluster
  source 1..26: mol_type = other DNA
ccatactcactcccggaggtacttct

SEQ ID NO: 63 - DNA, 663 bp, synthetic construct; identical to SEQ ID NO: 10
  misc_feature 1..663: note = synthetic nifY of full synthetic cluster
  source 1..663: mol_type = other DNA

SEQ ID NO: 64 - DNA, 40 bp, synthetic construct
  misc_feature 1..40: note = synthetic insulator 4 of full synthetic cluster
  source 1..40: mol_type = other DNA
catttttgccttgcgacagacctcctacttagattgccac

SEQ ID NO: 65 - DNA, 32 bp, synthetic construct
  misc_feature 1..32: note = synthetic nifE ribosome binding site (rbs, RBS) of full synthetic cluster
  source 1..32: mol_type = other DNA
actattcaatacatcactggaggttattacaa

SEQ ID NO: 66 - DNA, 1374 bp, synthetic construct; identical to SEQ ID NO: 12
  misc_feature 1..1374: note = synthetic nifE of full synthetic cluster
  source 1..1374: mol_type = other DNA

SEQ ID NO: 67 - DNA, 40 bp, synthetic construct
  misc_feature 1..40: note = synthetic insulator 5 of full synthetic cluster
  source 1..40: mol_type = other DNA
tgtcacgctaggaggcaattctataagaatgcacactgca

SEQ ID NO: 68 - DNA, 31 bp, synthetic construct
  misc_feature 1..31: note = synthetic nifN ribosome binding site (rbs, RBS) of full synthetic cluster
  source 1..31: mol_type = other DNA
cctaaacctaccacacctggaagaagtaatt

SEQ ID NO: 69 - DNA, 1386 bp, synthetic construct; identical to SEQ ID NO: 14
  misc_feature 1..1386: note = synthetic nifN of full synthetic cluster
  source 1..1386: mol_type = other DNA

SEQ ID NO: 70 - DNA, 27 bp, synthetic construct
  misc_feature 1..27: note = synthetic nifJ ribosome binding site (rbs, RBS) of full synthetic cluster
  source 1..27: mol_type = other DNA
ctagagattaaagaggagaaattaagc

SEQ ID NO: 71 - DNA, 3507 bp, synthetic construct
  misc_feature 1..3507: note = synthetic nifJ of full synthetic cluster
  source 1..3507: mol_type = other DNA
atgaaaactatggacggtaacgctgcggctgcatggattagctacgcctttaccgaagtggctgcgatctacccgattacgccgagcaccccgatggcggaaaatgtggacgaatgggctgcgcagggcaagaagaacctcttcggccagccggtgcgcctgatggagatgcagtcggaagcgggtgcagcaggtgctgtgcatggcgccttgcaagctggcgcactgacgaccacctacaccgcgtcgcagggcctgttgctgatgatcccaaacatgtacaaaatcgcgggtgaactgctgccgggtgtctttcatgtttcggcacgcgcactggccaccaatagcctcaacatctttggcgatcatcaggatgtaatggcggtgcgccaaacgggctgcgcgatgttggccgagaataacgtccagcaagttatggatttgtccgcggtagcccacttggcagcgatcaaaggtcgcattccgttcgtgaacttcttcgatggctttcgcaccagccacgaaatccagaagatcgaggttctggaatatgaacagctggccaccttgttggatcgtccggccctggacagcttccgccgtaacgcccttcacccggaccacccggtcatccgtggcaccgcccagaacccggacatctacttccaggaacgtgaggccggtaaccgtttctatcaggcgctcccggatattgtggaatcttacatgacccagatttctgccctgactggtcgcgagtatcacctgtttaactacactggtgctgcggatgcggagcgcgtgatcatcgcgatgggctctgtctgtgacaccgtccaagaggtggttgacacgctgaatgcagcgggtgagaaagttggtctgctctccgttcatcttttccgcccgttttcgttagcgcacttcttcgcccaactgccgaaaactgtacagcgtatcgcagtattggaccgtacgaaagagccaggtgctcaagcagagccgctgtgcctcgatgtgaagaatgccttttaccaccatgacgatgccccgttgattgtgggtggtcgctatgccttgggcggtaaggacgtgttgccgaacgatattgcggccgtgtttgataacctgaacaaaccgctgccgatggacggcttcacgctgggtatcgtggacgatgttaccttcacctctctcccgccagcgcagcagaccctggcggtttctcacgacggcatcacggcatgtaagttttggggcatgggctccgacggcacggttggtgcgaacaagtccgcgatcaagattatcggcgacaaaacgccactgtatgcgcaagcgtacttttcctacgactcgaagaagagcggtggtattaccgtcagccatctgcgttttggtgatcgcccgatcaactccccgtatttgatccatcgcgcggatttcatctcgtgcagccagcaaagctatgttgaacgctacgatctgctggatggccttaaaccgggtggcacctttctgctgaactgctcctggagcgatgccgaactggagcaacatctgccggtcggtttcaaacgttatctggcacgcgagaatatccacttctacactctcaacgctgtggacatcgcccgtgagcttggtttgggtggccgtttcaacatgctgatgcaggctgccttcttcaaactggccgcgatcattgacccgcagactgctgcggactatctgaagcaggctgttgagaaaagctatggcagcaaaggtgcggcggtcatcgagatgaaccagcgtgccatcgagcttggcatggccagcctgcaccaggtgacgatcccggcacattgggccaccctggatgagccagcggcgcaggcgtccgcgatgatgccggactttatccgcgacatcctgcaaccgatgaaccgtcagtgcggcgaccagcttccggtgtcggcttttgtcggcatggaagatggcaccttcccgtccggcacggccgcatgggagaaacgtggcatcgcccttgaggtgccagtctggcagccggaaggctgcacgcagtgcaaccagtgcgccttcatttgtccgcacgccgcgattcgtccggcgttgttgaatggcgaagagcatgatgctgccccggttggcctgctgagcaaaccggcacaaggcgctaaagaatatcactatcatctggcgattagcccgctggactgctccggctgtggcaactgcgttgacatttgtccagctcgtggcaaagcgttgaagatgcagtctctggatagccaacgccagatggctccggtgtgggattatgcgctggcgctgaccccgaagtctaacccgtttcgtaaaaccaccgtcaaaggctcgcagttcgaaaccccgctgctggagtttagcggtgcgtgcgctggttgtggcgaaacgccgtatgcgcgcctcattacccagctgtttggcgaccgcatgctgattgccaatgccaccggctgttccagcatctggggcgcatctgcgccgagcatcccgtataccaccaatcatcgtggtcatggtccggcctgggcgaatagcctgtttgaggacaatgccgaatttggtttaggtatgatgctgggcggtcaagctgtgcgtcaacagatcgcggacgatatgacggctgcgttagcgctcccggtttccgatgagctgagcgacgcgatgcgccagtggttggcgaaacaggacgagggtgaaggcacgcgtgagcgtgcggaccgtctgagcgagcgcttagccgcggagaaagagggcgttccgctgttagagcagctgtggcaaaatcgtgattactttgtgcgtcgcagccagtggattttcggcggtgacggctgggcctatgatattggcttcggtggcctggaccacgtcctcgccagcggtgaggatgtgaacattctggtatttgacaccgaagtctactcgaacaccggcggtcaaagcagcaaatcgaccccggtcgcggccatcgccaagttcgcggctcagggcaagcgcacccgcaagaaagacctgggtatgatggcgatgagctacggcaacgtctatgtagcccaggtggcgatgggtgcggataaagatcaaactctgcgcgccattgcggaagctgaagcgtggccaggcccgtcgctggtgattgcgtatgcggcctgcatcaatcatggcctgaaggccggtatgcgttgcagccaacgtgaggcgaagcgcgctgttgaggcgggctactggcacctgtggcgttatcacccgcagcgcgaagcggaaggcaagacgccgtttatgttagatagcgaagaaccggaagagtcgttccgtgactttctgttgggtgaggtgcgctacgcatccctgcacaagaccaccccgcacctcgccgatgcccttttcagccgtaccgaagaagatgcgcgtgcgcgctttgcgcaataccgtcgcctggctggcgaagagtaataa

SEQ ID NO: 72 - DNA, 37 bp, synthetic construct
  misc_feature 1..37: note = synthetic Enterobacteria phage T7 terminator 25 of full synthetic cluster
  source 1..37: mol_type = other DNA
tactctaaccccatcggccgtcttaggggttttttgt

SEQ ID NO: 73 - DNA, 30 bp, synthetic construct
  misc_feature 1..30: note = synthetic insulator 6 of full synthetic cluster
  source 1..30: mol_type = other DNA
ccgtggttgagtcagcgtcgagcacgcggc

SEQ ID NO: 74 - DNA, 23 bp, synthetic construct
  misc_feature 1..23: note = synthetic Enterobacteria phage T7 promoter mut 2 of full synthetic cluster
  source 1..23: mol_type = other DNA
taatacgactcactagagagaga

SEQ ID NO: 75 - DNA, 40 bp, synthetic construct
  misc_feature 1..40: note = synthetic insulator 7 of full synthetic cluster
  source 1..40: mol_type = other DNA
cgcgacttccagagaagaagactactgacttgagcgttcc

SEQ ID NO: 76 - DNA, 39 bp, synthetic construct
  misc_feature 1..39: note = synthetic nifB ribosome binding site (rbs, RBS) of full synthetic cluster
  source 1..39: mol_type = other DNA
ctctctgtaatacatcaaatcaatcataggagggctaaa

SEQ ID NO: 77 - DNA, 1407 bp, synthetic construct; identical to SEQ ID NO: 30
  misc_feature 1..1407: note = synthetic nifB of full synthetic cluster
  source 1..1407: mol_type = other DNA

SEQ ID NO: 78 - DNA, 40 bp, synthetic construct
  misc_feature 1..40: note = synthetic insulator 8 of full synthetic cluster
  source 1..40: mol_type = other DNA
actacgagatttgaggtaaaccaaataagcacgtagtggc

SEQ ID NO: 79 - DNA, 33 bp, synthetic construct
  misc_feature 1..33: note = synthetic nifQ ribosome binding site (rbs, RBS) of full synthetic cluster
  source 1..33: mol_type = other DNA
tgcgtttagcagttcccgtaggaatatttcctt

SEQ ID NO: 80 - DNA, 504 bp, synthetic construct; identical to SEQ ID NO: 32
  misc_feature 1..504: note = synthetic nifQ of full synthetic cluster
  source 1..504: mol_type = other DNA

SEQ ID NO: 81 - DNA, 29 bp, synthetic construct
  misc_feature 1..29: note = synthetic Enterobacteria phage T7 terminator 1 of full synthetic cluster
  source 1..29: mol_type = other DNA
tacatatcgggggggtaggggttttttgt

SEQ ID NO: 82 - DNA, 18 bp, synthetic construct
  misc_feature 1..18: note = synthetic insulator 9 of full synthetic cluster
  source 1..18: mol_type = other DNA
gtctgtagcacgtgcatc

SEQ ID NO: 83 - DNA, 23 bp, synthetic construct
  misc_feature 1..23: note = synthetic Enterobacteria phage T7 promoter mut 3 of full synthetic cluster
  source 1..23: mol_type = other DNA
taatacgactcactaatgggaga

SEQ ID NO: 84 - DNA, 34 bp, synthetic construct
  misc_feature 1..34: note = synthetic nifF ribosome binding site (rbs, RBS) of full synthetic cluster
  source 1..34: mol_type = other DNA
gacaagagtctcaattataaggaggctttactac

SEQ ID NO: 85 - DNA, 534 bp, synthetic construct
  misc_feature 1..534: note = synthetic nifF of full synthetic cluster
  source 1..534: mol_type = other DNA
atggcgaacatcggcatcttctttggtacggataccggcaaaacccgcaagattgcgaagatgattcacaaacagctgggcgagctggccgatgccccggttaacatcaatcgtaccactttggatgactttatggcttacccagtcctgttgctcggcacgccgacgcttggtgatggtcaactgccgggcttagaggcgggctgcgagagcgaaagctggtctgagtttatctccggtctggatgacgcttccctgaagggcaaaaccgtggcgctgtttggcctgggcgaccagcgtggttacccggacaacttcgtgtcgggtatgcgtccgctgttcgacgcgctgagcgcccgtggcgcccagatgattggtagctggccgaacgaaggttatgagtttagcgcatcgtccgcgctggaaggcgaccgcttcgtcggcttggtgctggatcaagacaatcagttcgaccagaccgaagcgcgcctggcgtcttggcttgaagagatcaaacgcaccgttctgtaataa

SEQ ID NO: 86 - DNA, 29 bp, synthetic construct
  misc_feature 1..29: note = synthetic Enterobacteria phage T7 terminator 1 of full synthetic cluster
  source 1..29: mol_type = other DNA
tacatatcgggggggtaggggttttttgt

SEQ ID NO: 87 - DNA, 18 bp, synthetic construct
  misc_feature 1..18: note = synthetic insulator 10 of full synthetic cluster
  source 1..18: mol_type = other DNA
ggtcattacaacggttat

SEQ ID NO: 88 - DNA, 23 bp, synthetic construct
  misc_feature 1..23: note = synthetic Enterobacteria phage T7 promoter mut 2 of full synthetic cluster
  source 1..23: mol_type = other DNA
taatacgactcactagagagaga

SEQ ID NO: 89 - DNA, 40 bp, synthetic construct
  misc_feature 1..40: note = synthetic insulator 11 of full synthetic cluster
  source 1..40: mol_type = other DNA
aacatagcgttccatgagggctagaattacctaccggcct

SEQ ID NO: 90 - DNA, 37 bp, synthetic construct
  misc_feature 1..37: note = synthetic nifU ribosome binding site (rbs, RBS) of full synthetic cluster
  source 1..37: mol_type = other DNA
cagatactgacaaataaaccagcgaaggaggttccta

SEQ ID NO: 91 - DNA, 825 bp, synthetic construct; identical to SEQ ID NO: 16
  misc_feature 1..825: note = synthetic nifU of full synthetic cluster
  source 1..825: mol_type = other DNA

SEQ ID NO: 92 - DNA, 40 bp, synthetic construct
  misc_feature 1..40: note = synthetic insulator 12 of full synthetic cluster
  source 1..40: mol_type = other DNA
cattgtaatagccaccaaaagagtgatgatagtcatgggt

SEQ ID NO: 93 - DNA, 39 bp, synthetic construct
  misc_feature 1..39: note = synthetic nifS ribosome binding site (rbs, RBS) of full synthetic cluster
  source 1..39: mol_type = other DNA
gatacccgtagaccattctgaaatcgaaggaggttttcc

SEQ ID NO: 94 - DNA, 1203 bp, synthetic construct
  misc_feature 1..1203: note = synthetic nifS of full synthetic cluster
  source 1..1203: mol_type = other DNA
atgaaacaagtgtacctggacaacaacgcgaccacccgcctggacccgatggttctggaagcgatgatgccgtttctcacggatttctatggcaatccgtccagcatccatgacttcggcatcccggcacaagcggcgctggaacgtgcgcaccagcaagctgcggcactgctgggcgcagagtacccgtctgaaatcattttcacgagctgtgcgaccgaggccactgcaaccgccattgcgtcggccatcgcgttattgccggaacgccgcgaaatcatcacctcggtagtggagcacccggctacgctggcggcgtgcgagcacctggaacgccaaggctatcgcatccatcgcattgcggtggatagcgaaggtgcgctggacatggcccagttccgtgcagcgctctcgccgcgtgtcgcgttggtgagcgtgatgtgggccaacaacgaaaccggcgtgctgttcccgattggcgaaatggccgagcttgcccacgagcagggcgctctgttccactgcgatgccgttcaggtcgttggcaaaatcccaattgctgttggccagacgcgcatcgacatgctgtcttgctccgcgcacaagtttcatggtccgaagggtgttggttgcttgtacttacgtcgtggcacgcgctttcgtccgctgcttcgcggtggccatcaagaatatggtcgccgtgccggcactgagaatatctgtggcatcgtcggcatgggcgctgcgtgcgaactggcgaacatccatctgccgggtatgacccatattggccagttacgcaatcgcctggagcaccgtctgctcgccagcgtgccgtccgtgatggttatgggcggtggtcagccgcgtgtaccgggtactgtcaacctggcgttcgagtttatcgaaggtgaagcgatcctgctcttgctgaaccaggctggcattgccgcaagctccggctccgcgtgtacctctggcagcttggagccgagccatgtgatgcgcgccatgaacattccatacaccgcggctcacggcaccattcgttttagcctgagccgttatacgcgcgagaaagagatcgactacgtcgttgcgaccctcccgccaatcattgatcgtctgcgtgccttgtccccgtattggcagaatggtaagccgcgtccggcagatgcagtctttaccccggtttacggttaa

SEQ ID NO: 95 - DNA, 40 bp, synthetic construct
  misc_feature 1..40: note = synthetic insulator 13 of full synthetic cluster
  source 1..40: mol_type = other DNA
gagttactggccctgatttctccgcttctaataccgcaca

SEQ ID NO: 96 - DNA, 34 bp, synthetic construct
  misc_feature 1..34: note = synthetic nifV ribosome binding site (rbs, RBS) of full synthetic cluster
  source 1..34: mol_type = other DNA
gcgactaggagcctaactcgccacaaggaaacat

SEQ ID NO: 97 - DNA, 1143 bp, synthetic construct; identical to SEQ ID NO: 20
  misc_feature 1..1143: note = synthetic nifV of full synthetic cluster
  source 1..1143: mol_type = other DNA

SEQ ID NO: 98 - DNA, 40 bp, synthetic construct
  misc_feature 1..40: note = synthetic insulator 14 of full synthetic cluster
  source 1..40: mol_type = other DNA
gactcaacacgctagggacgtgaagtcgattccttcgatg

SEQ ID NO: 99 - DNA, 35 bp, synthetic construct
  misc_feature 1..35: note = synthetic nifW ribosome binding site (rbs, RBS) of full synthetic cluster
  source 1..35: mol_type = other DNA
cagaaggcgagaactagatttaagggccattatag

SEQ ID NO: 100 - DNA, 258 bp, synthetic construct; identical to SEQ ID NO: 22
  misc_feature 1..258: note = synthetic nifW of full synthetic cluster
  source 1..258: mol_type = other DNA

SEQ ID NO: 101 - DNA, 40 bp, synthetic construct
  misc_feature 1..40: note = synthetic insulator 15 of full synthetic cluster
  source 1..40: mol_type = other DNA
tcgagaaacaaggcagttccgggctgaaagtagcgccggg

SEQ ID NO: 102 - DNA, 36 bp, synthetic construct
  misc_feature 1..36: note = synthetic nifZ ribosome binding site (rbs, RBS) of full synthetic cluster
  source 1..36: mol_type = other DNA
acaagtcccgtattataaccgcctaggaggtgttgg

SEQ ID NO: 103 - DNA, 447 bp, synthetic construct; identical to SEQ ID NO: 24
  misc_feature 1..447: note = synthetic nifZ of full synthetic cluster
  source 1..447: mol_type = other DNA

SEQ ID NO: 104 - DNA, 40 bp, synthetic construct
  misc_feature 1..40: note = synthetic insulator 16 of full synthetic cluster
  source 1..40: mol_type = other DNA
acgccacgcgtagtgagacatacacgttcgttgggttcac

SEQ ID NO: 105 - DNA, 34 bp, synthetic construct
  misc_feature 1..34: note = synthetic nifM ribosome binding site (rbs, RBS) of full synthetic cluster
  source 1..34: mol_type = other DNA
tcagagactgaagttattacccaggaggtctata

SEQ ID NO: 106 - DNA, 801 bp, synthetic construct; identical to SEQ ID NO: 26
  misc_feature 1..801: note = synthetic nifM of full synthetic cluster
  source 1..801: mol_type = other DNA

SEQ ID NO: 107 - DNA, 47 bp, synthetic construct
  misc_feature 1..47: note = synthetic Enterobacteria phage T7 wild type (WT) terminator of full synthetic cluster
  source 1..47: mol_type = other DNA
tagcataaccccttggggcctctaaacgggtcttgaggggttttttg

SEQ ID NO: 108 - DNA, 21 bp, synthetic construct
  misc_feature 1..21: note = synthetic Biobrick suffix (BBa_G000001) of full synthetic cluster
  source 1..21: mol_type = other DNA
tactagtagcggccgctgcag

SEQ ID NO: 109 - DNA, 12 bp, synthetic construct
  misc_feature 1..12: note = synthetic translational stop (BBa_B0042) of full synthetic cluster
  source 1..12: mol_type = other DNA
ttagttagttag

SEQ ID NO: 110 - DNA, 69 bp, synthetic construct; identical to SEQ ID NO: 42
  misc_feature 1..69: note = synthetic terminator (BBa_B0054) of full synthetic cluster
  source 1..69: mol_type = other DNA

SEQ ID NO: 111 - DNA, 20 bp, synthetic construct
  misc_feature 1..20: note = synthetic VR primer (BBa_G00102) of full synthetic cluster
  source 1..20: mol_type = other DNA
gctcactcaaaggcggtaat

SEQ ID NO: 112 - DNA, 41 bp, synthetic construct; identical to SEQ ID NO: 44
  misc_feature 1..41: note = synthetic terminator (BBa_B0062) of full synthetic cluster
  source 1..41: mol_type = other DNA

SEQ ID NO: 113 - DNA, 2037 bp, synthetic construct
  misc_feature 1..2037: note = synthetic pSC101 origin (BBa_150042) of full synthetic cluster
  source 1..2037: mol_type = other DNA
ctgtcagaccaagtttacgagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggacatgtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaac

SEQ ID NO: 114 - DNA, 769 bp, synthetic construct
  misc_feature 1..769: note = synthetic chloramphenicol resistance (BBa_P1004) of full synthetic cluster
  source 1..769: mol_type = other DNA
gttgatcgggcacgtaagaggttccaactttcaccataatgaaataagatcactaccgggcgtattttttgagttatcgagattttcaggagctaaggaagctaaaatggagaaaaaaatcacgggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaacgctcacccggagtttcgtatggccatgaaagacggtgagctggtgatctgggatagtgttcacccttgttacaccgttttccatgagcaaactgaaacgttttcgtccctctggagtgaataccacgacgatttccggcagtttctccacatatattcgcaagatgtggcgtgttacggtgaaaacctggcctatttccctaaagggtttattgagaatatgttttttgtctcagccaatccctgggtgagtttcaccagttttgatttaaacgtggccaatatggacaacttcttcgcccccgttttcacgatgggcaaatattatacgcaaggcgacaaggtgctgatgccgctggcgatccaggttcatcatgccgtttgtgatggcttccatgtcggccgcatgcttaatgaattacaacagtactgtgatgagtggcagggcggggcgtaataa

SEQ ID NO: 115 - DNA, 72 bp, synthetic construct; identical to SEQ ID NO: 47
  misc_feature 1..72: note = synthetic terminator (BBa_B0053) of full synthetic cluster
  source 1..72: mol_type = other DNA

SEQ ID NO: 116 - DNA, 20 bp, synthetic construct
  misc_feature 1..20: note = synthetic VF2 primer (BBa_G00101) of full synthetic cluster
  source 1..20: mol_type = other DNA
tgccacctgacgtctaagaa

SEQ ID NO: 117 - DNA, 78 bp, synthetic construct; identical to SEQ ID NO: 49
  misc_feature 1..78: note = synthetic terminator (BBa_B0055) of full synthetic cluster
  source 1..78: mol_type = other DNA

SEQ ID NO: 118 - DNA, 12 bp, synthetic construct
  misc_feature 1..12: note = synthetic translational stop (BBa_B0042) of full synthetic cluster
  source 1..12: mol_type = other DNA
ttagttagttag

SEQ ID NO: 119 - DNA, 19 bp, synthetic construct
  misc_feature 1..19: note = synthetic SynPrgH ribosome binding site (rbs, RBS) of bacterial type III secretion system (T3SS)
  source 1..19: mol_type = other DNA
attaaacaggataataagc

SEQ ID NO: 120 - DNA, 1182 bp, synthetic construct
  misc_feature 1..1182: note = synthetic SynPrgH of bacterial type III secretion system (T3SS)
  source 1..1182: mol_type = other DNA
atggaaactagcaaggagaaaacgattacgtcccctggtccgtatatcgttcgtctgttgaactcgtccctgaacggttgtgaatttccgctgctgactggtcgtacgctgttcgtcgtgggtcagagcgatgctctgaccgcgtctggtcagctgccggacattcctgccgactccttcttcatcccgctggatcatggcggtgttaatttcgagattcaagtggacactgacgcgacggaaatcatcctgcacgaactgaaagagggcaactccgagagccgctccgtgcaactgaacaccccgatccaagttggtgaactgctgattttgattcgtccggagagcgagccgtgggtgccggaacagccggagaagttggaaacttctgcgaaaaagaacgaaccgcgctttaaaaacggcatcgtcgcggcactggcgggtttctttatcctgggtatcggcacggttggcaccctgtggattctgaactcgccgcaacgtcaagcagccgaattggacagcctgttgggtcaggagaaggagcgttttcaggtgctgccgggtcgcgacaagatgctgtatgtcgccgcgcaaaacgaacgcgacaccctgtgggcacgtcaagtcctggcacgcggcgattacgataaaaacgcacgcgttattaatgaaaatgaggagaataaacgtatcagcatctggctggacacgtattatccacaactggcatattaccgtatccattttgatgaaccacgtaagccggtgttttggctgtcccgtcaacgcaacacgatgagcaagaaagagctggaggtgctgtcccagaaattgcgtgcgctgatgccgtacgccgacagcgtcaatattactctgatggatgacgtgaccgcagcaggccaagccgaggcaggtctgaaacaacaggcgctgccatacagccgccgtaaccacaaaggtggtgttacgttcgttattcagggcgccttggacgacgttgagattctgcgtgcgcgccagtttgtcgactcctattatcgtacctggggtggtcgttacgttcaattcgcaattgaattgaaagacgattggctgaaaggccgctcgttccaatacggtgcggaaggctacattaagatgagcccaggtcattggtattttccgtctcctctgtaatag

SEQ ID NO: 121 - DNA, 21 bp, synthetic construct
  misc_feature 1..21: note = synthetic SynPrgI ribosome binding site (rbs, RBS) of bacterial type III secretion system (T3SS)
  source 1..21: mol_type = other DNA
attaaagaggagaaattaagc

SEQ ID NO: 122 - DNA, 21 bp, synthetic construct
  misc_feature 1..21: note = synthetic SynPrgJ ribosome binding site (rbs, RBS) of bacterial type III secretion system (T3SS)
  source 1..21: mol_type = other DNA
attaaggaggataaattaagc

SEQ ID NO: 123 - DNA, 309 bp, synthetic construct
  misc_feature 1..309: note = synthetic SynPrgJ of bacterial type III secretion system (T3SS)
  source 1..309: mol_type = other DNA
atgtctatcgcgactatcgtgcctgaaaatgccgttattggtcaagcggtgaatattcgcagcatggaaacggacatcgtcagcttggacgaccgtttgctgcaagcattttcgggcagcgccatcgctaccgccgtcgataagcagaccattaccaatcgcattgaagaccctaatctggttaccgatccgaaggagctggcgattagccaggaaatgatttccgactacaatctgtacgtcagcatggttagcaccctgacgcgtaagggcgttggcgctgttgagactttgctgcgttcctgatag

SEQ ID NO: 124 - DNA, 19 bp, synthetic construct
  misc_feature 1..19: note = synthetic SynPrgK ribosome binding site (rbs, RBS) of bacterial type III secretion system (T3SS)
  source 1..19: mol_type = other DNA
attaaacaggataataagc

SEQ ID NO: 125 - DNA, 762 bp, synthetic construct
  misc_feature 1..762: note = synthetic SynPrgK of bacterial type III secretion system (T3SS)
  source 1..762: mol_type = other DNA
atgatccgccgttacctgtataccttcttgctggttatgactttggccggctgtaaagataaggatctgctgaaaggcttggaccaagagcaagcgaatgaggtcattgcggttctgcaaatgcacaacattgaggctaacaagattgatagcggcaagctgggttacagcattaccgtcgcggaaccggatttcaccgcggcagtgtattggattaagacctaccagttgccgcctcgcccgcgtgtcgaaatcgcccaaatgtttccggcagacagcctggttagctctccgcgtgcggagaaagcacgtctgtactcggcgattgaacagcgcctggagcagtcgctgcaaacgatggaaggtgttctgtcggcccgtgtccacatcagctatgatattgatgcgggcgagaacggtcgtccgcctaagccggtgcacctgtcggctttggcggtgtatgaacgcggttcccctctggcccatcagatttcggatattaagcgcttcctgaaaaacagcttcgcggatgttgactatgataacatcagcgtggttctgtccgagcgtagcgacgcacagttgcaggcgccgggcacgccggtcaagcgcaatagcttcgctacctcctggattgtgctgattatcctgttgtctgttatgagcgcgggtttcggtgtctggtactacaaaaatcactatgcgcgtaataagaaaggcattactgccgatgacaaggcaaagtccagcaacgagtaataa

SEQ ID NO: 126 - DNA, 19 bp, synthetic construct
  misc_feature 1..19: note = synthetic SynOrgA ribosome binding site (rbs, RBS) of bacterial type III secretion system (T3SS)
  source 1..19: mol_type = other DNA
gaaagaagggacagactag

SEQ ID NO: 127 - DNA, 603 bp, synthetic construct
  misc_feature 1..603: note = synthetic SynOrgA of bacterial type III secretion system (T3SS)
  source 1..603: mol_type = other DNA
atgattcgtcgtaaccgtcaaatgaaccgtcaaccactgccaattatctggcaacgcattattttcgacccactgtcctatattcacccacaacgtctgcaaatcgcgccggagatgatcgtgcgtccggcagcgcgtgcggcagcgaatgagctgattttggcggcgtggcgtttgaagaacggtgagaaggagtgcattcagaatagcctgacgcagctgtggttgcgtcaatggcgccgtctgccgcaggttgcttacctgctgggttgccacaagttgcgtgctgacctggcccgtcaaggtgctttattgggcctgccggactgggcgcaggcattcttggcgatgcaccagggtacgtccttgtcggtttgtaataaggcgccgaaccaccgtttcctgctgtccgttggttacgcccaactgaacgcgctgaatgagttcctgccggagagcttggcccaacgctttcctctgctgtttccaccgttcatcgaggaggcactgaaacaagatgcagtggagatgagcatcctgctgctggccctgcaatacgcgcaaaagtatcctaacaccgtcccggcgtttgcgtgctaataa

SEQ ID NO: 128 - DNA, 19 bp, synthetic construct
  misc_feature 1..19: note = synthetic SynOrgB ribosome binding site (rbs, RBS) of bacterial type III secretion system (T3SS)
  source 1..19: mol_type = other DNA
gaaagaagggacagactag

SEQ ID NO: 129 - DNA, 684 bp, synthetic construct
  misc_feature 1..684: note = synthetic SynOrgB of bacterial type III secretion system (T3SS)
  source 1..684: mol_type = other DNA
atgttgaaaaatatcccgattccatcccctctgtctccggttgaaggtatcctgattaaacgcaagacgttggagcgttacttctcgattgagcgcctggaacaacaggcgcatcagcgtgcaaagcgcattttgcgtgaggcagaagaagaagccaagaccctgcgcatgtatgcgtaccaggagggctacgagcagggcatgattgacgcactgcaacaggtggccgcctatttgaccgacaaccagacgatggcttggaaatggatggagaaaattcaaatctatgcgcgtgagttgttcagcgcggctgtcgatcacccggaaacgttgttgacggtgctggacgagtggctgcgtgatttcgataagccggaaggtcagctgtttttgaccctgccggtgaacgcaaagaaagatcatcagaaactgatggtgctgctgatggaaaattggccgggcaccttcaatctgaagtatcatcaggagcaacgttttatcatgtcctgtggcgatcagattgccgagttttcgccggaacaatttgttgaaacggcggttggtgttatcaagcaccatctggatgagctgcctcaggactgtcgcaccatttcggacaatgcgattaacgcgctgattgatgaatggaagacgaaaacccaagctgaagttattcgctgataa

SEQ ID NO: 130 - DNA, 36 bp, synthetic construct
  misc_feature 1..36: note = synthetic SynInvA ribosome binding site (rbs, RBS) of bacterial type III secretion system (T3SS)
  source 1..36: mol_type = other DNA
cttgggcacgcgtccattaaacaggagtaattaagc

SEQ ID NO: 131 - DNA, 2058 bp, synthetic construct
  misc_feature 1..2058: note = synthetic SynInvA of bacterial type III secretion system (T3SS)
  source 1..2058: mol_type = other DNA
atgctgctgtccctgctgaatagcgcgcgtctgcgtcctgagctgctgattctggttctgatggttatgatcatcagcatgttcgttatcccgttgccgacctatttggttgacttcttgatcgctttgaacattgtcctggcaattctggtgttcatgggctccttctacatcgaccgcattctgagcttcagcacctttccggcggttctgctgatcacgactctgttccgtttggcactgagcatcagcaccagccgcctgatcctgattgaagcagatgcgggtgagatcatcgcgacctttggtcagtttgtgatcggtgacagcctggcggttggtttcgtcgtattctccatcgtgacggtggtgcagtttatcgttattaccaagggcagcgaacgtgtggcggaggtcgccgctcgcttcagcctggacggcatgccgggtaaacagatgtctattgatgcagacctgaaagccggcattattgatgctgatgcagcgcgcgagcgccgcagcgtcctggagcgtgaaagccaactgtacggttccttcgacggtgccatgaagttcattaaaggtgatgcgattgcgggcatcattatcatcttcgttaacttcattggcggtatcagcgtcggtatgacccgtcatggtatggatctgagcagcgccctgagcacctacaccatgctgacgattggtgatggtctggttgcccaaattccggcgttgctgatcgcgatttctgcgggcttcatcgttacccgcgtcaacggtgatagcgataacatgggtcgtaacattatgacccagctgctgaataatccgtttgtcctggttgtaacggcgattttgaccatcagcatgggcacgctgccgggctttccgttgccggttttcgttattctgtctgttgtgctgtccgtcctgttttactttaagttccgcgaggcgaaacgtagcgctgcgaaaccaaaaacgagcaagggcgagcaaccgttgtccatcgaggagaaggaaggtagcagcctgggcctgattggcgacctggataaggttagcacggaaaccgtcccgctgattttgctggtgccgaaatcgcgtcgtgaggatctggagaaagcgcagctggcggaacgtctgcgcagccaattctttattgattatggtgtgcgtctgccagaagtactgctgcgtgacggtgagggtctggatgacaactctatcgtcctgctgattaatgagattcgcgttgaacagtttactgtctattttgacctgatgcgtgtggttaactacagcgacgaggtggtgagctttggcatcaacccgaccattcaccagcaaggttccagccagtacttttgggtgacccatgaggaaggcgaaaagctgcgcgagctgggctacgtcctgcgtaatgcactggacgaactgtaccactgtctggcggtgacgctggcacgcaatgtgaacgagtatttcggtatccaagaaacgaaacacatgctggaccaactggaagcaaagtttcctgacctgctgaaggaggttttgcgccacgccaccgtgcagcgcatttcggaagtgctgcaacgtctgctgtccgaacgcgtgagcgtccgtaacatgaagctgatcatggaagccctggcactgtgggctccgcgtgagaaagatgtgatcaatctggtggagcacatccgtggtgcgatggcgcgttatatctgccacaagttcgcaaatggtggtgaactgcgtgccgttatggtttccgccgaagttgaggatgtcattcgtaaaggcattcgtcaaacttctggctccacctttttgagcttggacccggaggcttcggcaaatctgatggacctgatcacgctgaagctggacgacctgttgattgcgcataaggacctggtcctgttgaccagcgttgacgtgcgtcgttttatcaagaaaatgattgaaggtcgttttccggatctggaggtcctgtccttcggtgagattgcagatagcaaaagcgtgaatgtcatcaaaaccatctga

SEQ ID NO: 132 - DNA, 36 bp, synthetic construct
  misc_feature 1..36: note = synthetic SynInvC ribosome binding site (rbs, RBS) of bacterial type III secretion system (T3SS)
  source 1..36: mol_type = other DNA
cttgggcacgcgtccattaaagaggaccaattaagc

SEQ ID NO: 133 - DNA, 1296 bp, synthetic construct
  misc_feature 1..1296: note = synthetic SynInvC of bacterial type III secretion system (T3SS)
  source 1..1296: mol_type = other DNA
atgaaaaccccacgtctgctgcaatacctggcctacccgcagaaaatcactggccctatcattgaagcagaactgcgtgatgttgcaattggtgaattgtgcgagatccgtcgcggctggcaccagaagcaggttgtggcccgtgcgcaagtggttggtttgcagcgcgaacgtaccgtcctgagcctgatcggcaatgcccaaggcctgagccgtgatgtggtcttgtacccgaccggccgtgctctgagcgcgtgggttggttacagcgttctgggcgcagtactggacccgacgggtaaaatcgttgaacgtttcaccccggaagtcgcaccgatttccgaggagcgcgttatcgacgtggcaccgccgagctacgcaagccgtgtcggtgtgcgcgaaccgctgatcacgggtgtccgcgcaattgatggtctgctgacgtgtggtgtgggccagcgtatgggtattttcgcaagcgcgggttgtggtaagaccatgttgatgcacatgctgatcgagcaaaccgaagcggatgtctttgttattggcctgattggcgagcgtggtcgtgaagttaccgaatttgtagacatgttgcgtgcatctcataagaaagaaaaatgtgtgctggtttttgccacgtccgacttcccaagcgttgaccgctgcaacgctgcccagctggcaacgacggtcgccgagtatttccgcgaccagggtaaacgtgttgtcctgtttatcgacagcatgacccgttatgcacgcgcgttgcgtgatgtcgcgctggcgagcggcgagcgtccggctcgccgtggttatccggcgtctgtgttcgacaatctgccgcgtttgctggagcgtccgggtgcgacgagcgagggtagcattaccgccttctataccgtcctgctggagtcggaagaagaagcggacccgatggcggacgagatccgttctattctggacggtcacctgtacctgtcccgcaaactggcgggtcagggtcattacccggctatcgatgtgctgaagagcgtgagccgtgtgtttggtcaagtgaccaccccgactcacgcggagcaagcgagcgcggtccgtaagctgatgacccgtctggaagagctgcaactgttcattgacctgggcgagtatcgtccgggcgagaacattgacaatgatcgtgcgatgcaaatgcgcgatagcctgaaggcgtggctgtgtcagcctgttgcgcaatacagcagcttcgatgatacgctgtccggcatgaacgcctttgcggatcagaactga

SEQ ID NO: 134 - DNA, 36 bp, synthetic construct
  misc_feature 1..36: note = synthetic SynInvE ribosome binding site (rbs, RBS) of bacterial type III secretion system (T3SS)
  source 1..36: mol_type = other DNA
tacttgggcacgcgtccattaattaggatcaatagc

SEQ ID NO: 135 - DNA, 1119 bp, synthetic construct
  misc_feature 1..1119: note = synthetic SynInvE of bacterial type III secretion system (T3SS)
  source 1..1119: mol_type = other DNA
atgattccgggcagcacctccggtatttcctttagccgtatcctgagccgtcagacctcccaccaggatgcaacccagcataccgacgcacaacaagcggaaattcaacaagcggcggaagatagctcgccgggtgcggaggttcagaaattcgtccagagcacggacgagatgtctgctgcgttggcgcagttccgcaatcgccgtgactatgagaaaaagagcagcaatttgtctaactccttcgagcgcgttctggaggacgaggcactgccgaaagcgaagcagattctgaaactgatcagcgtgcatggcggtgcgttggaggatttcctgcgtcaggcgcgcagcctgttcccggacccaagcgatctggtgctggttctgcgcgagctgttgcgtcgtaaggacctggaggagatcgtgcgtaagaagctggagagcctgctgaagcacgtggaggaacaaaccgacccgaaaaccctgaaggccggtattaactgcgcgctgaaggcgcgtctgtttggcaagacgctgtctctgaaacctggtctgctgcgtgccagctaccgccagttcatccaaagcgaaagccacgaagtcgagatttacagcgattggatcgccagctacggttatcagcgtcgcctggttgttctggatttcattgaaggcagcctgctgactgacatcgatgctaacgatgcaagctgctcccgtctggagtttggccaactgctgcgccgtctgacccagctgaaaatgttgcgtagcgccgacctgctgtttgtctcgacgttgctgtcttacagcttcacgaaagcatttaacgctgaggagagcagctggctgttgctgatgctgtctttgctgcaacagccgcacgaagtggatagcctgctggcggacattatcggtctgaatgcgctgctgttgtcccacaaagagcacgccagcttcctgcaaatcttctatcaggtctgtaaggcaatcccgtctagcctgttttatgaagagtactggcaagaagaactgctgatggcactgcgctccatgacggacattgcttacaaacacgaaatggccgaacaacgtcgtaccatcgaaaagctgtcctaa

SEQ ID NO: 136 - DNA, 36 bp, synthetic construct
  misc_feature 1..36: note = synthetic SynInvF ribosome binding site (rbs, RBS) of bacterial type III secretion system (T3SS)
  source 1..36: mol_type = other DNA
cttgggcacgcgtccattaaaaaggagtaattaagc

SEQ ID NO: 137 - DNA, 651 bp, synthetic construct
  misc_feature 1..651: note = synthetic SynInvF of bacterial type III secretion system (T3SS)
  source 1..651: mol_type = other DNA
atgagcttcagcgagagccgccacaatgaaaactgtctgattcaagaaggcgcactgctgttttgtgagcaagcagtcgtggcgcctgtcagcggtgatctggtttttcgtccgctgaaaatcgaggtcctgagcaagctgctggcgttcatcgacggcgcaggtctggtggatacgacctacgcggagtcggacaaatgggttctgctgtctccggagttccgtgctatttggcaagaccgtaaacgttgcgaatattggtttttgcagcagattatcaccccatctccggcgttcaacaaggttctggcactgttgcgtaagagcgaaagctattggttggtcggctacttgctggcccaaagcaccagcggcaatactatgcgtatgttgggtgaggattacggtgttagctacacgcatttccgccgtctgtgcagccgcgctctgggcggtaaggcgaaaagcgagctgcgcaattggcgcatggcccagtccctgctgaatagcgtggaaggtcatgaaaacatcacccagctggcggtcaaccacggttatagcagcccgtcccactttagctctgaaatcaaggagctgattggtgtttccccgcgtaagctgtctaacatcattcagctggccgacaaatga

SEQ ID NO: 138 - DNA, 36 bp, synthetic construct
  misc_feature 1..36: note = synthetic SynInvG ribosome binding site (rbs, RBS) of bacterial type III secretion system (T3SS)
  source 1..36: mol_type = other DNA
tgggcacgcgtccattaatgaggaaaaattattagc

SEQ ID NO: 139 - DNA, 1689 bp, synthetic construct
  misc_feature 1..1689: note = synthetic SynInvG of bacterial type III secretion system (T3SS)
  source 1..1689: mol_type = other DNA
atgaaaacgcacattctgttggcccgtgtgctggcttgcgcagctctggtgctggtcaccccaggttatagctccgagaagatcccggttacgggcagcggcttcgttgcaaaggacgattctctgcgcacctttttcgatgcgatggcactgcaattgaaggagccggtgattgtcagcaagatggcggctcgcaaaaagattaccggcaatttcgagttccacgatccaaacgcgctgctggagaaactgtccctgcaactgggtctgatctggtactttgatggtcaggcgatctacatctacgacgcgagcgaaatgcgtaatgcggttgtgagcctgcgtaatgtcagcctgaacgagttcaacaattttctgaagcgcagcggcctgtacaataagaactaccctctgcgtggtgataatcgtaaaggcaccttctatgtcagcggtccgccggtgtatgttgatatggttgtaaatgcggccaccatgatggacaaacagaatgatggcatcgagctgggtcgccaaaagatcggtgttatgcgcctgaacaacacttttgtgggcgaccgcacctacaacctgcgtgatcaaaagatggtcattccgggtattgctacggcaattgaacgcctgttgcaaggcgaagaacaaccgctgggtaacattgtaagctccgagcctccggccatgccggcctttagcgcaaacggcgagaaaggtaaggcagcgaattacgcgggtggtatgagcctgcaagaagcgctgaaacagaacgcagcggcaggcaacatcaaaattgtggcctatccggacaccaacagcctgctggtgaaaggtacggcggagcaggtgcatttcatcgagatgctggttaaagccctggacgtggcgaaacgtcacgttgaattgagcctgtggattgtggatttgaataagagcgacctggaacgcttgggcaccagctggagcggtagcatcaccatcggcgacaagctgggtgtgagcctgaaccagagcagcatctccacgctggacggtagccgctttattgcggcggtcaacgctctggaggaaaagaaacaggccactgttgtcagccgtccggttctgctgacccaggagaatgtcccggcgatttttgacaataatcgtactttctataccaaactgatcggtgaacgtaatgttgcattggaacacgtgacctatggcaccatgatccgtgtcctgccgcgtttcagcgcggacggccagattgagatgagcctggacatcgaagatggtaatgacaaaaccccgcagtctgatacgaccacctccgttgatgcgctgccagaagtgggtcgtaccctgatctcgacgattgcacgtgtcccgcatggtaaatctctgctggttggtggctacacgcgtgatgcaaacacggacacggtccaaagcatcccgttcctgggtaagctgccgctgattggctcgttgtttcgctacagcagcaaaaacaagtctaatgtcgtccgtgtctttatgattgagccgaaagaaatcgttgacccgctgaccccggatgccagcgagagcgttaacaacattctgaaacagtccggtgcgtggagcggtgacgataagctgcaaaagtgggttcgtgtgtatttggaccgtggtcaggaggccattaagtaa

SEQ ID NO: 140 - DNA, 36 bp, synthetic construct
  misc_feature 1..36: note = synthetic SynInvI ribosome binding site (rbs, RBS) of bacterial type III secretion system (T3SS)
  source 1..36: mol_type = other DNA
cttgggcacgcgtccattaaaaaggaccaattaagc

SEQ ID NO: 141 - DNA, 444 bp, synthetic construct
  misc_feature 1..444: note = synthetic SynInvI of bacterial type III secretion system (T3SS)
  source 1..444: mol_type = other DNA
atgcactctctgactcgtatcaaggtcctgcaacgtcgttgtaccgtgttccattctcagtgcgagtccattctgttgcgttatcaggacgaagatcgcggcttgcaggcggaggaggaggccatcctggaacagatcgcaggtctgaaactgctgttggacaccctgcgtgctgaaaatcgtcaactgagccgtgaagaaatctatacgctgctgcgcaaacagagcattgttcgtcgccagattaaggatctggagctgcaaatcattcagattcaagaaaagcgtagcgagctggaaaagaaacgtgaggaatttcaaaagaaaagcaaatactggctgcgtaaagaaggtaactaccagcgctggattatccgtcaaaaacgcttctacattcaacgtgagatccagcaagaggaggcggagagcgaagagatcatttaa

SEQ ID NO: 142 - DNA, 36 bp, synthetic construct
  misc_feature 1..36: note = synthetic SynInvJ ribosome binding site (rbs, RBS) of bacterial type III secretion system (T3SS)
  source 1..36: mol_type = other DNA
aaaaaaaagctctaaaagattaagagggggtaacat

SEQ ID NO: 143 - DNA, 1011 bp, synthetic construct
  misc_feature 1..1011: note = synthetic SynInvJ of bacterial type III secretion system (T3SS)
  source 1..1011: mol_type = other DNA
atgggtgacgtgagcgcggtgagcagcagcggtaacattctgctgccgcagcaggacgaggttggtggcctgtccgaagcgctgaagaaagcggttgaaaaacacaaaaccgaatacagcggtgacaagaaagatcgtgattatggtgacgcctttgttatgcacaaggaaaccgcgctgccgttgttgctggcagcttggcgccacggcgcaccggcgaaaagcgagcaccataacggtaacgtaagcggtctgcatcacaacggtaagagcgagctgcgtattgctgagaaactgctgaaggtgacggcggagaagagcgttggtctgattagcgctgaagcgaaggtggataaatctgcggcgctgctgtctagcaagaatcgtccgctggaatcggtcagcggcaaaaagttgtccgccgatctgaaagcagtggagtccgtgtccgaggtcacggataacgccaccggcatttcggatgacaacatcaaagcattgccgggtgacaataaggccatcgccggtgagggtgtgcgtaaagaaggtgcgccgctggcgcgtgacgtggctccggcacgcatggcggcagcaaatacgggcaagccggaggataaagaccacaagaaggtcaaggacgttagccagctgccgctgcaaccgactaccatcgccgatctgtctcaactgacgggtggcgatgaaaagatgccgctggcagcgcagtccaaaccgatgatgaccattttcccaaccgccgacggcgttaaaggtgaggacagctctctgacctatcgtttccagcgctggggcaacgattactccgtcaatatccaggcacgccaagcgggcgaatttagcctgattcctagcaatacccaggttgaacatcgtctgcacgaccaatggcaaaatggcaatccacaacgctggcatttgacgcgtgatgaccagcaaaacccgcaacagcaacagcatcgtcagcagtccggtgaagaggacgacgcgtaa

SEQ ID NO: 144 - DNA, 36 bp, synthetic construct
  misc_feature 1..36: note = synthetic SynSpaO ribosome binding site (rbs, RBS) of bacterial type III secretion system (T3SS)
  source 1..36: mol_type = other DNA
cttgggcacgcgtccatgaaagacaggacccactag

SEQ ID NO: 145 - DNA, 912 bp, synthetic construct
  misc_feature 1..912: note = synthetic SynSpaO of bacterial type III secretion system (T3SS)
  source 1..912: mol_type = other DNA
atgagcttgcgcgtacgccaaattgaccgccgtgaatggctgctggcccagactgcgaccgaatgtcagcgtcacggtcgtgaggccaccctggagtatccgacccgtcagggtatgtgggtccgcctgtctgatgccgaaaaacgctggtctgcgtggatcaaaccgggtgattggttggaacacgttagcccagcactggcgggtgccgcggtcagcgcaggcgcggagcacctggtggttccgtggctggcggcaaccgaacgcccgttcgagctgccggtcccgcacttgagctgccgtcgtctgtgcgtggagaacccggttccgggttccgcactgcctgagggcaagctgctgcacatcatgtcggatcgtggtggcctgtggtttgagcatctgccggagctgccggctgttggcggtggccgtccgaagatgctgcgttggccgctgcgtttcgttattggcagcagcgacacccagcgcagcctgctgggtcgtatcggtatcggtgatgttctgctgatccgcaccagccgtgctgaggtttactgttacgcgaagaaactgggccactttaatcgtgtggaaggtggcattattgtcgaaacgctggacattcaacatatcgaggaggagaacaacacgacggaaacggcggaaaccctgccgggtctgaatcaactgccggtgaagctggagttcgttctgtatcgtaaaaacgtgacgttggccgaactggaagcaatgggtcagcaacaactgctgtccttgccaaccaatgcggaactgaacgtcgaaatcatggcgaatggcgtgctgctgggtaacggcgaactggtgcagatgaatgacaccctgggtgtcgagattcatgagtggttgtccgagagcggtaatggcgagtag

SEQ ID NO: 146 - DNA, 36 bp, synthetic construct
  misc_feature 1..36: note = synthetic SynSpaP ribosome binding site (rbs, RBS) of bacterial type III secretion system (T3SS)
  source 1..36: mol_type = other DNA
cttgggcacgcgtccatgaaagaaacgacatactag

SEQ ID NO: 147 - DNA, 675 bp, synthetic construct
  misc_feature 1..675: note = synthetic SynSpaP of bacterial type III secretion system (T3SS)
  source 1..675: mol_type = other DNA
atgggtaacgatattagcttgattgcattgctggcgttttccaccctgctgccgttcatcatcgcgtctggtacttgcttcgtcaaattcagcatcgtctttgtgatggtgcgcaacgcgctgggtctgcaacaaattccaagcaatatgaccctgaatggcgtcgcactgctgctgtcgatgtttgttatgtggccgattatgcacgacgcgtatgtgtatttcgaggatgaagatgtgacctttaacgacatctccagcctgagcaagcatgttgatgagggcctggacggttatcgcgactacctgatcaagtattccgaccgtgagctggtgcagttctttgagaatgcccagttgaaacgtcagtacggtgaagaaacggaaaccgttaaacgtgacaaggacgagattgaaaagccgagcattttcgcactgttgcctgcttacgccttgagcgagattaagagcgcattcaaaattggtttttacctgtacctgccgttcgttgtggtcgatctggttgtctccagcgttctgctggccctgggcatgatgatgatgtccccggttaccatcagcacgccgatcaaactggtcctgtttgtggccctggatggttggacgctgctgtctaaaggcctgatcctgcaatacatggacatcgcgacctaa

SEQ ID NO: 148 - DNA, 36 bp, synthetic construct
  misc_feature 1..36: note = synthetic SynSpaQ ribosome binding site (rbs, RBS) of bacterial type III secretion system (T3SS)
  source 1..36: mol_type = other DNA
cttgggcacgcgtccattaagaaggaggaattaagc

SEQ ID NO: 149 - DNA, 261 bp, synthetic construct
  misc_feature 1..261: note = synthetic SynSpaQ of bacterial type III secretion system (T3SS)
  source 1..261: mol_type = other DNA
atggacgatctggttttcgccggcaacaaagccctgtacttggtgctgattctgtccggttggccgacgattgtcgcaaccattatcggtctgctggttggtctgtttcaaaccgtgacgcagttgcaggagcaaaccctgccgttcggtatcaagctgctgggtgtgtgtctgtgcctgtttttgctgtctggctggtatggcgaagttctgctgtcctacggccgtcaggtcatcttcctggctctggcgaaaggttaa

SEQ ID NO: 150 - DNA, 36 bp, synthetic construct; identical to SEQ ID NO: 144
  misc_feature 1..36: note = synthetic SynSpaR ribosome binding site (rbs, RBS) of bacterial type III secretion system (T3SS)
  source 1..36: mol_type = other DNA

SEQ ID NO: 151 - DNA, 792 bp, synthetic construct
  misc_feature 1..792: note = synthetic SynSpaR of bacterial type III secretion system (T3SS)
  source 1..792: mol_type = other DNA
atgttctatgcattgtattttgagatccaccatctggtggcgtccgcggctctgggttttgcgcgtgttgctccgatctttttctttctgccgttcctgaatagcggtgtcctgagcggtgctccgcgcaacgcgatcatcattctggtggcgctgggtgtatggccgcacgccctgaatgaggcgccaccgtttctgtctgtggcaatgattccgctggtcctgcaagaggcagccgtgggtgttatgctgggttgcctgttgtcctggccgttttgggttatgcacgcgttgggctgtatcattgataaccaacgcggtgcaaccctgtccagcagcattgatcctgcgaatggcatcgacaccagcgagatggccaatttcctgaacatgttcgcggctgtcgtgtatttgcagaacggtggcttggtcacgatggtggacgtgctgaataagtcttaccagctgtgtgatccgatgaacgagtgtacgccgagcctgcctccgttgctgaccttcattaatcaagtggcccagaacgcactggtgctggcgtccccggtcgttctggttctgctgctgtcggaagttttcctgggcctgctgtctcgttttgcaccgcaaatgaacgcgttcgccattagcctgactgttaaaagcggtattgcggttttgatcatgctgctgtatttcagcccggtcctgccggacaatgttctgcgtctgagctttcaggcgaccggcctgagcagctggttctacgaacgtggcgcaacgcatgtgctggaataa

SEQ ID NO: 152 - DNA, 36 bp, synthetic construct; identical to SEQ ID NO: 144
  misc_feature 1..36: note = synthetic SynSpaS ribosome binding site (rbs, RBS) of bacterial type III secretion system (T3SS)
  source 1..36: mol_type = other DNA

SEQ ID NO: 153 - DNA, 1071 bp, synthetic construct
  misc_feature 1..1071: note = synthetic SynSpaS of bacterial type III secretion system (T3SS)
  source 1..1071: mol_type = other DNA
atgtccagcaacaaaaccgaaaaaccgactaagaaacgtctggaggatagcgcaaagaaaggtcagagcttcaagagcaaggacctgattatcgcgtgcctgaccctgggtggtatcgcttatttggtgagctacggcagcttcaatgagtttatgggtatcattaagattatcatcgctgataactttgatcagtcgatggcagattatagcctggccgtgtttggtattggcctgaaatacctgattccgttcatgctgctgtgtttggtttgttccgcactgccggcactgctgcaagcgggcttcgttctggcaaccgaggccctgaagccgaatctgtccgccctgaacccggttgaaggcgcgaagaaactgttttccatgcgcaccgtcaaagacacggtcaagacgctgctgtatctgtcgagctttgtggttgcggcaatcatttgctggaaaaagtataaagtcgagattttcagccaactgaacggtaatatcgtgggtattgcggttatctggcgtgaattgctgctggcgttggttctgacctgtctggcgtgcgcgctgatcgtgctgttgctggatgctattgccgagtactttctgaccatgaaagatatgaagatggacaaagaagaagttaaacgcgagatgaaagagcaggagggtaacccggaggtgaagtctaaacgtcgtgaagtccacatggaaatcctgagcgaacaagtcaagtctgacattgaaaatagccgtctgattgtggcaaaccctacgcatattaccatcggcatctacttcaaaccggaactgatgccgattccaatgattagcgtctatgaaaccaatcaacgcgcgctggcggtccgtgcgtacgccgagaaagtgggtgttccggttattgtagacatcaagctggcgcgcagcctgttcaaaacgcaccgtcgttacgacctggtgagcctggaggagatcgacgaggttttgcgcctgctggtttggttggaagaggtcgaaaacgcaggcaaggatgtgatccaaccgcaggagaatgaagtgcgtcattaa

SEQ ID NO: 154 - DNA, 4075 bp, synthetic construct
  misc_feature 1..4075: note = synthetic Syn_PrgOrg operon of bacterial type III secretion system (T3SS)
  source 1..4075: mol_type = other DNA
gcatgcattaaagaggagaaattaagcatggctactccgtggtctggttatctggatgatgtttctgcaaaatttgacacgggtgttgacaacttgcaaacccaagttaccgaagccctggacaagctggctgcgaagccgtccgatccggcgctgctggcggcgtatcaatccaaactgtctgagtataacttgtaccgtaatgcgcagtcgaacacggtcaaggtcttcaaagatattgatgcagcgattattcagaactttcgttagtaacctaggattaaggaggataaattaagcatgtctatcgcgactatcgtgcctgaaaatgccgttattggtcaagcggtgaatattcgcagcatggaaacggacatcgtcagcttggacgaccgtttgctgcaagcattttcgggcagcgccatcgctaccgccgtcgataagcagaccattaccaatcgcattgaagaccctaatctggttaccgatccgaaggagctggcgattagccaggaaatgatttccgactacaatctgtacgtcagcatggttagcaccctgacgcgtaagggcgttggcgctgttgagactttgctgcgttcctgataggctagcattaaacaggataataagcatggaaactagcaaggagaaaacgattacgtcccctggtccgtatatcgttcgtctgttgaactcgtccctgaacggttgtgaatttccgctgctgactggtcgtacgctgttcgtcgtgggtcagagcgatgctctgaccgcgtctggtcagctgccggacattcctgccgactccttcttcatcccgctggatcatggcggtgttaatttcgagattcaagtggacactgacgcgacggaaatcatcctgcacgaactgaaagagggcaactccgagagccgctccgtgcaactgaacaccccgatccaagttggtgaactgctgattttgattcgtccggagagcgagccgtgggtgccggaacagccggagaagttggaaacttctgcgaaaaagaacgaaccgcgctttaaaaacggcatcgtcgcggcactggcgggtttctttatcctgggtatcggcacggttggcaccctgtggattctgaactcgccgcaacgtcaagcagccgaattggacagcctgttgggtcaggagaaggagcgttttcaggtgctgccgggtcgcgacaagatgctgtatgtcgccgcgcaaaacgaacgcgacaccctgtgggcacgtcaagtcctggcacgcggcgattacgataaaaacgcacgcgttattaatgaaaatgaggagaataaacgtatcagcatctggctggacacgtattatccacaactggcatattaccgtatccattttgatgaaccacgtaagccggtgttttggctgtcccgtcaacgcaacacgatgagcaagaaagagctggaggtgctgtcccagaaattgcgtgcgctgatgccgtacgccgacagcgtcaatattactctgatggatgacgtgaccgcagcaggccaagccgaggcaggtctgaaacaacaggcgctgccatacagccgccgtaaccacaaaggtggtgttacgttcgttattcagggcgccttggacgacgttgagattctgcgtgcgcgccagtttgtcgactcctattatcgtacctggggtggtcgttacgttcaattcgcaattgaattgaaagacgattggctgaaaggccgctcgttccaatacggtgcggaaggctacattaagatgagcccaggtcattggtattttccgtctcctctgtaatagaagcttattaaacaggataataagcatgatccgccgttacctgtataccttcttgctggttatgactttggccggctgtaaagataaggatctgctgaaaggcttggaccaagagcaagcgaatgaggtcattgcggttctgcaaatgcacaacattgaggctaacaagattgatagcggcaagctgggttacagcattaccgtcgcggaaccggatttcaccgcggcagtgtattggattaagacctaccagttgccgcctcgcccgcgtgtcgaaatcgcccaaatgtttccggcagacagcctggttagctctccgcgtgcggagaaagcacgtctgtactcggcgattgaacagcgcctggagcagtcgctgcaaacgatggaaggtgttctgtcggcccgtgtccacatcagctatgatattgatgcgggcgagaacggtcgtccgcctaagccggtgcacctgtcggctttggcggtgtatgaacgcggttcccctctggcccatcagatttcggatattaagcgcttcctgaaaaacagcttcgcggatgttgactatgataacatcagcgtggttctgtccgagcgtagcgacgcacagttgcaggcgccgggcacgccggtcaagcgcaatagcttcgctacctcctggattgtgctgattatcctgttgtctgttatgagcgcgggtttcggtgtctggtactacaaaaatcactatgcgcgtaataagaaaggcattactgccgatgacaaggcaaagtccagcaacgagtaataaggtaccgaaagaagggacagactagatgattcgtcgtaaccgtcaaatgaaccgtcaaccactgccaattatctggcaacgcattattttcgacccactgtcctatattcacccacaacgtctgcaaatcgcgccggagatgatcgtgcgtccggcagcgcgtgcggcagcgaatgagctgattttggcggcgtggcgtttgaagaacggtgagaaggagtgcattcagaatagcctgacgcagctgtggttgcgtcaatggcgccgtctgccgcaggttgcttacctgctgggttgccacaagttgcgtgctgacctggcccgtcaaggtgctttattgggcctgccggactgggcgcaggcattcttggcgatgcaccagggtacgtccttgtcggtttgtaataaggcgccgaaccaccgtttcctgctgtccgttggttacgcccaactgaacgcgctgaatgagttcctgccggagagcttggcccaacgctttcctctgctgtttccaccgttcatcgaggaggcactgaaacaagatgcagtggagatgagcatcctgctgctggccctgcaatacgcgcaaaagtatcctaacaccgtcccggcgtttgcgtgctaataaagatctgaaagaagggacagactagatgttgaaaaatatcccgattccatcccctctgtctccggttgaaggtatcctgattaaacgcaagacgttggagcgttacttctcgattgagcgcctggaacaacaggcgcatcagcgtgcaaagcgcattttgcgtgaggcagaagaagaagccaagaccctgcgcatgtatgcgtaccaggagggctacgagcagggcatgattgacgcactgcaacaggtggccgcctatttgaccgacaaccagacgatggcttggaaatggatggagaaaattcaaatctatgcgcgtgagttgttcagcgcggctgtcgatcacccggaaacgttgttgacggtgctggacgagtggctgcgtgatttcgataagccggaaggtcagctgtttttgaccctgccggtgaacgcaaagaaagatcatcagaaactgatggtgctgctgatggaaaattggccgggcaccttcaatctgaagtatcatcaggagcaacgttttatcatgtcctgtggcgatcagattgccgagttttcgccggaacaatttgttgaaacggcggttggtgttatcaagcaccatctggatgagctgcctcaggactgtcgcaccatttcggacaatgcgattaacgcgctgattgatgaatggaagacgaaaacccaagctgaagttattcgctgataaggatccggcatcaaataaaacgaaaggctcagtcgaaagactgggcctttcgttttatctgttgtttgtcggtgaacgctctcctgagtaggacaaatccgccgccctagaactagtccataagaatgcggccgc

SEQ ID NO: 155 - DNA, 12614 bp, synthetic construct
  misc_feature 1..12614: note = synthetic Syn_InvSpa operon of bacterial type III secretion system (T3SS)
  source 1..12614: mol_type = other DNA
ctcgagaaaaaaaagctctaaaagattaagagggggtaacatatgggtgacgtgagcgcggtgagcagcagcggtaacattctgctgccgcagcaggacgaggttggtggcctgtccgaagcgctgaagaaagcggttgaaaaacacaaaaccgaatacagcggtgacaagaaagatcgtgattatggtgacgcctttgttatgcacaaggaaaccgcgctgccgttgttgctggcagcttggcgccacggcgcaccggcgaaaagcgagcaccataacggtaacgtaagcggtctgcatcacaacggtaagagcgagctgcgtattgctgagaaactgctgaaggtgacggcggagaagagcgttggtctgattagcgctgaagcgaaggtggataaatctgcggcgctgctgtctagcaagaatcgtccgctggaatcggtcagcggcaaaaagttgtccgccgatctgaaagcagtggagtccgtgtccgaggtcacggataacgccaccggcatttcggatgacaacatcaaagcattgccgggtgacaataaggccatcgccggtgagggtgtgcgtaaagaaggtgcgccgctggcgcgtgacgtggctccggcacgcatggcggcagcaaatacgggcaagccggaggataaagaccacaagaaggtcaaggacgttagccagctgccgctgcaaccgactaccatcgccgatctgtctcaactgacgggtggcgatgaaaagatgccgctggcagcgcagtccaaaccgatgatgaccattttcccaaccgccgacggcgttaaaggtgaggacagctctctgacctatcgtttccagcgctggggcaacgattactccgtcaatatccaggcacgccaagcgggcgaatttagcctgattcctagcaatacccaggttgaacatcgtctgcacgaccaatggcaaaatggcaatccacaacgctggcatttgacgcgtgatgaccagcaaaacccgcaacagcaacagcatcgtcagcagtccggtgaagaggacgacgcgtaacttgggcacgcgtccattaagaaggaggaattaagcatggacgatctggttttcgccggcaacaaagccctgtacttggtgctgattctgtccggttggccgacgattgtcgcaaccattatcggtctgctggttggtctgtttcaaaccgtgacgcagttgcaggagcaaaccctgccgttcggtatcaagctgctgggtgtgtgtctgtgcctgtttttgctgtctggctggtatggcgaagttctgctgtcctacggccgtcaggtcatcttcctggctctggcgaaaggttaagacgtccttgggcacgcgtccattaaacaggagtaattaagcatgctgctgtccctgctgaatagcgcgcgtctgcgtcctgagctgctgattctggttctgatggttatgatcatcagcatgttcgttatcccgttgccgacctatttggttgacttcttgatcgctttgaacattgtcctggcaattctggtgttcatgggctccttctacatcgaccgcattctgagcttcagcacctttccggcggttctgctgatcacgactctgttccgtttggcactgagcatcagcaccagccgcctgatcctgattgaagcagatgcgggtgagatcatcgcgacctttggtcagtttgtgatcggtgacagcctggcggttggtttcgtcgtattctccatcgtgacggtggtgcagtttatcgttattaccaagggcagcgaacgtgtggcggaggtcgccgctcgcttcagcctggacggcatgccgggtaaacagatgtctattgatgcagacctgaaagccggcattattgatgctgatgcagcgcgcgagcgccgcagcgtcctggagcgtgaaagccaactgtacggttccttcgacggtgccatgaagttcattaaaggtgatgcgattgcgggcatcattatcatcttcgttaacttcattggcggtatcagcgtcggtatgacccgtcatggtatggatctgagcagcgccctgagcacctacaccatgctgacgattggtgatggtctggttgcccaaattccggcgttgctgatcgcgatttctgcgggcttcatcgttacccgcgtcaacggtgatagcgataacatgggtcgtaacattatgacccagctgctgaataatccgtttgtcctggttgtaacggcgattttgaccatcagcatgggcacgctgccgggctttccgttgccggttttcgttattctgtctgttgtgctgtccgtcctgttttactttaagttccgcgaggcgaaacgtagcgctgcgaaaccaaaaacgagcaagggcgagcaaccgttgtccatcgaggagaaggaaggtagcagcctgggcctgattggcgacctggataaggttagcacggaaaccgtcccgctgattttgctggtgccgaaatcgcgtcgtgaggatctggagaaagcgcagctggcggaacgtctgcgcagccaattctttattgattatggtgtgcgtctgccagaagtactgctgcgtgacggtgagggtctggatgacaactctatcgtcctgctgattaatgagattcgcgttgaacagtttactgtctattttgacctgatgcgtgtggttaactacagcgacgaggtggtgagctttggcatcaacccgaccattcaccagcaaggttccagccagtacttttgggtgacccatgaggaaggcgaaaagctgcgcgagctgggctacgtcctgcgtaatgcactggacgaactgtaccactgtctggcggtgacgctggcacgcaatgtgaacgagtatttcggtatccaagaaacgaaacacatgctggaccaactggaagcaaagtttcctgacctgctgaaggaggttttgcgccacgccaccgtgcagcgcatttcggaagtgctgcaacgtctgctgtccgaacgcgtgagcgtccgtaacatgaagctgatcatggaagccctggcactgtgggctccgcgtgagaaagatgtgatcaatctggtggagcacatccgtggtgcgatggcgcgttatatctgccacaagttcgcaaatggtggtgaactgcgtgccgttatggtttccgccgaagttgaggatgtcattcgtaaaggcattcgtcaaacttctggctccacctttttgagcttggacccggaggcttcggcaaatctgatggacctgatcacgctgaagctggacgacctgttgattgcgcataaggacctggtcctgttgaccagcgttgacgtgcgtcgttttatcaagaaaatgattgaaggtcgttttccggatctggaggtcctgtccttcggtgagattgcagatagcaaaagcgtgaatgtcatcaaaaccatctgaatttaaatcttgggcacgcgtccattaaaaaggagtaattaagcatgagcttcagcgagagccgccacaatgaaaactgtctgattcaagaaggcgcactgctgttttgtgagcaagcagtcgtggcgcctgtcagcggtgatctggtttttcgtccgctgaaaatcgaggtcctgagcaagctgctggcgttcatcgacggcgcaggtctggtggatacgacctacgcggagtcggacaaatgggttctgctgtctccggagttccgtgctatttggcaagaccgtaaacgttgcgaatattggtttttgcagcagattatcaccccatctccggcgttcaacaaggttctggcactgttgcgtaagagcgaaagctattggttggtcggctacttgctggcccaaagcaccagcggcaatactatgcgtatgttgggtgaggattacggtgttagctacacgcatttccgccgtctgtgcagccgcgctctgggcggtaaggcgaaaagcgagctgcgcaattggcgcatggcccagtccctgctgaatagcgtggaaggtcatgaaaacatcacccagctggcggtcaaccacggttatagcagcccgtcccactttagctctgaaatcaaggagctgattggtgtttccccgcgtaagctgtctaacatcattcagctggccgacaaatgaatttaaatgctagccttgggcacgcgtccattaaagaggaccaattaagcatgaaaaccccacgtctgctgcaatacctggcctacccgcagaaaatcactggccctatcattgaagcagaactgcgtgatgttgcaattggtgaattgtgcgagatccgtcgcggctggcaccagaagcaggttgtggcccgtgcgcaagtggttggtttgcagcgcgaacgtaccgtcctgagcctgatcggcaatgcccaaggcctgagccgtgatgtggtcttgtacccgaccggccgtgctctgagcgcgtgggttggttacagcgttctgggcgcagtactggacccgacgggtaaaatcgttgaacgtttcaccccggaagtcgcaccgatttccgaggagcgcgttatcgacgtggcaccgccgagctacgcaagccgtgtcggtgtgcgcgaaccgctgatcacgggtgtccgcgcaattgatggtctgctgacgtgtggtgtgggccagcgtatgggtattttcgcaagcgcgggttgtggtaagaccatgttgatgcacatgctgatcgagcaaaccgaagcggatgtctttgttattggcctgattggcgagcgtggtcgtgaagttaccgaatttgtagacatgttgcgtgcatctcataagaaagaaaaatgtgtgctggtttttgccacgtccgacttcccaagcgttgaccgctgcaacgctgcccagctggcaacgacggtcgccgagtatttccgcgaccagggtaaacgtgttgtcctgtttatcgacagcatgacccgttatgcacgcgcgttgcgtgatgtcgcgctggcgagcggcgagcgtccggctcgccgtggttatccggcgtctgtgttcgacaatctgccgcgtttgctggagcgtccgggtgcgacgagcgagggtagcattaccgccttctataccgtcctgctggagtcggaagaagaagcggacccgatggcggacgagatccgttctattctggacggtcacctgtacctgtcccgcaaactggcgggtcagggtcattacccggctatcgatgtgctgaagagcgtgagccgtgtgtttggtcaagtgaccaccccgactcacgcggagcaagcgagcgcggtccgtaagctgatgacccgtctggaagagctgcaactgttcattgacctgggcgagtatcgtccgggcgagaacattgacaatgatcgtgcgatgcaaatgcgcgatagcctgaaggcgtggctgtgtcagcctgttgcgcaatacagcagcttcgatgatacgctgtccggcatgaacgcctttgcggatcagaactgacttgggcacgcgtccatgaaagaaacgacatactagatgggtaacgatattagcttgattgcattgctggcgttttccaccctgctgccgttcatcatcgcgtctggtacttgcttcgtcaaattcagcatcgtctttgtgatggtgcgcaacgcgctgggtctgcaacaaattccaagcaatatgaccctgaatggcgtcgcactgctgctgtcgatgtttgttatgtggccgattatgcacgacgcgtatgtgtatttcgaggatgaagatgtgacctttaacgacatctccagcctgagcaagcatgttgatgagggcctggacggttatcgcgactacctgatcaagtattccgaccgtgagctggtgcagttctttgagaatgcccagttgaaacgtcagtacggtgaagaaacggaaaccgttaaacgtgacaaggacgagattgaaaagccgagcattttcgcactgttgcctgcttacgccttgagcgagattaagagcgcattcaaaattggtttttacctgtacctgccgttcgttgtggtcgatctggttgtctccagcgttctgctggccctgggcatgatgatgatgtccccggttaccatcagcacgccgatcaaactggtcctgtttgtggccctggatggttggacgctgctgtctaaaggcctgatcctgcaatacatggacatcgcgacctaagagctctgggcacgcgtccattaatgaggaaaaattattagcatgaaaacgcacattctgttggcccgtgtgctggcttgcgcagctctggtgctggtcaccccaggttatagctccgagaagatcccggttacgggcagcggcttcgttgcaaaggacgattctctgcgcacctttttcgatgcgatggcactgcaattgaaggagccggtgattgtcagcaagatggcggctcgcaaaaagattaccggcaatttcgagttccacgatccaaacgcgctgctggagaaactgtccctgcaactgggtctgatctggtactttgatggtcaggcgatctacatctacgacgcgagcgaaatgcgtaatgcggttgtgagcctgcgtaatgtcagcctgaacgagttcaacaattttctgaagcgcagcggcctgtacaataagaactaccctctgcgtggtgataatcgtaaaggcaccttctatgtcagcggtccgccggtgtatgttgatatggttgtaaatgcggccaccatgatggacaaacagaatgatggcatcgagctgggtcgccaaaagatcggtgttatgcgcctgaacaacacttttgtgggcgaccgcacctacaacctgcgtgatcaaaagatggtcattccgggtattgctacggcaattgaacgcctgttgcaaggcgaagaacaaccgctgggtaacattgtaagctccgagcctccggccatgccggcctttagcgcaaacggcgagaaaggtaaggcagcgaattacgcgggtggtatgagcctgcaagaagcgctgaaacagaacgcagcggcaggcaacatcaaaattgtggcctatccggacaccaacagcctgctggtgaaaggtacggcggagcaggtgcatttcatcgagatgctggttaaagccctggacgtggcgaaacgtcacgttgaattgagcctgtggattgtggatttgaataagagcgacctggaacgcttgggcaccagctggagcggtagcatcaccatcggcgacaagctgggtgtgagcctgaaccagagcagcatctccacgctggacggtagccgctttattgcggcggtcaacgctctggaggaaaagaaacaggccactgttgtcagccgtccggttctgctgacccaggagaatgtcccggcgatttttgacaataatcgtactttctataccaaactgatcggtgaacgtaatgttgcattggaacacgtgacctatggcaccatgatccgtgtcctgccgcgtttcagcgcggacggccagattgagatgagcctggacatcgaagatggtaatgacaaaaccccgcagtctgatacgaccacctccgttgatgcgctgccagaagtgggtcgtaccctgatctcgacgattgcacgtgtcccgcatggtaaatctctgctggttggtggctacacgcgtgatgcaaacacggacacggtccaaagcatcccgttcctgggtaagctgccgctgattggctcgttgtttcgctacagcagcaaaaacaagtctaatgtcgtccgtgtctttatgattgagccgaaagaaatcgttgacccgctgaccccggatgccagcgagagcgttaacaacattctgaaacagtccggtgcgtggagcggtgacgataagctgcaaaagtgggttcgtgtgtatttggaccgtggtcaggaggccattaagtaacttgggcacgcgtccatgaaagacaggacccactagatgtccagcaacaaaaccgaaaaaccgactaagaaacgtctggaggatagcgcaaagaaaggtcagagcttcaagagcaaggacctgattatcgcgtgcctgaccctgggtggtatcgcttatttggtgagctacggcagcttcaatgagtttatgggtatcattaagattatcatcgctgataactttgatcagtcgatggcagattatagcctggccgtgtttggtattggcctgaaatacctgattccgttcatgctgctgtgtttggtttgttccgcactgccggcactgctgcaagcgggcttcgttctggcaaccgaggccctgaagccgaatctgtccgccctgaacccggttgaaggcgcgaagaaactgttttccatgcgcaccgtcaaagacacggtcaagacgctgctgtatctgtcgagctttgtggttgcggcaatcatttgctggaaaaagtataaagtcgagattttcagccaactgaacggtaatatcgtgggtattgcggttatctggcgtgaattgctgctggcgttggttctgacctgtctggcgtgcgcgctgatcgtgctgttgctggatgctattgccgagtactttctgaccatgaaagatatgaagatggacaaagaagaagttaaacgcgagatgaaagagcaggagggtaacccggaggtgaagtctaaacgtcgtgaagtccacatggaaatcctgagcgaacaagtcaagtctgacattgaaaatagccgtctgattgtggcaaaccctacgcatattaccatcggcatctacttcaaaccggaactgatgccgattccaatgattagcgtctatgaaaccaatcaacgcgcgctggcggtccgtgcgtacgccgagaaagtgggtgttccggttattgtagacatcaagctggcgcgcagcctgttcaaaacgcaccgtcgttacgacctggtgagcctggaggagatcgacgaggttttgcgcctgctggtttggttggaagaggtcgaaaacgcaggcaaggatgtgatccaaccgcaggagaatgaagtgcgtcattaactgcaggtttaaactacttgggcacgcgtccattaattaggatcaatagcatgattccgggcagcacctccggtatttcctttagccgtatcctgagccgtcagacctcccaccaggatgcaacccagcataccgacgcacaacaagcggaaattcaacaagcggcggaagatagctcgccgggtgcggaggttcagaaattcgtccagagcacggacgagatgtctgctgcgttggcgcagttccgcaatcgccgtgactatgagaaaaagagcagcaatttgtctaactccttcgagcgcgttctggaggacgaggcactgccgaaagcgaagcagattctgaaactgatcagcgtgcatggcggtgcgttggaggatttcctgcgtcaggcgcgcagcctgttcccggacccaagcgatctggtgctggttctgcgcgagctgttgcgtcgtaaggacctggaggagatcgtgcgtaagaagctggagagcctgctgaagcacgtggaggaacaaaccgacccgaaaaccctgaaggccggtattaactgcgcgctgaaggcgcgtctgtttggcaagacgctgtctctgaaacctggtctgctgcgtgccagctaccgccagttcatccaaagcgaaagccacgaagtcgagatttacagcgattggatcgccagctacggttatcagcgtcgcctggttgttctggatttcattgaaggcagcctgctgactgacatcgatgctaacgatgcaagctgctcccgtctggagtttggccaactgctgcgccgtctgacccagctgaaaatgttgcgtagcgccgacctgctgtttgtctcgacgttgctgtcttacagcttcacgaaagcatttaacgctgaggagagcagctggctgttgctgatgctgtctttgctgcaacagccgcacgaagtggatagcctgctggcggacattatcggtctgaatgcgctgctgttgtcccacaaagagcacgccagcttcctgcaaatcttctatcaggtctgtaaggcaatcccgtctagcctgttttatgaagagtactggcaagaagaactgctgatggcactgcgctccatgacggacattgcttacaaacacgaaatggccgaacaacgtcgtaccatcgaaaagctgtcctaagtttaaaccttgggcacgcgtccatgaaagacaggacccactagatgagcttgcgcgtacgccaaattgaccgccgtgaatggctgctggcccagactgcgaccgaatgtcagcgtcacggtcgtgaggccaccctggagtatccgacccgtcagggtatgtgggtccgcctgtctgatgccgaaaaacgctggtctgcgtggatcaaaccgggtgattggttggaacacgttagcccagcactggcgggtgccgcggtcagcgcaggcgcggagcacctggtggttccgtggctggcggcaaccgaacgcccgttcgagctgccggtcccgcacttgagctgccgtcgtctgtgcgtggagaacccggttccgggttccgcactgcctgagggcaagctgctgcacatcatgtcggatcgtggtggcctgtggtttgagcatctgccggagctgccggctgttggcggtggccgtccgaagatgctgcgttggccgctgcgtttcgttattggcagcagcgacacccagcgcagcctgctgggtcgtatcggtatcggtgatgttctgctgatccgcaccagccgtgctgaggtttactgttacgcgaagaaactgggccactttaatcgtgtggaaggtggcattattgtcgaaacgctggacattcaacatatcgaggaggagaacaacacgacggaaacggcggaaaccctgccgggtctgaatcaactgccggtgaagctggagttcgttctgtatcgtaaaaacgtgacgttggccgaactggaagcaatgggtcagcaacaactgctgtccttgccaaccaatgcggaactgaacgtcgaaatcatggcgaatggcgtgctgctgggtaacggcgaactggtgcagatgaatgacaccctgggtgtcgagattcatgagtggttgtccgagagcggtaatggcgagtagagatctcttgggcacgcgtccattaaaaaggaccaattaagcatgcactctctgactcgtatcaaggtcctgcaacgtcgttgtaccgtgttccattctcagtgcgagtccattctgttgcgttatcaggacgaagatcgcggcttgcaggcggaggaggaggccatcctggaacagatcgcaggtctgaaactgctgttggacaccctgcgtgctgaaaatcgtcaactgagccgtgaagaaatctatacgctgctgcgcaaacagagcattgttcgtcgccagattaaggatctggagctgcaaatcattcagattcaagaaaagcgtagcgagctggaaaagaaacgtgaggaatttcaaaagaaaagcaaatactggctgcgtaaagaaggtaactaccagcgctggattatccgtcaaaaacgcttctacattcaacgtgagatccagcaagaggaggcggagagcgaagagatcatttaacttgggcacgcgtccatgaaagacaggacccactagatgttctatgcattgtattttgagatccaccatctggtggcgtccgcggctctgggttttgcgcgtgttgctccgatctttttctttctgccgttcctgaatagcggtgtcctgagcggtgctccgcgcaacgcgatcatcattctggtggcgctgggtgtatggccgcacgccctgaatgaggcgccaccgtttctgtctgtggcaatgattccgctggtcctgcaagaggcagccgtgggtgttatgctgggttgcctgttgtcctggccgttttgggttatgcacgcgttgggctgtatcattgataaccaacgcggtgcaaccctgtccagcagcattgatcctgcgaatggcatcgacaccagcgagatggccaatttcctgaacatgttcgcggctgtcgtgtatttgcagaacggtggcttggtcacgatggtggacgtgctgaataagtcttaccagctgtgtgatccgatgaacgagtgtacgccgagcctgcctccgttgctgaccttcattaatcaagtggcccagaacgcactggtgctggcgtccccggtcgttctggttctgctgctgtcggaagttttcctgggcctgctgtctcgttttgcaccgcaaatgaacgcgttcgccattagcctgactgttaaaagcggtattgcggttttgatcatgctgctgtatttcagcccggtcctgccggacaatgttctgcgtctgagctttcaggcgaccggcctgagcagctggttctacgaacgtggcgcaacgcatgtgctggaataaggatccggcatcaaataaaacgaaaggctcagtcgaaagactgggcctttcgttttatctgttgtttgtcggtgaacgctctcctgagtaggacaaatccgccgccctagaactagtccataagaatgcggccgc

SEQ ID NO: 156 - DNA, 23 bp, synthetic construct
  misc_feature 1..23: note = synthetic Enterobacteria phage T7 promoter seed sequence
  misc_difference 16..20: note = n = g, a, c or t
  source 1..23: mol_type = other DNA
taatacgactcactannnnnaga

SEQ ID NO: 157 - DNA, 23 bp, synthetic construct
  misc_feature 1..23: note = synthetic Enterobacteria phage T7 wild type (WT) promoter
  source 1..23: mol_type = other DNA
taatacgactcactatagggaga

SEQ ID NO: 158 - DNA, 23 bp, synthetic construct
  misc_feature 1..23: note = synthetic mutant Enterobacteria phage T7 promoter Mut1
  source 1..23: mol_type = other DNA
taatacgactcactacaggcaga

SEQ ID NO: 159 - DNA, 23 bp, synthetic construct
  misc_feature 1..23: note = synthetic mutant Enterobacteria phage T7 promoter Mut2
  source 1..23: mol_type = other DNA
taatacgactcactagagagaga

SEQ ID NO: 160 - DNA, 23 bp, synthetic construct
  misc_feature 1..23: note = synthetic mutant Enterobacteria phage T7 promoter Mut3
  source 1..23: mol_type = other DNA
taatacgactcactaatgggaga

SEQ ID NO: 161 - DNA, 23 bp, synthetic construct
  misc_feature 1..23: note = synthetic mutant Enterobacteria phage T7 promoter Mut4
  source 1..23: mol_type = other DNA
taatacgactcactataggtaga

SEQ ID NO: 162 - DNA, 23 bp, synthetic construct
  misc_feature 1..23: note = synthetic mutant Enterobacteria phage T7 promoter Mut5
  source 1..23: mol_type = other DNA
taatacgactcactaaagggaga

SEQ ID NO: 163 - DNA, 23 bp, synthetic construct
  misc_feature 1..23: note = synthetic mutant Enterobacteria phage T7 promoter Mut6
  source 1..23: mol_type = other DNA
taatacgactcactattgggaga

SEQ ID NO: 164 - DNA, 26 bp, synthetic construct
  misc_feature 1..26: note = synthetic Klebsiella oxytoca nif operon deletion mutant NF8 deletion region
  modified_base 26: mod_base = OTHER; note = c attached to SEQ ID NO: 165 by a Km cassette
  source 1..26: mol_type = other DNA
gcaggagaactaaaggcccggcatgc

SEQ ID NO: 165 - DNA, 26 bp, synthetic construct
  misc_feature 1..26: note = synthetic Klebsiella oxytoca nif operon deletion mutant NF8 deletion region
  modified_base 1: mod_base = OTHER; note = g attached to SEQ ID NO: 164 by a Km cassette
  source 1..26: mol_type = other DNA
gcatgcaagcgcccatggccccggca

SEQ ID NO: 166 - DNA, 46 bp, synthetic construct
  misc_feature 1..46: note = synthetic Klebsiella oxytoca nif in-frame deletion mutant UC12 deletion region
  source 1..46: mol_type = other DNA
cataaacaggcacggctggtggtaccccatcaggtgccccgcgtca

SEQ ID NO: 167 - DNA, 46 bp, synthetic construct
  misc_feature 1..46: note = synthetic Klebsiella oxytoca nif operon deletion mutant NF9 deletion region
  source 1..46: mol_type = other DNA
atggccccggcaggcgcaatgcatgcgacgctcttccccacgttac

SEQ ID NO: 168 - DNA, 46 bp, synthetic construct
  misc_feature 1..46: note = synthetic Klebsiella oxytoca nif operon deletion mutant UC14 deletion region
  source 1..46: mol_type = other DNA
gacgctcttccccacgttacgcatgcgatccggacccgcgccgcta

SEQ ID NO: 169 - DNA, 46 bp, synthetic construct
  misc_feature 1..46: note = synthetic Klebsiella oxytoca nif operon deletion mutant UC15 deletion region
  source 1..46: mol_type = other DNA
ctcttccccacgttacgctcgcatgcgatccggacccgcgccgcta

SEQ ID NO: 170 - DNA, 46 bp, synthetic construct
  misc_feature 1..46: note = synthetic Klebsiella oxytoca nif operon deletion mutant NF10 deletion region
  source 1..46: mol_type = other DNA
cggacccgcgccgctagccggcatgcatctttggcagcagagccag

SEQ ID NO: 171 - DNA, 46 bp, synthetic construct
  misc_feature 1..46: note = synthetic Klebsiella oxytoca nif operon deletion mutant NF11 deletion region
  source 1..46: mol_type = other DNA
cggacccgcgccgctagccggcatgcagcctcggcggctacccgtt

SEQ ID NO: 172 - DNA, 46 bp, synthetic construct
  misc_feature 1..46: note = synthetic Klebsiella oxytoca nif operon deletion mutant NF12 deletion region
  source 1..46: mol_type = other DNA
actggttatcctgatccagcgcatgcctttgcactaccgcggccca

SEQ ID NO: 173 - DNA, 46 bp, synthetic construct
  misc_feature 1..46: note = synthetic Klebsiella oxytoca nif operon deletion mutant NF13 deletion region
  source 1..46: mol_type = other DNA
ccgttaacgcctacagcacggcatgcctttgcactaccgcggccca

SEQ ID NO: 174 - DNA, 40 bp, synthetic construct
  misc_feature 1..40: note = synthetic Klebsiella oxytoca nif operon deletion mutant NF14 deletion region
  source 1..40: mol_type = other DNA
attcagggacgcgggttgccatgtgattatgcgacgtctt

SEQ ID NO: 175 - DNA, 46 bp, synthetic construct
  misc_feature 1..46: note = synthetic Klebsiella oxytoca nif operon deletion mutant NF24 deletion region
  source 1..46: mol_type = other DNA
gtcaaacaccagaatattgagagctctgtcgtttctgtgacaaagc

SEQ ID NO: 176 - DNA, 46 bp, synthetic construct
  misc_feature 1..46: note = synthetic Klebsiella oxytoca nif operon deletion mutant NF25 deletion region
  source 1..46: mol_type = other DNA
cggacccgcgccgctagccggcatgcctttgcactaccgcggccca

SEQ ID NO: 177 - DNA, 46 bp, synthetic construct
  misc_feature 1..46: note = synthetic Klebsiella oxytoca whole nif deletion mutant NF26 deletion region
  source 1..46: mol_type = other DNA
gtcaaacaccagaatattgactcgagatgtgattatgcgacgtctt

SEQ ID NO: 178 - DNA, 23 bp, synthetic construct
  misc_feature 1..23: note = synthetic wild type (WT) Enterobacteria phage T7 promoter part number SBa_000443
  source 1..23: mol_type = other DNA
taatacgactcactatagggaga

SEQ ID NO: 179 - DNA, 23 bp, synthetic construct
  misc_feature 1..23: note = synthetic Enterobacteria phage T7 promoter 1 part number SBa_000444
  source 1..23: mol_type = other DNA
taatacgactcactacaggcaga

SEQ ID NO: 180 - DNA, 23 bp, synthetic construct
  misc_feature 1..23: note = synthetic Enterobacteria phage T7 promoter 2 part number SBa_000445
  source 1..23: mol_type = other DNA
taatacgactcactagagagaga

SEQ ID NO: 181 - DNA, 23 bp, synthetic construct
  misc_feature 1..23: note = synthetic Enterobacteria phage T7 promoter 3 part number SBa_000446
  source 1..23: mol_type = other DNA
taatacgactcactaatgggaga

SEQ ID NO: 182 - DNA, 23 bp, synthetic construct
  misc_feature 1..23: note = synthetic Enterobacteria phage T7 promoter 4 part number SBa_000447
  source 1..23: mol_type = other DNA
taatacgactcactattgggaga

SEQ ID NO: 183 - DNA, 48 bp, synthetic construct
  misc_feature 1..48: note = synthetic Enterobacteria phage T7 wild type (WT) terminator part number SBa_000450
  source 1..48: mol_type = other DNA
tagcataaccccttggggcctctaaacgggtcttgaggggttttttgt

SEQ ID NO: 184 - DNA, 29 bp, synthetic construct
  misc_feature 1..29: note = synthetic Enterobacteria phage T7 terminator part number SBa_000451
  source 1..29: mol_type = other DNA
tacatatcgggggggtaggggttttttgt

SEQ ID NO: 185 - DNA, 37 bp, synthetic construct; identical to SEQ ID NO: 72
  misc_feature 1..37: note = synthetic Enterobacteria phage T7 terminator part number SBa_000452
  source 1..37: mol_type = other DNA

SEQ ID NO: 186 - DNA, 50 bp, synthetic construct; identical to SEQ ID NO: 55
  misc_feature 1..50: note = synthetic insulator part number SBa_000453
  source 1..50: mol_type = other DNA

SEQ ID NO: 187 - DNA, 50 bp, synthetic construct; identical to SEQ ID NO: 58
  misc_feature 1..50: note = synthetic insulator part number SBa_000454
  source 1..50: mol_type = other DNA

SEQ ID NO: 188 - DNA, 40 bp, synthetic construct; identical to SEQ ID NO: 61
  misc_feature 1..40: note = synthetic insulator part number SBa_000455
  source 1..40: mol_type = other DNA

SEQ ID NO: 189 - DNA, 40 bp, synthetic construct; identical to SEQ ID NO: 64
  misc_feature 1..40: note = synthetic insulator part number SBa_000456
  source 1..40: mol_type = other DNA

SEQ ID NO: 190 - DNA, 40 bp, synthetic construct; identical to SEQ ID NO: 67
  misc_feature 1..40: note = synthetic insulator part number SBa_000457
  source 1..40: mol_type = other DNA

SEQ ID NO: 191 - DNA, 30 bp, synthetic construct; identical to SEQ ID NO: 73
  misc_feature 1..30: note = synthetic insulator part number SBa_000458
  source 1..30: mol_type = other DNA

SEQ ID NO: 192 - DNA, 40 bp, synthetic construct; identical to SEQ ID NO: 75
  misc_feature 1..40: note = synthetic insulator part number SBa_000459
  source 1..40: mol_type = other DNA

SEQ ID NO: 193 - DNA, 40 bp, synthetic construct; identical to SEQ ID NO: 78
  misc_feature 1..40: note = synthetic insulator part number SBa_000460
  source 1..40: mol_type = other DNA

SEQ ID NO: 194 - DNA, 18 bp, synthetic construct
  misc_feature 1..18: note = synthetic insulator part number SBa_000461
  source 1..18: mol_type = other DNA
gtctgtagcacgtgcatc

SEQ ID NO: 195 - DNA, 18 bp, synthetic construct
  misc_feature 1..18: note = synthetic insulator part number SBa_000462
  source 1..18: mol_type = other DNA
ggtcattacaacggttat

SEQ ID NO: 196 - DNA, 40 bp, synthetic construct; identical to SEQ ID NO: 89
  misc_feature 1..40: note = synthetic insulator part number SBa_000463
  source 1..40: mol_type = other DNA

SEQ ID NO: 197 - DNA, 40 bp, synthetic construct; identical to SEQ ID NO: 92
  misc_feature 1..40: note = synthetic insulator part number SBa_000464
  source 1..40: mol_type = other DNA

SEQ ID NO: 198 - DNA, 40 bp, synthetic construct; identical to SEQ ID NO: 95
  misc_feature 1..40: note = synthetic insulator part number SBa_000465
  source 1..40: mol_type = other DNA

SEQ ID NO: 199 - DNA, 40 bp, synthetic construct; identical to SEQ ID NO: 98
  misc_feature 1..40: note = synthetic insulator part number SBa_000466
  source 1..40: mol_type = other DNA

SEQ ID NO: 200 - DNA, 40 bp, synthetic construct; identical to SEQ ID NO: 101
  misc_feature 1..40: note = synthetic insulator part number SBa_000467
  source 1..40: mol_type = other DNA

SEQ ID NO: 201 - DNA, 40 bp, synthetic construct; identical to SEQ ID NO: 104
  misc_feature 1..40: note = synthetic insulator part number SBa_000468
  source 1..40: mol_type = other DNA

SEQ ID NO: 202 - DNA, 40 bp, synthetic construct; identical to SEQ ID NO: 36
  misc_feature 1..40: note = synthetic insulator part number SBa_000508
  source 1..40: mol_type = other DNA

SEQ ID NO: 203 - DNA, 110 bp, synthetic construct
  misc_feature 1..110: note = synthetic ribosome binding site (RBS) part number SBa_000475
  source 1..110: mol_type = other DNA
ttaaagaggagaaattaagcatgaaaactatggacggtaacgctgcggctgcatggattagctacgcctttaccgaagtggctgcgatctacccgattacgccgagcacc

SEQ ID NO: 204 - DNA, 126 bp, synthetic construct
  misc_feature 1..126: note = synthetic ribosome binding site (RBS) part number SBa_000469
  source 1..126: mol_type = other DNA
tactagagacaataaactaacataaggaggataaatatgaccatgcgtcagtgcgcgatttatggcaaaggtggtattggcaaaagcacgacgacccagaacttggtggcggccctggccgagatg

SEQ ID NO: 205 - DNA, 174 bp, synthetic construct
  misc_feature 1..174: note = synthetic ribosome binding site (RBS) part number SBa_000470
  source 1..174: mol_type = other DNA
gcgtgcgtacaccttaatcaccgcttcatgctaaggtcctggctgcatgcaaaaattcacatccctatctagcggaggagccggatgatgactaatgctactggcgaacgtaacctggcactgattcaagaagtactggaagtgttcccggaaaccgcgcgcaaagagcgccgt

SEQ ID NO: 206 - DNA, 174 bp, synthetic construct
  misc_feature 1..174: note = synthetic ribosome binding site (RBS) part number SBa_000471
  source 1..174: mol_type = other DNA
caaacaccccatgtcgatactgaacgaatcgacgcacactcccttccttgcaatctcatactctcaaaaattaggcgaggtaacatgtctcaaactatcgataaaatcaactcttgttacccgctgttcgagcaggacgaatatcaggaactgttccgtaacaaacgtcagctg

SEQ ID NO: 207 - DNA, 156 bp, synthetic construct
  misc_feature 1..156: note = synthetic ribosome binding site (RBS) part number SBa_000472
  source 1..156: mol_type = other DNA
cctgattgtatccgcatctgatgctaccgtggttgagttaccatactcactcccggaggtacttctatgtctgacaatgataccctgttttggcgcatgctggcgctgtttcagtcgctgccggatttgcagccggctcaaatcgtcgattggctg

SEQ ID NO: 208 - DNA, 162 bp, synthetic construct
  misc_feature 1..162: note = synthetic ribosome binding site (RBS) part number SBa_000473
  source 1..162: mol_type = other DNA
catttttgccttgcgacagacctcctacttagattgccacactattcaatacatcactggaggttattacaaatgaagggtaacgagattcttgctctgctggacgaaccggcctgtgaacacaaccataaacagaaatccggctgtagcgccccaaagccg

SEQ ID NO: 209 - DNA, 161 bp, synthetic construct
  misc_feature 1..161: note = synthetic ribosome binding site (RBS) part number SBa_000474
  source 1..161: mol_type = other DNA
tgtcacgctaggaggcaattctataagaatgcacactgcacctaaacctaccacacctgaggagagtaattatggcagacattttccgcactgataagccgttggctgtgtcgccgatcaagaccggccagccgctgggtgcgatcctggcgtccctgggt

SEQ ID NO: 210 - DNA, 167 bp, synthetic construct
  misc_feature 1..167: note = synthetic ribosome binding site (RBS) part number SBa_000476
  source 1..167: mol_type = other DNA
aacatagcgttccatgagggctagaattacctaccggcctcagatactgacaaataaaccagcgaaggaggttcctaatgtggaactacagcgagaaagtcaaggaccatttcttcaatccgcgcaacgcgcgtgttgtggataacgcaaatgcggtgggcgacgtc

SEQ ID NO: 211 - DNA, 169 bp, synthetic construct
  misc_feature 1..169: note = synthetic ribosome binding site (RBS) part number SBa_000477
  source 1..169: mol_type = other DNA
cattgtaatagccaccaaaagagtgatgatagtcatgggtgatacccgtagaccattctgaaatcgaaggaggttttccatgaaacaagtgtacctggacaacaacgcgaccacccgcctggacccgatggttctggaagcgatgatgccgtttctcacggatttctat

SEQ ID NO: 212 - DNA, 164 bp, synthetic construct
  misc_feature 1..164: note = synthetic ribosome binding site (RBS) part number SBa_000478
  source 1..164: mol_type = other DNA
gagttactggccctgatttctccgcttctaataccgcacagcgactaggagcctaactcgccacaaggaaacatatggagcgcgtcttgatcaacgatactaccctgcgtgatggcgaacaatctccgggcgtagcgtttcgtacctccgagaaagttgccatc

SEQ ID NO: 213 - DNA, 165 bp, synthetic construct
  misc_feature 1..165: note = synthetic ribosome binding site (RBS) part number SBa_000479
  source 1..165: mol_type = other DNA
gactcaacacgctagggacgtgaagtcgattccttcgatgcagaaggcgagaactagatttaagggccattatagatggagtggttttaccagattccgggtgtagacgaattgcgcagcgctgaatccttctttcagttcttcgcggttccataccagccggaa

SEQ ID NO: 214 - DNA, 166 bp, synthetic construct
  misc_feature 1..166: note = synthetic ribosome binding site (RBS) part number SBa_000480
  source 1..166: mol_type = other DNA
tcgagaaacaaggcagttccgggctgaaagtagcgccgggacaagtcccgtattataaccgcctaggaggtgttggatgcgcccgaaattcaccttctctgaagaggtccgcgtagttcgcgcgattcgtaatgatggcaccgtggcgggttttgcgccaggtgcg

SEQ ID NO: 215 - DNA, 164 bp, synthetic construct
  misc_feature 1..164: note = synthetic ribosome binding site (RBS) part number SBa_000481
  source 1..164: mol_type = other DNA
acgccacgcgtagtgagacatacacgttcgttgggttcactcagagactgaagttattacccaggaggtctataatgaatccgtggcagcgctttgcccgtcaacgccttgctcgcagccgctggaaccgtgatccggctgctctcgacccagccgatacccca

SEQ ID NO: 216 - DNA, 124 bp, synthetic construct
  misc_feature 1..124: note = synthetic ribosome binding site (RBS) part number SBa_000482
  source 1..124: mol_type = other DNA
gacaagagtctcaattataaggaggctttactacatggcgaacatcggcatcttctttggtacggataccggcaaaacccgcaagattgcgaagatgattcacaaacagctgggcgagctggcc

SEQ ID NO: 217 - DNA, 169 bp, synthetic construct
  misc_feature 1..169: note = synthetic ribosome binding site (RBS) part number SBa_000483
  source 1..169: mol_type = other DNA
cgcgacttccagagaagaagactactgacttgagcgttccctctctgtaatacatcaaatcaatcataggagggctaaaatgacctcttgttcgtcgttttctggcggtaaagcgtgccgtccggccgatgactccgcgctgactccgctggtggccgacaaggcagct

SEQ ID NO: 218 - DNA, 152 bp, synthetic construct
  misc_feature 1..152: note = synthetic ribosome binding site (RBS) part number SBa_000475
  source 1..152: mol_type = other DNA
aactacgagatttgaggtaaaccaaataagcacgtagtggcattaaagaggagaaattaagcatgccgccattggactggttgcgtcgtttgtggttactctatcacgccggcaaaggcagctttccgcttcgtatgggcttgtcgccgcgt

SEQ ID NO: 219 - DNA, 1761 bp, synthetic construct
  misc_feature 1..1761: note = synthetic P-tac promoter and lacI cassette part number SBa_000561
  source 1..1761: mol_type = other DNA
caattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttgacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggcggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatt

SEQ ID NO: 220 - DNA, 740 bp, synthetic construct
  misc_feature 1..740: note = synthetic P-tet promoter and tetR cassette part number SBa_000562
  source 1..740: mol_type = other DNA
ttattaggacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctggacatcgaggcctttcctgtgtgagctagcactgtacctaggactgagctagccgtcaattttttccctatcagtgatagagattgacatccctatcagtgatagatataatgagcac

SEQ ID NO: 221 - DNA, 702 bp, synthetic construct
  misc_feature 1..702: note = synthetic red fluorescent protein (mRFP) fluorescsnt reporter part number SBa_000484
  source 1..702: mol_type = other DNA
atgtccagattagataaaagtaaagttgcgagctctgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaa

SEQ ID NO: 222 - DNA, 2652 bp, synthetic construct
  misc_feature 1..2652: note = synthetic Enterobacteria phage T7* variant RNA polymerase (RNAP) part number SBa_000514
  source 1..2652: mol_type = other DNA
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgacaaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagagttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtacgattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 223 - DNA, 3504 bp, synthetic construct; identical to SEQ ID NO: 2
  misc_feature 1..3504: note = synthetic Klebsiella pneumoniae nifJ
  source 1..3504: mol_type = other DNA

SEQ ID NO: 224 - DNA, 882 bp, synthetic construct; identical to SEQ ID NO: 4
  misc_feature 1..882: note = synthetic Klebsiella pneumoniae nifH
  source 1..882: mol_type = other DNA

SEQ ID NO: 225 - DNA, 1452 bp, synthetic construct
  misc_feature 1..1452: note = synthetic Klebsiella pneumoniae nifI
  source 1..1452: mol_type = other DNA
atgatgactaatgctactggcgaacgtaacctggcactgattcaagaagtactggaagtgttcccggaaaccgcgcgcaaagagcgccgtaaacacatgatggtttctgacccggaaatggaatctgtgggtaaatgcatcatctctaatcgcaaatctcagccgggtgtcatgactgttcgtggctgtgcgtacgcaggttctaaaggtgtcgtattcggcccgatcaaagatatggcgcatatctctcatggcccggtaggctgtggccagtactctcgcgcgggacgtcgtaactactacacgggcgtttctggcgttgactctttcggcacgctgaacttcacctctgacttccaggaacgtgacatcgttttcggtggcgataaaaagctgtccaaactgatcgaagaaatggaactgctgttcccgctgactaaaggcattactatccaaagcgaatgtccggtgggtctgatcggtgatgacatcagcgcggtcgcaaacgcatcttccaaagccctggataagccggtgatcccggttcgttgcgagggcttccgcggcgtttctcagtctctgggtcatcacatcgcaaacgatgttgtgcgtgactggattctgaacaaccgtgaaggtcagccttttgaaaccaccccttatgacgttgcgattattggcgactataacatcggcggcgacgcctgggcatcccgcatcctgctggaggagatgggtctgcgtgttgtcgcacagtggtctggcgatggcaccctggttgaaatggaaaacaccccgtttgttaaactgaacctggttcactgctaccgctccatgaactacattgcccgtcacatggaagaaaaacatcagatcccttggatggaatacaacttcttcggtccgactaaaatcgcagaatccctgcgtaaaatcgccgatcagtttgatgataccattcgcgcgaacgctgaagcagtaattgcgcgctacgaaggccagatggcagcaatcattgctaagtaccgtccgcgcctggaaggtcgtaaagtgctgctgtacatgggtggtctgcgtccacgtcatgtgatcggtgcctacgaggacctgggcatggagatcatcgcagcgggttacgaatttgcacacaacgacgactatgatcgtacgctgccagacctgaaagaaggtacgctgctgtttgacgacgccagctcttatgaactggaagccttcgtgaaagcgctgaaaccagacctgatcggctccggcatcaaggaaaaatacattttccagaaaatgggcgtgccgttccgccagatgcactcctgggactactccggtccgtaccacggctacgacggtttcgctatcttcgctcgtgacatggatatgaccctgaataacccagcgtggaatgaactgaccgcaccgtggctgaaatctgcataa

SEQ ID NO: 226 - DNA, 1563 bp, synthetic construct
  misc_feature 1..1563: note = synthetic Klebsiella pneumoniae nifK
  source 1..1563: mol_type = other DNA
atgtctcaaactatcgataaaatcaactcttgttacccgctgttcgagcaggacgaatatcaggaactgttccgtaacaaacgtcagctggaagaagcgcacgacgcacagcgcgtgcaggaagtgttcgcatggaccaccaccgcggaatacgaagctctgaacttccagcgcgaagccctgacggttgatccggcgaaagcgtgccagcctctgggtgcggttctgtgcagcctgggttttgccaacaccctgccgtatgtccacggttcccagggctgcgtagcctacttccgtacctatttcaaccgccactttaaagaaccaatcgcgtgcgtgtccgacagcatgacggaggacgcggcagttttcggtggtaacaacaacatgaacctgggcctgcaaaatgcttccgcactgtacaaaccggaaatcatcgcagtgtctaccacctgcatggcagaggttattggtgatgatctgcaagcatttattgccaacgcaaagaaagacggtttcgttgacagctctatcgcggttccgcacgctcataccccgtccttcatcggttctcacgtaactggttgggacaacatgttcgaaggcttcgcaaaaacttttaccgcagactatcaaggccaaccgggtaaactgccgaagctgaacctggtgaccggctttgaaacctacctgggcaactttcgtgtcctgaagcgcatgatggagcagatggcggttccgtgttctctgctgtctgacccgtctgaggttctggacactccagcggacggccactatcgcatgtattctggtggcaccactcagcaggaaatgaaagaggccccagacgcgattgacaccctgctgctgcaaccgtggcagctgctgaaaagcaagaaagttgttcaggaaatgtggaaccagccggcaacggaagttgcaatcccgctgggtctggcagctactgacgaactgctgatgaccgtgtcccaactgagcggcaaaccaatcgcggatgctctgaccctggaacgcggtcgcctggtggacatgatgctggacagccacacgtggctgcatggcaagaaatttggcctgtacggtgacccggacttcgtaatgggcctgacccgtttcctgctggaactgggctgcgagccgactgttatcctgtctcacaacgctaacaaacgttggcagaaggccatgaacaaaatgctggatgcgagcccatacggccgtgatagcgaagtgttcatcaactgcgacctgtggcatttccgctctctgatgtttacgcgtcagccggatttcatgatcggtaactcttacggcaaattcatccagcgtgacactctggccaaaggcaaagcgtttgaagtgccgctgattcgtctgggctttccgctgttcgaccgtcaccacctgcaccgccagaccacctggggttacgaaggcgcgatgaacatcgtaactactctggtaaacgcagtactggaaaagctggacagcgatacttcccagctgggcaaaaccgactattctttcgatctggttcgttaa

SEQ ID NO: 227 - DNA, 663 bp, synthetic construct
  misc_feature 1..663: note = synthetic Klebsiella pneumoniae nifY
  source 1..663: mol_type = other DNA
atgtctgacaatgataccctgttttggcgcatgctggcgctgtttcagtcgctgccggatttgcagccggctcaaatcgtcgattggctggcgcaggaatccggcgaaaccctgacgccggagcgccttgccaccctgacccaaccgcaactcgcggcgtcgttcccatccgcgacggcagtgatgagcccggctcgctggagccgcgttatggcttctctgcaaggcgccctcccagcccacttgcgcatcgtacgtccggcgcagcgtaccccgcaactgctcgccgcgttttgcagccaagacggccttgttatcaatggtcatttcggccagggtcgtctgttcttcatttacgcctttgacgagcagggcggctggctgtatgacttgcgccgctatccgagcgcaccgcaccagcaggaagcgaatgaggtgcgtgctcgtctgattgaagattgccagctgctgttctgccaggagattggcggtccggcagcagcgcgtctgatccgccaccgcatccatccgatgaaggcgcagccgggtactacgattcaggcgcagtgtgaagctatcaacaccctgctggccggtcgcctgccgccgtggctcgccaaacgtttgaaccgtgataacccgctggaagagcgtgtgttttaa

SEQ ID NO: 228 - DNA, 1374 bp, synthetic construct; identical to SEQ ID NO: 12
  misc_feature 1..1374: note = synthetic Klebsiella pneumoniae nifE
  source 1..1374: mol_type = other DNA

SEQ ID NO: 229 - DNA, 1386 bp, synthetic construct; identical to SEQ ID NO: 14
  misc_feature 1..1386: note = synthetic Klebsiella pneumoniae nifN
  source 1..1386: mol_type = other DNA

SEQ ID NO: 230 - DNA, 825 bp, synthetic construct; identical to SEQ ID NO: 16
  misc_feature 1..825: note = synthetic Klebsiella pneumoniae nifU
  source 1..825: mol_type = other DNA

SEQ ID NO: 231 - DNA, 1203 bp, synthetic construct; identical to SEQ ID NO: 94
  misc_feature 1..1203: note = synthetic Klebsiella pneumoniae nifS
  source 1..1203: mol_type = other DNA

SEQ ID NO: 232 - DNA, 1143 bp, synthetic construct
  misc_feature 1..1143: note = synthetic Klebsiella pneumoniae nifV
  source 1..1143: mol_type = other DNA
atggagcgcgtcttgatcaacgatactaccctgcgtgatggcgaacaatctccgggcgtagcgtttcgtacctccgagaaagttgccatcgcggaggcactgtacgctgcgggtatcaccgcgatggaagtcggcactccggcgatgggtgatgaagagatcgcccgcattcagctggtgcgtcgtcaactgccggacgcgacgcttatgacctggtgccgtatgaacgctctggaaatccgtcagagcgcggatctgggtattgactgggtggatatctcgatcccagcatccgacaagctgcgtcagtacaagctgcgtgagccgctggccgtgctgctggagcgccttgcgatgtttatccatctggcccacacgttaggcctcaaagtatgtattggttgcgaggatgcgagccgtgcgtctggtcagaccctgcgcgccattgccgaggtggcccagcaatgcgcggctgcgcgcttgcgttacgctgacaccgtgggcctgctggacccgttcaccaccgcagcccagatcagcgccctgcgtgacgtttggtcgggcgagatcgagatgcatgctcacaatgatctgggcatggctaccgcgaacacgctggcggcagtttcggctggcgccacgtcggtgaacactaccgtcctcggtctgggtgaacgtgcaggcaacgcagccctggaaaccgttgcgctgggcctggaacgctgcctgggcgtggaaaccggcgtccatttcagcgcgctcccagcgctctgtcagcgcgtcgcggaggctgcacagcgcgcaatcgacccgcaacagccgctggtgggtgaattggttttcacccacgagtctggtgttcacgttgcggcgctgctgcgcgacagcgaatcctatcaatctattgccccaagcctcatgggccgtagctaccgtctggtgctcggcaagcattcgggtcgtcaggctgtcaacggtgttttcgaccagatgggttaccacctgaatgcggcgcagatcaatcagttgctgccggccattcgccgcttcgccgagaattggaaacgctctccgaaagactacgaactggttgcgatctatgacgaattgtgcggtgaatccgcccttcgtgctcgcggctaa

SEQ ID NO: 233 - DNA, 258 bp, synthetic construct; identical to SEQ ID NO: 22
  misc_feature 1..258: note = synthetic Klebsiella pneumoniae nifW
  source 1..258: mol_type = other DNA

SEQ ID NO: 234 - DNA, 447 bp, synthetic construct; identical to SEQ ID NO: 24
  misc_feature 1..447: note = synthetic Klebsiella pneumoniae nifZ
  source 1..447: mol_type = other DNA

SEQ ID NO: 235 - DNA, 801 bp, synthetic construct; identical to SEQ ID NO: 26
  misc_feature 1..801: note = synthetic Klebsiella pneumoniae nifM
  source 1..801: mol_type = other DNA

SEQ ID NO: 236 - DNA, 531 bp, synthetic construct; identical to SEQ ID NO: 28
  misc_feature 1..531: note = synthetic Klebsiella pneumoniae nifF
  source 1..531: mol_type = other DNA

SEQ ID NO: 237 - DNA, 1407 bp, synthetic construct; identical to SEQ ID NO: 30
  misc_feature 1..1407: note = synthetic Klebsiella pneumoniae nifB
  source 1..1407: mol_type = other DNA

SEQ ID NO: 238 - DNA, 504 bp, synthetic construct; identical to SEQ ID NO: 32
  misc_feature 1..504: note = synthetic Klebsiella pneumoniae nifQ
  source 1..504: mol_type = other DNA

SEQ ID NO: 239 - protein, 206 aa, synthetic construct
  REGION 1..206: note = synthetic chimeric sigma factor ECF11_ECF02
MRITASLRTFCHLSTPHSDSTTSRLWIDEVTAVARQRDRDSFMRIYDHFAPRLLRYLTGLNVPEGQAEELVQEVLLKLWHKAESFDPSKASLGTWLFRIARNLYIDSVRKDRGWVQVQNSLEQLERLEAISNPENLMLSEELRQIVFRTIESLPEDLRMAITLRELDGLSYEEIAAIMDCPVGTVRSRIFRAREAIDNKVQPLIRR

SEQ ID NO: 240 - protein, 187 aa, synthetic construct
  REGION 1..187: note = synthetic chimeric sigma factor ECF02_ECF11
MSEQLTDQVLVERVQKGDQKAFNLLVVRYQHKVASLVSRYVPSGDVPDVVQEAFIKAYRALDSFRGDSAFYTWLYRIAVNTAKNYLVAQGRRPPSSDVDAIEAENFEQLERLEAPVDRTLDYSQRQEQQLNSAIQNLPTDQAKVLRMSYFEALSHREISERLDMPLGTVKSCLRLAFQKLRSRIEES

SEQ ID NO: 241 - protein, 883 aa, unidentified
  REGION 1..883: note = Enterobacteria phage T7 RNA polymerase (RNAP)
  source 1..883: note = Enterobacteria phage T7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 242 - DNA, 48 bp, synthetic construct
  misc_feature 1..48: note = synthetic Enterobacteria phage T7 stem loop terminator seed sequence
  misc_difference 3..6: note = n = g, a, c or t
  source 1..48: mol_type = other DNA
tannnnaaccsswwsssssstcwwwwcgsssssswwssggttttttgt

SEQ ID NO: 243 - DNA, 29 bp, synthetic construct
  misc_feature 1..29: note = synthetic Enterobacteria phage T7 terminator 52
  source 1..29: mol_type = other DNA
tataaaacggggggctaggggttttttgt

SEQ ID NO: 244 - DNA, 48 bp, synthetic construct
  misc_feature 1..48: note = synthetic Enterobacteria phage T7 terminator 23
  source 1..48: mol_type = other DNA
tactcgaacccctagcccgctcttatcgggcggctaggggttttttgt

SEQ ID NO: 245 - DNA, 37 bp, synthetic construct
  misc_feature 1..37: note = synthetic Enterobacteria phage T7 terminator 72
  source 1..37: mol_type = other DNA
tagcagaaccgctaacgggggcgaaggggttttttgt

SEQ ID NO: 246 - DNA, 48 bp, synthetic construct; identical to SEQ ID NO: 244
  misc_feature 1..48: note = synthetic Enterobacteria phage T7 terminator 48
  source 1..48: mol_type = other DNA

SEQ ID NO: 247 - DNA, 29 bp, synthetic construct
  misc_feature 1..29: note = synthetic Enterobacteria phage T7 terminator 1
  source 1..29: mol_type = other DNA
tacatatcgggggggtaggggttttttgt

SEQ ID NO: 248 - DNA, 29 bp, synthetic construct
  misc_feature 1..29: note = synthetic Enterobacteria phage T7 terminator 2
  source 1..29: mol_type = other DNA
tacatatcgggggggtaggggttttttgt

SEQ ID NO: 249 - DNA, 48 bp, synthetic construct; identical to SEQ ID NO: 183
  misc_feature 1..48: note = synthetic Enterobacteria phage T7 wild type (WT) terminator
  source 1..48: mol_type = other DNA

SEQ ID NO: 250 - DNA, 47 bp, synthetic construct
  misc_feature 1..47: note = synthetic Enterobacteria phage T7 terminator 31
  source 1..47: mol_type = other DNA
taccctaaccccttccccggtcaatcggggcggatggggttttttgt

SEQ ID NO: 251 - DNA, 47 bp, synthetic construct
  misc_feature 1..47: note = synthetic Enterobacteria phage T7 terminator 58
  source 1..47: mol_type = other DNA
tagaccaaccccttgcggcctcaatcgggggggatggggttttttgt

SEQ ID NO: 252 - DNA, 37 bp, synthetic construct; identical to SEQ ID NO: 72
  misc_feature 1..37: note = synthetic Enterobacteria phage T7 terminator 25
  source 1..37: mol_type = other DNA

SEQ ID NO: 253 - DNA, 48 bp, synthetic construct
  misc_feature 1..48: note = synthetic Enterobacteria phage T7 terminator 17
  source 1..48: mol_type = other DNA
tacctcaaccccttccgccctcatatcgcggggcatgcggttttttgt

SEQ ID NO: 254 - protein, 39 aa, synthetic construct
  REGION 1..39: note = synthetic Enterobacteria phage T7 RNA polymerase (RNAP) specificity loop sequence
VWQEYKKPIQTRLNLMFLGQFRLQPTINTNKDSEIDAHK

SEQ ID NO: 255 - protein, 39 aa, synthetic construct
  REGION 1..39: note = synthetic Enterobacteria phage T3 RNA polymerase (RNAP) specificity loop sequence
VWQEYKKPIQKRLDMIFLGQFRLQPTINTNKDSEIDAHK

SEQ ID NO: 256 - DNA, 23 bp, synthetic construct
  misc_feature 1..23: note = synthetic Enterobacteria phage T3 RNA polymerase (RNAP) promoter sequence
  source 1..23: mol_type = other DNA
taataaccctcactatagggaga

SEQ ID NO: 257 - protein, 39 aa, synthetic construct
  REGION 1..39: note = synthetic Enterobacteria phage K1F RNA polymerase (RNAP) specificity loop sequence
VWQEYKKPIQTRLNLMFLGSFNLQPTVNTNKDSEIDAHK

SEQ ID NO: 258 - DNA, 23 bp, synthetic construct
  misc_feature 1..23: note = synthetic Enterobacteria phage K1F RNA polymerase (RNAP) promoter sequence
  source 1..23: mol_type = other DNA
taataactatcactatagggaga

SEQ ID NO: 259 - protein, 39 aa, synthetic construct
  REGION 1..39: note = synthetic Enterobacteria phage N4 RNA polymerase (RNAP) specificity loop sequence
VWQEYKKPIQTRIDCVILGTHRMALTINTNKDSEIDAHK

SEQ ID NO: 260 - DNA, 23 bp, synthetic construct
  misc_feature 1..23: note = synthetic Enterobacteria phage N4 RNA polymerase (RNAP) promoter sequence
  source 1..23: mol_type = other DNA
taataacccacactatagggaga

SEQ ID NO: 261 - DNA, 498 bp, unidentified
  misc_feature 1..498: note = transcriptional activator sicA
  source 1..498: mol_type = unassigned DNA; note = Salmonella typhimurium
atggattatcaaaataatgtcagcgaagaacgtgttgcggaaatgatttgggatgccgttagtgaaggcgccacgctaaaagacgttcatgggatccctcaagatatgatggacggtttatatgctcatgcttatgagttttataaccagggacgactggatgaagctgagacgttctttcgtttcttatgcatttatgatttttacaatcccgattacaccatgggactggcggcagtatgccaactgaaaaaacaatttcagaaagcatgtgacctttatgcagtagcgtttacgttacttaaaaatgattatcgccccgttttttttaccgggcagtgtcaattattaatgcgtaaggcagcaaaagccagacagtgttttgaacttgtcaatgaacgtactgaagatgagtctctgcgggcaaaagcgttggtctatctggaggcgctaaaaacggcggagacagagcagcacagtgaacaagaaaaggaataa

SEQ ID NO: 262 - DNA, 498 bp, synthetic construct
  misc_feature 1..498: note = synthetic Salmonella typhimurium mutant transcriptional activator sicA*
  source 1..498: mol_type = other DNA
atggattatcaaaataatgtcagcgaagaacgtgttgcggaaatgatttgggatgccgttagtgaaggcgccacgctaaaagacgttcatgggatccctcaagatatgatggacggtttatatgctcatgcttatgagttttataaccagggacgactggatgaagctgagacgttctttcgttacttatgcatttatgatttttacaatcccgattacaccatgggactggcggcagtatgccaactgaaaaaacaatttcagaaagcatgtgacctttatgcagtagcgtttacgttacttaaaaatgattatcgccccgttttttttaccgggcagtgtcaattattaatgcgtaaggcagcaaaagccagacagtgttttgaacttgtcaatgaacgtactgaagatgagtctctgcgggcaaaagcgttggtctatctggaggcgctaaaaacggcggagacagagcagcacagtgaacaagaaaaggaataa

SEQ ID NO: 263 - DNA, 750 bp, unidentified
  misc_feature 1..750: note = transcriptional activator invF with new start codon
  source 1..750: mol_type = unassigned DNA; note = Salmonella typhimurium
atgctaaatacgcaggaagtacttaaagaaggagagaagcggaaaatccgcagcccggaagcatggtttatacagacgtgttccgcgcaaaagctgcatatgtcattttctgaaagccgacacaatgaaaattgcctgattcaggaaggcgcgctgcttttttgcgagcaggccgttgtcgcaccagtatcaggagacctggtttttcgaccgttaaaaattgaagtactcagcaaattactggcatttatcgatggcgcaggattagtggacacgacatatgctgaatccgataaatgggttttgctgagtcctgagtttcgcgctatttggcaagatcgtaaacgctgcgagtactggtttttgcagcaaattattacgccttctccggccttcaataaggtactggcgctgttacgaaaaagcgagagttactggttggttggctatttactcgctcagtcaaccagcggcaacacgatgagaatgctgggagaagactatggcgtttcttatacccattttcgtcgtttgtgcagcagagcgttgggcggaaaagcgaagagtgaattacgaaactggcgtatggcgcaatcgctgctgaatagtgtagaaggccacgagaacatcacccaattagccgttaatcatggttactcatcgccttcacatttttctagtgagatcaaagagctgatcggcgtttcgccgcggaaattatcaaatattattcaattggcagacaaatga

SEQ ID NO: 264 - DNA, 163 bp, synthetic construct
  misc_feature 1..163: note = synthetic promoter psicA
  source 1..163: mol_type = other DNA
ccacaagaaacgaggtacggcattgagccgcgtaaggcagtagcgatgtattcattgggcgttttttgaatgttcactaaccaccgtcggggtttaataactgcatcagataaacgcagtcgttaagttctacaaagtcggtgacagataacaggagtaagta

SEQ ID NO: 265 - DNA, 468 bp, Shigella flexneri
  misc_feature 1..468: note = transcriptional activator ipgC
  source 1..468: mol_type = unassigned DNA
atgtctttaaatatcaccgaaaatgaaagcatctctactgcagtaattgatgcaattaactctggcgctacactgaaagatattaatgcaattcctgatgatatgatggatgacatttattcatatgcttatgacttttacaacaaaggaagaatagaggaagctgaagttttcttcaggtttttatgtatatacgacttttacaatgtagactacattatgggactcgcagctatttatcagataaaagaacagttccaacaagcagcagacctttatgctgtcgcttttgcattaggaaaaaatgactatacaccagtattccatactggacaatgtcagcttcggttgaaagcccccttaaaagctaaagagtgcttcgaactcgtaattcaacacagcaatgatgaaaaattaaaaataaaagcacaatcatacttggacgcaattcaggatatcaaggagtaa

SEQ ID NO: 266 - DNA, 756 bp, synthetic construct
  misc_feature 1..756: note = synthetic codon optimized Shigella flexneri mxiE
  source 1..756: mol_type = other DNA
atgagtaaatataaaggcctgaacaccagcaacatgttctacatctacagctctggtcatgaaccggtgaacgttgaactggtgaaagataaagaacgtaacatcatcgaactggcaccggcgtggaaaggctttttctttgtgcgtaaccagaacatcaaattcagcgataacgttaactaccactaccgcttcaacatcaactcttgcgcaaaattcctggcgttttgggattattttagcggcgccctggttgaacattctcacgcagaaaaatgcatccatttctaccacgaaaacgatctgcgtgatagctgtaatacggaatctatgctggataaactgatgctgcgcttcatttttagtagcgatcagaacgtgtctaatgccctggcaatgatccgtatgaccgaaagttatcatctggttctgtacctgctgcgtacgattgaaaaagaaaaagaagtgcgcatcaaaagcctgaccgaacactatggcgtttctgaagcgtactttcgtagtctgtgtcgcaaagcgctgggtgccaaagtgaaagaacagctgaacacgtggcgcctggtgaatggcctgctggatgttttcctgcataaccagaccattacgagcgcggccatgaacaatggttatgcgtctaccagtcacttcagcaatgaaattaaaacgcgtctgggctttagtgcccgcgaactgagcaacatcaccttcctggtgaagaaaattaatgaaaaaatctaa

SEQ ID NO: 267 - DNA, 350 bp, synthetic construct
  misc_feature 1..350: note = synthetic promoter pipaH9.8
  source 1..350: mol_type = other DNA
gcgaaaatgacatcaaaaacgccattaacctgatgttctggggaatataaatgtcaggctagggtcaaaaatcgtggcgttgacaaaatggctgcgttacgtcattgagcatatccaggactggccggcaaaccgggtacgcgatctgttgccttggaaagttgatctgacctctcagtaaatatcaatacggttctgacgagccgcttaccgttcaaatatgaagtacgatgtttaactaaccgaaaaacaagaacaatacggtgcaaacaggccattcacggttaactgaaacagtatcgtttttttacagccaattttgtttatccttattataataaaaaagtgct

SEQ ID NO: 268 - DNA, 350 bp, synthetic construct
  misc_feature 1..350: note = synthetic promoter pipaH9.8* with mutation
  source 1..350: mol_type = other DNA
gcgaaaatgacatcaaaaacgccattaacctgatgttctggggaatataaatgtcaggctagggtcaaaaatcgtggcgttgacaaaatggctgcgttacgtcattgagcatatccaggactggccggcaaaccgggtacgcgatctgttgccttggaaagttgatctgacctctcagtaaatatcaatacggttctgacgagccgcttaccgttcaaatatgaagtacgatgtttaactaaccgaaaaacaagaacaatacggtgcaaacaggccattcacggttaactgaaacagtatcgtttttttacagccaattttgtttatccttattaagataaaaaagtgct

SEQ ID NO: 269 - DNA, 438 bp, Pseudomonas aeruginosa
  misc_feature 1..438: note = transcriptional activator exsC
  source 1..438: mol_type = unassigned DNA
atggatttaacgagcaaggtcaaccgactgcttgccgagttcgcaggccgtatcggtttgccttccctgtccctcgacgaggagggcatggcgagcctcctgttcgacgaacaggtgggcgtcaccctgttgctgctcgccgagcgcgagcgtctgttgctggaggccgatgtggcgggcatcgatgtgctgggcgaggggatctttcgccagctcgccagcttcaaccgccattggcaccgtttcgatctgcatttcggcttcgacgagctgaccggcaaggtccagttgtatgcgcagattctcgcagcgcaactgaccctcgaatgcttcgaggcgaccttggccaatctgctcgatcacgccgagttctggcagcgcctgctgccgtgcgacagtgatcgcgaggcggtcgctgcggtcggcatgagggtttga

SEQ ID NO: 270 - DNA, 831 bp, Pseudomonas aeruginosa
  misc_feature 1..831: note = transcriptional activator exsD
  source 1..831: mol_type = unassigned DNA
atggagcaggaagacgataagcagtactcccgagaagcggtgttcgctggcaggcgggtatccgtggtgggctcggacgcccgctcgcggggtcgggtgccgggttacgcatcgagcagtttgtatcgtgagtccggaatcatcagtgcgcggcaactggcgttgctgcagcggatgctgccgcgcctgcggctggagcaactgttccgctgcgagtggttgcagcagcgcctggcgcgcggcctggcgctggggcgcgaagaggtgcggcagattctcctctgcgcggcgcaggacgacgacggctggtgctccgaactgggcgaccgggtcaacctcgccgtgccgcagtcgatgatcgactgggtcctgctgccggtctatggctggtgggaaagcctgctcgaccaggcgatccccggctggcgcctgtcgctggtggagctggagacccagtcccggcaactgcgagtcaagtccgaattctggtcccgcgtggccgagctggagccggagcaggcccgcgaggaactggccagggtcgccaagtgccaggcgcgcacccaggaacaggtggccgaactggccggcaagctggagacggcttcggcactggcgaagagcgcctggccgaactggcagcggggcatggcgacgctgctcgccagcggcgggctggccggcttcgagccgatccccgaggtcctcgaatgcctctggcaacctctctgccggctggacgacgacgtcggcgcggcggacgccgtccaggcctggctgcacgaacgcaacctgtgccaggcacaggatcacttctactggcagagctga

SEQ ID NO: 271 - DNA, 837 bp, Pseudomonas aeruginosa
  misc_feature 1..837: note = transcriptional activator exsA
  source 1..837: mol_type = unassigned DNA
atgcaaggagccaaatctcttggccgaaagcagataacgtcttgtcattggaacattccaactttcgaatacagggtaaacaaggaagagggcgtatatgttctgctcgagggcgaactgaccgtccaggacatcgattccactttttgcctggcgcctggcgagttgcttttcgtccgccgcggaagctatgtcgtaagtaccaagggaaaggacagccgaatactctggattccattatctgcccagtttctacaaggcttcgtccagcgcttcggcgcgctgttgagtgaagtcgagcgttgcgacgagcccgtgccgggcatcatcgcgttcgctgccacgcctctgctggccggttgcgtcaaggggttgaaggaattgcttgtgcatgagcatccgccgatgctcgcctgcctgaagatcgaggagttgctgatgctcttcgcgttcagtccgcaggggccgctgctgatgtcggtcctgcggcaactgagcaaccggcatgtcgagcgtctgcagctattcatggagaagcactacctcaacgagtggaagctgtccgacttctcccgcgagttcggcatggggctgaccaccttcaaggagctgttcggcagtgtctatggggtttcgccgcgcgcctggatcagcgagcggagaatcctctatgcccatcagttgctgctcaacagcgacatgagcatcgtcgacatcgccatggaggcgggcttttccagtcagtcctatttcacccagagctatcgccgccgtttcggctgcacgccgagccgctcgcggcaggggaaggacgaatgccgggctaaaaataactga

SEQ ID NO: 272 - DNA, 80 bp, synthetic construct
  misc_feature 1..80: note = synthetic pesxD promoter
  source 1..80: mol_type = other DNA
gaaggacgaatgccgggctaaaaataactgacgttttttgaaagcccggtagcggctgcatgagtagaatcggcccaaat

SEQ ID NO: 273 - DNA, 80 bp, synthetic construct
  misc_feature 1..80: note = synthetic pexsC promoter
  source 1..80: mol_type = other DNA
gatgtggcttttttcttaaaagaaaagtctctcagtgacaaaagcgatgcatagcccggtgctagcatgcgctgagcttt

SEQ ID NO: 274 - DNA, 702 bp, synthetic construct
  misc_feature 1..702: note = synthetic red fluorescence protein (rfp, RFP)
  source 1..702: mol_type = other DNA
atggcttcctccgaagacgttatcaaagagttcatgcgtttcaaagttcgtatggaaggttccgttaacggtcacgagttcgaaatcgaaggtgaaggtgaaggtcgtccgtacgaaggtacgcagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtactaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctgcagcaaacgacgaaaactacgcttaa

SEQ ID NO: 275 - DNA, 43 bp, synthetic construct
  misc_feature 1..43: note = synthetic T5 phage buffer sequence
  source 1..43: mol_type = other DNA
agttcgatgagagcgataaccctctacaaataattttgtttaa

SEQ ID NO: 276 - DNA, 42 bp, synthetic construct
  misc_feature 1..42: note = synthetic T5 phage buffer sequence
  source 1..42: mol_type = other DNA
ataaattgataaacaaaaacctctacaaataattttgtttaa

SEQ ID NO: 277 - DNA, 43 bp, synthetic construct
  misc_feature 1..43: note = synthetic T5 phage buffer sequence
  source 1..43: mol_type = other DNA
ataaatttgagagaggagttcctctacaaataattttgtttaa

SEQ ID NO: 278 - DNA, 43 bp, synthetic construct
  misc_feature 1..43: note = synthetic T5 phage buffer sequence
  source 1..43: mol_type = other DNA
attaaagaggagaaattaaccctctacaaataattttgtttaa

SEQ ID NO: 279 - DNA, 43 bp, synthetic construct
  misc_feature 1..43: note = synthetic T7 phage buffer sequence
  source 1..43: mol_type = other DNA
aaacctaatggatcgaccttcctctacaaataattttgtttaa

SEQ ID NO: 280 - DNA, 42 bp, synthetic construct
  misc_feature 1..42: note = synthetic T7 phage buffer sequence
  source 1..42: mol_type = other DNA
atcgagagggacacggcgacctctacaaataattttgtttaa

SEQ ID NO: 281 - DNA, 42 bp, synthetic construct
  misc_feature 1..42: note = synthetic T7 phage buffer sequence
  source 1..42: mol_type = other DNA
gctaggtaacactagcagccctctacaaataattttgtttaa

SEQ ID NO: 282 - DNA, 42 bp, synthetic construct
  misc_feature 1..42: note = synthetic T7 phage buffer sequence
  source 1..42: mol_type = other DNA
atgaaacgacagtgagtcacctctacaaataattttgtttaa

SEQ ID NO: 283 - DNA, 43 bp, synthetic construct
  misc_feature 1..43: note = synthetic T7 phage buffer sequence
  source 1..43: mol_type = other DNA
agggagaccacaacggtttccctctacaaataattttgtttaa

SEQ ID NO: 284 - DNA, 43 bp, synthetic construct
  misc_feature 1..43: note = synthetic high-transcription escape buffer sequence
  source 1..43: mol_type = other DNA
attaaaaaacctgctaggatcctctacaaataattttgtttaa

SEQ ID NO: 285 - DNA, 43 bp, synthetic construct
  misc_feature 1..43: note = synthetic high-transcription escape buffer sequence
  source 1..43: mol_type = other DNA
ataaaggaaaacggtcaggtcctctacaaataattttgtttaa

SEQ ID NO: 286 - DNA, 43 bp, synthetic construct
  misc_feature 1..43: note = synthetic high-transcription escape buffer sequence
  source 1..43: mol_type = other DNA
ataggttaaaagcctgtcatcctctacaaataattttgtttaa

SEQ ID NO: 287 - DNA, 48 bp, synthetic construct
  misc_feature 1..48: note = synthetic carbon utilization buffer sequence
  source 1..48: mol_type = other DNA
acaataaaaaatcatttacatgtttcctctacaaataattttgtttaa

SEQ ID NO: 288 - DNA, 48 bp, synthetic construct
  misc_feature 1..48: note = synthetic carbon utilization buffer sequence
  source 1..48: mol_type = other DNA
agaagcagcgcgcaaaaatcagctgcctctacaaataattttgtttaa

SEQ ID NO: 289 - DNA, 48 bp, synthetic construct
  misc_feature 1..48: note = synthetic carbon utilization buffer sequence
  source 1..48: mol_type = other DNA
atgagttcatttcagacaggcaaatcctctacaaataattttgtttaa

SEQ ID NO: 290 - DNA, 48 bp, synthetic construct
  misc_feature 1..48: note = synthetic carbon utilization buffer sequence
  source 1..48: mol_type = other DNA
aacttgcagttatttactgtgattacctctacaaataattttgtttaa

SEQ ID NO: 291 - DNA, 48 bp, synthetic construct
  misc_feature 1..48: note = synthetic carbon utilization buffer sequence
  source 1..48: mol_type = other DNA
agccacaaaaaaagtcatgttggttcctctacaaataattttgtttaa

SEQ ID NO: 292 - DNA, 53 bp, synthetic construct
  misc_feature 1..53: note = synthetic carbon utilization buffer sequence
  source 1..53: mol_type = other DNA
acacagtcacttatcttttagttaaaaggtcctctacaaataattttgtttaa

SEQ ID NO: 293 - DNA, 43 bp, synthetic construct
  misc_feature 1..43: note = synthetic anti-escaping buffer sequence
  source 1..43: mol_type = other DNA
atccggaatcctcttcccggcctctacaaataattttgtttaa

SEQ ID NO: 294 - DNA, 48 bp, synthetic construct
  misc_feature 1..48: note = synthetic anti-escaping buffer sequence
  source 1..48: mol_type = other DNA
aacaaaataaaaaggagtcgctcaccctctacaaataattttgtttaa

SEQ ID NO: 295 - DNA, 43 bp, synthetic construct
  misc_feature 1..43: note = synthetic T5 phage buffer sequence
  source 1..43: mol_type = other DNA
agttcgatgagagcgataacagttccagattcaggaactataa

SEQ ID NO: 296 - DNA, 42 bp, synthetic construct
  misc_feature 1..42: note = synthetic T5 phage buffer sequence
  source 1..42: mol_type = other DNA
ataaattgataaacaaaaaagttccagattcaggaactataa

SEQ ID NO: 297 - DNA, 43 bp, synthetic construct
  misc_feature 1..43: note = synthetic T5 phage buffer sequence
  source 1..43: mol_type = other DNA
ataaatttgagagaggagttagttccagattcaggaactataa

SEQ ID NO: 298 - DNA, 43 bp, synthetic construct
  misc_feature 1..43: note = synthetic T5 phage buffer sequence
  source 1..43: mol_type = other DNA
attaaagaggagaaattaacagttccagattcaggaactataa

SEQ ID NO: 299 - DNA, 43 bp, synthetic construct
  misc_feature 1..43: note = synthetic T5 phage buffer sequence
  source 1..43: mol_type = other DNA
aaacctaatggatcgaccttagttccagattcaggaactataa

SEQ ID NO: 300 - DNA, 42 bp, synthetic construct
  misc_feature 1..42: note = synthetic T7 phage buffer sequence
  source 1..42: mol_type = other DNA
atcgagagggacacggcgaagttccagattcaggaactataa

SEQ ID NO: 301 - DNA, 42 bp, synthetic construct
  misc_feature 1..42: note = synthetic T7 phage buffer sequence
  source 1..42: mol_type = other DNA
gctaggtaacactagcagcagttccagattcaggaactataa

SEQ ID NO: 302 - DNA, 42 bp, synthetic construct
  misc_feature 1..42: note = synthetic T7 phage buffer sequence
  source 1..42: mol_type = other DNA
atgaaacgacagtgagtcaagttccagattcaggaactataa

SEQ ID NO: 303 - DNA, 43 bp, synthetic construct
  misc_feature 1..43: note = synthetic T7 phage buffer sequence
  source 1..43: mol_type = other DNA
agggagaccacaacggtttcagttccagattcaggaactataa

SEQ ID NO: 304 - DNA, 43 bp, synthetic construct
  misc_feature 1..43: note = synthetic high-transcription escape buffer sequence
  source 1..43: mol_type = other DNA
attaaaaaacctgctaggatagttccagattcaggaactataa

SEQ ID NO: 305 - DNA, 43 bp, synthetic construct
  misc_feature 1..43: note = synthetic high-transcription escape buffer sequence
  source 1..43: mol_type = other DNA
ataaaggaaaacggtcaggtagttccagattcaggaactataa

SEQ ID NO: 306 - DNA, 43 bp, synthetic construct
  misc_feature 1..43: note = synthetic high-transcription escape buffer sequence
  source 1..43: mol_type = other DNA
ataggttaaaagcctgtcatagttccagattcaggaactataa

SEQ ID NO: 307 - DNA, 48 bp, synthetic construct
  misc_feature 1..48: note = synthetic carbon utilization buffer sequence
  source 1..48: mol_type = other DNA
acaataaaaaatcatttacatgtttagttccagattcaggaactataa

SEQ ID NO: 308 - DNA, 48 bp, synthetic construct
  misc_feature 1..48: note = synthetic carbon utilization buffer sequence
  source 1..48: mol_type = other DNA
agaagcagcgcgcaaaaatcagctgagttccagattcaggaactataa

SEQ ID NO: 309 - DNA, 48 bp, synthetic construct
  misc_feature 1..48: note = synthetic carbon utilization buffer sequence
  source 1..48: mol_type = other DNA
atgagttcatttcagacaggcaaatagttccagattcaggaactataa

SEQ ID NO: 310 - DNA, 48 bp, synthetic construct
  misc_feature 1..48: note = synthetic carbon utilization buffer sequence
  source 1..48: mol_type = other DNA
aacttgcagttatttactgtgattaagttccagattcaggaactataa

SEQ ID NO: 311 - DNA, 48 bp, synthetic construct
  misc_feature 1..48: note = synthetic carbon utilization buffer sequence
  source 1..48: mol_type = other DNA
agccacaaaaaaagtcatgttggttagttccagattcaggaactataa

SEQ ID NO: 312 - DNA, 53 bp, synthetic construct
  misc_feature 1..53: note = synthetic carbon utilization buffer sequence
  source 1..53: mol_type = other DNA
acacagtcacttatcttttagttaaaaggtagttccagattcaggaactataa

SEQ ID NO: 313 - DNA, 43 bp, synthetic construct
  misc_feature 1..43: note = synthetic anti-escaping buffer sequence
  source 1..43: mol_type = other DNA
atccggaatcctcttcccggagttccagattcaggaactataa

SEQ ID NO: 314 - DNA, 48 bp, synthetic construct
  misc_feature 1..48: note = synthetic anti-escaping buffer sequence
  source 1..48: mol_type = other DNA
aacaaaataaaaaggagtcgctcacagttccagattcaggaactataa

SEQ ID NO: 315 - DNA, 30 bp, synthetic construct
  misc_feature 1..30: note = synthetic stem loops buffer sequence
  source 1..30: mol_type = other DNA
gatcaccagggggatcccccggtgaaggat

SEQ ID NO: 316 - DNA, 38 bp, synthetic construct
  misc_feature 1..38: note = synthetic stem loops buffer sequence
  source 1..38: mol_type = other DNA
gatcgcccaccggcagctgccggtgggcgatcaaggat

SEQ ID NO: 317 - DNA, 45 bp, synthetic construct
  misc_feature 1..45: note = synthetic stem loops buffer sequence
  source 1..45: mol_type = other DNA
gatcatcggtagagttaatattgagcagatcccccggtgaaggat

SEQ ID NO: 318 - DNA, 36 bp, synthetic construct
  misc_feature 1..36: note = synthetic stem loops buffer sequence
  source 1..36: mol_type = other DNA
attgatctggttattaaaggtaatcgggtcatttta

SEQ ID NO: 319 - DNA, 41 bp, synthetic construct
  misc_feature 1..41: note = synthetic stem loops buffer sequence
  source 1..41: mol_type = other DNA
gttctccacgggtgggatgagcccctcgtggtggaaatgcg

SEQ ID NO: 320 - DNA, 27 bp, synthetic construct
  misc_feature 1..27: note = synthetic stem loops buffer sequence
  source 1..27: mol_type = other DNA
agcatgaggtaaagtgtcatgcaccaa

SEQ ID NO: 321 - DNA, 38 bp, synthetic construct
  misc_feature 1..38: note = synthetic stem loops buffer sequence
  source 1..38: mol_type = other DNA
acgtcgacttatctcgagtgagatattgttgacggtac

SEQ ID NO: 322 - DNA, 37 bp, synthetic construct
  misc_feature 1..37: note = synthetic stem loops buffer sequence
  source 1..37: mol_type = other DNA
acgtcgacttatctcgagtgagataagttgacggtac

SEQ ID NO: 323 - DNA, 51 bp, synthetic construct
  misc_feature 1..51: note = synthetic stem loops buffer sequence
  source 1..51: mol_type = other DNA
acgtcgacttatctcgagactgcagttcaatagagatattgttgacggtac

SEQ ID NO: 324 - DNA, 52 bp, synthetic construct
  misc_feature 1..52: note = synthetic stem loops (ribozyme) buffer sequence
  source 1..52: mol_type = other DNA
gactgtcaccggatgtgctttccggtctgatgagtccgtgaggacgaaacag

SEQ ID NO: 325 - DNA, 53 bp, synthetic construct
  misc_feature 1..53: note = synthetic stem loops buffer sequence
  source 1..53: mol_type = other DNA
gatcaccagggggatcccccggtgaaggatcctctacaaataattttgtttaa

SEQ ID NO: 326 - DNA, 61 bp, synthetic construct
  misc_feature 1..61: note = synthetic stem loops buffer sequence
  source 1..61: mol_type = other DNA
gatcgcccaccggcagctgccggtgggcgatcaaggatcctctacaaataattttgtttaa

SEQ ID NO: 327 - DNA, 68 bp, synthetic construct
  misc_feature 1..68: note = synthetic stem loops buffer sequence
  source 1..68: mol_type = other DNA
gatcatcggtagagttaatattgagcagatcccccggtgaaggatcctctacaaataattttgtttaa

SEQ ID NO: 328 - DNA, 59 bp, synthetic construct
  misc_feature 1..59: note = synthetic stem loops buffer sequence
  source 1..59: mol_type = other DNA
attgatctggttattaaaggtaatcgggtcattttacctctacaaataattttgtttaa

SEQ ID NO: 329 - DNA, 64 bp, synthetic construct
  misc_feature 1..64: note = synthetic stem loops buffer sequence
  source 1..64: mol_type = other DNA
gttctccacgggtgggatgagcccctcgtggtggaaatgcgcctctacaaataattttgtttaa

SEQ ID NO: 330 - DNA, 50 bp, synthetic construct
  misc_feature 1..50: note = synthetic stem loops buffer sequence
  source 1..50: mol_type = other DNA
agcatgaggtaaagtgtcatgcaccaacctctacaaataattttgtttaa

SEQ ID NO: 331 - DNA, 61 bp, synthetic construct
  misc_feature 1..61: note = synthetic stem loops buffer sequence
  source 1..61: mol_type = other DNA
acgtcgacttatctcgagtgagatattgttgacggtaccctctacaaataattttgtttaa

SEQ ID NO: 332 - DNA, 60 bp, synthetic construct
  misc_feature 1..60: note = synthetic stem loops buffer sequence
  source 1..60: mol_type = other DNA
acgtcgacttatctcgagtgagataagttgacggtaccctctacaaataattttgtttaa

SEQ ID NO: 333 - DNA, 74 bp, synthetic construct
  misc_feature 1..74: note = synthetic stem loops buffer sequence
  source 1..74: mol_type = other DNA
acgtcgacttatctcgagactgcagttcaatagagatattgttgacggtaccctctacaaataattttgtttaa

SEQ ID NO: 334 - DNA, 75 bp, synthetic construct
  misc_feature 1..75: note = synthetic stem loops (ribozyme) buffer sequence
  source 1..75: mol_type = other DNA
gactgtcaccggatgtgctttccggtctgatgagtccgtgaggacgaaacagcctctacaaataattttgtttaa

SEQ ID NO: 335 - DNA, 72 bp, synthetic construct
  misc_feature 1..72: note = synthetic Ptac controller #1 promoter sequence, Ptac (SBa_00512 sequence
  source 1..72: mol_type = other DNA
tattctgaaatgagctgttgacaattaatcatcggctcgtataatgtgtggaattgtgagcggataacaatt

SEQ ID NO: 336 - DNA, 75 bp, synthetic construct
  misc_feature 1..75: note = synthetic controller #3 promoter sequence, Ptac plus OR1 and OR2 (SBa_000506) sequence
  source 1..75: mol_type = other DNA
tattaacaccgtgcgtgttgacagctatacctctggcggttataatgctagcggaattgtgagcggataacaatt

SEQ ID NO: 337 - DNA, 36 bp, synthetic construct
  misc_feature 1..36: note = synthetic randomized sequence
  misc_difference 21..28: note = n = g, a, c or t
  source 1..36: mol_type = other DNA
cttgggcacgcgtccattaannaggannaattaagc

SEQ ID NO: 338 - DNA, 36 bp, synthetic construct
  misc_feature 1..36: note = synthetic randomized sequence
  misc_difference 19..26: note = n = g, a, c or t
  source 1..36: mol_type = other DNA
tgggcacgcgtccattaannaggannaattattagc

SEQ ID NO: 339 - DNA, 36 bp, synthetic construct
  misc_feature 1..36: note = synthetic randomized sequence
  misc_difference 23..30: note = n = g, a, c or t
  source 1..36: mol_type = other DNA
tacttgggcacgcgtccattaannaggannaatagc

SEQ ID NO: 340 - DNA, 36 bp, synthetic construct
  misc_feature 1..36: note = synthetic randomized sequence
  misc_difference 21..28: note = n = g, a, c or t
  source 1..36: mol_type = other DNA
cttgggcacgcgtccattaanaaggagnaattaagc

SEQ ID NO: 341 - DNA, 36 bp, synthetic construct
  misc_feature 1..36: note = synthetic randomized sequence
  misc_difference 20..29: note = n = g, a, c or t
  source 1..36: mol_type = other DNA
cttgggcacgcgtccattantaaggaggnattaagc

SEQ ID NO: 342 - DNA, 34 bp, synthetic construct; identical to SEQ ID NO: 37
  misc_feature 1..34: note = synthetic weak ribosome binding site (RBS) (SBa_000507)
  source 1..34: mol_type = other DNA

SEQ ID NO: 343 - DNA, 123 bp, synthetic construct
  misc_feature 1..123: note = synthetic N-terminal degradation tag (SBa_000509)
  source 1..123: mol_type = other DNA
ttgtttatcaagcctgcggatctccgcgaaattgtgacttttccgctatttagcgatcttgttcagtgtggctttccttcaccggcagcagattacgttgaacagcgcatcgatctgggtggc

SEQ ID NO: 344 - DNA, 41 bp, synthetic construct
  misc_feature 1..41: note = synthetic Enterobacteria phage T7 terminator seed sequence
  misc_difference 3..16: note = n = g, a, c or t
  source 1..41: mol_type = other DNA
tannnaaccsswwssnsssstcwwwcgsssssswwssgttt

SEQ ID NO: 345 - skipped

(19) United States
(12) Patent Application Publication
(10) Pub. No.: US 2026/0015620 A1
(43) Pub. Date: Jan. 15, 2026

(54) SYNTHETIC GENE CLUSTERS

(71) Applicant: The Regents of the University of California, Oakland, CA (US)

(72) Inventors: Ethan Mirsky, San Francisco, CA (US); Karsten Temme, San Francisco, CA (US); Christopher A Voigt, Belmont, MA (US); Dehua Zhao, Allston, MA (US)

(73) Assignee: The Regents of the University of California, Oakland, CA (US)

(21) Appl. No.: 19/327,026

(22) Filed: Sep. 12, 2025

Related U.S. Application Data
(63) Continuation of application No. 18/978,750, filed on Dec. 12, 2024.
Application No. 18/978,750 is a continuation of application No. 16/671,036, filed on Oct. 31, 2019, now Pat. No. 12,209,245.
Application No. 16/671,036 is a division of application No. 15/706,353, filed on Sep. 15, 2017, now Pat. No. 10,662,432.
Application No. 15/706,353 is a division of application No. 15/288,916, filed on Oct. 7, 2016, now Pat. No. 9,957,509.
Application No. 15/288,916 is a continuation of application No. 14/126,307, filed on Jul. 25, 2014, now Pat. No. 9,512,431.
(60) Provisional application No. 61/497,781, filed on Jun. 16, 2011.

(51) Int. Cl. C12N 15/63; C07K 14/255; C07K 14/26; C12N 9/02; C12N 15/52; C12N 15/67; C12N 15/74; G16B 25/00; G16B 30/00; G16B 30/10; G16B 30/20

(52) U.S. Cl. CPC C12N 15/635; C07K 14/255; C07K 14/26; C12N 9/0095; C12N 15/52; C12N 15/63; C12N 15/67; C12N 15/74; C12Y 118/06001; G16B 25/00; G16B 30/00; G16B 30/10; G16B 30/20

(57) ABSTRACT

Methods for making synthetic gene clusters are described.

### CROSS-REFERENCES TO RELATED APPLICATIONS

The present application is a Continuation of U.S. application Ser. No. 18/978,750, filed Dec. 12, 2024, which is a Continuation of U.S. application Ser. No. 16/671,036, filed Oct. 31, 2019, which is a Division of U.S. application Ser. No. 15/706,353, filed Sep. 15, 2017, which is a Division of U.S. application Ser. No. 15/288,916, filed Oct. 7, 2016, which is a Continuation of U.S. application Ser. No. 14/126,307, filed Jun. 14, 2012, which is a US National Stage (371) of International Application No. PCT/US2012/042502, filed Jun. 14, 2012, which claims benefit of priority to U.S. Provisional Patent Application No. 61/497,781, filed Jun. 16, 2011, each of which is incorporated by reference.

### STATEMENT AS TO RIGHTS TO INVENTIONS MADE UNDER FEDERALLY SPONSORED RESEARCH AND DEVELOPMENT

This invention was made with government support under grant nos. CFF0943385 and EEC0540879 awarded by the National Science Foundation and grant no. R01 AI067699, awarded by the National Institutes of Health. The government has certain rights in the invention.

### REFERENCE TO SEQUENCE LISTING

The instant application contains a Sequence Listing which has been submitted electronically in XML file format and is hereby incorporated by reference in its entirety. Said XML copy, created on Sep. 10, 2025, is named 081906-1521322-209360US_SL.xml and is 587,314 bytes in size.

### BACKGROUND OF THE INVENTION

Genetically programming cells require sensors to receive information, circuits to process the inputs, and actuators to link the circuit output to a cellular response (Andrianantoandro E, et al., Mol Syst Biol 2 (2006); Chin J W Curr Opin Struct Biol 16: 551-556 (2006); Voigt C A Curr Opin Biotech 17: 548-557 (2006); Tan C, Mol Biosyst 3: 343-353 (2007)). In this paradigm, sensing, signal integration, and actuation are encoded by distinct ‘devices’ comprised of genes and regulatory elements (Knight T K, Sussman G J Unconventional Models of Computation 257-272 (1997); Endy D Nature 438: 449-453 (2005)). These devices communicate with one another through changes in gene expression and activity. For example, when a sensor is stimulated, this may lead to the activation of a promoter, which then acts as the input to a circuit.

### BRIEF SUMMARY OF THE INVENTION

Embodiments of the present invention provide a polynucleotide comprising a synthetic operon, wherein the operon comprises at least two coding sequences under the control of a heterologous transcriptional regulatory sequence, wherein each coding sequence is operably linked to a heterologous ribosome binding site (RBS). In some embodiments, the coding sequences are from the same native operon and the heterologous RBSs regulate translation of the coding sequences in a ratio that is substantially similar to the ratio of native translation from the native operon. In some embodiments, the coding sequences are from different native operons and the heterologous RBSs regulate translation of the coding sequences in a ratio that is substantially similar to the ratio of native translation from the native operon. In some embodiments, the coding sequences are from the same native operon and the coding sequences in the operon comprise one or more altered codon compared to the native operon. In some embodiments, codons of one or more coding sequence have been selected for maximal distance from codon usage of a corresponding coding sequence in the native operon.

In some embodiments, at least two coding sequences encode different proteins encoded by the Klebsiella pneumoniae nif gene cluster. In some embodiments, the proteins are selected from the group consisting of nifJ, nifH, nifD, nifK, nifY, nifE, nifN, nifU, nifS, nifV, nifW, nifZ, niM, nifF, nifB, and nifQ (e.g., wherein the coding sequences are substantially identical to those listed in FIG. 18). In some embodiments, the operon comprises coding sequences for Klebsiella pneumoniae nifH, nifD, nifK, and nifY. In some embodiments, the operon comprises coding sequences for Klebsiella pneumoniae nifE and nifN. In some embodiments, the operon comprises coding sequences for Klebsiella pneumoniae nifU, nifS, nifV, nifW, nifZ, and nifM. In some embodiments, the operon comprises coding sequences for Klebsiella pneumoniae nifB and nfQ.

In some embodiments, at least two coding sequences encode different proteins of the Salmonella typhimurium Type III secretion system. In some embodiments, the proteins are selected from the group consisting of PrgH, PrgI, PrgJ, PrgK, OrgA, OrgB, InvA, InvC, InvE, InvF, InvG, InvI, InvJ, SpaO, SpaP, SpaQ, SpaR, and SpaS (e.g., wherein the coding sequences are substantially identical to those listed in FIG. 24). In some embodiments, the operon comprises coding sequences for Salmonella typhimurium PrgH, PrgI, PrgJ, PrgK, OrgA, and OrgB. In some embodiments, the operon comprises coding sequences for Salmonella typhimurium InvA, InvC, InvE, InvF, InvG, InvI, InvJ, SpaO, SpaP, SpaQ, SpaR, and SpaS.

Embodiments of the present invention also provide for a host cell (optionally isolated) comprising a polynucleotide as described above or elsewhere herein. In some embodiments, the host cell is a prokaryotic or eukaryotic cell (including but not limited to a mammalian or plant or fungal cell).

Embodiments of the present invention also provide a system comprising a set of two or more different synthetic operons, the two or more operons each comprising at least two coding sequences under the control of a heterologous transcriptional regulatory sequence, wherein each coding sequence is operably linked to a heterologous ribosome binding site (RBS), wherein the transcriptional regulatory sequence of each operon in the set is controlled by the same transcriptional activator or repressor polypeptide(s).

In some embodiments, the system further comprises an expression cassette comprising a promoter operably linked to a polynucleotide encoding the transcriptional activator or repressor polypeptide(s). In some embodiments, the promoter of the expression cassette is an inducible promoter. In some embodiments, the polynucleotide in the expression cassette encodes a transcriptional repressor. In some embodiments, the polynucleotide in the expression cassette encodes a transcriptional activator. In some embodiments, the transcriptional activator is an RNA polymerase (RNAP). In some embodiments, the RNAP is T7 RNAP or is substantially similar to T7 RNAP.

In some embodiments, the transcriptional regulatory sequences of at least two of the operons are different.

In some embodiments, the coding sequences in the operons are organized such that coding sequences having substantially similar native expression are grouped into the same operon. In some embodiments, the transcriptional regulatory sequence of at least two operons have different promoters that are differentially regulated by T7 RNA polymerase and wherein the different strength of the promoters correspond to the relative strength of native promoters of the coding sequences.

In some embodiments, the expression cassette and the synthetic operons are expressed in a cell. In some embodiments, the cell is from a different species than the species from which the native operon was isolated. In some embodiments, the cell is from the same species from which the native operon was isolated.

In some embodiments, the system encodes a nitrogenase. In some embodiments, the system comprises a first operon comprising coding sequences for Klebsiella pneumoniae nifH, nifD, nifK, and nifY; a second operon comprising coding sequences for Klebsiella pneumoniae nifE and nifN; a third operon comprising coding sequences for Klebsiella pneumoniae nifU, nifS, nifV, nifW, nifZ, and nifM; and a fourth operon comprising coding sequences for Klebsiella pneumoniae nifB and nufQ. In some embodiments, the first, second, third, and fourth operon comprising a T7 RNA polymerase (RNAP) promoter and the system further comprises an expression cassette comprising a promoter operably linked to a polynucleotide encoding an RNAP substantially identical to T7 RNA polymerase (RNAP).

In some embodiments, the system encodes a type III secretion system. In some embodiments, the type III secretion system is a Salmonella typhimurium type III secretion system. In some embodiments, the system comprises a first operon comprising coding sequences for Salmonella typhimurium PrgH, PrgI, PrgJ, PrgK, OrgA, and OrgB and a second operon comprising coding sequences for Salmonella typhimurium InvA, InvC, InvE, InvF, InvG, InvI, InvJ, SpaO, SpaP, SpaQ, SpaR, and SpaS.

Embodiments of the present invention also provide a method for replacing native regulation of a set of genes collectively associated with a function with synthetic regulation. In some embodiments, the method comprises providing coding sequences for a set of polypeptides encoded by genes collectively associated with a function; changing codon identity within at least one coding sequence, thereby removing at least one regulatory sequence within the coding sequence; organizing the coding sequences into one or more synthetic operon(s); operably linking one or more heterologous transcriptional regulatory sequence to the operon(s), thereby controlling the magnitude of gene expression from the operon(s); and expressing the one or more synthetic operon(s) in a cell under the control of a polypeptide that binds directly or indirectly to the heterologous transcriptional regulatory sequence.

In some embodiments, the polypeptide is heterologous to the cell.

In some embodiments, the providing comprises obtaining the gene nucleotide sequences and eliminating non-coding sequences.

In some embodiments, the set of genes is from a gene cluster. In some embodiments, the set of genes are from a prokaryote. In some embodiments, the genes are from a native operon.

In some embodiments, the at least one regulatory sequence is identified using computation. In some embodiments, the computation comprises searches of coding sequences for ribosome binding sites, terminators, and/or promoters.

In some embodiments, removing the at least one regulatory sequence comprises replacement of native codons in the coding sequence with non-native synonymous codons. In some embodiments, the removing comprises selecting non-native codons having maximal distance from codons of the native coding sequence. In some embodiments, the removing comprises selecting non-native codons for optimal expression in a host cell.

In some embodiments, the method further comprises identifying and removing one or more of transposon insertion sites, sites that promote recombination, sites for cleavage by restriction endonucleases, and sites that are methylated.

In some embodiments, the organizing comprises grouping coding sequences into operons based on substantially similar native expression level.

In some embodiments, the organizing comprises ordering coding sequences within operons such that the highest expressing gene (based on native expression) occurs first and the lowest expressing gene (based on native expression) occurs last. In some embodiments, organization is based on native temporal expression, function, ease of manipulation of DNA, and/or experimental design. In some embodiments, magnitude of expression of coding sequences substantially correspond to the ratio of proteins encoded by the coding sequences as measured in the native system. In some embodiments, magnitude of expression of coding sequences is determined by computation. In some embodiments, the computation comprises a numerical optimization algorithm.

In some embodiments, the numerical optimization algorithm a Nelder-Mead algorithm, a Newton's method, a quasi-Newton method, a conjugate gradient method, an interior point method, a gradient descent, a subgradient method, a ellipsoid method, a Frank-Wolfe method, an interpolation method and pattern search methods, or an ant colony model.

In some embodiments, the heterologous transcriptional regulatory sequence(s) comprise a T7 RNAP promoter(s).

In some embodiments, the heterologous transcriptional regulatory sequence(s) comprise an inducible promoter.

In some embodiments, the method further comprises operably linking a heterologous ribosomal binding site (RBS) to one or more coding sequence in the synthetic operon. In some embodiments, different RBSs are operably linked to different coding sequences. In some embodiments, the RBSs regulate translation of the coding sequences in a ratio that is substantially similar to the ratio of native translation from the native operon.

In some embodiments, the method further comprises operably linking a heterologous transcriptional terminator sequence to one or more coding sequence in the synthetic operon. In some embodiments, the terminators are T7 RNAP terminators. In some embodiments, terminators for different operons are different.

In some embodiments, the method further comprises operably linking a buffer sequences between two functional sequences in an operon wherein the functional sequences are selected from the group consisting of a promoter, ribosome binding site, coding sequence, and terminator. In some embodiments, the buffer sequence is selected from the group consisting of a random sequence, a UP-region of a promoter, an extended 5-UTR sequence, and a RNAase cleavage site.

In some embodiments, the operons are expressed from a plasmid. In some embodiments, the plasmid has a low copy origin of replication.

In some embodiments, the polypeptide that binds directly or indirectly to the heterologous transcriptional regulatory sequence is expressed from a control expression cassette, the expression cassette comprising a control promoter operably linked to a polynucleotide sequence encoding the polypeptide. In some embodiments, the expression cassette is contained in a control plasmid separate from a plasmid containing the operons. In some embodiments, the control promoter is an inducible promoter.

In some embodiments, the heterologous polypeptide comprises an RNA polymerase (RNAP). In some embodiments, the RNAP is T7 RNAP. In some embodiments, the expression cassette is an environmental sensor.

Embodiments of the invention also provide for a method for determining an experimentation point for controlling the magnitude of expression of two or more genes (e.g., within a synthetic operon). In some embodiments, the method comprises: receiving one or more input data points, wherein the input data points provide information about one or more regulatory elements and a system property; and determining, with a computer, a next data point using a computational method, wherein the next data point provides information about the one or more regulatory elements.

In some embodiments, the method further comprises using the next data point for further experimentation to optimize expression of the two or more genes. In some embodiments, the regulatory elements include, e.g., ribosomal binding sites and/or transcriptional regulatory elements.

In some embodiments, the computational method is a numerical analysis technique. In some embodiments, the numerical optimization method is the Nelder-Mead algorithm, the Newton's method, the quasi-Newton method, a conjugate gradient method, an interior point method, a gradient descent, a subgradient method, a ellipsoid method, the Frank-Wolfe method, an interpolation method and pattern search methods, or an ant colony model. In some embodiments, the numerical optimization method used to determine the next data point for further experimentation requires considering the reflection point, expansion point, or contraction point based on the one or more input data points.

In some embodiments, the computational method is a design of experiments (DoE) method.

Embodiments of the invention also provide for a computer program product comprising a tangible computer readable medium storing a plurality of instructions for controlling a processor to perform an operation for determining an experimentation point for controlling the magnitude of expression of two or more genes, the instructions comprising receiving one or more input data points, wherein the input data points provide information about one or more regulatory elements and a system property; and determining, with a computer, a next data point using a computational method, wherein the next data point provides information about the one or more regulatory elements.

### Definitions

A recitation of “a”, “an” or “the” is intended to mean “one or more” unless specifically indicated to the contrary.

A polynucleotide or polypeptide sequence is “heterologous to” an organism or a second sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a T7 RNA polymerase promoter operably linked to a synthetic nif operon).

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence. In the context of a ribosomal binding site (RBS) and coding sequences, the term refers to the functional linkage of the RBS to the coding sequence wherein the RBS recruits ribosomes for translation of the coding sequence on an RNA.

A “cognate pair” as used herein refers to a sequence-specific DNA binding polypeptide and a target DNA sequence that is bound by the particular sequence-specific DNA binding polypeptide. For sequence-specific DNA binding polypeptides that bind more than one target nucleic acid, the cognate pair can be formed with the sequence-specific DNA binding polypeptide and any one of the target DNA sequences the polypeptide binds.

“Orthogonal” transcriptional systems refer to systems (e.g., one, two, three, or more) of transcriptional regulatory elements comprising target DNA sequences regulated by their cognate sequence-specific DNA binding polypeptide such that the sequence-specific DNA binding polypeptides in the system do not have “cross-talk,” i.e., the sequence-specific DNA binding polypeptides do not interfere or regulate transcriptional regulatory elements in the system other than the transcriptional regulatory elements containing the cognate target DNA sequence of the sequence-specific DNA binding polypeptide.

“Sequence-specific DNA binding polypeptides” refer to polypeptides that bind DNA in a nucleotide sequence specific manner. Exemplary sequence-specific DNA binding polypeptides include, but are not limited to transcription factors (e.g., transcriptional activators), RNA polymerases, and transcriptional repressors.

A “transcriptional activator” refers to a polypeptide, which when bound to a promoter sequence, activates or increases transcription of an RNA comprising the operably-linked coding sequence. In some embodiments, the transcriptional activator bound to a target sequence in a promoter can assist recruitment of RNA polymerase to the promoter. A “transcriptional repressor” refers to a polypeptide, which when bound to a promoter sequence, blocks or decreases transcription of an RNA comprising the operably-linked coding sequence. In some embodiments, the transcriptional repressor blocks recruitment of the RNA polymerase to the promoter or blocks the RNA polymerase's movement along the promoter.

The term “coding sequence” as used herein refers to a nucleotide sequence beginning at the codon for the first amino acid of an encoded protein and ending with the codon for the last amino acid and/or ending in a stop codon.

The term “host cell” refers to any cell capable of replicating and/or transcribing and/or translating a heterologous gene. Thus, a “host cell” refers to any prokaryotic cell (including but not limited to E. coli) or eukaryotic cell (including but not limited to yeast cells, mammalian cells, avian cells, amphibian cells, plant cells, fish cells, and insect cells), whether located in vitro or in vivo. For example, host cells may be located in a transgenic animal or transgenic plant. prokaryotic cell (including but not limited to E. coli) or eukaryotic cells (including but not limited to yeast cells, mammalian cells, avian cells, amphibian cells, plant cells, fish cells, and insect cells).

“Transcriptional regulatory elements” refer to any nucleotide sequence that influences transcription initiation and rate, or stability and/or mobility of a transcript product. Regulatory sequences include, but are not limited to, promoters, promoter control elements, protein binding sequences, 5′ and 3′ UTRs, transcriptional start sites, termination sequences, polyadenylation 5 sequences, introns, etc. Such transcriptional regulatory sequences can be located either 5′-, 3′-, or within the coding region of the gene and can be either promote (positive regulatory element) or repress (negative regulatory element) gene transcription.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, and mRNA encoded by a gene.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Examples of algorithms that are suitable for determining percent sequence identity and sequence similarity are the BLAST and BLAST 2.0 algorithms, which are described in Altschul et al., Nucleic Acids Res. 25:3389-3402 (1997), and Altschul et al., J. Mol. Biol. 215:403-410 (1990), respectively. Software for performing BLAST analyses is publicly available on the Web through the National Center for Biotechnology Information ([[www.]]ncbi.nlm.nih.gov[[/]]). This algorithm involves first identifying high scoring sequence pairs (HSPs) by identifying short wordlength (W) in the query sequence, which either match or satisfy some positive-valued threshold score (T) when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, (1989)) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity to a designated reference sequence. Alternatively, percent identity can be any integer from 25% to 100%, for example, at least: 25%, 30%, 35%, 40%, 45%, 50%, 55%, 60%, 65%, 70%, 75%, 80%, 85%, 90%, 95%, or 99% compared to a reference sequence using the programs described herein; preferably BLAST using standard parameters, as described below. One of skill will recognize that the percent identity values above can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Percent identity of polypeptides can be any integer from 40% to 100%, for example, at least 40%, 45%, 50%, 55%, 60%, 65%, 70%, 75%, 80%, 85%, 90%, 95%, or 99%. In some embodiments, polypeptides that are “substantially similar” share sequences as noted above except that residue positions that are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, aspartic acid-glutamic acid, and asparagine-glutamine.

### BRIEF DESCRIPTION OF THE DRAWINGS

FIG. 1 depicts a scheme illustrating nifE and nifN genes under the control of unique T7 promoters.

FIG. 2 illustrates the quantitatively measurement of the capacity of the synthetic operon to complement a nifEN knockout strain and recover the ability to fix nitrogen.

FIG. 3 illustrates a library of wild-type (SEQ ID NO:157) and mutant T7 promoters (SEQ ID NOS:158-163) and their strength to control gene expression. T7 promoter seed sequence=SEQ ID NO:156.

FIGS. 4A-4B illustrates strengths of T7 promoters to control nifE and nifN genes in selected mutant strains. FIG. 4A depict strengths of three strains. FIG. 4B depict the calculated Reflection coordinates.

FIG. 5 illustrates the nitrogen fixation in the Reflection strain and the initial strains.

FIG. 6 illustrates the method of refactoring nitrogen fixation.

FIG. 7 illustrates the nif gene cluster from Klebsiella oxytoca.

FIG. 8 depicts a scheme of a fluorescent reporter plasmid in which the 150 bp surrounding a gene's start codon (from −60 to +90) was fused to the mRFP gene and expressed under the control of the Ptac promoter.

FIG. 9 illustrates the measured fluorescence by flow cytometry.

FIG. 10 illustrates the multiple clones used to identify the synthetic ribosome binding site that best matched the native ribosome binding site.

FIG. 11 illustrates the chimeric operons.

FIG. 12 lists the errors in the fully synthetic operons.

FIG. 13 shows that each synthetic operon required different levels of IPTG concentration for optimal function. It also shows the performance of individual operons in the T7 Wires system under Ptac promoter control.

FIG. 14 shows a table of the control of the synthetic operons in the system.

FIG. 15 shows nitrogen fixation from a full synthetic cluster expressed in a complete nif knockout strain.

FIG. 16 illustrates the use of either controller #1 or controller #2 to produce the same performance from the full synthetic cluster.

FIG. 17 depicts a detailed schematic of the full synthetic cluster.

FIG. 18 shows DNA sequences for native genes (SEQ ID NOS:1, 3, 5, 7, 9, 11, 13, 15, 17, 19, 21, 23, 25, 27, 29 and 31) and synthetic genes (SEQ ID NOS:2, 4, 6, 8, 10, 12, 14, 16, 18, 20, 22, 24, 26, 28, 30, and 32), as well as the percent common nucleotide and codon identities between each pair.

FIG. 19 shows the names and sequences of parts of the synthetic controller (SEQ ID 20 NOS:33-51).

FIG. 20 lists the names, sequences and strengths of each components of the full cluster (SEQ ID NOS:52-118).

FIG. 21 shows a diagram of the RBS test vector.

FIGS. 22A-22B depict schematics of the inv-spa and prg-org operons and the FIG. 22A shows a schematic of Δprg-org Salmonella SL1344 knock-out strain. The inv-spa and prg-org operons are boxed. FIG. 22B shows a schematic of the prg-org operon test vector and reporter plasmid. The control plasmid and reporter plasmid are on the right.

FIGS. 23A-23B show a western blot of secreted protein expressed from the synthetic prg-org operon in Δprg-org knock-out strain. FIG. 23A shows that the Δprg-org knock-out strain does not express the prg-org operon. FIG. 23B shows that the synthetic refactored prg-org operon in Salmonella Δprg-org cells can be controlled by the addition of IPTG.

FIG. 24 shows the synthetic RBS and synthetic operon sequences of the T3SS (SEQ ID NOS:119-155).

FIG. 25 shows a block diagram of a computer system.

FIG. 26 illustrates the process of refactoring a gene cluster. The wild-type K. oxytoca nitrogen fixation gene cluster is shown at top. The genes are colored by function: blue (nitrogenase), green (co-factor biosynthesis, shading corresponds to operons), yellow (e-transport), and grey (unknown). The thin arrows show the length and orientation of the seven operons and a horizontal bar indicates overlapping genes. The recoded genes are shown as dashed lines. The symbols used to define the refactored cluster and controller are defined in FIGS. 29 and 30, respectively

FIGS. 27A-27C illustrates the robustness of the nitrogen fixation pathway to changes in the expression of component proteins. FIG. 27A The pathway for nitrogenase maturation is shown and proteins are coloured by function (FIG. 26). The metal clusters are synthesized by the biosynthetic pathway (23, 24). Nitrogen fixation catalyzed by the matured nitrogenase is shown with its in vivo electron transport chain. FIG. 27B The tolerance of nitrogenase activity to changes in the expression of component proteins are shown. Activity is measured via an acetylene reduction assay and the % compared to wild-type K. oxytoca is presented. Wild-type operons are expressed from a Ptac promoter on a low copy plasmid. The promoter activity is calculated as the output of the Ptac promoter at a given concentration of IPTG and compared to a constitutive promoter. The effect of not including NifY (−Y) and NifX (−X) are shown in red. FIG. 27C The comparison of the strength of wild-type (black) and synthetic (white) ribosome binding sites (RBSs) is shown. The RBSs were measured through an in-frame transcriptional fusion (−60 to +90) with mRFP. The strength is measured as the geometric average from a distribution of cells measured by flow cytometry. The synthetic RBSs of nifF and nifQ are not intended to match the wild-type measurement. Error bars represent the standard deviation of at least three experiments performed on different days.

FIGS. 28A-28C illustrates converting to T7* RNAP Control. FIG. 28A Nitrogenase activity is shown as a function of promoter strength for each refactored operon in respective K. oxytoca knockout strains (ΔnifHDKTY, ΔnifENX, ΔnifJ, ΔnifBQ, ΔnifF, and ΔnifUSVWZM). Vertical dashed lines indicate strength of the mutant T7 promoter that controls each operon in the complete refactored gene cluster. FIG. 28B A controller plasmid decouples operon expression from the inducible promoter. A T7 RNAP variant (T7*) was designed to reduce toxicity. A set of 4 mutated T7 promoters were used to control the expression of each operon (part numbers and sequences for mutants 1-4 are listed in the Materials and Methods section). Ptac activity under 1 mM IPTG induction is indicated by a dashed horizontal line. FIG. 28C Nitrogenase activity is compared for each refactored operon under the control of the Ptac promoter at the optimal IPTG concentration (black) and the controller (part D) with 1 mM IPTG and expression controlled by different T7 promoters (white). The T7 promoters used are WT for operons HDKY, EN and J; promoter 2 for operons BQ and USVWZM; and promoter 3 for F. Error bars represent the standard deviation of at least three experiments performed on different days.

FIG. 29 shows a comprehensive schematic for the complete refactored gene cluster and controller. Each of the 89 parts is represented according to the SBOL visual standard (sbolstandard.org), and the SynBERC Registry part number (registry.synberc.org) and part activity are shown. The T7 promoter strengths are measured with red fluorescent protein (mRFP) and reported in REU (see, Materials and Methods). Terminator strengths are measured in a reporter plasmid and reported as the fold-reduction in mRFP expression when compared to a reporter without a terminator. The RBS strength is reported in as arbitrary units of expression from the induced Ptac promoter (1 mM IPTG) and a fusion gene between the first 90 nucleotides of the gene and red fluorescent protein. The nucleotide numbers for the plasmids containing the refactored cluster and controller are shown. The codon identity of each recoded gene as compared to wild-type is shown as a percent.

FIGS. 30A-30D shows the regulation of the complete refactored gene cluster. FIG. 30A Nitrogenase activity for the three controllers are shown: IPTG-inducible, aTc-inducible, and IPTG ANDN aTc logic. The gas chromatography trace is shown for each as well as the calculated percent of wild-type activity, (7.4%±2.4%, 7.2%±1.7% and 6.6%±1.7% respectively). Standard deviation is calculated using data from at least two experiments performed on different days. FIG. 30B 15N incorporation into cell biomass is shown. Nitrogen fixation from N2 gas by the refactored gene cluster was traced using 15N2 and measured using isotope ratio mass spectronomy (IRMS). Data are represented as the fraction of cellular nitrogen that is 15N. The standard deviation represents two experiments performed on different days. FIG. 30C The effect of ammonia on regulation of nitrogenase expression is shown. Acetylene reduction traces shown with (red) and without (blue) addition of 17.5 mM ammonium acetate for wild type cells (left) and cells bearing synthetic nif system (right). The synthetic system was induced by Controller #1 using 1 mM IPTG and exhibited nitrogenase activity of 1.1%±0.5% and 6.1%±0.4% with and without ammonium acetate respectively. FIG. 30D T7* RNAP expression of Controller #1 corresponding to Part C is shown. Strains carrying Controller #1 and a RFP reporter plasmid were characterized under 1 mM IPTG induction with or without addition of ammonium acetate.

FIG. 31 shows the nif operon deletions used in this study. The solid lines show the region of deleted nif operons. The dashed line in NF25 shows the retained nifLA operon.

FIG. 32 depicts a table of construction and verification of all K. oxytoca nif gene deletion mutants (SEQ ID NOS:164-177).

FIGS. 33A-33B shows promoter characterization using Relative Expression Units. FIG. 33A Conversion of arbitrary units into Relative Expression Units (REU). Promoters were characterized using mRFP1 fluorescent reporter protein in N155 (Measured Promoters). Data were first normalized by the fluorescence of N110 (Internal Standard) and then scaled by the fluorescence of N155(J23100) to account for RBS differences between N155 and N110 (RBS Adjustment). To directly compare our measurements to expression levels of the Kelly et al. standards, we further multiplied by the ratio of N110 fluorescence to the fluorescence of a Kelly standard plasmid expressing mRFP1 (RFP Promoter Standard). A final conversion factor is applied to compare all measurements to the Kelly et al. J23101-EGFP promoter standard based on a strong linear correlation of promoter strength (RPU) between constructs expressing mRFP and EGFP. Solid and dashed boxes were drawn to indicate which plasmids were measured at different facilities. Asterisked and non-asterisked units were measured in different facilities and correspond to the conversion factors directly above. FIG. 33B Promoter characterization for Ptac promoter (left) and Ptet promoter (right). The promoter strengths of Ptac promoter and Ptet promoter were measured under varied concentrations of inducers (IPTG or aTc). The strengths of T7 promoters (WT and mutants, FIG. 28B) are shown as horizontal dotted lines.

FIGS. 34A-34B illustrates debugging of the refactored operons. FIG. 34A The process is shown for the identification of problem sequences within a refactored operon. After design and synthesis, the problematic DNA is crossed with wild-type to create a chimeric library, which is screened. This is done iteratively to reduce the size of the problematic region until the specific errors are identified. FIG. 34B The debugging process led to the correction of RBS strengths (red arrows), the recoded sequence of nifH, and numerous nucleotide errors found in the sequenced cluster in the database. Amino acid mutations to correct errors in the synthetic sequence are shown.

FIG. 35 depicts a table of DNA sequence errors in nif cluster sequence X13303.1.

FIG. 36 shows cell growth supported by nitrogen fixation. The dotted line indicates initial seeding density of OD600 0.5. Wild-type Klebsiella grew to an OD600 2.57±0.07 after 36 hours of incubation in depression conditions. Eliminating the full nif cluster severely inhibited cell growth (Δnif, OD600 0.76±0.02). Complementing the knockout strain with the refactored cluster and Controller #1 under 1 mM IPTG induction yielded growth of OD600 1.10±0.03.

FIG. 37 shows expression of synthetic synthetic nifH variants. Western blot assay to detect the expression of synthetic nifHᵥ₁ (left) and synthetic synthetic nifHᵥ₂ (right). All constructs bore P_{tac}-nifHDK with the synthetic gene indicated. Cultures were induced with 50 μM IPTG.

FIG. 38 depicts a table of DNA sequences of synthetic parts (SEQ ID NOS:178-238).

FIG. 39 shows maps of key plasmids. SBOL graphical notation is used to describe genetic parts: the BioBrick prefix and suffix are open squares, and terminators are in the shape of a T.

### DETAILED DESCRIPTION

### I. Introduction

The present invention relates to gene cluster engineering. It has been discovered how to recombinantly and computationally manipulate and select native gene cluster coding sequences and heterologous regulatory sequences such that the coding sequences are under control of heterologous regulation and produce the functional product of the gene cluster (e.g., a native operon). By eliminating native regulatory elements outside of, and within, coding sequences of gene clusters, and subsequently adding synthetic regulatory systems, the functional products of complex genetic operons and other gene clusters can be controlled and/or moved to heterologous cells, including cells of different species other than the species from which the native genes were derived.

As demonstrated below, the inventors have re-engineered the Klebsiella oxytoca Nif gene cluster as well as a Salmonella Type III protein secretion system, thereby generating functional products (e.g., nitrogen fixing enzymes and peptide secretion complexes, respectively) under control of a heterologous regulatory system. Once re-engineered, the synthetic gene clusters can be controlled by genetic circuits or other inducible regulatory systems, thereby controlling the products' expression as desired.

### II. Generation of Synthetic Gene Clusters

It is believed that the methods described herein can be used and adapted to re-engineer regulation of essentially any operon or other gene cluster. Generally, the native operons or gene clusters to be engineered will have the same functional product in the native host. For example, in some embodiments, at least a majority of the gene products within the native operon or gene cluster to be re-engineered will each function to produce a specific product or function of the native host. Functional products can include, for example, multi-component enzymes, membrane-associated complexes, including but not limited to complexes that transport biological molecules across membranes, or other biologically active complexes. For example, in some embodiments, the functional products are, e.g., a Type III protein secretion system, a bacterial microcompartment, a gas vesicle, a magnetosome, a cellulosome, an alkane degradation pathway, a nitrogen fixation complex, a polybiphenyl degradation complex, a pathway for biosynthesis of Poly (3-hydroxbutyrate), nonribosomal peptide biosynthesis enzymes, polyketide biosynthesis gene cluster products, a terpenoid biosynthesis pathway, an oligosaccharide biosynthesis pathway, an indolocarbazole biosynthesis pathway, a photosynthetic light harvesting complex, a stressosome, or a quorum sensing cluster. See, Fischbach and Voigt, Biotechnol. J., 5:1277-1296 (2010), which is incorporated by reference, for a detailed description and examples of each.

Native operons or gene clusters used in embodiments of the present invention can be derived (originated) from prokaryotes or eukaryotes.

As used herein, “native” is intended to refer to the host cell or host genome from which an operon or gene cluster is originally derived (e.g., as the operon is found in nature). Thus, “native expression” of an operon refers to the specific expression levels and patterns of a set of genes in an operon or gene cluster in a native host.

An operon refers to a unit of DNA comprising multiple separate coding sequences under the control of a single promoter. The separate coding sequences are typically expressed within a single RNA molecule and subsequently translated separately, e.g., with varying translation levels due to the strength of ribosomal binding sites (RBSs) associated with the particular coding sequences. Operons are most typically found in prokaryotic cells.

Gene clusters refer to sets of genes having a common function or function product. Genes are typically found within physical proximity to each other within genomic DNA (e.g., within one centiMorgan (cM)). Gene clusters can occur in prokaryotic or eukaryotic cells.

### A. Coding Sequences

Once a native operon or gene cluster has been identified for re-engineering, the coding sequences to be re-engineered can be identified. Generally, it will be desirable to start with only the coding sequences from the native operon or gene cluster, thereby removing native promoters and other non-coding regulatory sequences. Depending on the function of the various gene products of the native operon or gene cluster, in some embodiments all of the coding sequences of a native operon or gene cluster are re-engineered.

Alternatively, one or more coding sequences can be omitted from the re-engineering process. For example, it may be known that one or more of the gene products in a native operon or gene cluster do not contribute to the function product of the operon or may not be necessary for generation of the operon's or cluster's product. For example, as described in the examples below, in re-engineering the Nif operon, the nifT gene had no known function and notably it was known that elimination of nifT did not to significantly affect the ultimate function of the operon, i.e., nitrogen fixation. Thus, nifT was not included in the re-engineering process.

In some embodiments, the operon or gene cluster will include coding sequences for regulatory proteins that regulate expression or activity of one or more of the other products of the operon or gene cluster. In such embodiments, it can be desirable to omit such regulatory proteins from the re-engineering process because synthetic regulation will be employed instead. For example, as described in the examples below, in re-engineering the nif operon, nifL and nifA were known to act as regulatory genes for the nif operon and thus were omitted so that synthetic regulation could be instead used.

Once the set of gene products to be re-engineered has been identified, one can start with the native coding sequence, or the amino acid sequences of the gene products. For example, in some embodiments, the amino acid sequences of the gene products can be used to produce a synthetic coding sequence for expression in the host cell in which the re-engineered products are to be ultimately expressed.

In some embodiments, the native coding sequences of the set of gene products to be re-engineered are used as a starting point. In this case, in some embodiments, sequences not essential to production of the gene products is eliminated. For example, ribosome binding sites, terminators, or promoters within the coding sequences can be eliminated. In some embodiments, the nucleotide sequences of the coding sequences are analyzed using an algorithm (i.e., in a computer) to identify ribosome binding sites, terminators, or promoters within the sequence(s).

Nonessential regulatory sequences within the coding sequences can be reduced or eliminated by altering the codons of the native coding sequence(s). Regulatory sequences comprising codons can be disrupted, for example, by changing the codons to synonymous codons (i.e., encoding the same amino acid) thereby leaving the encoded amino acid sequence intact while changing the coding sequence. One or more codons of one or more coding sequences can be altered.

In some embodiments, at least 5%, 10%, 15%, 20% or more codons of one or more native coding sequence to be inserted into a synthetic operon are replaced. In some embodiments, at least 5%, 10%, 15%, 20%, 30%, 40%, 50% or more codons of each of the native coding sequences to be inserted into a synthetic operon are replaced.

In some embodiments, replacement codons can be selected, for example, to be significantly divergent from the native codons. The codon changes can result in codon optimization for the host cell, i.e., the cell in which the polynucleotide is to be expressed for testing and/or for ultimate expression. Methods of codon optimization are known (e.g., Sivaraman et al., Nucleic Acids Res. 36:e16 (2008); Mirzahoseini, et al., Cell Journal (Yakhteh) 12(4):453 Winter 2011; U.S. Pat. No. 6,114,148) and can include reference to commonly used codons for a particular host cell. In some embodiments, one or more codon is randomized, i.e., a native codon is replaced with a random codon encoding the same amino acid. This latter approach can help to remove any cis-acting sequences involved in the native regulation of the polypeptide. In some embodiments, codons are selected to create a DNA sequence that is maximally distant from the native sequence. In some embodiments, an algorithm is used to eliminate transcriptionally functional sequences in a gene encoding the polypeptide. For example, in some embodiments, ribosome binding sites, transcriptional regulatory elements, terminators, or other DNA sequences bound by proteins are removed from the native coding sequence. Notably, the functional sequences removed can be functional in the native species (from which the sequence was originally derived), in the heterologous host cell, or both. In some embodiments, optimizing comprises removal of sequences in the native coding sequence that are functional for heterologous transcriptional activators or repressors to be used to regulate the synthetic operons to be generated.

Generation of synthetic coding sequences, as well as the remaining portions of the synthetic operon, in many cases will be performed de novo from synthetic oligonucleotides. Thus, in some embodiments, codons are selected to create a DNA sequence that does not generate difficulties for oligonucleotide production or combination. Thus, in some embodiments, codon sequences are avoided that would result in generation of oligonucleotides that form hairpins.

In some embodiments, as noted above, codon alteration will depend on the host cell used. Host cells can be any prokaryotic cell (including but not limited to E. coli) or eukaryotic cell (including but not limited to yeast cells, mammalian cells, avian cells, amphibian cells, plant cells, fish cells, and insect cells).

Nonessential regulatory sequences within native sequences can be identified, in some embodiments, using an algorithm performed by a processor executing instructions encoded on a computer-readable storage medium. For example, in some embodiments, ribosome binding sites are identified using a thermodynamic model that calculates the free energy of the ribosome binding to mRNA. In some embodiments, promoters are identified with an algorithm using a position weighted matrix. In some embodiments, transcriptional terminators are identified by an algorithm that identifies hairpins and/or poly-A tracks within sequences. In some embodiments, an algorithm identifies other transcriptionally functional sequences, including but not limited to transposon insertion sites, sites that promote recombination, sites for cleavage by restriction endonucleases, and/or sequences that are methylated.

In view of the alterations described above, in some embodiments, a coding sequence in a synthetic operon of the invention is less than 90, 85, 80, 75, or 70% identical to the native coding sequence. In some embodiments, the coding sequence encodes a protein sequence that is identical to the native protein or is at least 80, 85, 90 or 95% identical to the native protein. In some embodiments, less than 70%, 60%, or 50% of codons in one, two or more coding sequences in a synthetic operon are identical to the codons in the native coding sequence.

B. Organizing Coding Sequences into Synthetic Operons

Once coding sequences have been selected (e.g., and substantially “cleaned” of native or spurious regulatory sequences), the coding sequences are organized into one or more synthetic operon(s). Organization of the synthetic operon(s) includes insertion of various heterologous transcriptional and translational sequences between, before, and/or after the coding sequences so that expression of each coding sequence is controlled as desired. Thus, for example, 5′ promoter sequences can be selected to drive expression of an operon RNA comprising the coding sequences of the operon. Selection of one or more terminator of appropriate strength will also affect expression levels. Moreover, the order of the coding sequences within a synthetic operon and/or selection of RBSs for the coding sequences allows for control of relative translation rates of each coding sequence, thereby allowing several levels of control for absolute and relative levels of the final protein products.

Because each synthetic operon can have its own promoter, different synthetic operons can be expressed at different strengths. Thus, in some embodiments, coding sequences are organized into different operons based on the relative native expression levels. Said another way, in some embodiments, coding sequences are organized into operons by grouping coding sequences expressed at substantially the same native level in a particular synthetic operon.

Moreover, because coding sequences at the 5′ (front) end of an RNA can be expressed at a higher level than coding sequences further 3′, in some embodiments, coding sequences are ordered within a synthetic operon such that the highest expressing coding sequence (in the native context) occurs first and the lowest expressing gene occurs last. In some embodiments, organization of genes within operons is based on native temporal expression, function, ease of manipulation of DNA, and/or experimental design.

In designing the transcriptional (e.g., promoters) and translational (e.g., RBSs) controls of the synthetic operons, the ratio of proteins measured in the native system can be considered. Thus, in some embodiments, two or more coding sequences that are expressed in a native context at substantially the same level and/or that are desirably expressed in an approximately 1:1 ratio to achieve functionality (e.g., where two or more members are part of a functional complex in a 1:1 ratio) are placed in proximity to each other within a synthetic operon. “Proximity” will generally mean that coding sequences are adjacent to each other in the synthetic operon.

In some embodiments, relative expression levels of coding sequences within and, in some embodiments, between synthetic operons is determined by testing one or more test operons for desired expression and/or desired functionality and then improving expression based on the initial results. While this method can be performed in a “trial and error” basis, in some embodiments, a numerical optimization method is employed to guide selection of regulatory elements in order to alter gene expression and to improve desired system properties. Such methods, for example, can be performed by a processor executing instructions encoded on a computer-readable storage medium (discussed further below). Exemplary numerical optimization methods include but are not limited to, a a Nelder-Mead algorithm, a Newton's method, a quasi-Newton method, a conjugate gradient method, an interior point method, a gradient descent, a subgradient method, a ellipsoid method, a Frank-Wolfe method, an interpolation method and pattern search methods, or an ant colony model. In some embodiments, a computational design of experiments (DoE) method is employed to alter gene expression and to improve desired system properties in the synthetic operons.

Transcriptional regulatory elements, ribosomal binding sites, terminators, and other sequences affecting transcription or translation can be selected from existing collections of such sequences, and/or can be generated by screening of libraries generated by design or by random mutation. Exemplary regulatory sequences include cis-acting nucleotide sequences bound by a sequence-specific DNA binding polypeptide, e.g., a transcriptional activator or a transcriptional repressor. Exemplary transcriptional activators include, but are not limited to, sigma factors, RNA polymerases (RNAPs) and chaperone-assisted activators. In some embodiments, the transcriptional activator/cis-acting sequence cognate pair will be orthogonal to the host cell. Said another way, the regulatory sequence will not be bound by other host cells proteins except for the heterologous transcriptional activator that binds the cis-acting sequence.

i. Sigma Factors

In some embodiments, the sequence-specific DNA binding polypeptide is a sigma (σ) factor and the regulatory sequence of the synthetic operon comprises the sigma factor's cognate cis-acting nucleotide sequence. Sigma factors recruit RNA polymerase (RNAP) to specific promoter sequences to initiate transcription. The σ70 family consist of 4 groups: Group 1 are the housekeeping σs and are essential; groups 2-4 are alternative σs that direct cellular transcription for specialized needs (Gruber and Gross, Annu. Rev. Microbiol., 57:441-466 (2003)). Group 4 σs (also known as ECF σs; extracytoplasmic function) constitute the largest and most diverse group of σs, and have been classified into 43 subgroups (Staron et al., Mol Microbiol 74(3): 557-81 (2009)).

In some embodiments, the set of sequence-specific DNA-binding polypeptides comprise multiple sigma factors. In some embodiments, the set comprises sigma factors from Group 1, Group 2, Group 3, and/or Group 4 Sigma factors. The ECF subgroup of Group 4 is thought to recognize different promoter sequences, making these σs particularly useful for constructing orthogonal σ-promoter systems. However, it will be appreciated that any group of sigma factors can be used according to the methods of the embodiments of the invention to develop cognate pairs.

TABLE 1

| Group | | | | | |
|---|---|---|---|---|---|
| Nr^{a} | ID^{b} | GI^{c} | SPECIES^{d} | CLASS^{d} | PHYLUM^{d} |
| ECF01 | >3473 | 109899616 | Pseudoalteromonas atlantica T6c | Gammaproteobacteria | Proteobacteria |
| ECF01 | >4085 | 114562024 | Shewanella frigidimarina NCIMB 400 | Gammaproteobacteria | Proteobacteria |
| ECF02 | >2817 | 16130498 | Escherichia coli K12 | Gammaproteobacteria | Proteobacteria |
| ECF02 | >915 | 119774011 | Shewanella amazonensis SB2B | Gammaproteobacteria | Proteobacteria |
| ECF03 | >1198 | 29350055 | Bacteroides thetaiotaomicron VPI-5482 | | Bacteroidetes |
| ECF03 | >1244 | 34541012 | Porphyromonas gingivalis W83 | | Bacteroidetes |
| ECF04 | >1609 | 21673117 | Chlorobium tepidum TLS | | Chlorobi |
| ECF04 | >1617 | 68549683 | Pelodictyon phaeoclathratiforme BU-1 | | Chlorobi |
| ECF05 | >965 | 28868416 | Pseudomonas syringae pv. tomato str. DC3000 | Gammaproteobacteria | Proteobacteria |
| ECF05 | >1054 | 67154316 | Azotobacter vinelandii AvOP | Gammaproteobacteria | Proteobacteria |
| ECF06 | >3576 | 15595669 | Pseudomonas aeruginosa PAO1 | Gammaproteobacteria | Proteobacteria |
| ECF06 | >853 | 26987094 | Pseudomonas putida KT2440 | Gammaproteobacteria | Proteobacteria |
| ECF07 | >980 | 67154823 | Azotobacter vinelandii AvOP | Gammaproteobacteria | Proteobacteria |
| ECF07 | >1134 | 15598606 | Pseudomonas aeruginosa PAO1 | Gammaproteobacteria | Proteobacteria |
| ECF08 | >3580 | 15595872 | Pseudomonas aeruginosa PAO1 | Gammaproteobacteria | Proteobacteria |
| ECF08 | >3627 | 70730114 | Pseudomonas fluorescens Pf-5 | Gammaproteobacteria | Proteobacteria |
| ECF09 | >3581 | 15597622 | Pseudomonas aeruginosa PAO1 | Gammaproteobacteria | Proteobacteria |
| ECF09 | >1009 | 70730971 | Pseudomonas fluorescens Pf-5 | Gammaproteobacteria | Proteobacteria |
| ECF10 | >3486 | 77360766 | Pseudoalteromonas haloplanktis TAC125 | Gammaproteobacteria | Proteobacteria |
| ECF10 | >2914 | 88706154 | gamma proteobacterium KT 71 | Gammaproteobacteria | Proteobacteria |
| ECF11 | >3726 | 28868260 | Pseudomonas syringae pv. tomato str. DC3000 | Gammaproteobacteria | Proteobacteria |
| ECF11 | >987 | 28899132 | Vibrio parahaemolyticus RIMD 2210633 | Gammaproteobacteria | Proteobacteria |
| ECF12 | >807 | 86158800 | Anaeromyxobacter dehalogenans 2CP-C | Deltaproteobacteria | Proteobacteria |
| ECF12 | >808 | 108762328 | Myxococcus xanthus DK 1622 | Deltaproteobacteria | Proteobacteria |
| ECF13 | >1146 | 33152898 | Haemophilus ducreyi 35000HP | Gammaproteobacteria | Proteobacteria |
| ECF13 | >1025 | 37524103 | Photorhabdus luminescens subsp. laumondii TTO1 | Gammaproteobacteria | Proteobacteria |
| ECF14 | >3200 | 15608361 | Mycobacterium tuberculosis H37Rv | | Actinobacteria |
| ECF14 | >1324 | 21223516 | Streptomyces coelicolor A3(2) | | Actinobacteria |
| ECF15 | >436 | 77464848 | Rhodobacter sphaeroides 2.4.1 | Alphaproteobacteria | Proteobacteria |
| ECF15 | >524 | 16127705 | Caulobacter crescentus CB15 | Alphaproteobacteria | Proteobacteria |
| ECF16 | >3622 | 104782321 | Pseudomonas entomophila L48 | Gammaproteobacteria | Proteobacteria |
| ECF16 | >973 | 161378140 | Pseudomonas putida KT2440 | Gammaproteobacteria | Proteobacteria |
| ECF17 | >1691 | 15607875 | Mycobacterium tuberculosis H37Rv | | Actinobacteria |
| ECF17 | >1458 | 21221399 | Streptomyces coelicolor A3(2) | | Actinobacteria |
| ECF18 | >4451 | 21230791 | Xanthomonas campestris pv. campestris str. ATCC | Gammaproteobacteria | Proteobacteria |
| | | | 33913 | | |
| ECF18 | >4438 | 21242133 | Xanthomonas axonopodis pv. citri str. 306 | Gammaproteobacteria | Proteobacteria |
| ECF19 | >3197 | 15607586 | Mycobacterium tuberculosis H37Rv | | Actinobacteria |
| ECF19 | >1315 | 21219164 | Streptomyces coelicolor A3(2) | | Actinobacteria |
| ECF20 | >992 | 70731405 | Pseudomonas fluorescens Pf-5 | Gammaproteobacteria | Proteobacteria |
| ECF20 | >2913 | 88706222 | gamma proteobacterium KT 71 | Gammaproteobacteria | Proteobacteria |
| ECF21 | >1280 | 29350128 | Bacteroides thetaiotaomicron VPI-5482 | | Bacteroidetes |
| ECF21 | >2825 | 89889680 | Flavobacteria bacterium BBFL7 | | Bacteroidetes |
| ECF22 | >4450 | 21232074 | Xanthomonas campestris pv. campestris str. ATCC | Gammaproteobacteria | Proteobacteria |
| | | | 33913 | | |
| ECF22 | >1147 | 21243541 | Xanthomonas axonopodis pv. citri str. 306 | Gammaproteobacteria | Proteobacteria |
| ECF23 | >231 | 15895043 | Clostridium acetobutylicum ATCC 824 | | Firmicutes |
| ECF23 | >1851 | 30261806 | Bacillus anthracis str. Ames | | Firmicutes |
| ECF24 | >69 | 16079737 | Bacillus subtilis subsp. subtilis str. 168 | | Firmicutes |
| ECF24 | >1034 | 32470052 | Escherichia coli | Gammaproteobacteria | Proteobacteria |
| ECF25 | >1645 | 170078575 | Synechococcus sp. PCC 7002 | | Cyanobacteria |
| ECF25 | >1643 | 17230772 | Nostoc sp. PCC 7120 | | Cyanobacteria |
| ECF26 | >4464 | 58581966 | Xanthomonas olyzae pv. oryzae KACC10331 | Gammaproteobacteria | Proteobacteria |
| ECF26 | >837 | 77459110 | Pseudomonas fluorescens PfO-1 | Gammaproteobacteria | Proteobacteria |
| ECF27 | >4265 | 21222299 | Streptomyces coelicolor A3(2) | | Actinobacteria |
| ECF27 | >1331 | 31795084 | Mycobacterium bovis AF2122/97 | | Actinobacteria |
| ECF28 | >1088 | 114563849 | Shewanella frigidimarina NCIMB 400 | Gammaproteobacteria | Proteobacteria |
| ECF28 | >1040 | 15641058 | Vibrio cholerae O1 biovar eltor str. N16961 | Gammaproteobacteria | Proteobacteria |
| ECF29 | >371 | 13476734 | Mesorhizobium loti MAFF303099 | Alphaproteobacteria | Proteobacteria |
| ECF29 | >2688 | 71281387 | Colwellia psychrerythraea 34H | Gammaproteobacteria | Proteobacteria |
| ECF30 | >35 | 16079766 | Bacillus subtilis subsp. subtilis str. 168 | | Firmicutes |
| ECF30 | >83 | 18309341 | Clostridium perfringens str. 13 | | Firmicutes |
| ECF31 | >2963 | 85713274 | Idiomarina baltica OS145 | Gammaproteobacteria | Proteobacteria |
| ECF31 | >34 | 16080921 | Bacillus subtilis subsp. subtilis str. 168 | | Firmicutes |
| ECF32 | >1122 | 4581629 | Erwinia amylovora | Gammaproteobacteria | Proteobacteria |
| ECF32 | >3724 | 28868612 | Pseudomonas syringae pv. tomato str. DC3000 | Gammaproteobacteria | Proteobacteria |
| ECF33 | >375 | 27378153 | Bradyrhizobium japonicum USDA 110 | Alphaproteobacteria | Proteobacteria |
| ECF33 | >423 | 39934888 | Rhodopseudomonas palustris CGA009 | Alphaproteobacteria | Proteobacteria |
| ECF34 | >3302 | 77164965 | Nitrosococcus oceani ATCC 19707 | Gammaproteobacteria | Proteobacteria |
| ECF34 | >1384 | 21218750 | Streptomyces coelicolor A3(2) | | Actinobacteria |
| ECF35 | >3582 | 15598092 | Pseudomonas aeruginosa PAO1 | Gammaproteobacteria | Proteobacteria |
| ECF35 | >1119 | 24375055 | Shewanella oneidensis MR-1 | Gammaproteobacteria | Proteobacteria |
| ECF36 | >3196 | 15609206 | Mycobacterium tuberculosis H37Rv | | Actinobacteria |
| ECF36 | >1595 | 21219385 | Streptomyces coelicolor A3(2) | | Actinobacteria |
| ECF37 | >3390 | 89094252 | Oceanospirillum sp. MED92 | Gammaproteobacteria | Proteobacteria |
| ECF37 | >2513 | 83718468 | Burkholderia thailandensis E264 | Betaproteobacteria | Proteobacteria |
| ECF38 | >1322 | 21222029 | Streptomyces coelicolor A3(2) | | Actinobacteria |
| ECF38 | >1442 | 152967344 | Kineococcus radiotolerans SRS30216 | | Actinobacteria |
| ECF39 | >1438 | 21223369 | Streptomyces coelicolor A3(2) | | Actinobacteria |
| ECF39 | >2973 | 84494624 | Janibacter sp. HTCC2649 | | Actinobacteria |
| ECF40 | >3198 | 15610550 | Mycobacterium tuberculosis H37Rv | | Actinobacteria |
| ECF40 | >1380 | 62389491 | Colynebacterium glutamicum ATCC 13032 | | Actinobacteria |
| ECF41 | >491 | 16127496 | Caulobacter crescentus CB15 | Alphaproteobacteria | Proteobacteria |
| ECF41 | >1141 | 77459658 | Pseudomonas fluorescens PfO-1 | Gammaproteobacteria | Proteobacteria |
| ECF42 | >3583 | 15596548 | Pseudomonas aeruginosa PAO1 | Gammaproteobacteria | Proteobacteria |
| ECF42 | >4454 | 77747962 | Xanthomonas campestris pv. campestris str. ATCC | Gammaproteobacteria | Proteobacteria |
| | | | 33913 | | |
| ECF43 | >4437 | 21244845 | Xanthomonas axonopodis pv. citri str. 306 | Gammaproteobacteria | Proteobacteria |
| ECF43 | >3477 | 109897287 | Pseudoalteromonas atlantica T6c | Gammaproteobacteria | Proteobacteria |

In addition to native sigma factors, chimeric or other variant sigma factors can also be used in the method of the invention. For example, in some embodiments, one or more sigma factor are submitted to mutation to generate library of sigma factor variants and the resulting library can be screen for novel DNA binding activities.

In some embodiments, chimeric sigma factors formed from portions of two or more sigma factors can be used. Accordingly, embodiments of the invention provide for generating a library of polynucleotides encoding chimeric sigma factors, wherein the chimeric sigma factors comprise a domain from at least two different sigma factors, wherein each of the domains bind to the −10 or −35 region of a regulatory element; and expressing chimeric sigma factors from the library of polynucleotides, thereby generating a library of chimeric sigma factors. For example, in some embodiments, chimeric sigma factors are generated comprising a “Region 2” from a first sigma factor and a “Region 4” from a second sigma factor, thereby generating chimeric sigma factors with novel DNA binding activities. “Region 2” of sigma factors is a conserved domain that recognizes −10 regions of promoters. “Region 4” is a conserved domain of sigma factors that recognizes −35 regions of promoters. It will be appreciated that chimeric sigma factors can be generated from any two native sigma factors that bind different target DNA sequences (e.g., different promoter sequences). It has been found that chimeric sigma factors formed from the ECF2 and ECF11 subgroups have unique DNA binding activities useful for generating orthogonal sets as described herein. Exemplary chimeric sigma factors include, but are not limited to, ECF11_ECF02 (containing amino acids 1-106 from ECF02_2817 and 122-202 from ECF11_3726) and ECF02_ECF11 (containing amino acids 1-121 from ECF11_3726 and 107-191 from ECF02_2817).

The ECF11_ECF02 amino acid sequence is as follows (SEQ ID NO:239):

| | |
|---|---|
| 1 | MRITASLRTFCHLSTPHSDSTTSRLWIDEVTAVARQRDRDSFMRIYDHFAPRLLRYLTGL |
| 61 | NVPEGQAEELVQEVLLKLWHKAESFDPSKASLGTWLFRIARNLYIDSVRKDRGWVQVQNS |
| 121 | LEQLERLEAISNPENLMLSEELRQIVERTIESLPEDLRMAITLRELDGLSYEEIAAIMDC |
| 181 | PVGTVRSRIFRAREAIDNKVQPLIRR* |

The ECF02_ECF11 amino acid sequence is as follows (SEQ ID NO:240):

| | |
|---|---|
| 1 | MSEQLTDQVLVERVQKGDQKAFNLLVVRYQHKVASLVSRYVPSGDVPDVVQEAFIKAYRA |
| 61 | LDSFRGDSAFYTWLYRIAVNTAKNYLVAQGRRPPSSDVDAIEAENFEQLERLEAPVDRTL |
| 121 | DYSQRQEQQLNSAIQNLPTDQAKVLRMSYFEALSHREISERLDMPLGTVKSCLRLAFQKL |
| 181 | RSRIEES* |

ii. RNA Polymerases

In some embodiments, the sequence-specific DNA-binding polypeptide is a polypeptide having DNA binding activity and that is a variant of the T7 RNA polymerase (RNAP) and the RNAP's cognate cis-acting sequence (e.g., a promoter recognized by the RNAP) is operably linked to the synthetic operon to control the operon's expression. The T7 RNAP amino acid sequence (SEQ ID NO:241) is as follows:

| | |
|---|---|
| 1 | mntiniaknd fsdielaaip fntladhyge rlareqlale hesyemgear frkmferqlk |
| 61 | agevadnaaa kplittllpk miarindwfe evkakrgkrp tafqflqeik peavayitik |
| 121 | ttlacltsad nttvqavasa igraiedear fgrirdleak hfkknveeql nkrvghvykk |
| 181 | afmqvveadm lskgllggea wsswhkedsi hvgvrcieml iestgmvslh rqnagvvgqd |
| 241 | setielapey aeaiatraga lagispmfqp cvvppkpwtg itgggywang rrplalvrth |
| 301 | skkalmryed vympevykai niaqntawki nkkvlavanv itkwkhcpve dipaiereel |
| 361 | pmkpedidmn pealtawkra aaavyrkdka rksrrislef mleqankfan hkaiwfpynm |
| 421 | dwrgrvyays mfnpqgndmt kglltlakgk pigkegyywl kihgancagv dkvpfperik |
| 481 | fieenhenim acaksplent wwaeqdspfc flafcfeyag vqhhglsync slplafdgsc |
| 541 | sgiqhfsaml rdevggravn llpsetvgdi ygivakkvne ilqadaingt dnevvtvtde |
| 601 | ntgeisekvk lgtkalagqw laygvtrsvt krsvmtlayg skefgfrqqv ledtiqpaid |
| 661 | sgkglmftqp nqaagymakl iwesysvtvv aaveamnwlk saakllaaev kdkktgeilr |
| 721 | krcavhwvtp dgfpvwqeyk kpiqtrlnlm flgqfrlqpt intnkdseid ahkqesgiap |
| 781 | nfvhsqdgsh lrktvvwahe kygiesfali hdsfgtipad aanlfkavre tmvdtyescd |
| 841 | vladfydqfa dqlhesqldk mpalpakgnl nlrdilesdf afa |

The T7 RNAP promoter has also been characterized (see, e.g., Rong et al., Proc. Natl. Acad. Sci. USA, 95(2):515-519 (1998)) and is well known.

Methods have been discovered for generating orthogonal pairs of RNAP variants and target promoter variants. Due to toxicity of expression of native T7 RNAP, a series of mutations and modifications can be designed such that a library of RNAP variants can be expressed and tested for activity in cells without excessive toxicity. Accordingly, embodiments of the invention provide for one or more of the following modifications (and thus, for example, an embodiment of the invention provides for host cells comprising expression cassettes, or nucleic acids comprising expression cassettes, wherein the expression cassette encodes a RNAP variant substantially identical to T7 RNAP, wherein the expression cassette comprises one or more of the following): Expression of the T7 RNAP variant can be expressed from a low copy plasmid.

Expression of the RNAP can be controlled by a separately encoded protein from a separate vector, thereby blocking expression of the RNAP until a second vector is added to the cells promoting RNAP expression;

Translational control: a GTG start codon; weak ribosomal binding sites, and/or random DNA spacers to insulate RNAP expression can be used;

A molecular tag to promote rapid degradation of the RNAP. For example, an Lon N-terminal tag will result in rapid degradation of the tagged RNAP by the Lon protease system.

A mutated RNAP active site (e.g., within amino acids 625-655 of T7 RNAP). For example, it ha been discovered that a mutation of the position corresponding to amino acid 632 (R632) of T7 RNAP can be mutated to reduce the RNAP's activity. In some embodiments, the RNAP contains a mutation corresponding to R632S.

Moreover, a variety of mutant T7 promoters have been discovered that can be used in a genetic circuit. Thus, in some embodiments, the regulatory sequence of a synthetic operon comprises a mutant sequence as set forth in the table below (SEQ ID NOS:156-163).

| | | Strength |
|---|---|---|
| Promoter | Sequence | (2009.10.02 to |
| Name | TAATACGACTCACTANNNNNAGA | 2009.10.09) |
| WT | TAATACGACTCACTATAGGGAGA | 5263 |
| Mut1 | TAATACGACTCACTACAGGCAGA | 365 |
| Mut2 | TAATACGACTCACTAGAGAGAGA | 366 |
| Mut3 | TAATACGACTCACTAATGGGAGA | 577 |
| Mut4 | TAATACGACTCACTATAGGTAGA | 1614 |
| Mut5 | TAATACGACTCACTAAAGGGAGA | 1018 |
| Mut6 | TAATACGACTCACTATTGGGAGA | 3216 |

A number of different stem loop structures that function as terminators for T7 RNAP have also been discovered. See, Table directly below (SEQ ID NOS:242-253). Accordingly, an embodiment of the invention provides for a synthetic operon comprising a promoter functional to a native T7 RNAP or an RNAP substantially identical thereto, wherein the operably linked polynucleotide comprises a terminator selected from the table directly below. Terminators with different sequences can be selected for different transcupts to avoid homologous recombination.

| Term- | Sequence | Strength |
|---|---|---|
| inator | TANNNNAACCSSWWSSSSSSTCWWW | (2009.12.16 |
| Name | WCGSSSSSSWWSSGGTTTTTTGT | Assay) |
| 52 | TATAAAACGGGGGGCTAGGGGTTTTTT | 107 |
| | GT | |
| 23 | TACTCGAACCCCTAGCCCGCTCTTATC | 714 |
| | GGGCGGCTAGGGGTTTTTTGT | |
| 72 | TAGCAGAACCGCTAACGGGGGCGAAG | 1051 |
| | GGGTTTTTTGT | |
| 48 | TACTCGAACCCCTAGCCCGCTCTTATC | 1131 |
| | GGGCGGCTAGGGGTTTTTTGT | |
| 1 | TACATATCGGGGGGGTAGGGGTTTTTT | 1297 |
| | GT | |
| 2 | TACATATCGGGGGGGTAGGGGTTTTTT | 1333 |
| | GT | |
| WT | TAGCATAACCCCTTGGGGCCTCTAAAC | 1395 |
| | GGGTCTTGAGGGGTTTTTTGT | |
| 31 | TACCCTAACCCCTTCCCCGGTCAATCG | 1586 |
| | GGGCGGATGGGGTTTTTTGT | |
| 58 | TAGACCAACCCCTTGCGGCCTCAATCG | 1608 |
| | GGGGGGATGGGGTTTTTTGT | |
| 25 | TACTCTAACCCCATCGGCCGTCTTAGG | 1609 |
| | GGTTTTTTGT | |
| 17 | TACCTCAACCCCTTCCGCCCTCATATC | 1887 |
| | GCGGGGCATGCGGTTTTTTGT | |

In some embodiments, RNAP variants can be designed comprising an altered specificity loop (corresponding to positions between 745 and 761). Thus in some embodiments, an RNAP is provided that is identical or substantially identical to T7 or T3 RNAP but has a Loop Sequence selected from those in the tables directly below between positions 745 and 761. Loop Sequences=SEQ ID NOS:254, 255, 257 and 259. Promoter Sequences=SEQ ID NOS:157, 256, 258 and 260.

A

| RNAP | | RNAP | Promoter | | |
|---|---|---|---|---|---|
| Family | Scaffold | Plasmid | Plasmid | Loop Sequence | Promoter Sequence |
| T7 | N249 | N249 | N155 | VWQEYKKPIQTRLNLMFLGQFRLQP | TAATACGACTCACTATA |
| | | | | TINTNKDSEIDAHK | GGGAGA |
| T3 | N115 | N377: | N352 | VWQEYKKPIQKRLDMIFLGQFRLQP | TAATAACCCTCACTATA |
| | | 115 | | TINTNKDSEIDAHK | GGGAGA |
| K1F | N115 | N421: | N353 | VWQEYKKPIQTRLNLMFLGSFNLQP | TAATAACTATACTATA |
| | | 115 | | TVNTNKDSEIDAHK | GGGAGA |
| N4 | N77 | W78 | W74 | VWQEYKKPIQTRIDCVILGTHRMAL | TAATACCCACACTATA |
| | | | | TINTNKDSEIDAHK | GGGAGA |

B

| | T7 | T3 | K1F | N4 |
|---|---|---|---|---|
| | promoter | promoter | promoter | promoter |
| T7 | 2177 | 24 | 17 | 14 |
| RNAP | | | | |
| T3 | 83 | 1062 | 14 | 14 |
| RNAP | | | | |
| K1F | 45 | 26 | 463 | 13 |
| RNAP | | | | |
| N4 | 51 | 147 | 46 | 2616 |
| RNAP | | | | |

iii. Activators Requiring Chaperones

In some embodiments, the set of sequence-specific DNA-binding polypeptides comprise polypeptides having DNA binding activity and that require a separate chaperone protein to bind the sequence-specific DNA-binding polypeptide for the sequence-specific DNA-binding polypeptide to be active. Exemplary transcriptional activators requiring a chaperone for activity include, but are not limited to activator is substantially similar to InvF from Salmonella typhimurium, MxiE from Shigella flexneri, and ExsA from Pseudomonas aeruginosa. These listed activators require binding of SicA from Salmonella typhimurium, JpgC from Shigella flexneri, or ExsC from Psuedomonas aeruginosa, respectively, for activation.

Sequence information for the above components are provides as follows (SEQ ID NOS:260-273):

| Name | Type | DNA sequence encoding the named polypeptide | Optional Mutation |
|---|---|---|---|
| sicA | Gene | atggattatcaaaataatgtcagcgaagaacgtgttgcggaaatgatttgggatgccgttagtgaag | |
| | | gcgccacgctaaaagacgttcatgggatccctcaagatatgatggacggtttatatgctcatgctta | |
| | | tgagttttataaccagggacgactggatgaagctgagacgttctttcgtttcttatgcatttatgatttt | |
| | | tacaatcccgattacaccatgggactggcggcagtatgccaactgaaaaaacaatttcagaaagc | |
| | | atgtgacctttatgcagtagcgtttacgttacttaaaaatgattatcgccccgttttttttaccgggcagt | |
| | | gtcaattattaatgcgtaaggcagcaaaagccagacagtgttttgaacttgtcaatgaacgtactga | |
| | | agatgagtctctgcgggcaaaagcgttggtctatctggaggcgctaaaaacggcggagacagag | |
| | | cagcacagtgaacaagaaaaggaataa | |
| sicA* | Mutant sicA | atggattatcaaaataatgtcagcgaagaacgtgttgcggaaatgatttgggatgccgttagtgaag | The large “t” of the sicA |
| | | gcgccacgctaaaagacgttcatgggatccctcaagatatgatggacggtttatatgctcatgctta | sequence above was |
| | | tgagttttataaccagggacgactggatgaagctgagacgttctttcgttacttatgcatttatgattt | mutated to “a” by error- |
| | | ttacaatcccgattacaccatgggactggcggcagtatgccaactgaaaaaacaatttcagaaagc | prone PCR. This |
| | | atgtgacctttatgcagtagcgtttacgttacttaaaaatgattatcgccccgtttatttaccgggcagt | mutation was made to |
| | | gtcaattattaatgcgtaaggcagcaaaagccagacagtgttttgaacttgtcaatgaacgtactga | reduce cross talk |
| | | agatgagtctctgcgggcaaaagcgttggtctatctggaggcgctaaaaacggcggagacagag | between SicA and |
| | | cagcacagtgaacaagaaaaggaataa | mxiE. |
| invF | Gene with | atgctaaatacgcaggaagtacttaaagaaggagagaagcggaaaatccgcagcccggaagca | The accepted start |
| | new start | tggtttatacagacgtgttccgcgcaaaagctgcatatgtcattttctgaaagccgacacaatga | codon (the large “atg”) |
| | codon | aaattgcctgattcaggaaggcgcgctgcttttttgcgagcaggccgttgtcgcaccagtatcagg | was determined to be |
| | | agacctggtttttcgaccgttaaaaattgaagtactcagcaaattactggcatttatcgatggcgcag | incorrect and a correct |
| | | gattagtggacacgacatatgctgaatccgataaatgggttttgctgagtcctgagtttcgcgctattt | upstream start codon |
| | | ggcaagatcgtaaacgctgcgagtactggtttttgcagcaaattattacgccttctccggccttcaat | was found. |
| | | aaggtactggcgctgttacgaaaaagcgagagttactggttggttggctatttactcgctcagtcaa | |
| | | ccagcggcaacacgatgagaatgctgggagaagactatggcgtttcttatacccattttcgtcgttt | |
| | | gtgcagcagagcgttgggcggaaaagcgaagagtgaattacgaaactggcgtatggcgcaatc | |
| | | gctgctgaatagtgtagaaggccacgagaacatcacccaattagccgttaatcatggttactcatc | |
| | | gccttcacatttttctagtgagatcaaagagctgatcggcgtttcgccgcggaaattatcaaatattat | |
| | | tcaattggcagacaaatga | |
| psicA | Promoter | ccacaagaaacgaggtacggcattgagccgcgtaaggcagtagcgatgtattcattgggcgttttt | |
| | | tgaatgttcactaaccaccgtcggggtttaataactgcatcagataaacgcagtcgttaagttctaca | |
| | | aagtcggtgacagataacaggagtaagta | |
| ipgC | Gene | atgtctttaaatatcaccgaaaatgaaagcatctctactgcagtaattgatgcaattaactctggcgct | |
| | | acactgaaagatattaatgcaattcctgatgatatgatggatgacatttattcatatgcttatgactttta | |
| | | caacaaaggaagaatagaggaagctgaagttttcttcaggtttttatgtatatacgacttttacaatgt | |
| | | agactacattatgggactcgcagctatttatcagataaaagaacagttccaacaagcagcagacct | |
| | | ttatgctgtcgcttttgcattaggaaaaaatgactatacaccagtattccatactggacaatgtcagct | |
| | | tcggttgaaagcccccttaaaagctaaagagtgcttcgaactcgtaattcaacacagcaatgatga | |
| | | aaaattaaaaataaaagcacaatcatacttggacgcaattcaggatatcaaggagtaa | |
| mxiE | Gene with | atgagtaaatataaaggcctgaacaccagcaacatgttctacatctacagctctggtcatgaacc | The wide type gene has |
| | codon | ggtgaacgttgaactggtgaaagataaagaacgtaacatcatcgaactggcaccggcgtggaaa | “ttttttttt” in this enlarged |
| | optimi- | ggctttttctttgtgcgtaaccagaacatcaaattcagcgataacgttaactaccactacc | sequence region. One |
| | zation | gcttcaacatcaactcttgcgcaaaattcctggcgttttgggattattttagcggcgccctggttgaac | more “t” was added to |
| | | attctcacgcagaaaaatgcatccatttctaccacgaaaacgatctgcgtgatagctgtaatacgga | make “tttttttttt” and then |
| | | atctatgctggataaactgatgctgcgcttcatttttagtagcgatcagaacgtgtctaatgccctggc | the entire gene was |
| | | aatgatccgtatgaccgaaagttatcatctggttctgtacctgctgcgtacgattgaaaaagaaaaa | codon optimized by |
| | | gaagtgcgcatcaaaagcctgaccgaacactatggcgtttctgaagcgtactttcgtagtctgtgtc | GenScript. The |
| | | gcaaagcgctgggtgccaaagtgaaagaacagctgaacacgtggcgcctggtgaatggcctgc | additional “t” was added |
| | | tggatgttttcctgcataaccagaccattacgagcgcggccatgaacaatggttatgcgtctaccag | to make this ORF in- |
| | | tcacttcagcaatgaaattaaaacgcgtctgggctttagtgcccgcgaactgagcaacatcaccttc | frame. In addition, the |
| | | ctggtgaagaaaattaatgaaaaaatctaa | wide-type gene starts |
| | | | with “g” and this |
| | | | synthetic gene starts |
| | | | with “a.” |
| pipaH9.8 | Promoter | gcgaaaatgacatcaaaaacgccattaacctgatgttctggggaatataaatgtcaggctagggtc | |
| | | aaaaatcgtggcgttgacaaaatggctgcgttacgtcattgagcatatccaggactggccggcaa | |
| | | accgggtacgcgatctgttgccttggaaagttgatctgacctctcagtaaatatcaatacggttctga | |
| | | cgagccgcttaccgttcaaatatgaagtacgatgtttaactaaccgaaaaacaagaacaatacggt | |
| | | gcaaacaggccattcacggttaactgaaacagtatcgttatttacagccaattttgtttatccttattat | |
| | | aataaaaaagtgct | |
| pipaH9.8* | Promoter | gcgaaaatgacatcaaaaacgccattaacctgatgttctggggaatataaatgtcaggctagggtc | The enlarged “ta” above |
| | with | aaaaatcgtggcgttgacaaaatggctgcgttacgtcattgagcatatccaggactggccggcaa | ofpipaH9.8 was |
| | mutation | accgggtacgcgatctgttgccttggaaagttgatctgacctctcagtaaatatcaatacggttctga | mutated to “ag” by |
| | | cgagccgcttaccgttcaaatatgaagtacgatgtttaactaaccgaaaaacaagaacaatacggt | saturation mutagenesis. |
| | | gcaaacaggccattcacggttaactgaaacagtatcgttatttacagccaattttgtttatccttatta | This mutation was made |
| | | agataaaaaagtgct | to reduce leaky |
| | | | expression of pipaH9.8. |
| exsC | Gene | atggatttaacgagcaaggtcaaccgactgcttgccgagttcgcaggccgtatcggtttgccttcc | |
| | | ctgtccctcgacgaggagggcatggcgagcctcctgttcgacgaacaggtgggcgtcaccctgt | |
| | | tgctgctcgccgagcgcgagcgtctgttgctggaggccgatgtggcgggcatcgatgtgctggg | |
| | | cgaggggatctttcgccagctcgccagcttcaaccgccattggcaccgtttcgatctgcatttcggc | |
| | | ttcgacgagctgaccggcaaggtccagttgtatgcgcagattctcgcagcgcaactgaccctcga | |
| | | atgcttcgaggcgaccttggccaatctgctcgatcacgccgagttctggcagcgcctgctgccgt | |
| | | gcgacagtgatcgcgaggcggtcgctgcggtcggcatgagggtttga | |
| exsD | Gene | atggagcaggaagacgataagcagtactcccgagaagcggtgttcgctggcaggcgggtatcc | |
| | | gtggtgggctcggacgcccgctcgcggggtcgggtgccgggttacgcatcgagcagtttgtatc | |
| | | gtgagtccggaatcatcagtgcgcggcaactggcgttgctgcagcggatgctgccgcgcctgcg | |
| | | gctggagcaactgttccgctgcgagtggttgcagcagcgcctggcgcgcggcctggcgctggg | |
| | | gcgcgaagaggtgcggcagattctcctctgcgcggcgcaggacgacgacggctggtgctccga | |
| | | actgggcgaccgggtcaacctcgccgtgccgcagtcgatgatcgactgggtcctgctgccggtc | |
| | | tatggctggtgggaaagcctgctcgaccaggcgatccccggctggcgcctgtcgctggtggagc | |
| | | tggagacccagtcccggcaactgcgagtcaagtccgaattctggtcccgcgtggccgagctgga | |
| | | gccggagcaggcccgcgaggaactggccagggtcgccaagtgccaggcgcgcacccagga | |
| | | acaggtggccgaactggccggcaagctggagacggcttcggcactggcgaagagcgcctggc | |
| | | cgaactggcagcggggcatggcgacgctgctcgccagcggcgggctggccggcttcgagccg | |
| | | atccccgaggtcctcgaatgcctctggcaacctctctgccggctggacgacgacgtcggcgcgg | |
| | | cggacgccgtccaggcctggctgcacgaacgcaacctgtgccaggcacaggatcacttctactg | |
| | | gcagagctga | |
| exsA | Gene | atgcaaggagccaaatctcttggccgaaagcagataacgtcttgtcattggaacattccaactttcg | |
| | | aatacagggtaaacaaggaagagggcgtatatgttctgctcgagggcgaactgaccgtccagga | |
| | | catcgattccactttttgcctggcgcctggcgagttgcttttcgtccgccgcggaagctatgtcgtaa | |
| | | gtaccaagggaaaggacagccgaatactctggattccattatctgcccagtttctacaaggcttcgt | |
| | | ccagcgcttcggcgcgctgttgagtgaagtcgagcgttgcgacgagcccgtgccgggcatcatc | |
| | | gcgttcgctgccacgcctctgctggccggttgcgtcaaggggttgaaggaattgcttgtgcatgag | |
| | | catccgccgatgctcgcctgcctgaagatcgaggagttgctgatgctcttcgcgttcagtccgcag | |
| | | gggccgctgctgatgtcggtcctgcggcaactgagcaaccggcatgtcgagcgtctgcagctatt | |
| | | catggagaagcactacctcaacgagtggaagctgtccgacttctcccgcgagttcggcatgggg | |
| | | ctgaccaccttcaaggagctgttcggcagtgtctatggggtttcgccgcgcgcctggatcagcga | |
| | | gcggagaatcctctatgcccatcagttgctgctcaacagcgacatgagcatcgtcgacatcgccat | |
| | | ggaggcgggcttttccagtcagtcctatttcacccagagctatcgccgccgtttcggctgcacgcc | |
| | | gagccgctcgcggcaggggaaggacgaatgccgggctaaaaataactga | |
| pexsD | Promoter | gaaggacgaatgccgggctaaaaataactgacgttttttgaaagcccggtagcggctgcatgagt | |
| | | agaatcggcccaaat | |
| pexsC | Promoter | gatgtggcttttttcttaaaagaaaagtctctcagtgacaaaagcgatgcatagcccggtgctagca | |
| | | tgcgctgagcttt | |
| rfp | Gene | atggcttcctccgaagacgttatcaaagagttcatgcgtttcaaagttcgtatggaaggttccgttaa | |
| | | cggtcacgagttcgaaatcgaaggtgaaggtgaaggtcgtccgtacgaaggtacgcagaccgct | |
| | | aaactgaaagttaccaaaggtggtccgctgccgttcgcttgggacatcctgtccccgcagttccag | |
| | | tacggttccaaagcttacgttaaacacccggctgacatcccggactacctgaaactgtccttcccg | |
| | | gaaggtttcaaatgggaacgtgttatgaacttcgaagacggtggtgttgttaccgttacccaggact | |
| | | cctccctgcaagacggtgagttcatctacaaagttaaactgcgtggtactaacttcccgtccgacg | |
| | | gtccggttatgcagaaaaaaaccatgggttgggaagcttccaccgaacgtatgtacccggaagac | |
| | | ggtgctctgaaaggtgaaatcaaaatgcgtctgaaactgaaagacggtggtcactacgacgctga | |
| | | agttaaaaccacctacatggctaaaaaaccggttcagctgccgggtgcttacaaaaccgacatca | |
| | | aactggacatcacctcccacaacgaagactacaccatcgttgaacagtacgaacgtgctgaaggt | |
| | | cgtcactccaccggtgctgcagcaaacgacgaaaactacgcttaa | |

### C. Controlling Operon Expression

As noted above, the one or more synthetic operons are controlled by regulatory elements responsive to a sequence-specific DNA binding polypeptide (e.g., a transcriptional activator). Where more than one operon is used, it can be desirable that each operon be responsive to the same transcriptional activator, albeit with a different regulatory sequence that controls the “strength” of expression of a particular operon. As noted above, in some embodiments, the transcriptional activator is a T7 RNAP or a variant thereof.

Expression of the sequence-specific DNA binding polypeptide can be controlled on a separate expression cassette, the expression cassette comprising a promoter operably linked to a polynucleotide encoding the sequence-specific DNA binding polypeptide. In some embodiments, the promoter is inducible, thereby imparting control of expression of the operon based on the inducer. Exemplary inducible promoters (with inducer in parentheses) include, e.g., Ptac (IPTG), Ptrc (IPTG), Pbad (arabinose), Ptet (aTc), Plux (AI-1). Alternatively, in some embodiments, the promoter is constitutive

Expression of the sequence-specific DNA binding polypeptide can be controlled on a separate expression cassette, the expression cassette comprising a promoter operably linked to a polynucleotide encoding the sequence-specific DNA binding polypeptide. In some embodiments, the promoter is inducible, thereby imparting control of expression of the operon based on the inducer. Exemplary inducible promoters (with inducer in parentheses) include, e.g., Ptac (IPTG), Ptrc (IPTG), Pbad (arabinose), Ptet (aTc), Plux (AI-1). Alternatively, in some embodiments, the promoter is constitutive.

In some embodiments, additional “buffer” nucleotide sequences are inserted between promoters and ribosomal binding sites, between coding sequences and terminators, and/or between coding sequences and a subsequent ribosomal binding site. These sequences act as “buffers” in that they reduce or eliminate regulatory cross-talk between different coding sequences. In some embodiments, the spacer forms a stem loop, is a native sequence from a metabolic pathway, or is from a 5′-UTR, e.g., obtained from a phage. In some embodiments, the stem loop is a ribozyme. In some embodiments, the ribozyme is RiboJ. In some embodiments, the buffer sequence is selected from sequences of a given length with nucleotides selected at random. In some embodiments, the buffer sequence is a UP-region of a promoters. UP regions can positively influence promoter strength and are generally centered at position −50 of a promoter (as measured from the start of transcription). See, e.g., Estrem, et al., PNAS, 95 (11): 9761-9766 (1988). In some embodiments, the buffer sequence is an extended 5-UTR sequence.

Exemplary buffer sequences include those listed in the table below (SEQ ID NOS:274-333, respectively):

| Sources | Sequences |
|---|---|
| T5 phage | agttcgatgagagcgataaccctctacaaataattttgtttaa |
| T5 phage | ataaattgataaacaaaaacctctacaaataattttgtttaa |
| T5 phage | ataaatttgagagaggagttcctctacaaataattttgtttaa |
| T5 phage | attaaagaggagaaattaaccctctacaaataattttgtttaa |
| T5 phage | aaacctaatggatcgaccttcctctacaaataattttgtttaa |
| T7 phage | atcgagagggacacggcgacctctacaaataattttgtttaa |
| T7 phage | gctaggtaacactagcagccctctacaaataattttgtttaa |
| T7 phage | atgaaacgacagtgagtcacctctacaaataattttgtttaa |
| T7 phage | agggagaccacaacggtttccctctacaaataattttgtttaa |
| High-transcription | attaaaaaacctgctaggatcctctacaaataattttgtttaa |
| escape | |
| High-transcription | ataaaggaaaacggtcaggtcctctacaaataattttgtttaa |
| escape | |
| High-transcription | ataggttaaaagcctgtcatcctctacaaataattttgtttaa |
| escape | |
| Carbon utilization | acaataaaaaatcatttacatgtttcctctacaaataattttgtttaa |
| Carbon utilization | agaagcagcgcgcaaaaatcagctgcctctacaaataattttgtttaa |
| Carbon utilization | atgagttcatttcagacaggcaaatcctctacaaataattttgtttaa |
| Carbon utilization | aacttgcagttatttactgtgattacctctacaaataattttgtttaa |
| Carbon utilization | agccacaaaaaaagtcatgttggttcctctacaaataattttgtttaa |
| Carbon utilization | acacagtcacttatcttttagttaaaaggtcctctacaaataattttgtttaa |
| Anti-escaping | atccggaatcctcttcccggcctctacaaataattttgtttaa |
| sequences | |
| | aacaaaataaaaaggagtcgctcaccctctacaaataattttgtttaa |
| T5 phage | agttcgatgagagcgataacagttccagattcaggaactataa |
| T5 phage | ataaattgataaacaaaaaagttccagattcaggaactataa |
| T5 phage | ataaatttgagagaggagttagttccagattcaggaactataa |
| T5 phage | attaaagaggagaaattaacagttccagattcaggaactataa |
| T5 phage | aaacctaatggatcgaccttagttccagattcaggaactataa |
| T7 phage | atcgagagggacacggcgaagttccagattcaggaactataa |
| T7 phage | gctaggtaacactagcagcagttccagattcaggaactataa |
| T7 phage | atgaaacgacagtgagtcaagttccagattcaggaactataa |
| T7 phage | agggagaccacaacggtttcagttccagattcaggaactataa |
| High-transcription | attaaaaaacctgctaggatagttccagattcaggaactataa |
| escape | |
| High-transcription | ataaaggaaaacggtcaggtagttccagattcaggaactataa |
| escape | |
| High-transcription | ataggttaaaagcctgtcatagttccagattcaggaactataa |
| escape | |
| Carbon utilization | acaataaaaaatcatttacatgtttagttccagattcaggaactataa |
| Carbon utilization | agaagcagcgcgcaaaaatcagctgagttccagattcaggaactataa |
| Carbon utilization | atgagttcatttcagacaggcaaatagttccagattcaggaactataa |
| Carbon utilization | aacttgcagttatttactgtgattaagttccagattcaggaactataa |
| Carbon utilization | agccacaaaaaaagtcatgttggttagttccagattcaggaactataa |
| Carbon utilization | acacagtcacttatcttttagttaaaaggtagttccagattcaggaactataa |
| Anti-escaping | atccggaatcctcttcccggagttccagattcaggaactataa |
| sequences | |
| | aacaaaataaaaaggagtcgctcacagttccagattcaggaactataa |
| Stem loops | gatcaccagggggatcccccggtgaaggat |
| Stem loops | gatcgcccaccggcagctgccggtgggcgatcaaggat |
| Stem loops | gatcatcggtagagttaatattgagcagatcccccggtgaaggat |
| Stem loops | attgatctggttattaaaggtaatcgggtcatttta |
| Stem loops | gttctccacgggtgggatgagcccctcgtggtggaaatgcg |
| Stem loops | agcatgaggtaaagtgtcatgcaccaa |
| Stem loops | acgtcgacttatctcgagtgagatattgttgacggtac |
| Stem loops | acgtcgacttatctcgagtgagataagttgacggtac |
| Stem loops | acgtcgacttatctcgagactgcagttcaatagagatattgttgacggtac |
| Stem loops | gactgtcaccggatgtgctttccggtctgatgagtccgtgaggacgaaacag |
| (Ribozyme) | |
| Stem loops | gatcaccagggggatcccccggtgaaggatcctctacaaataattttgataa |
| Stem loops | Gatcgcccaccggcagctgccggtgggcgatcaaggatcctctacaaataatt |
| | ttgtttaa |
| Stem loops | gatcatcggtagagttaatattgagcagatcccccggtgaaggatcctctacaaa |
| | taattttgtttaa |
| Stem loops | attgatctggttattaaaggtaatcgggtcattttacctctacaaataattttgtttaa |
| Stem loops | Gttctccacgggtgggatgagcccctcgtggtggaaatgcgcctctacaaataa |
| | ttttgtttaa |
| Stem loops | agcatgaggtaaagtgtcatgcaccaacctctacaaataattttgtttaa |
| Stem loops | Acgtcgacttatctcgagtgagatattgttgacggtaccctctacaaataattttgt |
| | ttaa |
| Stem loops | Acgtcgacttatctcgagtgagataagttgacggtaccctctacaaataattttgtt |
| | taa |
| Stem loops | acgtcgacttatctcgagactgcagttcaatagagatattgttgacggtaccctct |
| | acaaataattttgtttaa |
| Stem loops | gactgtcaccggatgtgctttccggtctgatgagtccgtgaggacgaaacagcc |
| (Ribozyme) | tctacaaataattttgtttaa |

The synthetic operons and/or the expression cassette for expressing the sequence-specific DNA binding polypeptide can be carried on one or more plasmids, e.g., in a cell. In some embodiments, the operon and the expression cassette are on different plasmids. In some embodiments, the expression cassette plasmid and/or operon plasmid(s) are low copy plasmids. Low copy plasmids can include, for example, an origin of replication selected from PSC101, PSC101*, F-plasmid, R6K, or IncW.

### II. Synthetic Operons

Embodiments of the present invention also provide for synthetic operons, for example as generated by the methods described herein.

### IV Systems of Synthetic Operons

Embodiments of the invention also provide for systems comprising synthetic operons and one or more controlling expression cassettes, wherein the expression cassette encodes a sequence-specific DNA binding polypeptide controlling expression of the synthetic operon(s). In some embodiments, the controlling expression cassette(s) are genetic circuits. For example, the expression cassettes can be designed to act as logic gates, pulse generators, oscillators, switches, or memory devices. In some embodiments, the controlling expression cassette are linked to a promoter such that the expression cassette functions as an environmental sensor. In some embodiments, the environmental sensor is an oxygen, temperature, touch, osmotic stress, membrane stress, or redox sensor.

As explained above, in some embodiments, the expression cassette encodes T7 RNAP or a functional variant thereof. In some embodiments, the T7 RNAP is the output of the genetic circuit(s).

The operons and expression cassettes can be expressed in a cell. Thus in some embodiments, a cell contains the systems of the invention. Any type of host cell can comprise the system.

### V Computation

In some aspects, the invention utilizes a computer program product that determines experimental values for controlling the magnitude of expression of two or more genes. This may be used for example to optimize a system property (e.g. nitrogen fixation levels). In one embodiment, the program code receives one or more input data points, wherein the input data points provide information about one or more regulatory elements and a system property. It then uses a computational method to determine a next data point. In one aspect, the computational method may be a design of experiments (DoE) method.

In some embodiments, the program code-generated next data point can then be used for further experimentation, e.g., to see if the suggested next data point results in optimized expression level for two or more genes, leading to an improvement in a desired system property. In one aspect, the generation of next data points is repeated until a desired system property level is obtained. In another aspect, the next data points are iteratively generated until the magnitude of expression of two or more genes reaches a desired level.

In some embodiments, the computer program code may use a computational method that employ numerical analysis or optimization algorithms. In some aspects, the numerical optimization methods may use the is the Nelder-Mead algorithm, the Newton's method, the quasi-Newton method, the conjugate gradient method, an interior point method, a gradient descent, a subgradient method, a ellipsoid method, the Frank-Wolfe method, an interpolation method and pattern search methods, or an ant colony model

In one specific embodiment, the computer program to generate the next data point for experimentation uses the Nelder-Mead algorithm. The computer-implemented method will receive one or more input data points and calculate the reflection point, expansion point or contraction point to computationally determine the next data point to experiment with, based on the input data points.

In one implementation of the Nelder-Mead algorithm, the program code will take the received input data points as the simplex vertices of an n-dimensional space, having n+1 simplex vertices. Then the objective function will be evaluated for each vertex of the simplex, and the algorithm uses this information to propose a sequence of new coordinates for evaluation. New coordinates will be determined by the computer code according to the following algorithmic logic:
1. Order the simplex vertices: f(x₁)≤f(x₂)≤ . . . ≤f(xₙ₊₁)
2. Calculate x₀ the center of gravity of all points except: xₙ₊₁
3. Calculate a Reflection coordinate: xᵣ=xₒ+α(xₒ−xₙ₊₁)
4. Calculate an Expansion coordinate: xₑ=xₒ+r(xₒ−xₙ₊₁)
5. Calculate a Contraction coordinate: x_{c}=xₙ₊₁+ρ(xₒ−xₙ₊₁)
6. Calculate Reduction coordinates: xᵢ=xₗ+σ(xᵢ−xₗ) for all i∈{2, . . . , n+1}

The objective function is evaluated at these points and used to determine a new simplex according to the following criteria:
1. If the Reflection, Expansion or Contraction coordinates are better than the worst simplex point, xₙ₊₁, define a new simplex by replacing the worst simplex point with the best of the three (Reflection, Expansion or Contraction).
2. Otherwise, define a new simplex by combining the best simplex point with the Reduction coordinates.

In one embodiment, a computer program product is provided comprising a tangible computer readable medium storing a plurality of instructions for controlling a processor to perform an operation for determining an experimentation point for controlling the magnitude of expression of two or more genes, the instructions comprising receiving one or more input data points, wherein the input data points provide information about one or more regulatory elements and a system property; and determining, with a computer, a next data point using a computational method, wherein the next data point provides information about the one or more regulatory elements.

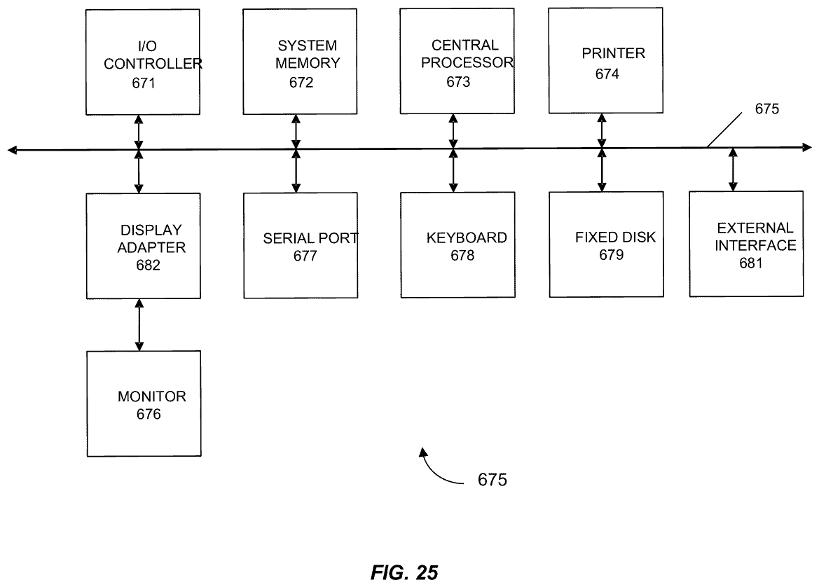

FIG. 25 shows a block diagram of an example computer system 600 usable with system and methods according to embodiments of the present invention. The computer system 600 can be used to run the program code for various method claims according to embodiments of the present invention.

Any of the computer systems mentioned herein may utilize any suitable number of subsystems. Examples of such subsystems are shown in FIG. 25 in computer apparatus 600. In some embodiments, a computer system includes a single computer apparatus, where the subsystems can be the components of the computer apparatus. In other embodiments, a computer system can include multiple computer apparatuses, each being a subsystem, with internal components.

The subsystems shown in FIG. 25 are interconnected via a system bus 675. Additional subsystems such as a printer 674, keyboard 678, fixed disk 679, monitor 676, which is coupled to display adapter 682, and others are shown. Peripherals and input/output (I/O) devices, which couple to I/O controller 671, can be connected to the computer system by any number of means known in the art, such as serial port 677. For example, serial port 677 or external interface 681 can be used to connect computer system 600 to a wide area network such as the Internet, a mouse input device, or a scanner. The interconnection via system bus 675 allows the central processor 673 to communicate with each subsystem and to control the execution of instructions from system memory 672 or the fixed disk 679, as well as the exchange of information between subsystems. The system memory 672 and/or the fixed disk 679 may embody a computer readable medium. Any of the values mentioned herein can be output from one component to another component and can be output to the user.

A computer system can include a plurality of the same components or subsystems, e.g., connected together by external interface 681 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present invention can be implemented in the form of control logic using hardware and/or using computer software in a modular or integrated manner. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer program product (e.g. a hard drive, a CD, or an entire computer system), and may be present on or within different computer program products within a system or network. A computer system may include a monitor, printer, or other suitable display for providing any of the results mentioned herein to a user.

Any of the methods described herein may be totally or partially performed with a computer system including a processor, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, circuits, or other means for performing these steps.

The specific details of particular embodiments may be combined in any suitable manner or varied from those shown and described herein without departing from the spirit and scope of embodiments of the invention.

The above description of exemplary embodiments of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications to thereby enable others skilled in the art to best utilize the invention in various embodiments and with various modifications as are suited to the particular use contemplated.

### EXAMPLES

The following examples are offered to illustrate, but not to limit the claimed invention.

### Example 1: Use of the Nelder-Mead method to optimize efficiency of operon discovery

This examples illustrates how to recombinant and computationally manipulate and select native gene cluster coding sequences and heterologous regulatory sequences. We have termed this process “refactoring”, which comprises optimization of multiple genes, regulation of the gene cluster, and establishment of the genetic context for the biological circuit. Refactoring complex gene clusters and engineering metabolic pathways requires numerous iterations between design, construction and evaluation in order to improve a desired system property, e.g. higher product titers, lower toxicity, or improved nitrogen fixation.

One common way to affect these properties is to modify gene expression levels within the system, even if the direct relationship between gene expression and the system property is unknown. Making quantitative changes to gene expression can be achieved through the use of regulatory elements, e.g. promoters and ribosome binding sites, that exhibit rationally predictable behavior.

It is possible to utilize numerical optimization methods to guide selection of regulatory elements in order to alter gene expression and to improve desired system properties. One relevant algorithm is the Nelder-Mead method, a nonlinear optimization algorithm that minimizes an objective function in multidimensional space. We use the Nelder-Mead method to optimize a system property where each dimension in algorithmic space corresponds to expression of a gene in the engineered system. Points in this space represent a particular combination of expression levels for the genes in the system. As a result, each point may be considered a uniquely engineered strain. The algorithm is used to suggest new coordinates in space that improve the system property. New strains can be engineered by modifying regulatory elements to attain the suggested levels of gene expression. After evaluating the performance of the new strains, the algorithm can be used to predict subsequent modifications. This process iterates until the system property has been improved a desired amount.

The Nelder-Mead method relies on the concept of a simplex, which is an object in N dimensional space having N+1 vertices. The objective function is evaluated at each vertex of the simplex, and the algorithm uses this information to propose a sequence of new coordinates for evaluation. New coordinates are proposed according to the following process:
1. Order the simplex vertices: f(x₁)≤f(x₂)≤ . . . ≤f(xₙ₊₁)
2. Calculate x₀, the center of gravity of all points except xₙ₊₁
3. Calculate a Reflection coordinate: xᵣ=x₀+α(xₒ−xₙ₊₁)
4. Calculate an Expansion coordinate: xₑ=xₒ+r(xₙ−xₙ₊₁)
5. Calculate a Contraction coordinate: x_{c}=xₙ₊₁+ρ(xₒ−xₙ₊₁)
6. Calculate Reduction coordinates: xᵢ=xₗ+σ(xᵢ−xₗ) for all i∈ {0.2, . . . , n+1}

The objective function is evaluated at these points and used to determine a new simplex according to the following criteria:
1. If the Reflection, Expansion or Contraction coordinates are better than the worst simplex point, xₙ₊₁, define a new simplex by replacing the worst simplex point with the best of the three (Reflection, Expansion or Contraction).
2. Otherwise, define a new simplex by combining the best simplex point with the Reduction coordinates.

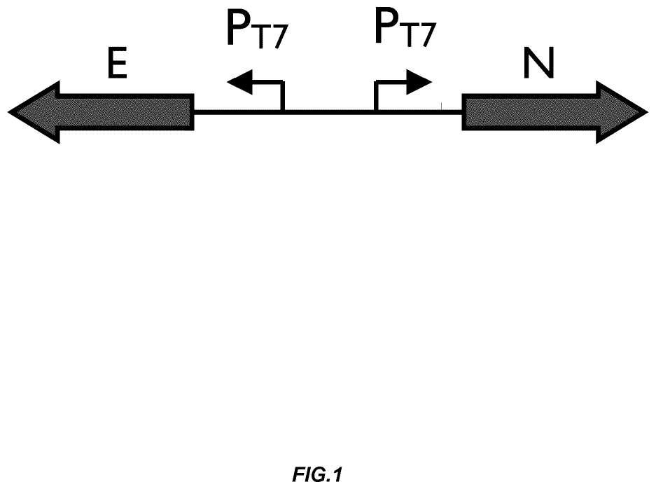

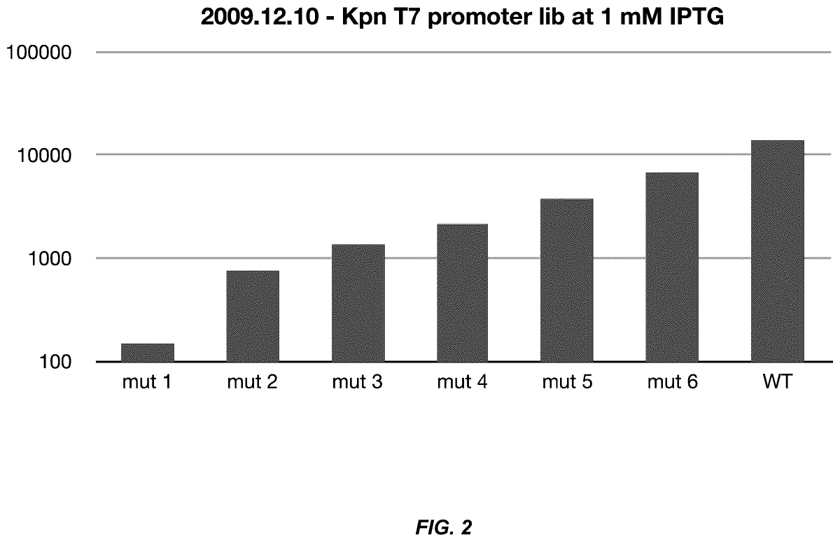

These steps constitute an iteration of the algorithm. The newly defined simplex becomes the seed for generating new coordinates during the next iteration of the algorithm. Iterations typically continue until one of the coordinates in the simplex crosses a desired threshold for objective function evaluation. We have optimized the performance of a nitrogen fixation operon by varying the selection of promoters that control expression of individual genes. We initially refactored the nifEN operon so that each gene was expressed under the control of a unique T7 promoter (FIG. 1). To assess the impact of refactoring the nifEN operon, we quantitatively measured the capacity of the synthetic operon to complement a nifEN knockout strain and recover the ability to fix nitrogen (FIG. 2). Our refactored system showed limited ability to fix nitrogen (20% of wild-type activity).

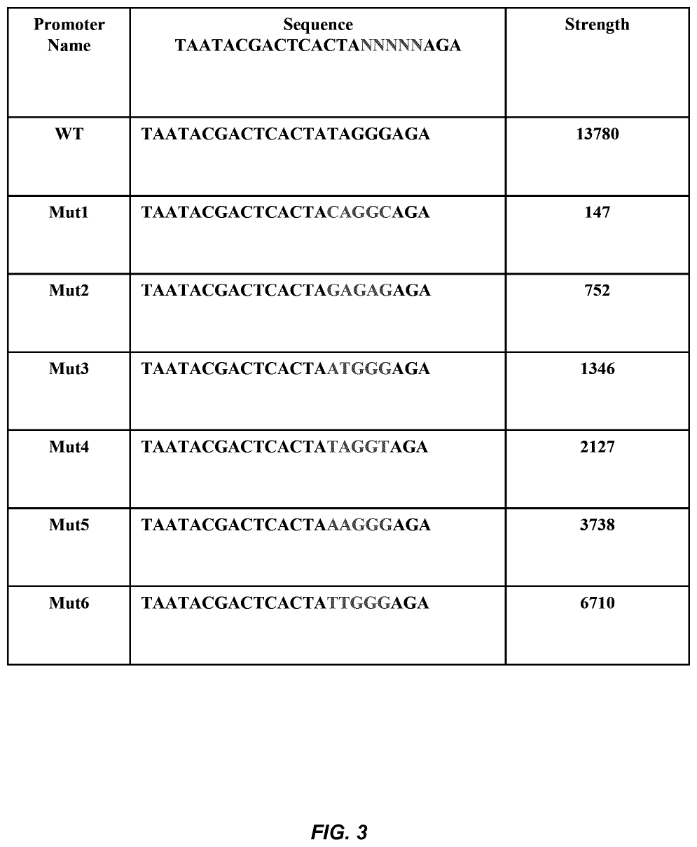

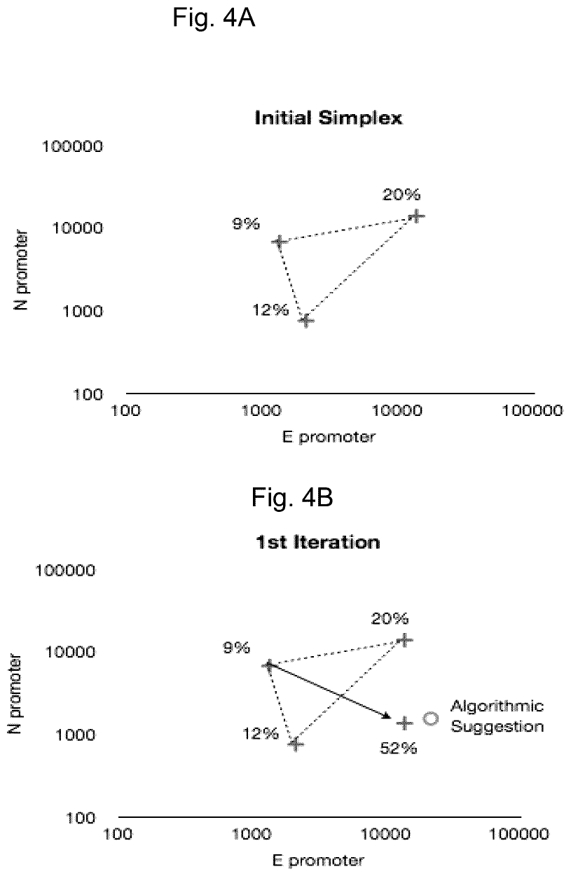

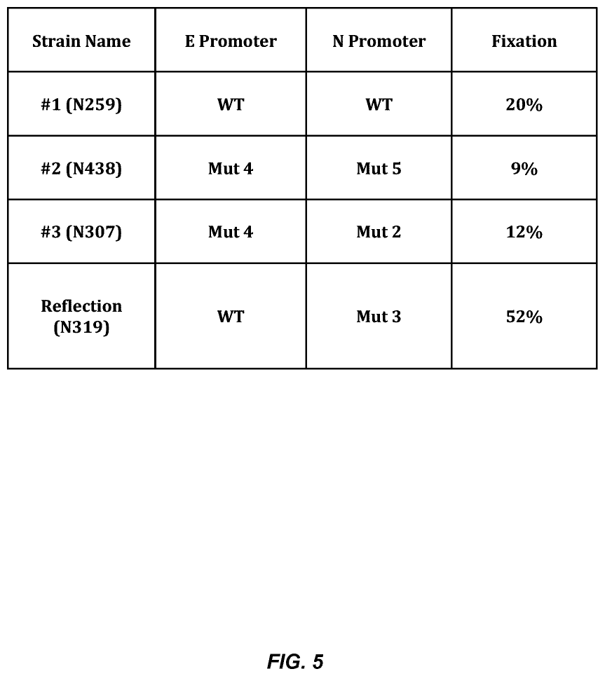

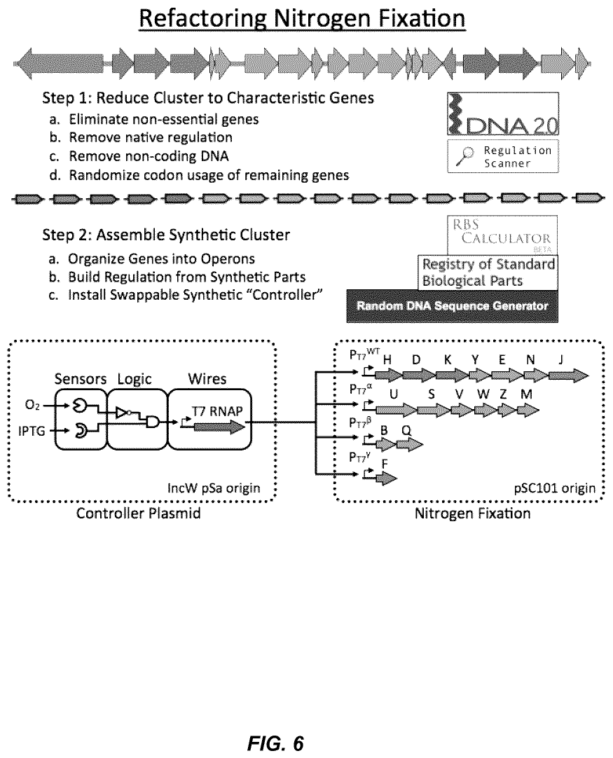

We subsequently applied the Nelder-Mead method to optimize nifE and nifN gene expression with the goal of improving nitrogen fixation rates. Our algorithmic space consisted of two dimensions, nifE and nifN expression. Our coordinate system was scaled to the strength of the promoters controlling these genes. To enable varied levels of gene expression, we generated and characterized a library of mutant T7 promoters (FIG. 3). Our library covers three order of magnitude of gene expression (This is the same library that is described in U.S. Provisional Patent Application No. 61/493,733. Here, it is characterized for behavior in Klebsiella oxytoca). We then randomly selected mutants from the library of T7 promoters to generate two additional strains with rationally altered levels of nifE and nifN expression. The strength of T7 promoters used in these three strains defined our initial simplex. We evaluated nitrogen fixation for each strain in the simplex (strain 1: 20%, strain 2: 9%, strain 3: 12%) and used the algorithm to calculate Reflection coordinates (FIG. 4). To construct the strain that matched the Reflection coordinates, we chose promoters from our library nearest to the coordinates in strength. We evaluated nitrogen fixation in this Reflection strain and found that it significantly outperformed (52%) our initial strains (FIG. 5).

Our improved strain had surprising results and surpassed expectations, and performed sufficiently for downstream applications. To reach higher levels of gene expression, stronger promoters can be engineered and used in the methods of the invention. Alternatively, complimentary changes to multiple regulatory elements, e.g., the promoter and ribosome binding site for a given gene, can be used to achieve desired expression levels. This involves describing the strengths of each type of element in common units of expression. This example demonstrates that new strains can be engineered by modifying regulatory elements to attain the desired levels of gene expression. The example also illustrates the use of numerical optimization methods, such as, but not limited to the Nelder-Mead method, to guide selection of regulatory elements in order to alter gene expression and to improve desired system properties.

### Example 2: Refactoring Nitrogen Fixation

This example demonstrates the method of refactoring the nitrogen fixation gene cluster. The method includes steps that comprise:: 1) removing host regulation and implement synthetic, orthogonal regulation; 2) tracking the contribution of each regulatory part to gene cluster function; 3) promoting modularity and integration with synthetic circuits; and 4) creating a platform amenable to rational optimization. In certain embodiments, the method of refactoring nitrogen fixation comprises reducing cluster to characteristic genes and assembling synthetic cluster.

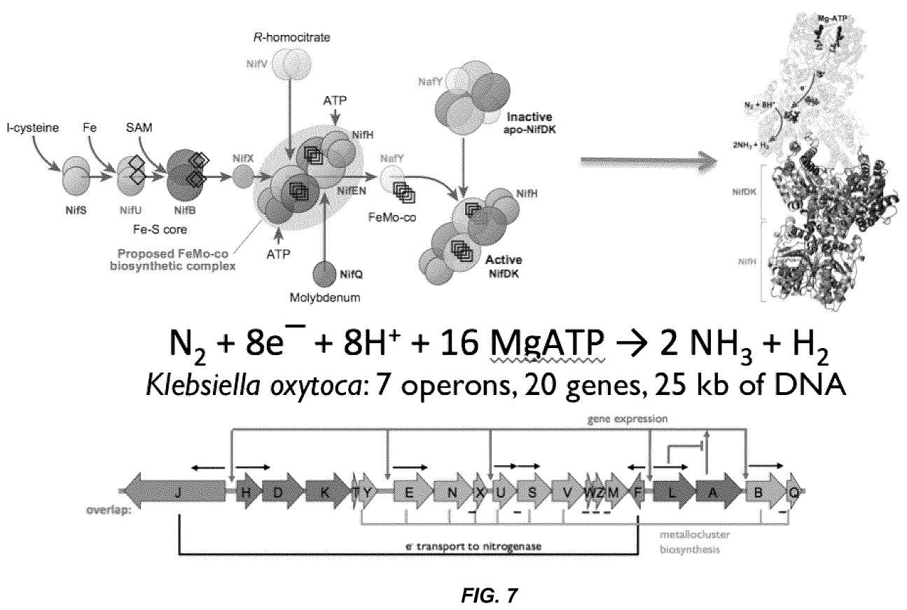

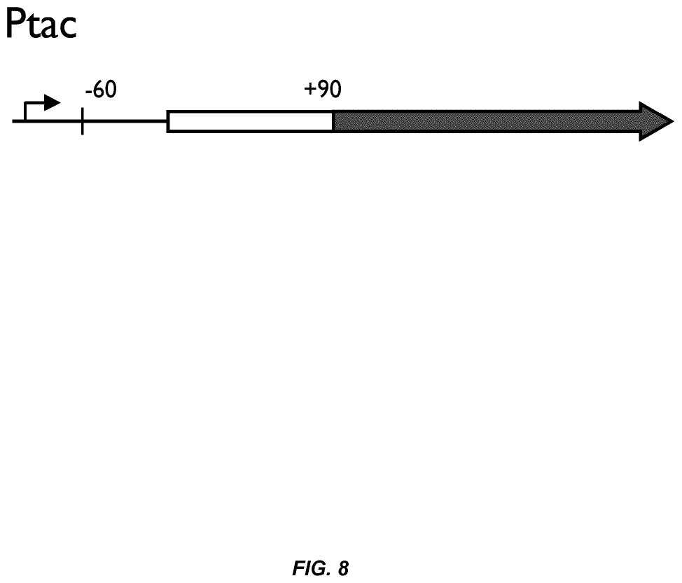

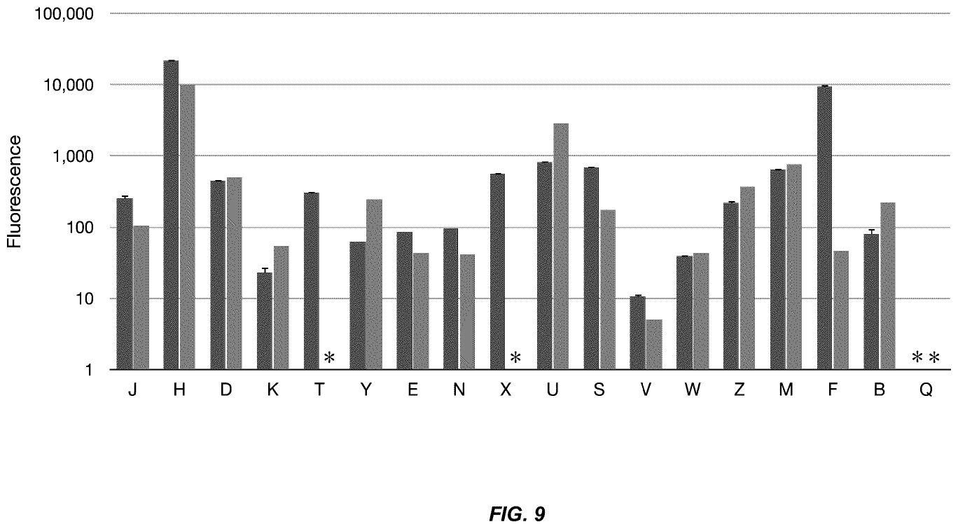

The nif gene cluster from Klebsiella oxytoca has been one of the primary models for study of the nitrogenase enzyme (FIG. 7; see, Rubio and Ludden, Maturation of Nitrogenase: a Biochemical Puzzle, J. Bacteriology, 2005). It is a concise gene cluster, encompassing 20 genes in 7 operons within 25 kb of DNA. The nitrogenase enzyme is composed of two major units, Component I and Component II, that interact to facilitate the reduction of multiply bonded gases like N2. Within the enzyme complex, multiple Fe—S clusters are responsible for active site chemistry and electron transfer to the active site. The majority of the genes in the gene cluster are involved in Fe—S cluster biosynthesis, chaperoning and insertion into the final enzyme complex.

Nearly every nif gene produces a protein with a function known to be essential to nitrogenase assembly or function (see, Simon, Homer and Roberts, Perturbation of nifT expression in Klebsiella pneumoniae has limited effect on nitrogen fixation, J. Bacteriology, 1996 and Gosink, Franklin and Roberts, The product of the Klebsiella pneumoniae nifX gene is a negative regulator of the nitrogen fixation (nif) regulon, J Bacteriology, 1990). Two genes, nifL and nifA, encode the master regulatory proteins. The nifT gene has no known function, and eliminating it has little effect on nitrogen fixation. Additionally, while elimination of nifX has minor effect on nitrogen fixation, its overexpression detrimentally reduces enzyme activity. For these reasons, we chose to eliminate nifL, nifA, nifT and nifX from our refactored gene cluster.

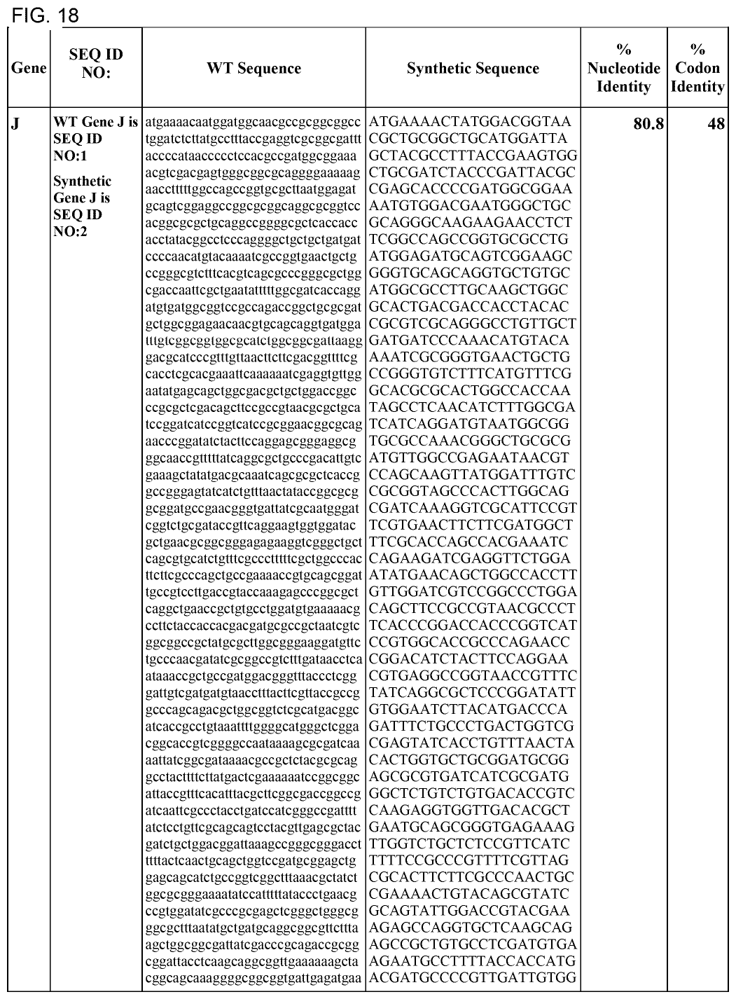

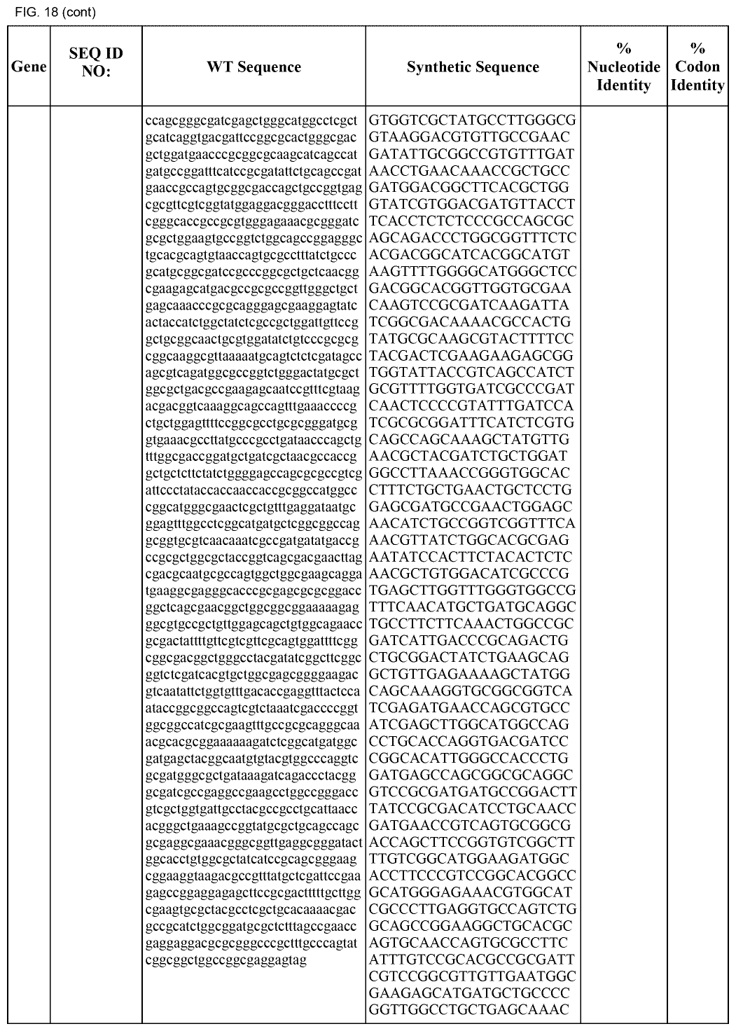

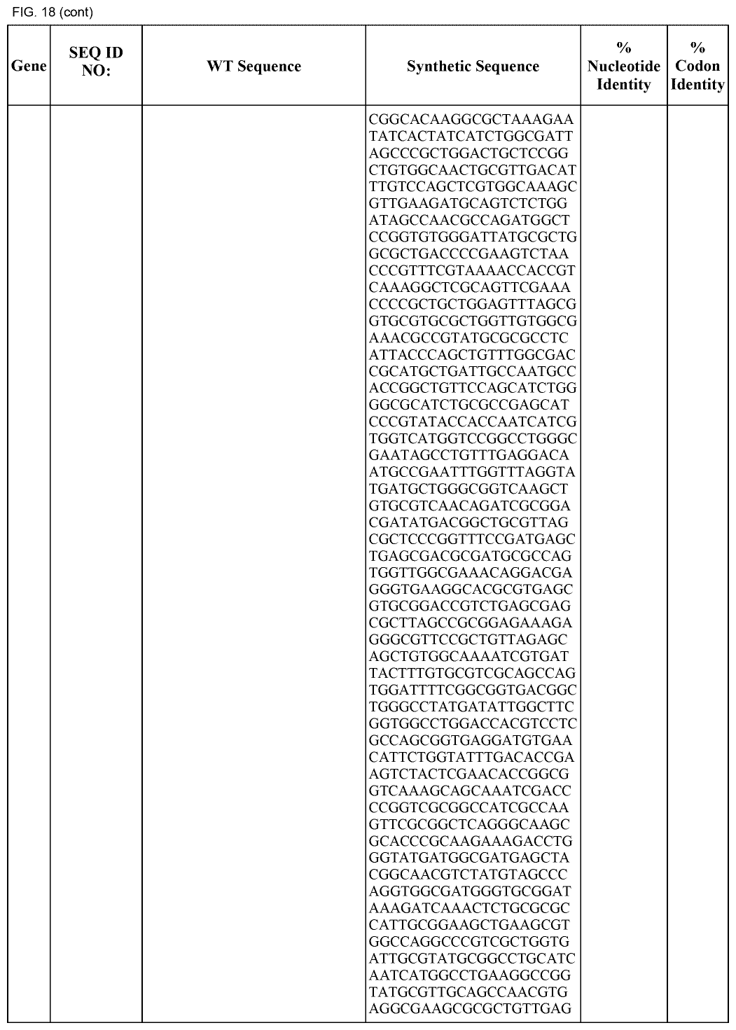

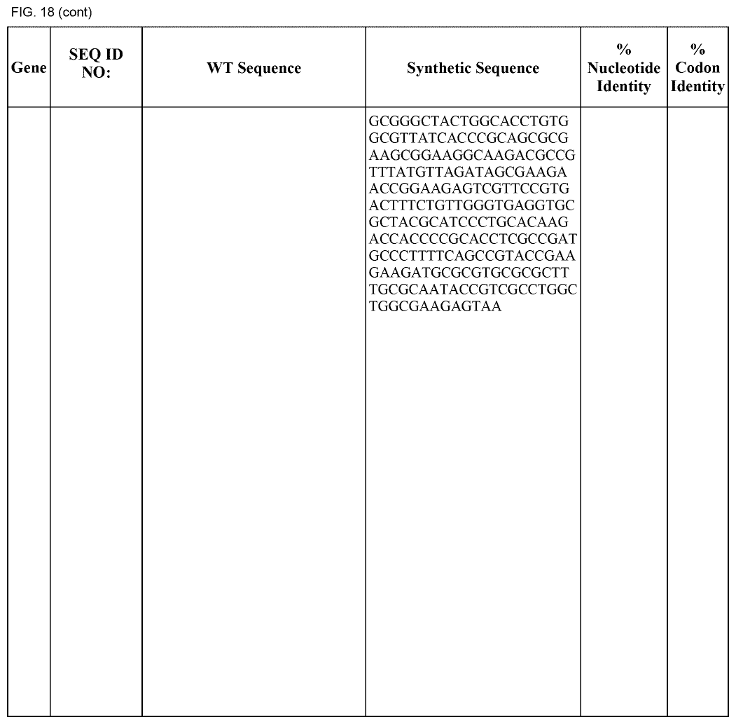

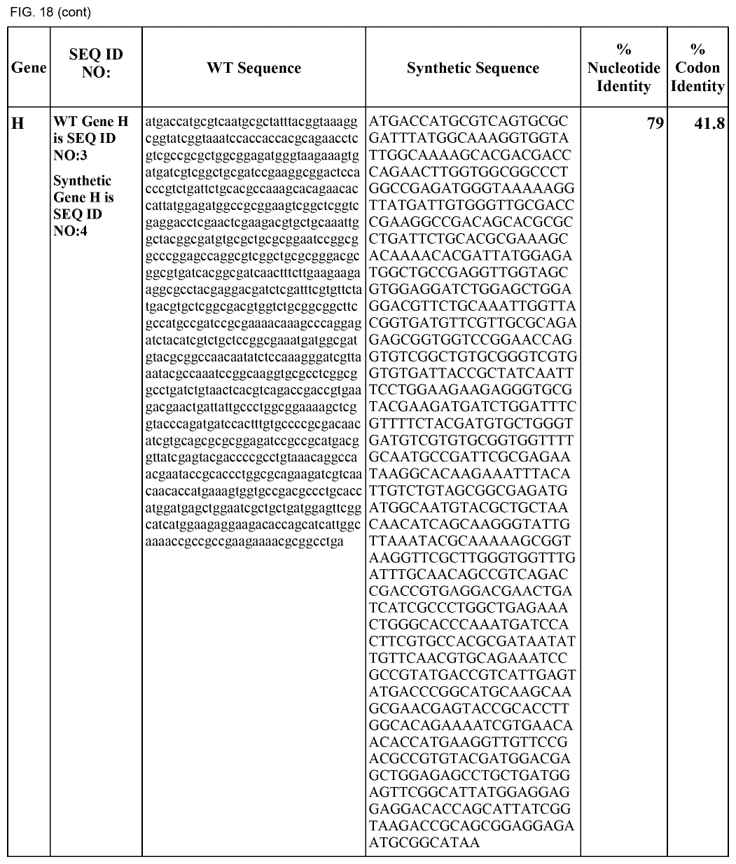

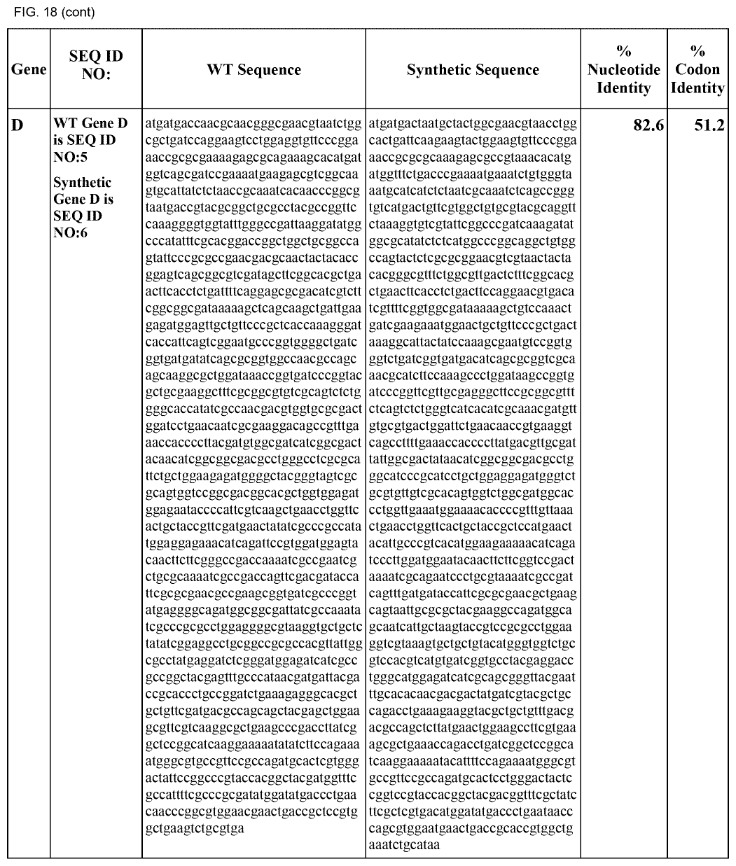

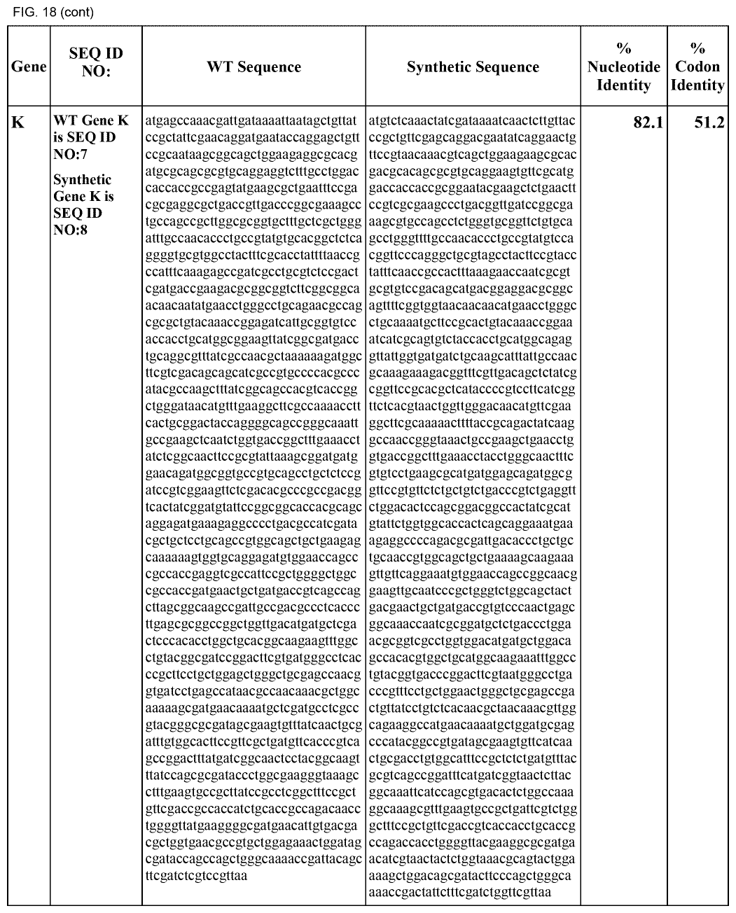

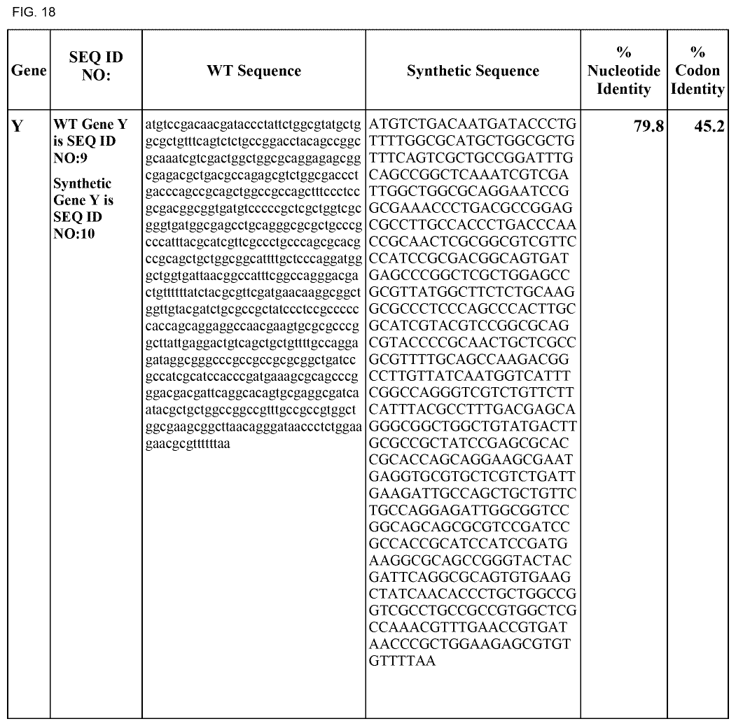

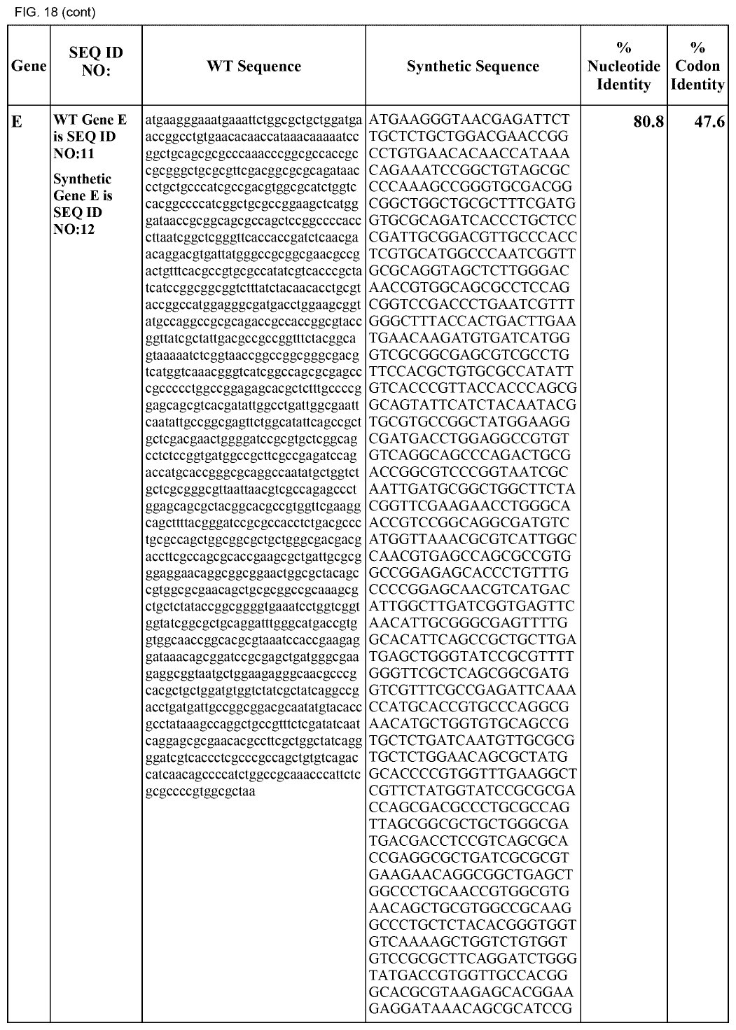

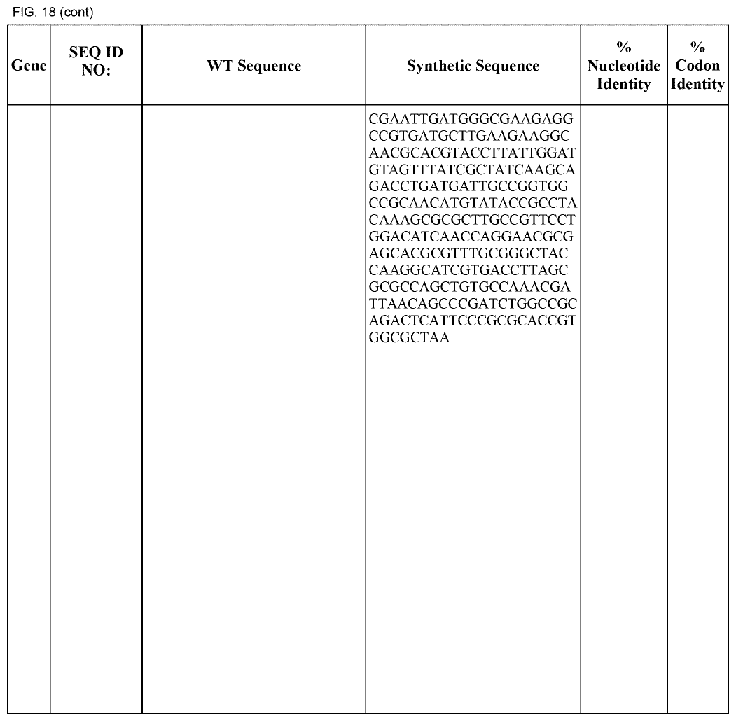

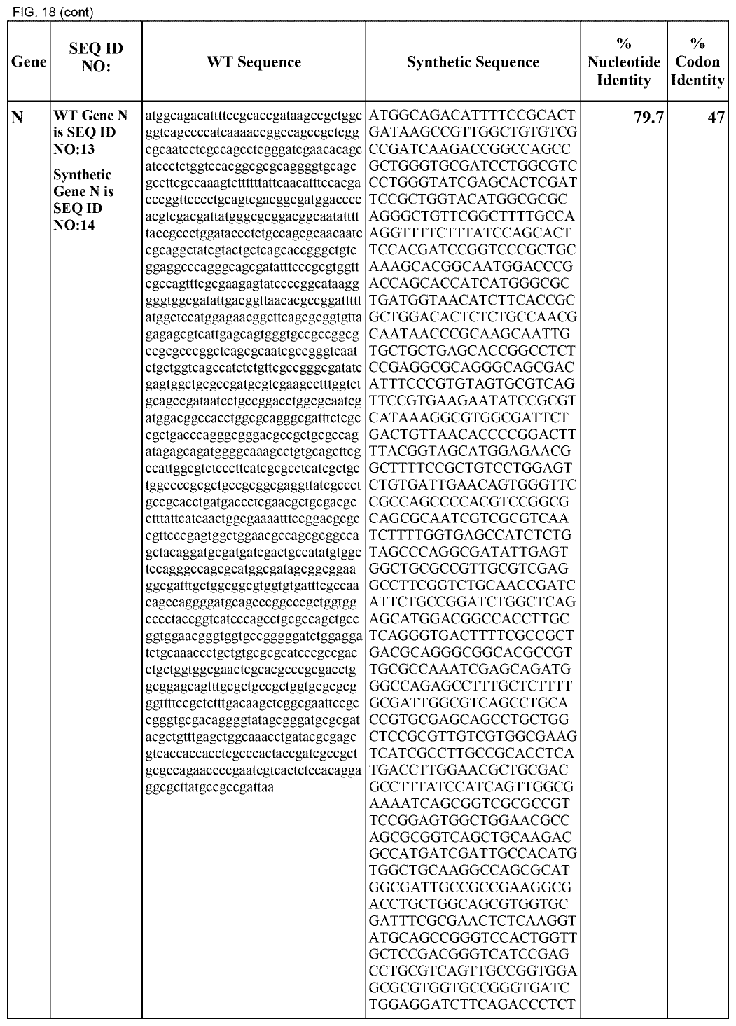

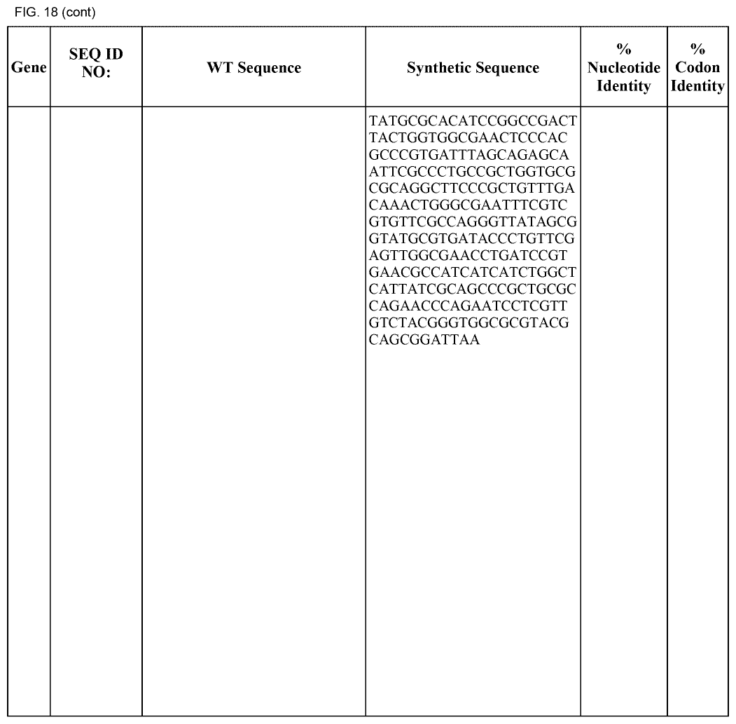

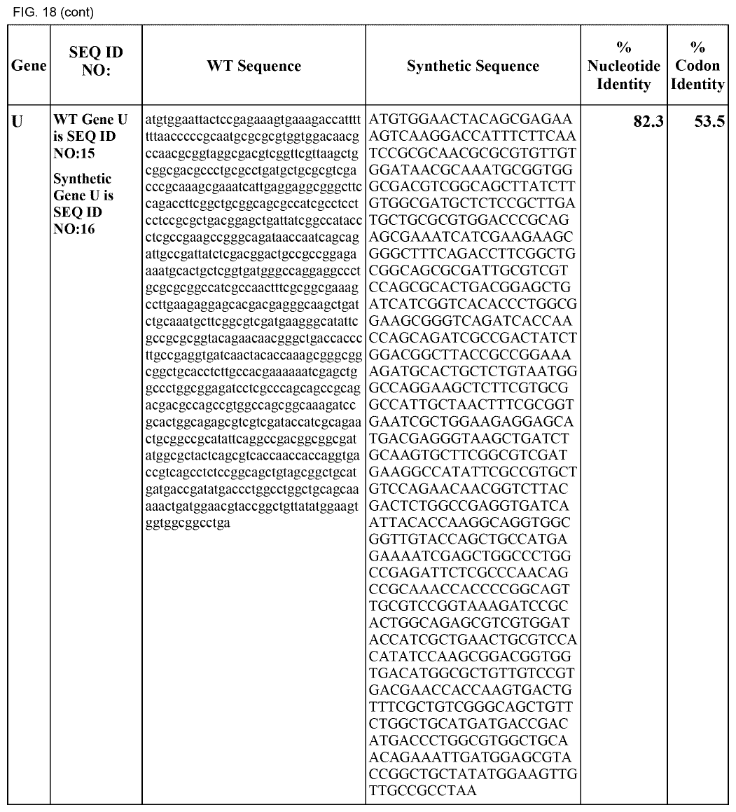

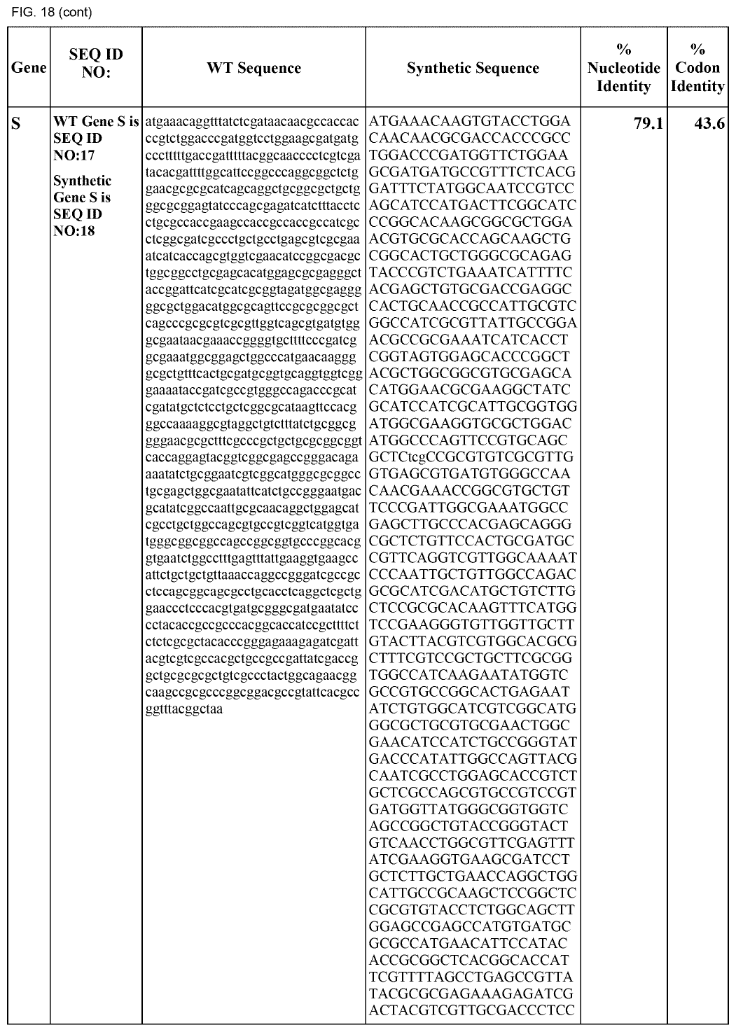

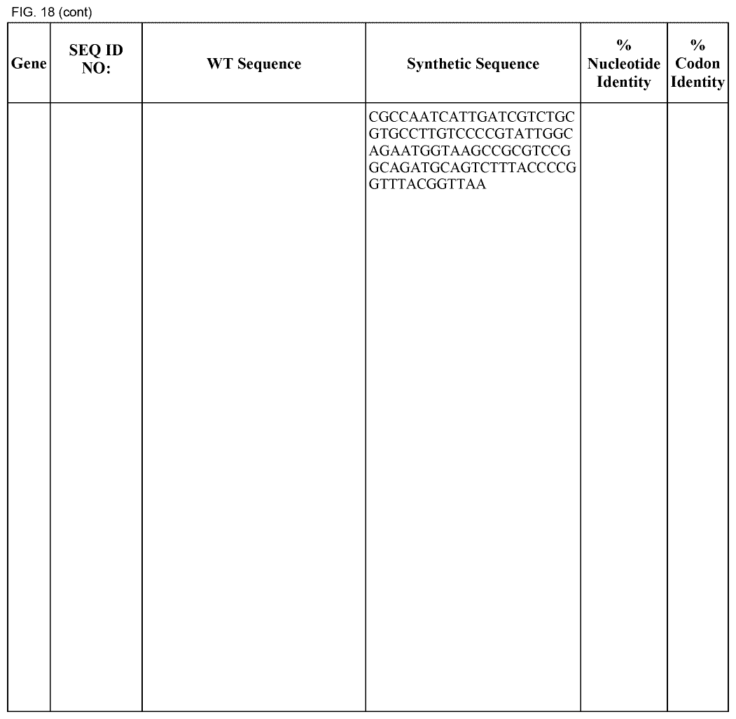

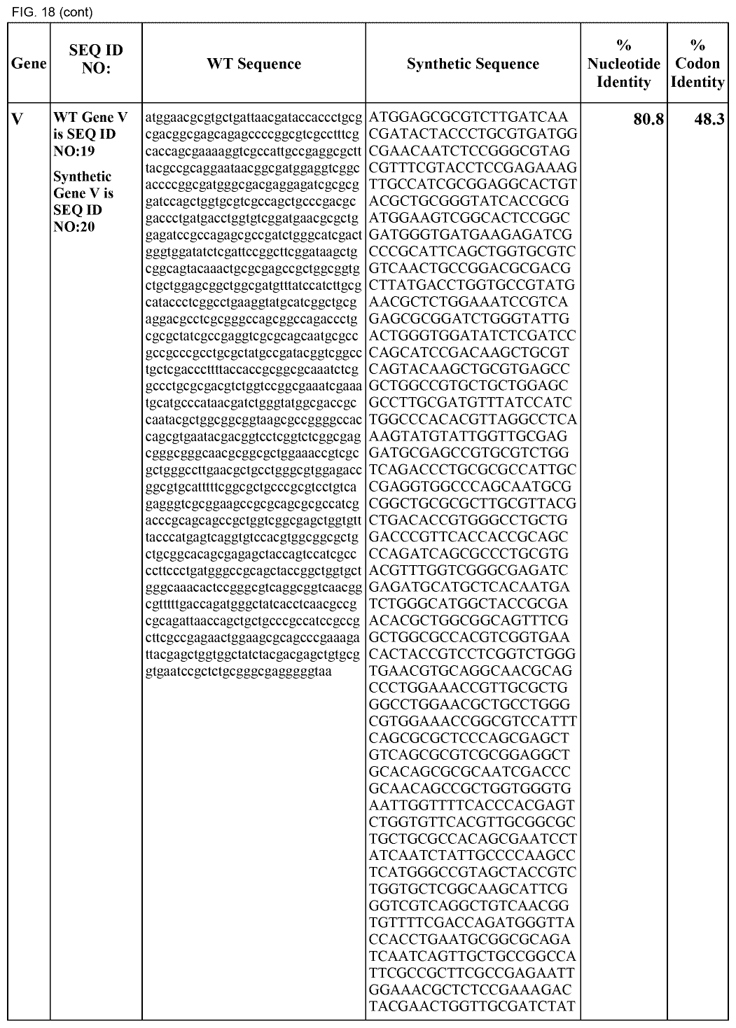

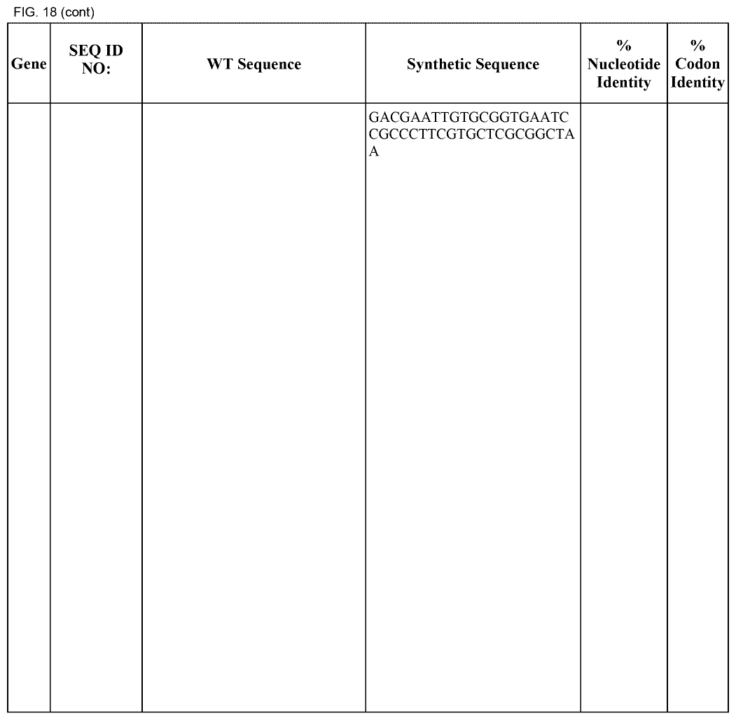

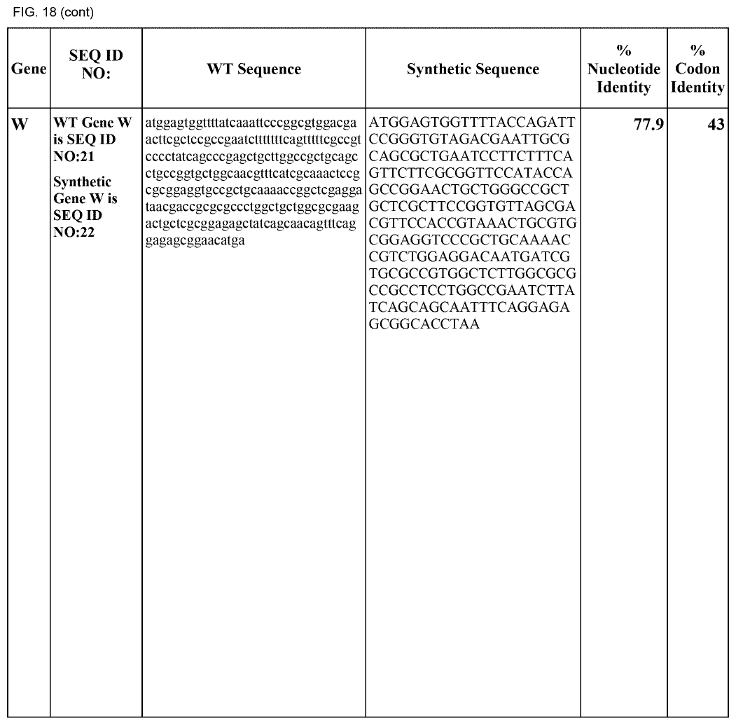

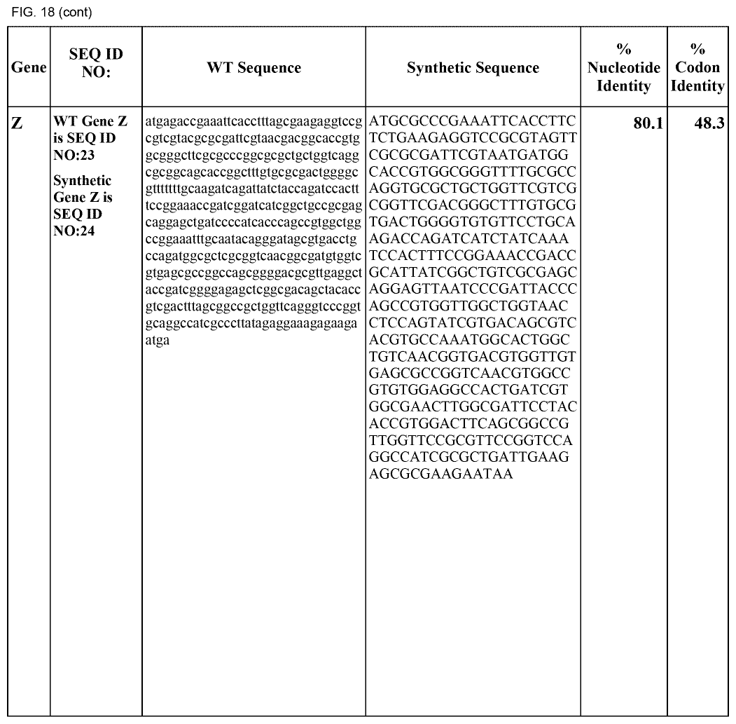

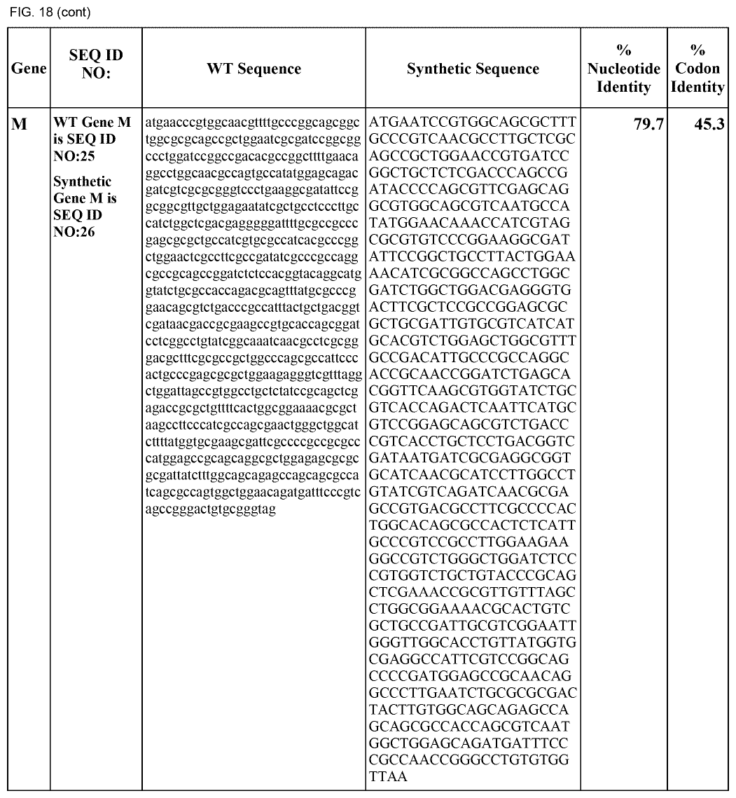

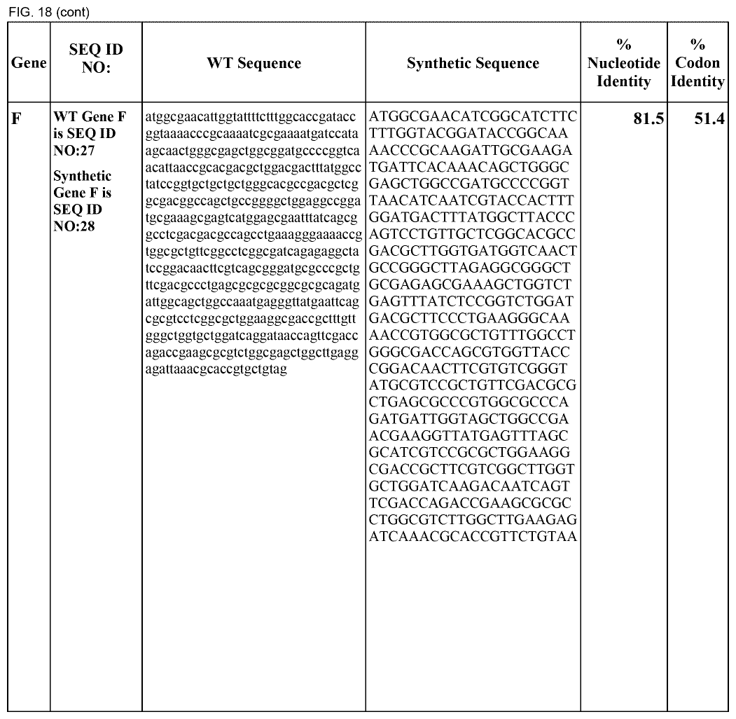

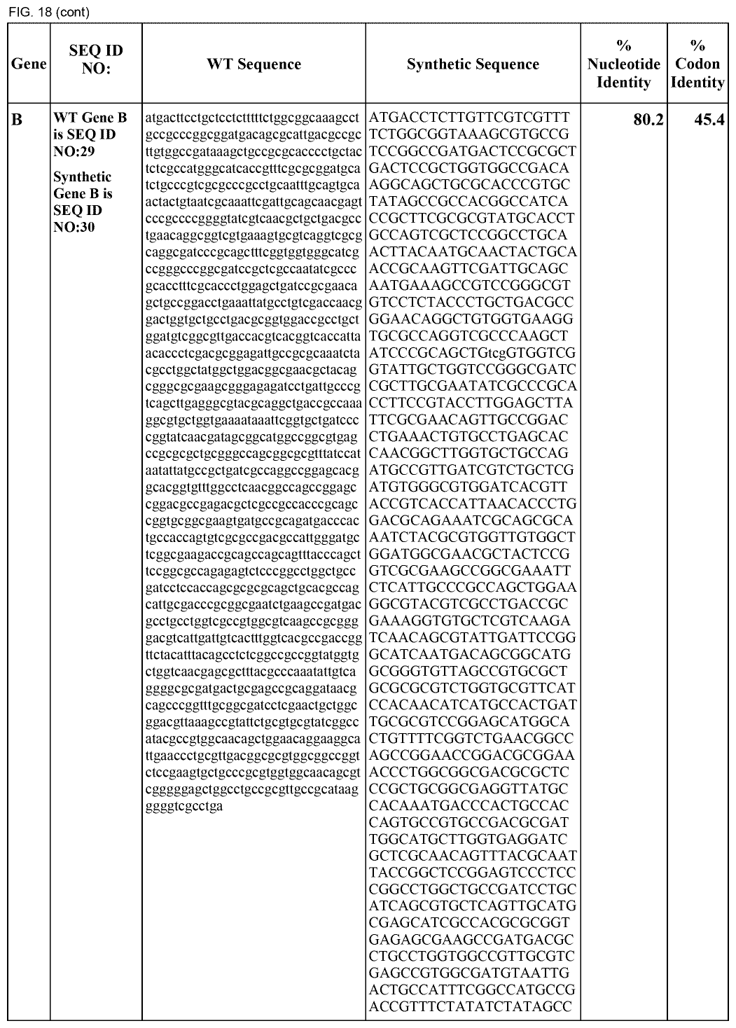

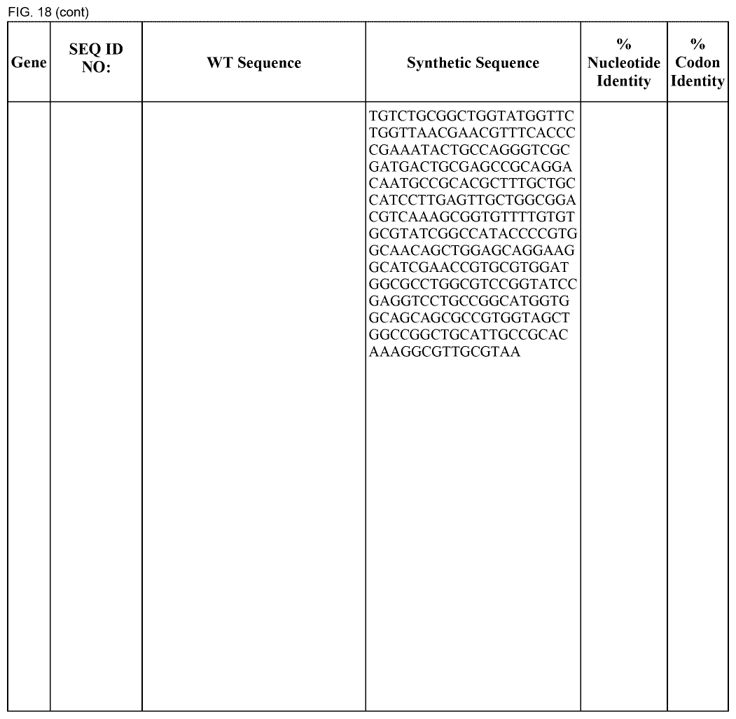

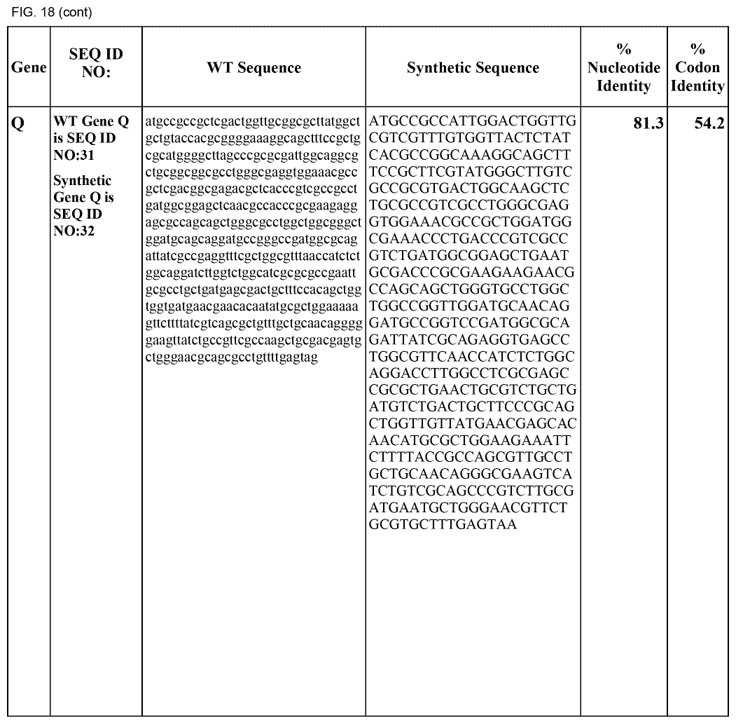

We designed synthetic genes by codon randomizing the DNA encoding each amino acid sequence. Protein coding sequences were based on the sequence deposited in the NCBI database (X13303; see, Arnold et al., Nucleotide sequence of a 24,206-base-pair DNA fragment carrying the entire nitrogen fixation gene cluster of Klebsiella pneumoniae. JMB, 1988). Codon selection was performed by DNA2.0 using an internal algorithm and two guiding criteria. We specified that our genes express reasonably well in both E. coli and Klebsiella. Also, we specified that our codon usage be as divergent as possible from the codon usage in the native gene. While designing synthetic genes, we scanned each proposed sequence for a list of undesired features and rejected any in which a feature was found. The feature list includes restriction enzyme recognition sites, transposon recognition sites, repetitive sequences, sigma 54 and sigma 70 promoters, cryptic ribosome binding sites, and rho independent terminators. FIG. 18 shows DNA sequences for native genes and synthetic genes, as well as the percent common nucleotide and codon identities between each pair.

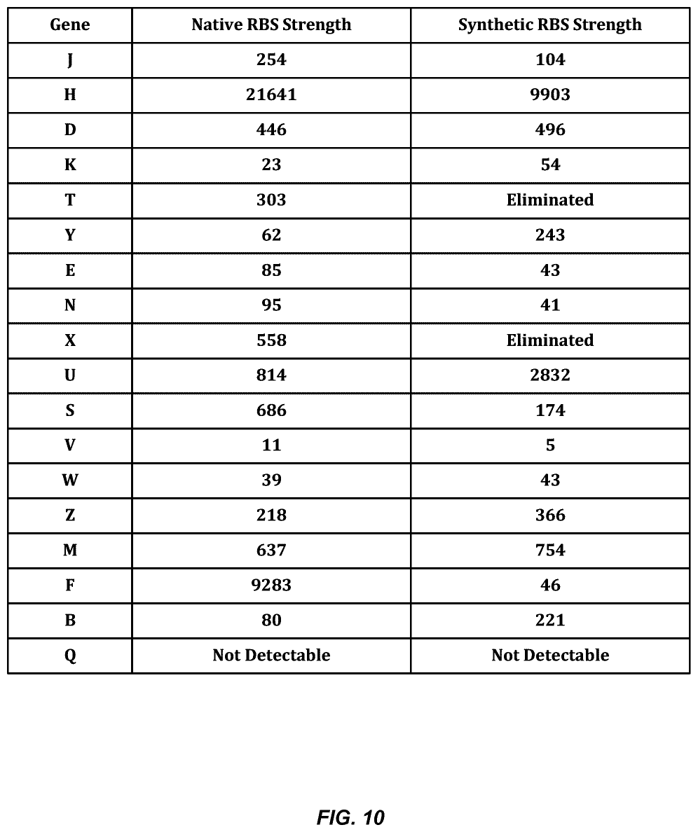

Synthetic ribosome binding sites were chosen to match the strength of each corresponding native ribosome binding site. To characterize the strength of a given native ribosome binding site, we constructed a fluorescent reporter plasmid in which the 150 bp surrounding a gene's start codon (from −60 to +90) were fused to the mRFP gene (FIG. 8). The chimera was expressed under control of the Ptac promoter, and fluorescence was measured via flow cytometry (FIG. 9). To generate synthetic ribosome binding sites, we constructed a library of reporter plasmids using 150 bp (−60 to +90) of a synthetic expression cassette. Briefly, a synthetic expression cassette consisted of a random DNA spacer, a degenerate sequence encoding an RBS library, and the coding sequence for each synthetic gene. We screened multiple clones to identify the synthetic ribosome binding site that best matched the native ribosome binding site (FIG. 10).

We constructed synthetic operons that consisted of the same genes as the native operons. This strategy enabled us to knock out a native operon from Klebsiella and complement the deletion using the synthetic counterpart.

Each synthetic operon consisted of a Ptac promoter followed by synthetic gene expression cassettes (random DNA spacer, synthetic rbs, synthetic coding sequence) and a transcription terminator. The random DNA spacer serves to insulate the expression of each synthetic coding sequence from preceding cassettes. Each synthetic operon was scanned to remove unintended regulatory sequences (similar to the process used during synthetic gene design and synthesis).

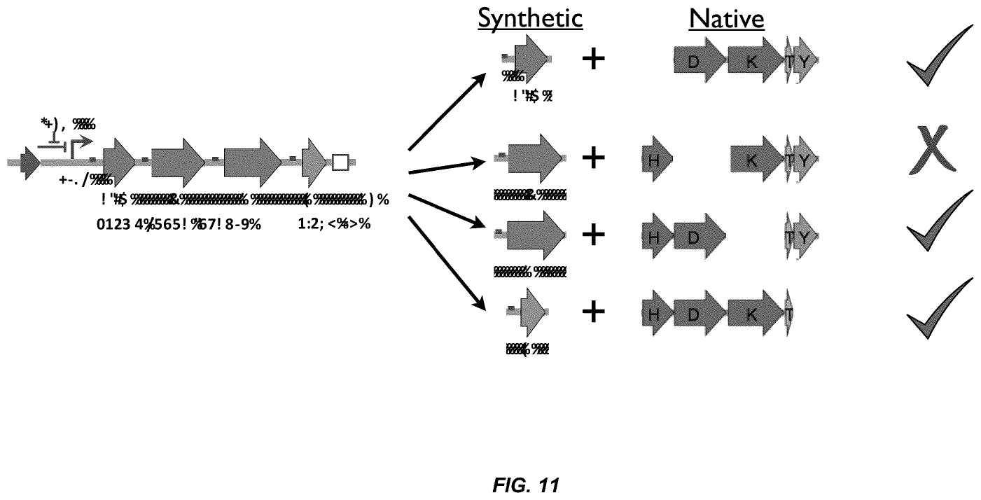

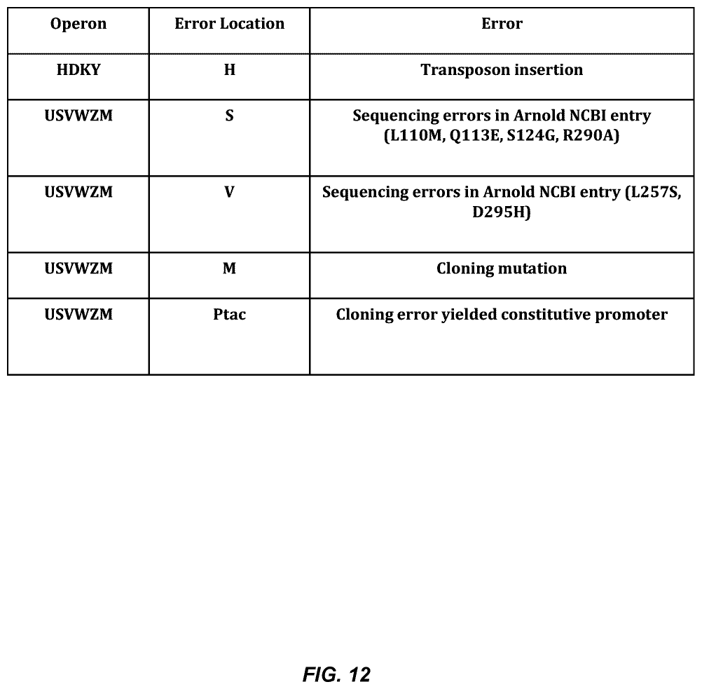

In two cases, we encountered synthetic operons that showed no functional complementation in the corresponding knockout strain (nifHDKTY and nifUSVWZM). To debug the synthetic operons, we broke the operon into constituent gene expression cassettes. We then constructed chimeric operons, wherein some cassettes had synthetic components and other cassettes were native genes and their ribosome binding sites (FIG. 11). This strategy enabled us to test each chimeric operon for functional complementation and quickly identify the problematic synthetic expression cassettes. With further analysis of problematic expression cassettes, we were able to diagnose and correct errors in the fully synthetic operons. FIG. 12 illustrates a list of errors in the two operons.

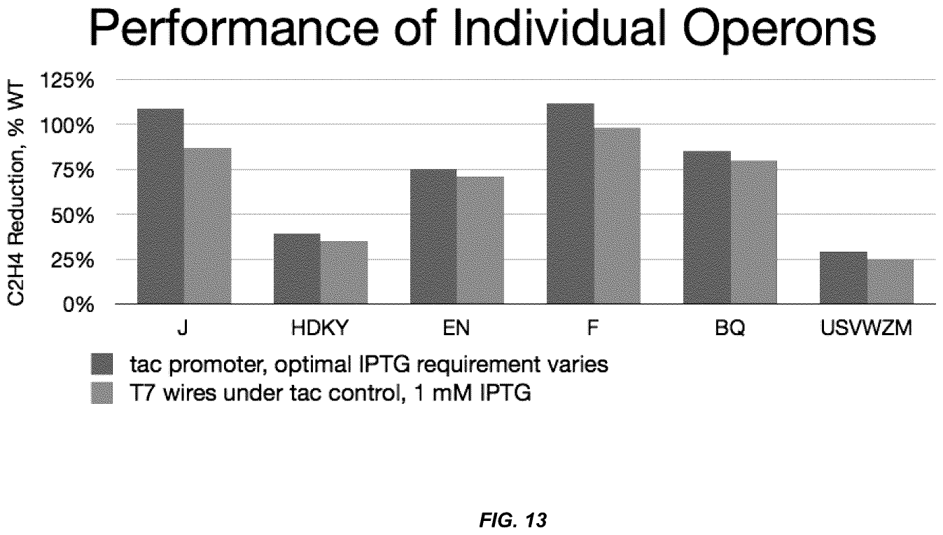

Each synthetic operon was initially designed to be controlled by a Ptac inducible promoter. By titrating IPTG concentration, we could precisely specify promoter strength and corresponding synthetic operon expression. This enabled us to vary expression level to identify optimal operon function. We found that each synthetic operon required different levels of IPTG concentration for optimal function (FIG. 13).

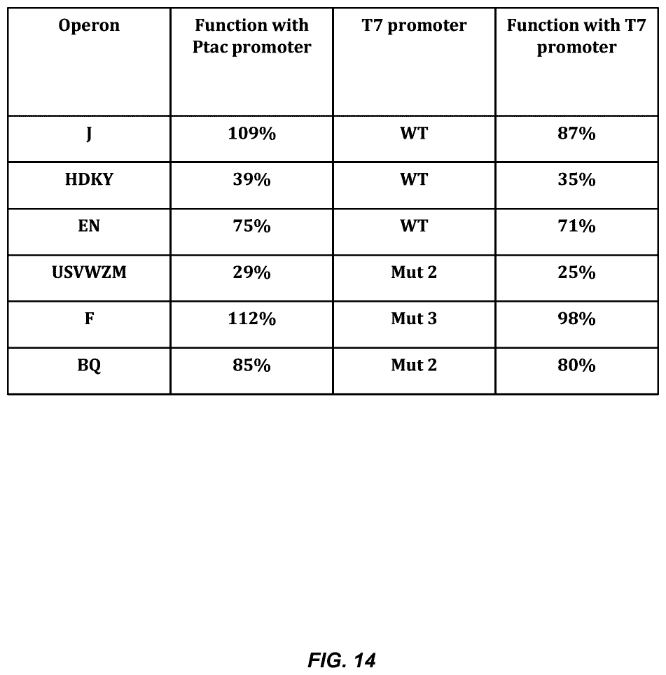

We utilized the T7 Wires system to decouple the Ptac promoter from each synthetic operon. By inserting the wire between the promoter and transcriptional unit, we achieved two significant milestones. First, we gained the ability to modulate the transcriptional signal through the use of various mutant T7 promoters. This allowed us to shift optimal operon function to a single inducer concentration by selecting corresponding mutant T7 promoters. Second, we modularized control of the synthetic operon (FIG. 14). That is, any genetic circuit can control the synthetic operon provided that it can produce the necessary T7 RNAP concentration to drive each wire.

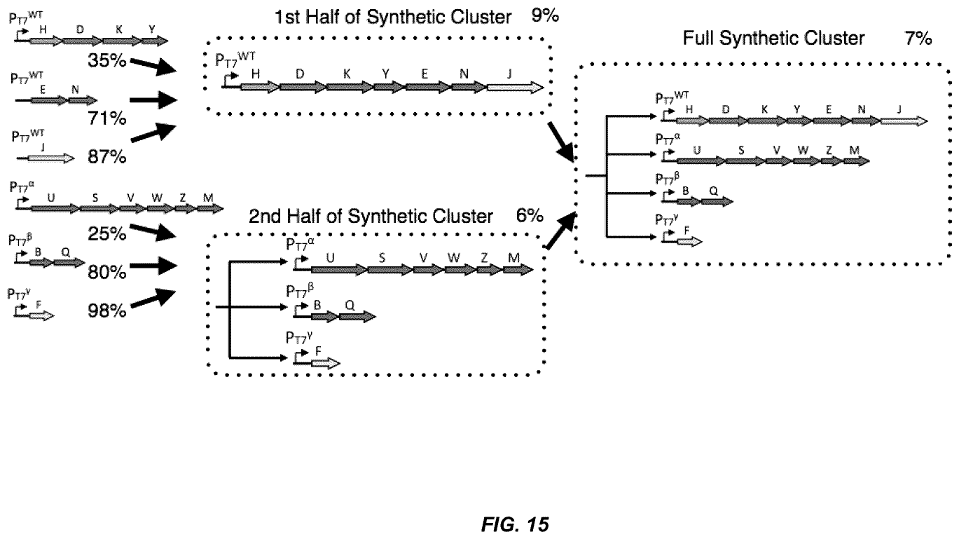

We adopted a hierarchical approach to assembling individual operons into a fully synthetic cluster. First, we assembled three operons into half clusters (nifJ-nijHDKY-nifEN and nifUSVWZM-nifF-nifBQ) and demonstrated the ability of each synthetic half cluster to complement function in a corresponding knockout strain. Next, we combined the two half clusters into a full synthetic cluster and demonstrated nitrogen fixation in a complete nif knockout strain (FIG. 15).

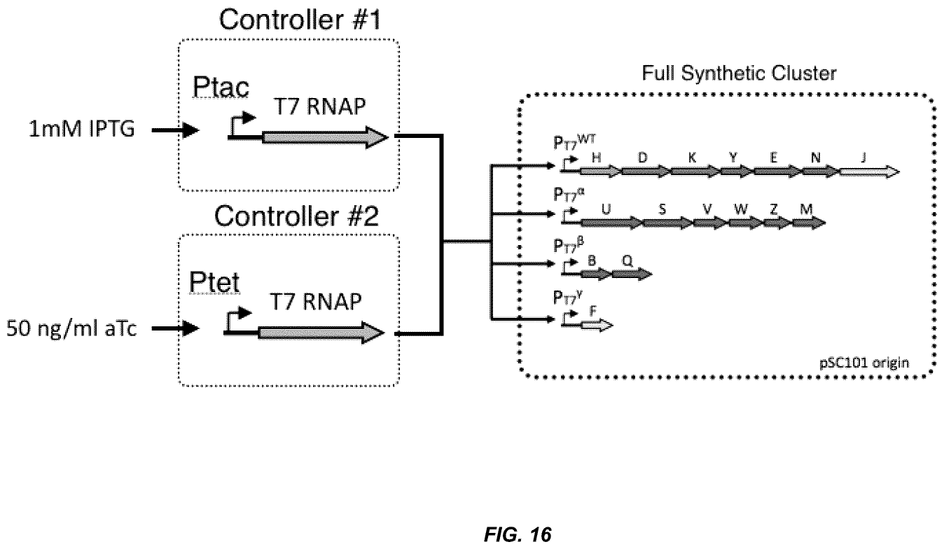

We have shown that the use of T7 Wires produces a modular synthetic gene cluster. We have demonstrated that the use of either controller #1 or controller #2 produces the same functional performance from the synthetic cluster (FIG. 16). In controller #1, T7 RNAP is under control of the Ptac promoter. In controller #2, T7 RNAP is under control of the Ptet promoter.

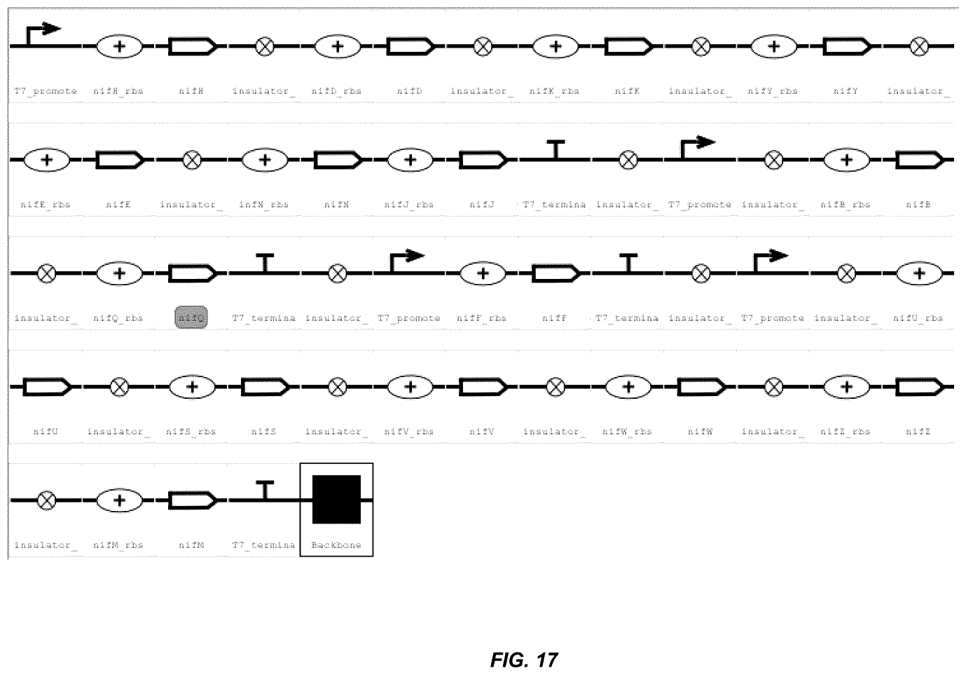

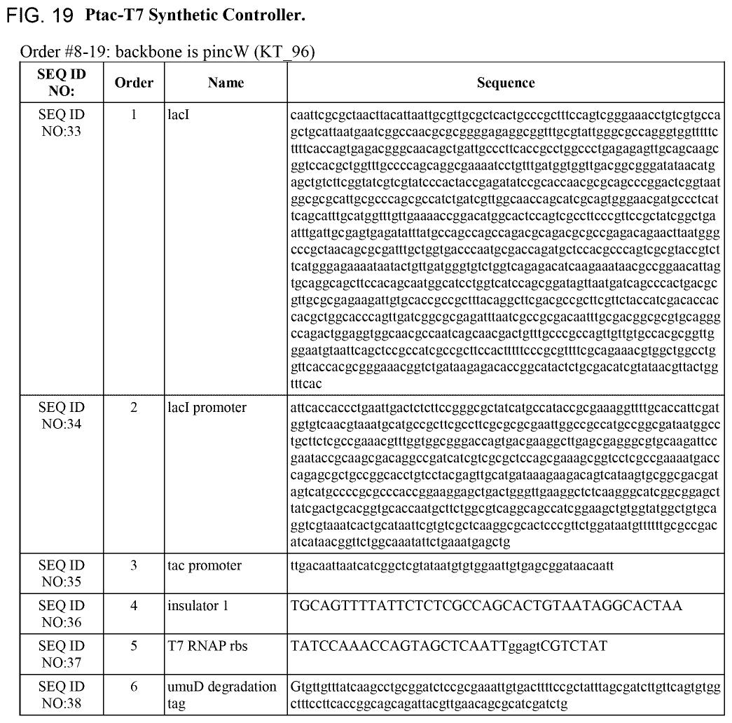

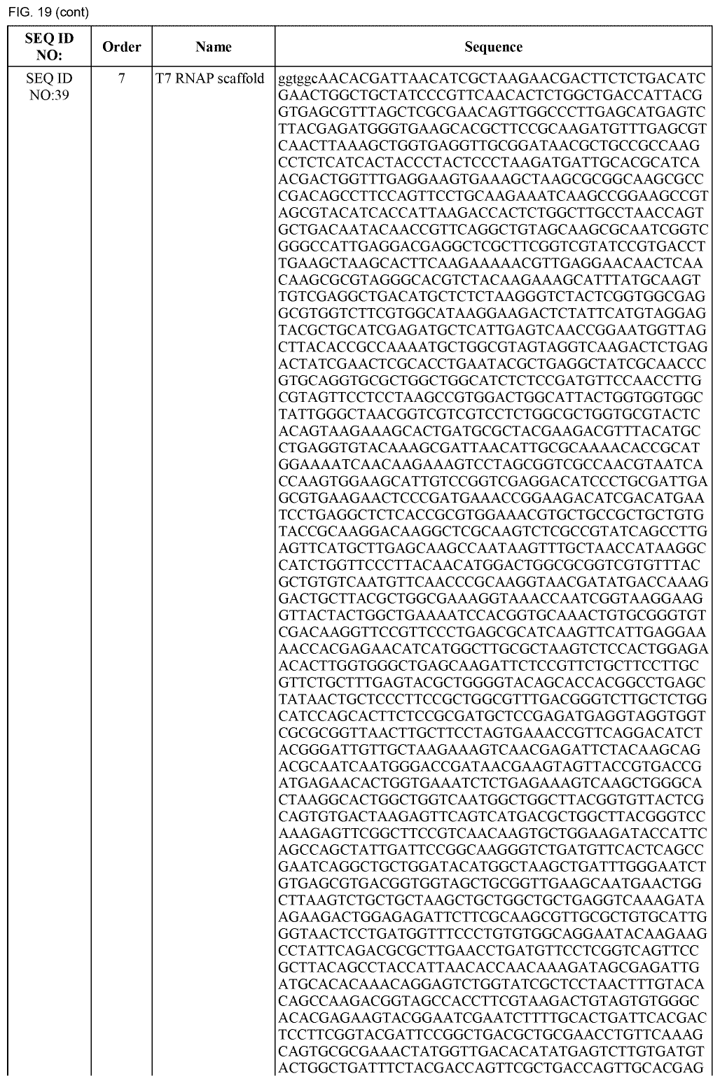

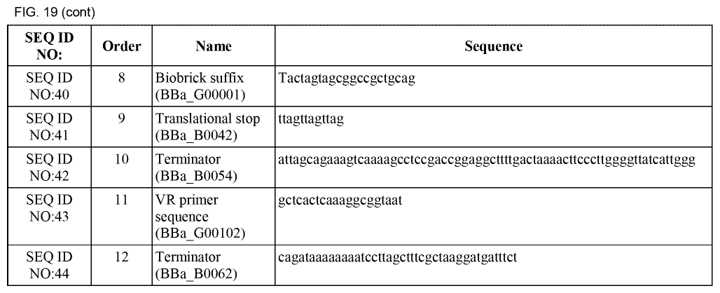

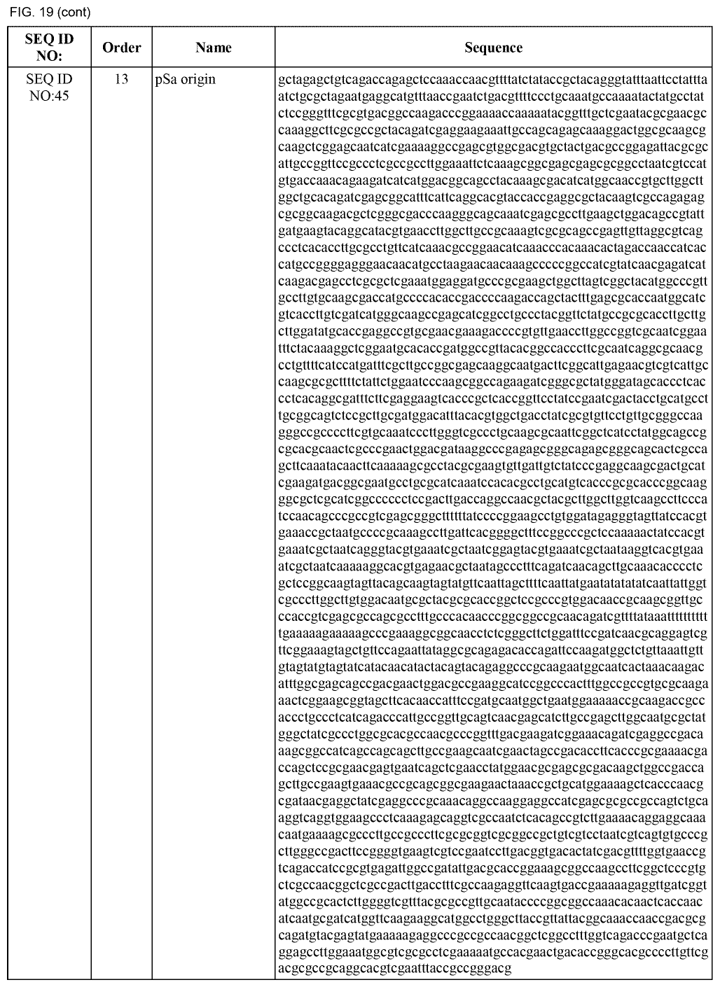

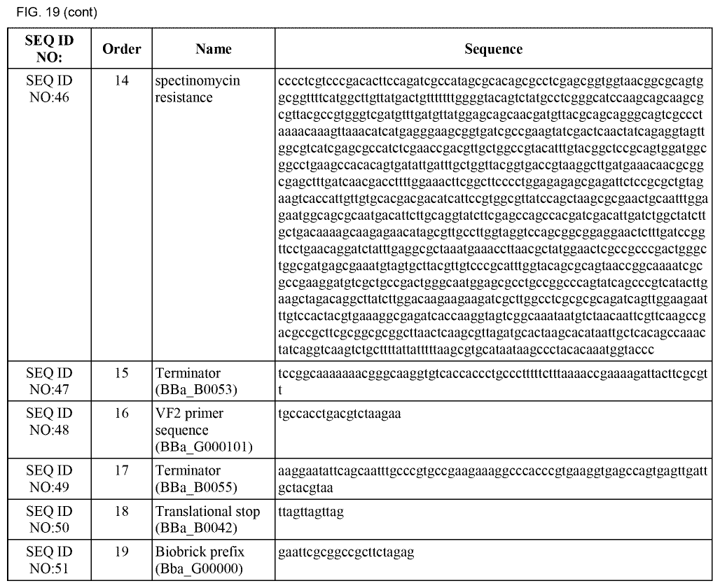

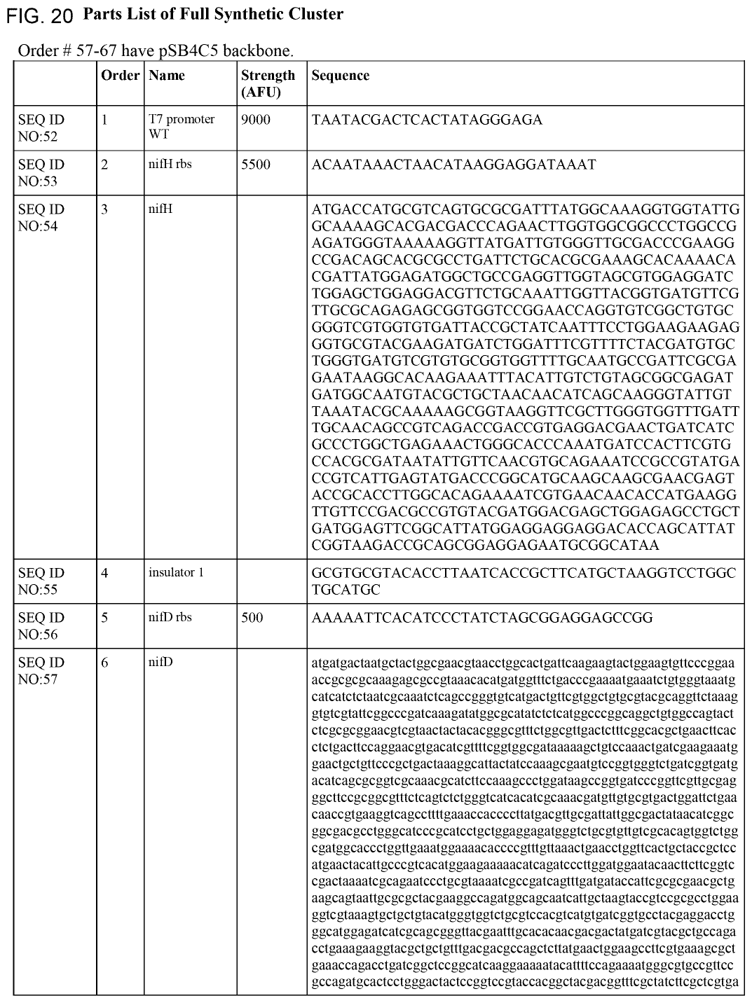

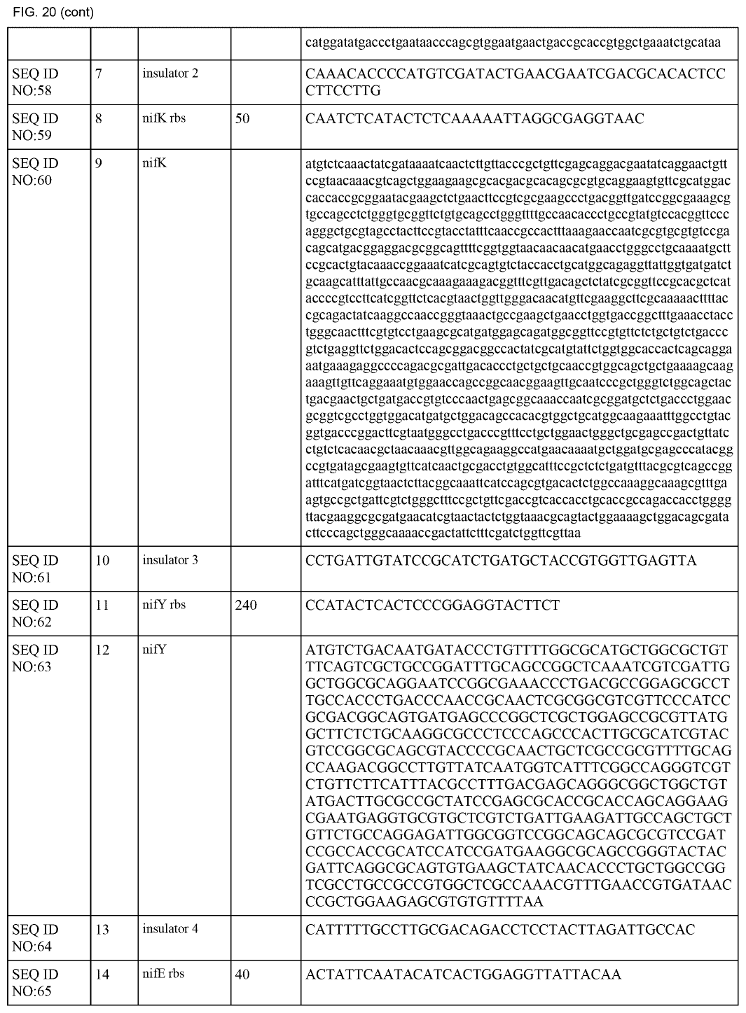

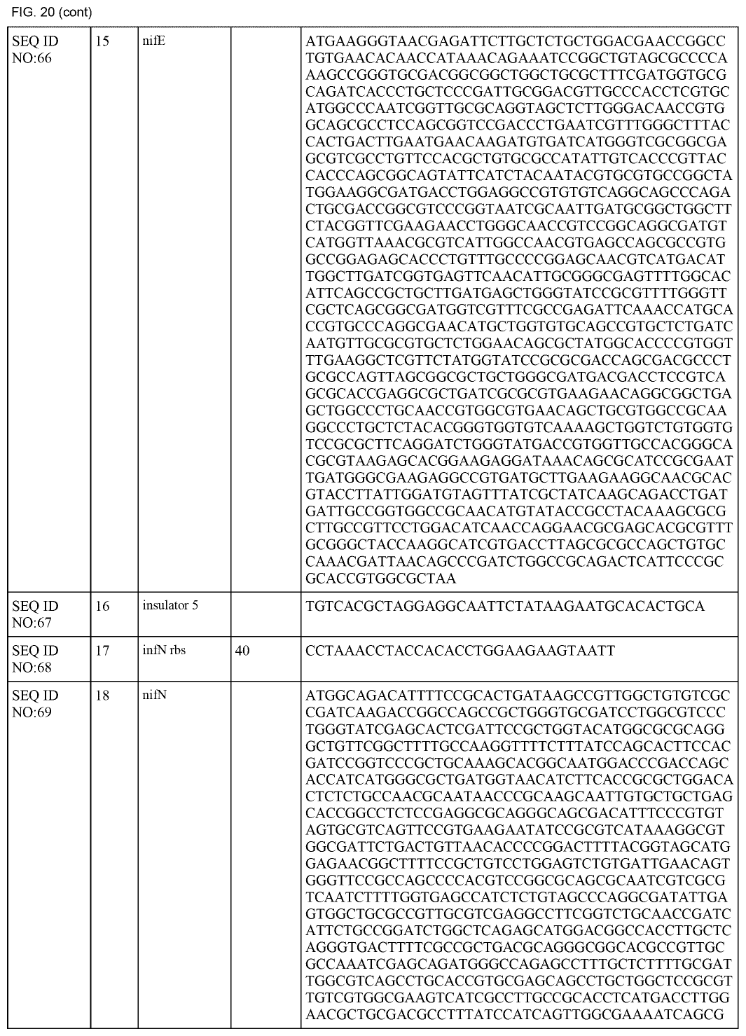

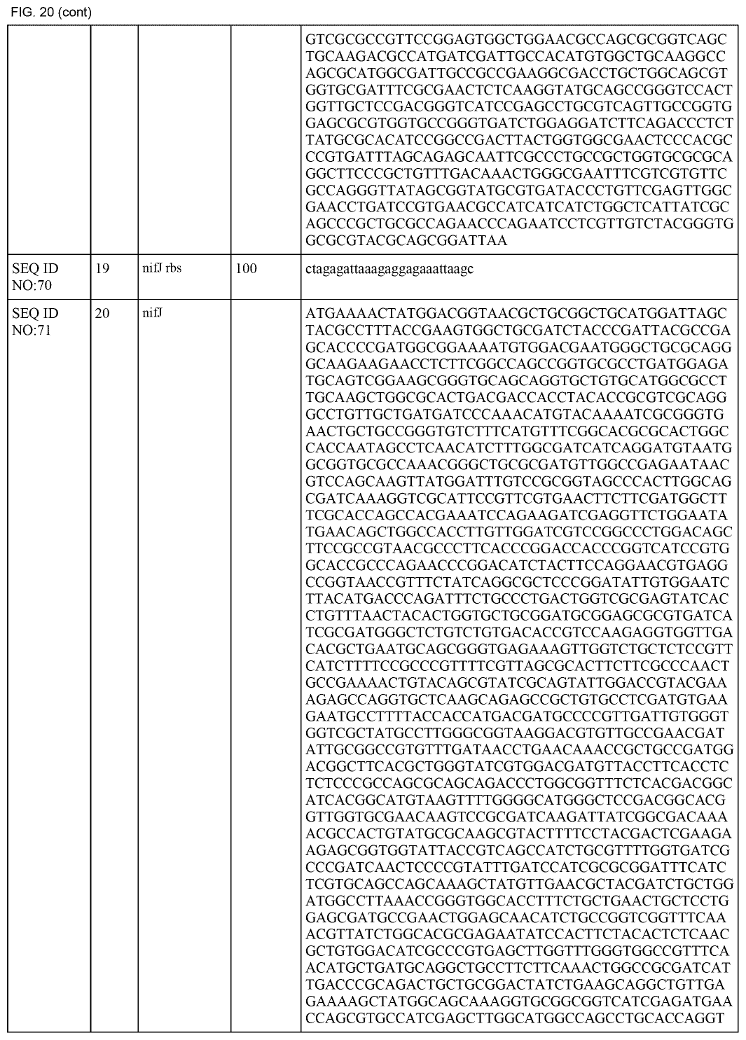

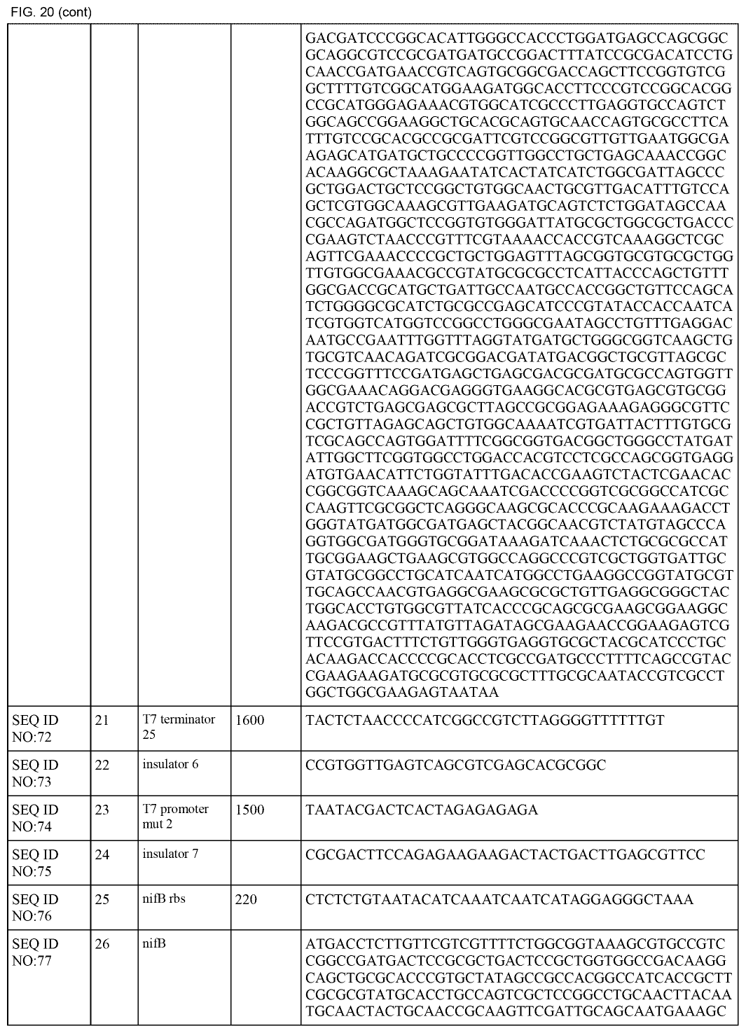

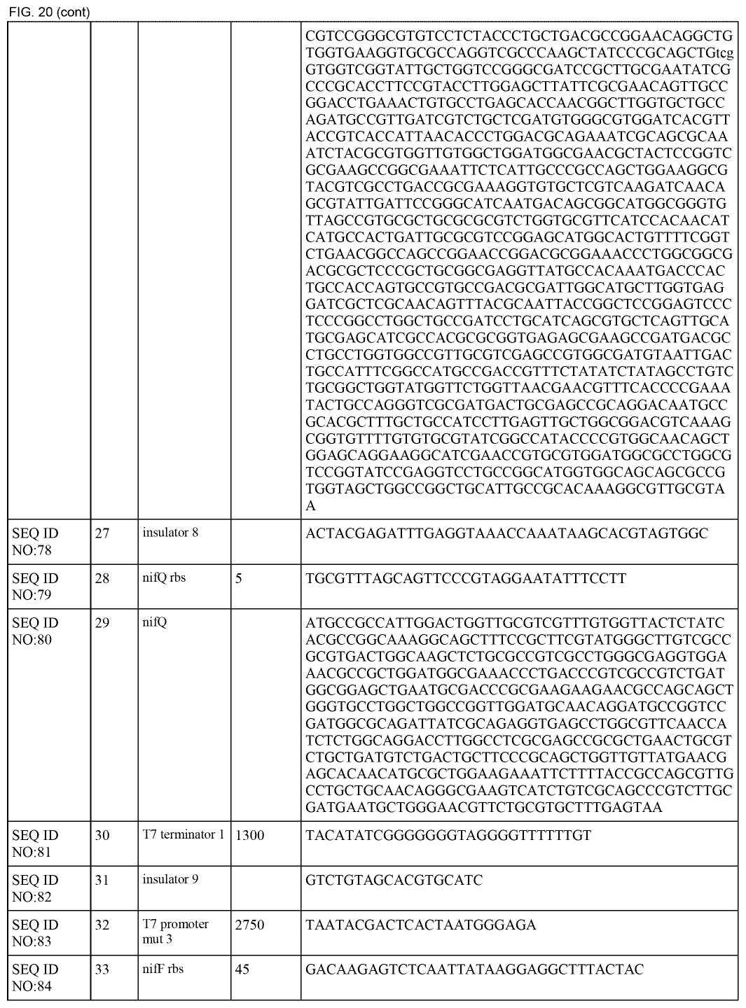

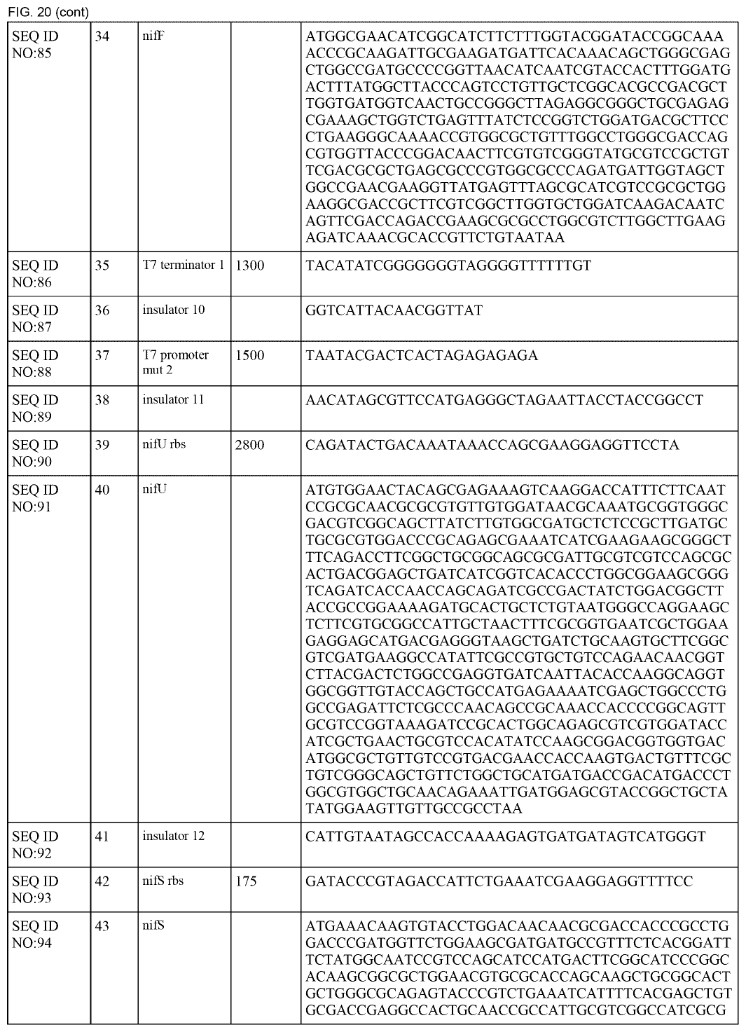

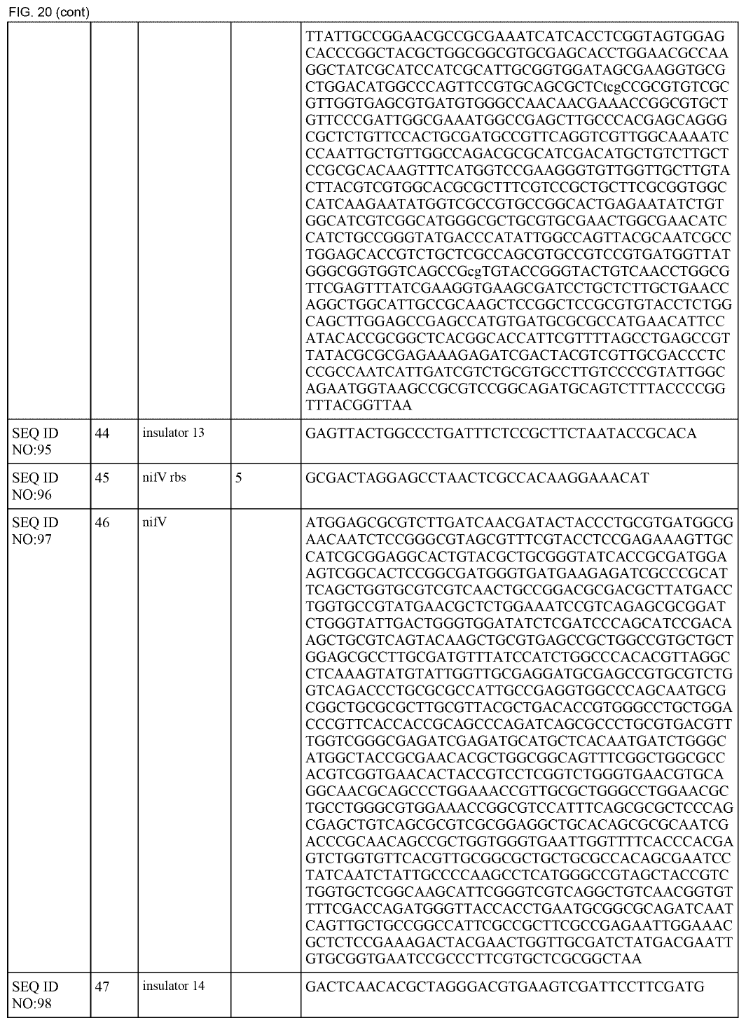

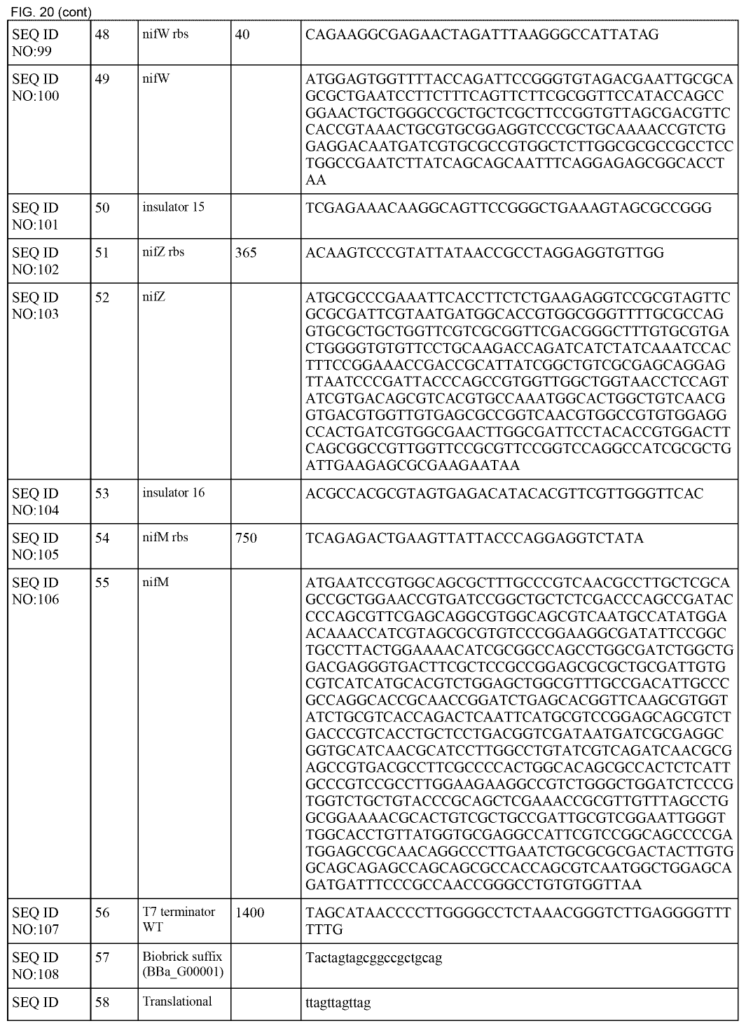

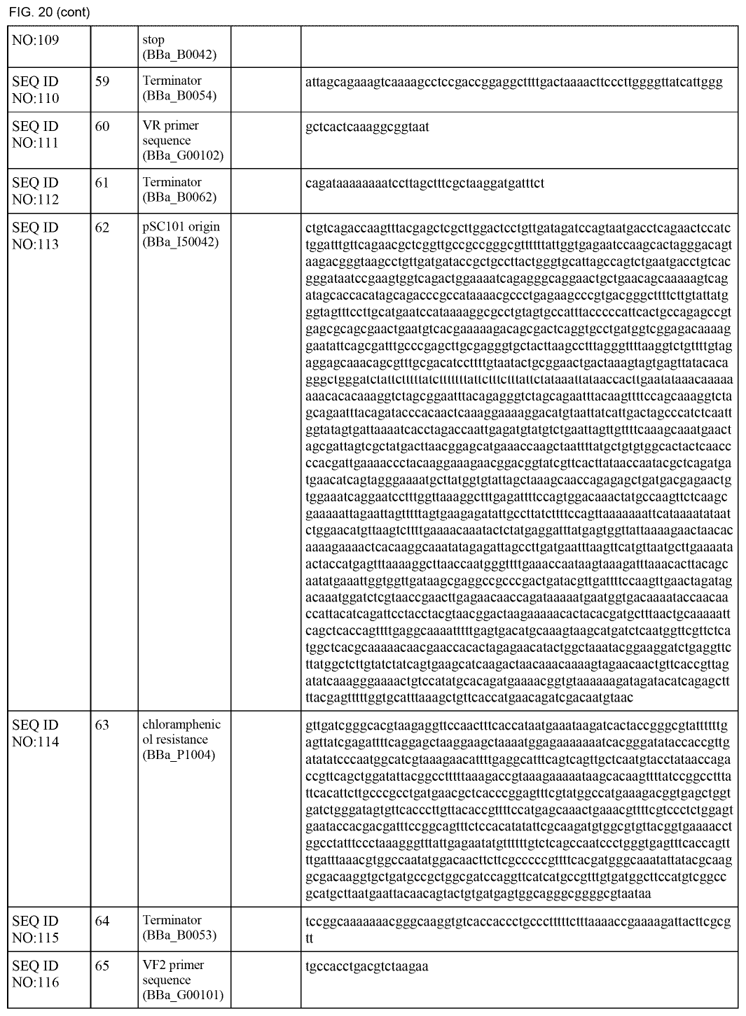

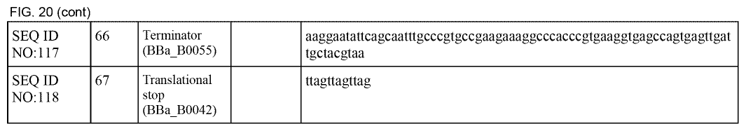

FIG. 17 shows a schematic of the full biological cluster, with each part detailed. FIG. 19 shows the parts list of the synthetic controllers. FIG. 20 shows names, sequences and strengths of each component of the full cluster.

We have further demonstrated that complex genetic circuits can be used to produce functional performance of the synthetic gene cluster. We constructed a genetic circuit encoding the logic “A and not B” and used this circuit to control T7 RNAP. In this circuit, the “A and not B” logic corresponds to the presence or absence of the inducers, IPTG and aTc, such that the cell computes “IPTG and not aTc.” The circuit was constructed by modifying controller #1 to include the cIrepressor binding sites OR1 and OR2 in the Ptac promoter to produce controller #3. Additionally, plasmid pNOR1020 (see, e.g., Tamsir and Voigt Nature 469:212-215 (2011)) encodes the repressor cI under control of the Ptet promoter. When pNOR1020 and controller #3 are co-transformed, they produce the logic circuit “IPTG and not aTc.”

Ptac controller #1 promoter sequence (SEQ ID NO:
334):
tattctgaaatgagctgttgacaattaatcatcggctcgtataatgt
gtggaattgtgagcggataacaatt
Controller #3 promoter sequence (SEQ ID NO: 335):
tattaacaccgtgcgtgttgacagctatacctctggcggttataatg
ctagcggaattgtgagcggataacaatt

| | | Synthetic |
|---|---|---|
| | Expected Logic | Nitrogen Fixation |
| Input | Output | Performance (% WT) |
| No inducer | 0 | <0.5% |
| 1 mM IPTG | 1 | 9% |
| 50 ng/ml aTc | 0 | <0.5% |
| 1 mM IPTG and 50 ng/ml aTc | 0 | <0.5% |

In this experiment, we also included controller #1 as a performance reference. Under inducing conditions (1 mM IPTG), controller #1 exhibits 12% of WT fixation.

### Example 3: Refactoring the Bacterial Type III Secretion System (T3SS)

This example illustrates the use of the method described herein to completely refactor the Bacterial type III secretion system (T3SS). This example also illustrates that the refactored synthetic operons of T3SS are controllable and function independently of all native control and regulation.

Bacterial type III secretion systems (T3SS) are valuable because, unlike conventionally used Sec and Tat pathways, they translocate polypeptides through both inner and outer membranes. This enables the delivery of protein directly to culture media, which can be one of the critical requirements in engineered bacterial technology. For example, toxic proteins can be removed from the cytoplasm without being allowed in the periplasm and functional enzymes (e.g., cellulases) which need to work outside the cell, can be delivered directly into the media.

However, the difficulty with utilizing T3SS in engineered bacterial systems is twofold. T3SS generally exist in pathogenic bacteria which utilize these mechanisms for invasion of host cells. Thus, T3SS are very tightly regulated in the cell and are difficult to control independently. Because of this, we chose to use methods of the present invention to completely refactor T3SS and test the function of the refactored operons in knockout cells.

The term “refactoring” refers to a process that involves optimization of multiple genes, regulation of a gene cluster, and establishment of the genetic context for a biological circuit. Refactoring complex gene clusters and engineering biological pathways requires numerous iterations between design, construction and evaluation in order to improve a desired system property. Briefly, refactoring includes breaking down a biological system into its component parts and rebuilding it synthetically. It also involves removing all native control and regulation of the biological system in order to replace it with a mechanism that provides independent control.

This example illustrates a method of recoding 18 genes of the bacterial type III secretion systems. The term “recoding” refers to a method of removing or replacing sequence of a gene in order to reduce or eliminate any native regulation elements, while also preserving the protein sequence encoded by the gene. The genes of the type III secretion system were recoded using an algorithm provided by DNA2.0 (Menlo Park, CA) in which individual codons of each gene are re-selected such that the gene encodes the same protein, but with maximum dissimilarity with the native sequence.

The 18 genes are arranged in two bacterial operons. Each gene is a recoded version of a native gene from Salmonella typhimurium. Each gene is coupled to a synthetic ribosome binding site (RBS) sequence that sets an appropriate expression level for each individual gene. Details of the synthetic RBS selection are described below. The operons can be induced with any desired 20promoter. In this example, simple inducible promoters are used. The recoded T3SS operons can be attached to any genetic control circuit as needed.

To select a synthetic RBS sequence that best matches the native expression level of each of the 18 genes of the bacterial type III secretion systems, we measured the expression of each gene in the natural system. We cloned the 36-base region upstream on the start codon, along with the 36-bases of coding region fused to an RFP (Red Fluorescent Protein). This was cloned into a plasmid with a constitutive promoter.

This construct was transformed into Salmonella typhimurium SL1344 and grown overnight at 37° C. in PI-1 inducing media (LB with 17 g/L NaCl). The culture was subcultured into fresh inducing media to an OD260 of 0.025, grown for 2 hours at 37° C. until cells reached log-phase. Fluorescence was measured on a cytometer. The geometric mean of RFP fluorescence across at least 10,000 cells was used as the measure of protein expression.

To find ribosomal binding sequences to test, we utilized the Ribosome Binding Site Calculator (voigtlab.ucsfedu/software), identified known RBS sequences from the Registry of Standard Biological Parts (partsregistry.org/Main_Page), and generated a series of randomized sequences. The randomized sequences comprise the following formats:

(SEQ ID NO: 336)
CTTGGGCACGCGTCCATTAANNAGGANNAATTAAGC;
(SEQ ID NO: 337)
TGGGCACGCGTCCATTAANNAGGANNAATTATTAGC;
(SEQ ID NO: 338)
TACTTGGGCACGCGTCCATTAANNAGGANNAATAGC;
(SEQ ID NO: 339)
CTTGGGCACGCGTCCATTAANAAGGAGNAATTAAGC;
(SEQ ID NO: 340)
CTTGGGCACGCGTCCATTANTAAGGAGGNATTAAGC.

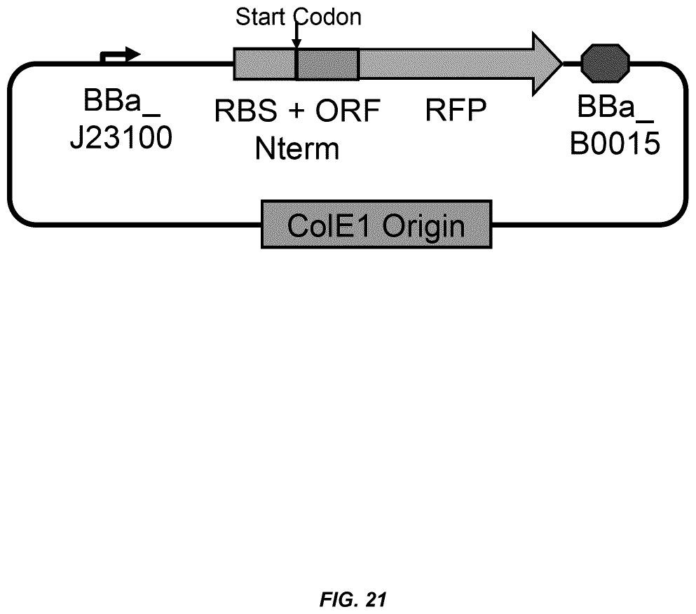

All RBS sequences were cloned into the RBS test vector (FIG. 21) along with the first 36 bases of the synthetic gene they were generated to drive. We followed the same experimental procedure used to measure the expression of each gene in the natural system. Of the randomized RBS, 12-48 colonies of each randomized sequence was tested. The synthetic construct that best matched the native expression level was selected and sequenced. This sequence was then used in the construction of the refactored operons.

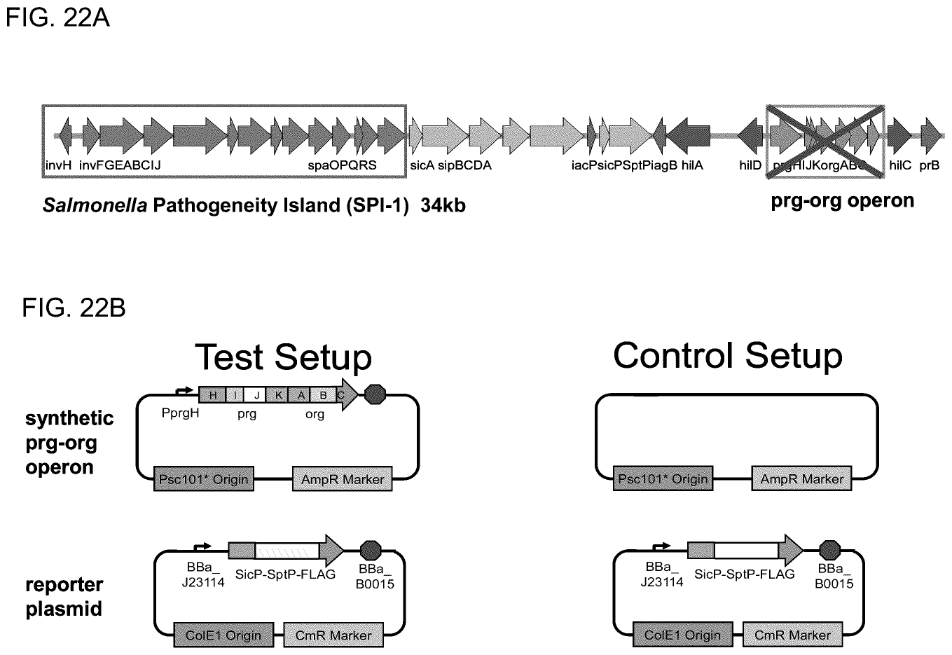

Two operons were assembled. The first, “prg-org” contains 6 genes, and the second “inv-spa” contains 13 genes. These genes are allocated to each operon in the same manner as in the wild-type system. However, the order of genes in each operon is arranged on the basis of measured expression level from strongest to weakest. Operons were assembled by placing the selected synthetic RBS in front of its corresponding synthetic gene sequence. Restriction enzyme binding sites were added between genes or pairs of genes in order to facilitate future manipulation. The entire sequence was synthesized by DNA2.0. The synthetic operon was cloned into a low-copy test vector and placed under the control of an inducible promoter (e.g., pTac or pBad—IPTG or Arabinose induction). A reporter plasmid was created containing a native Salmonella secretable effector protein which was fused to a FLAG epitope tag for identification. This reporter was placed under a strong constitutive promoter. FIG. 22B shows a schematic of the prg-org operon test vector and a reporter plasmid.

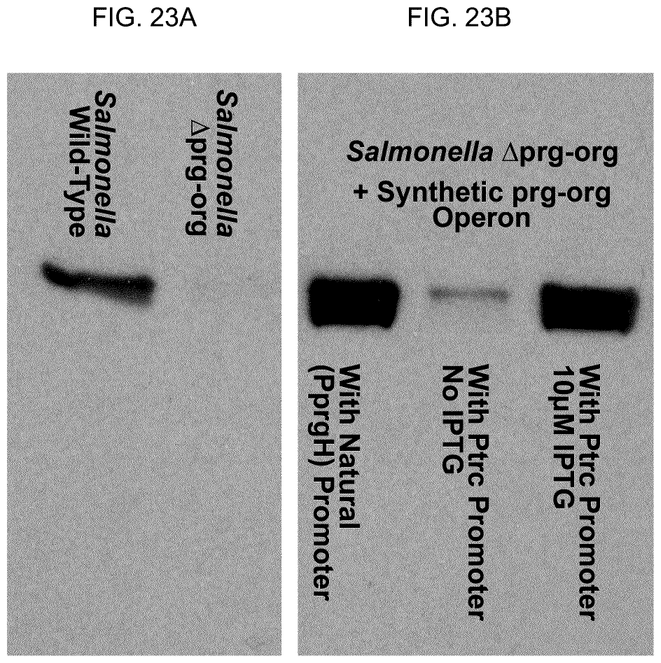

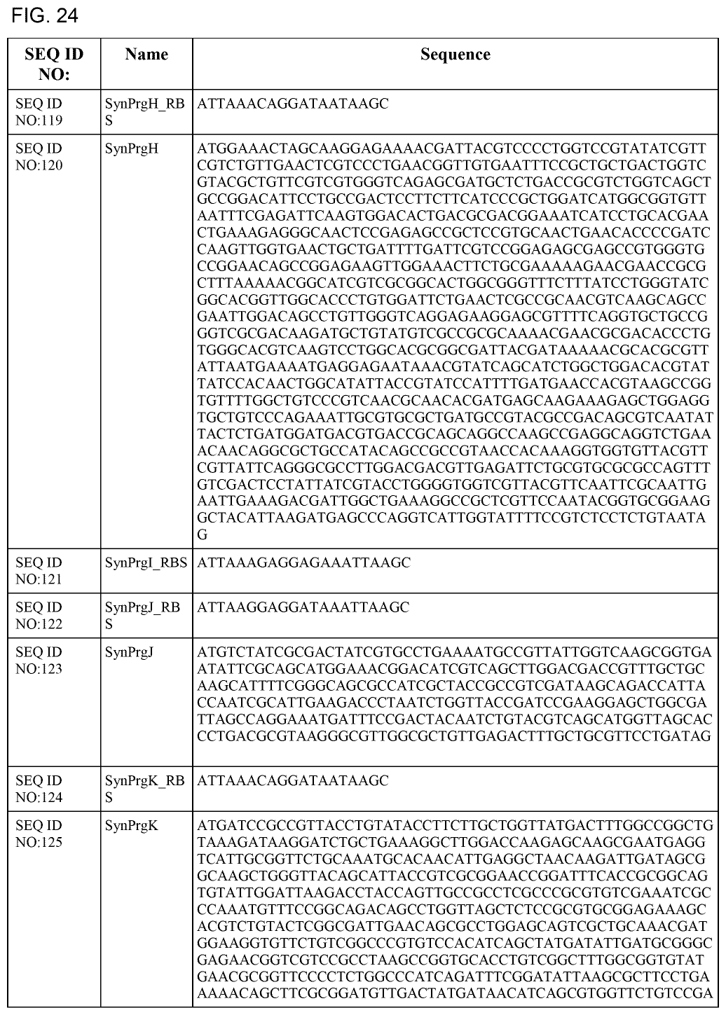

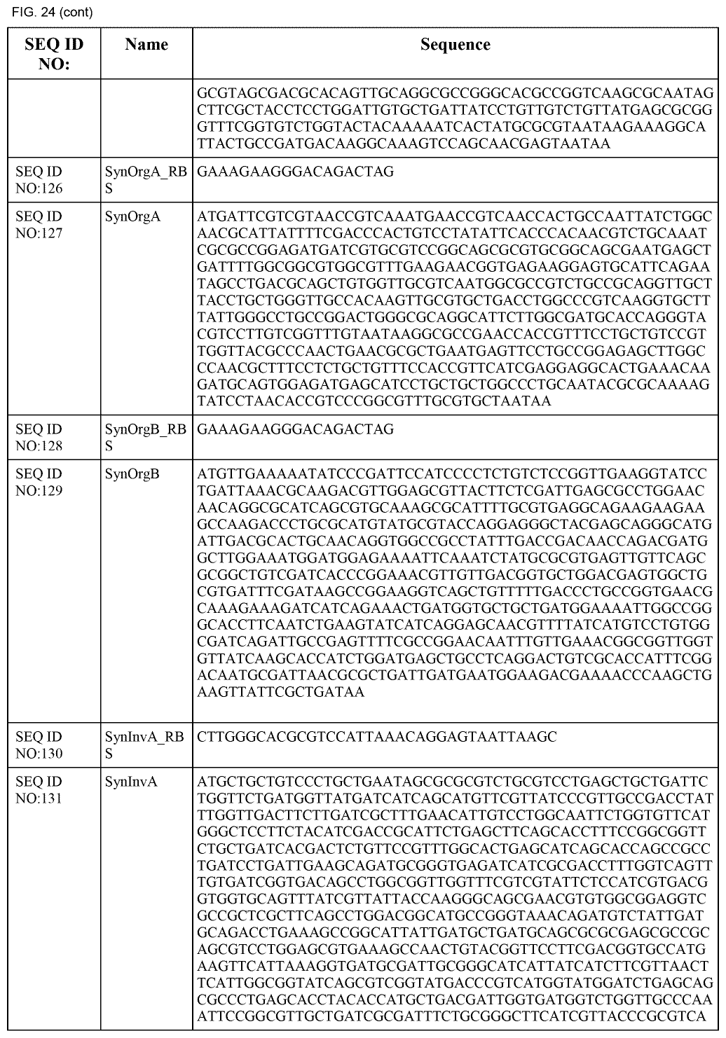

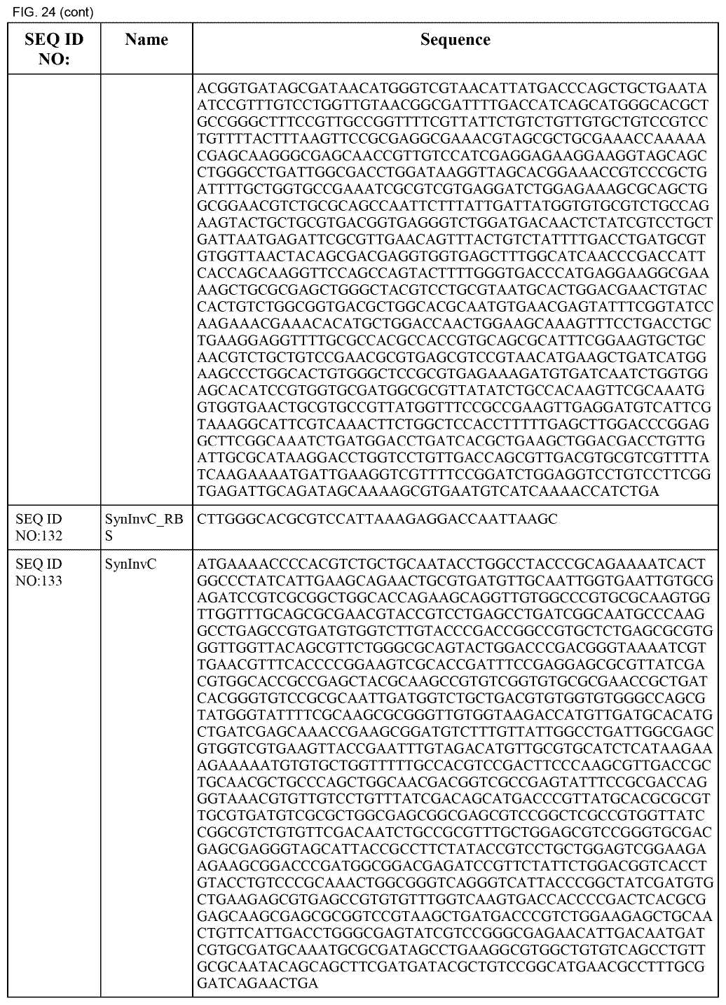

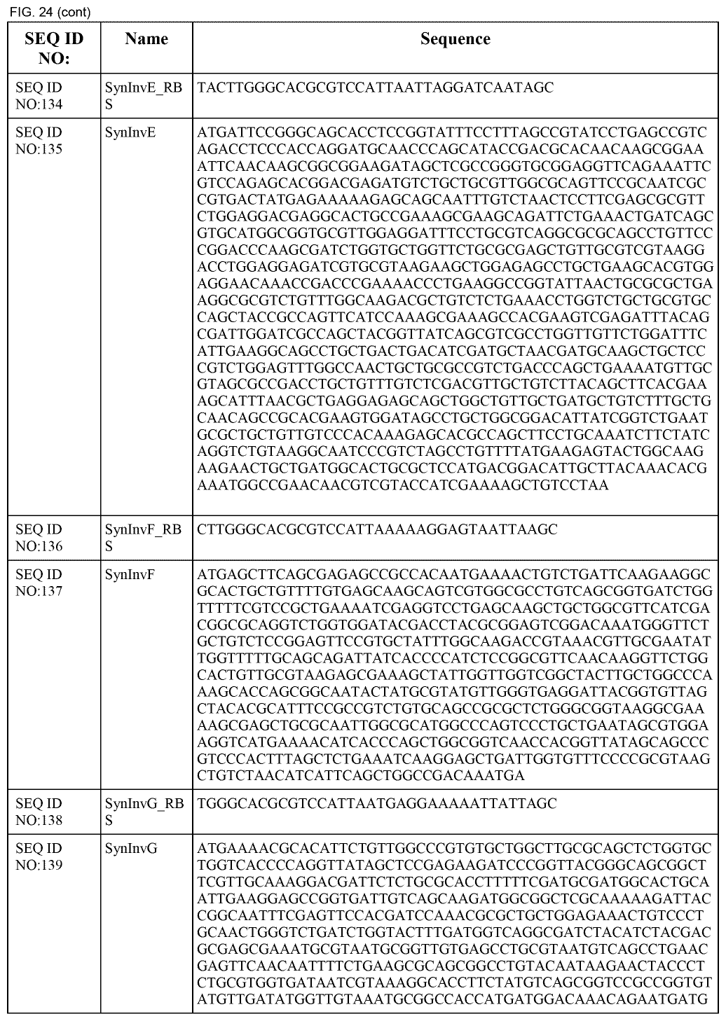

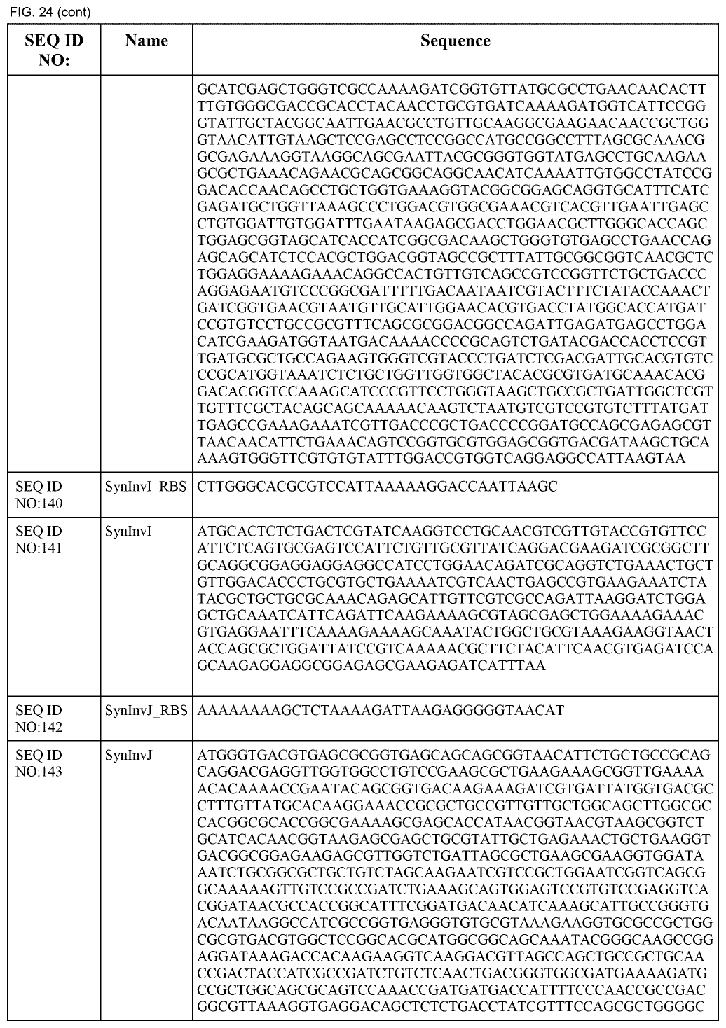

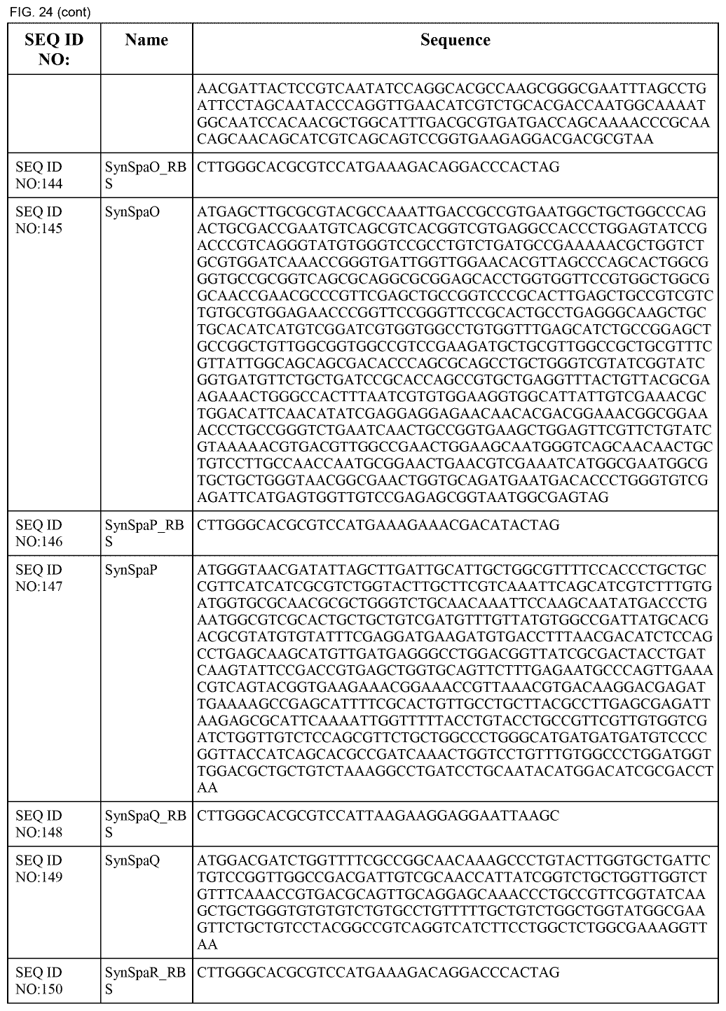

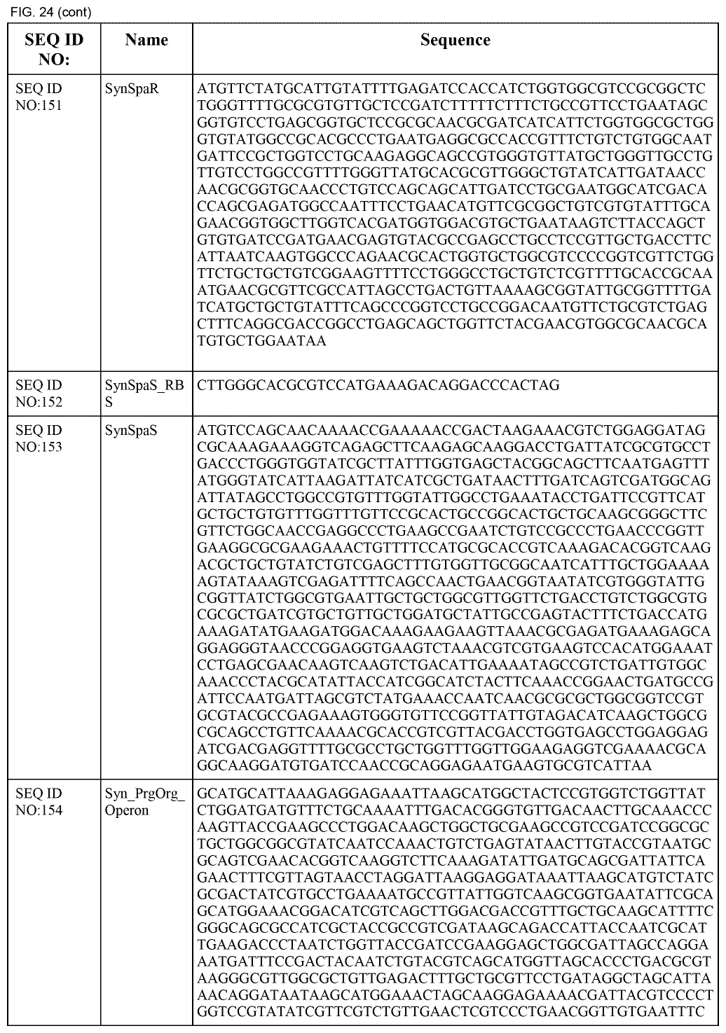

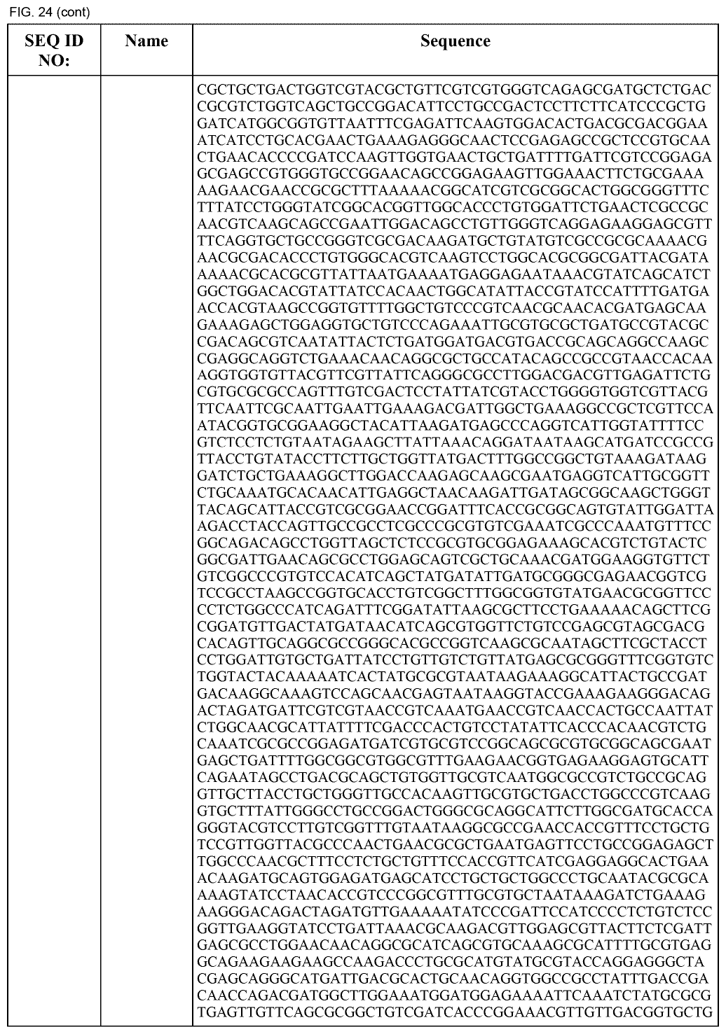

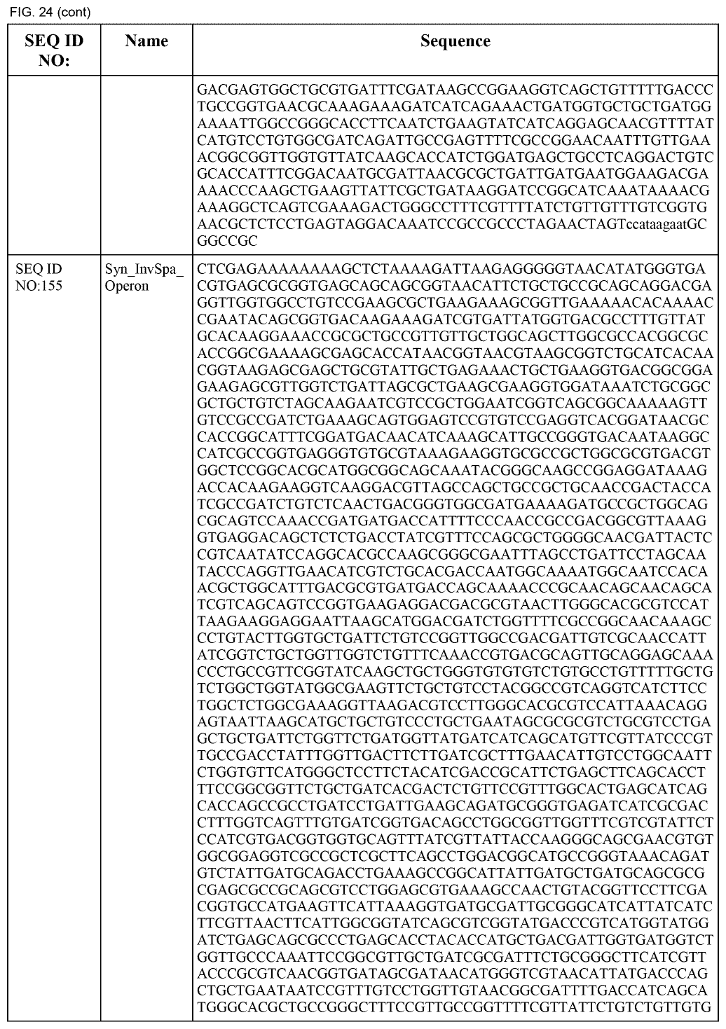

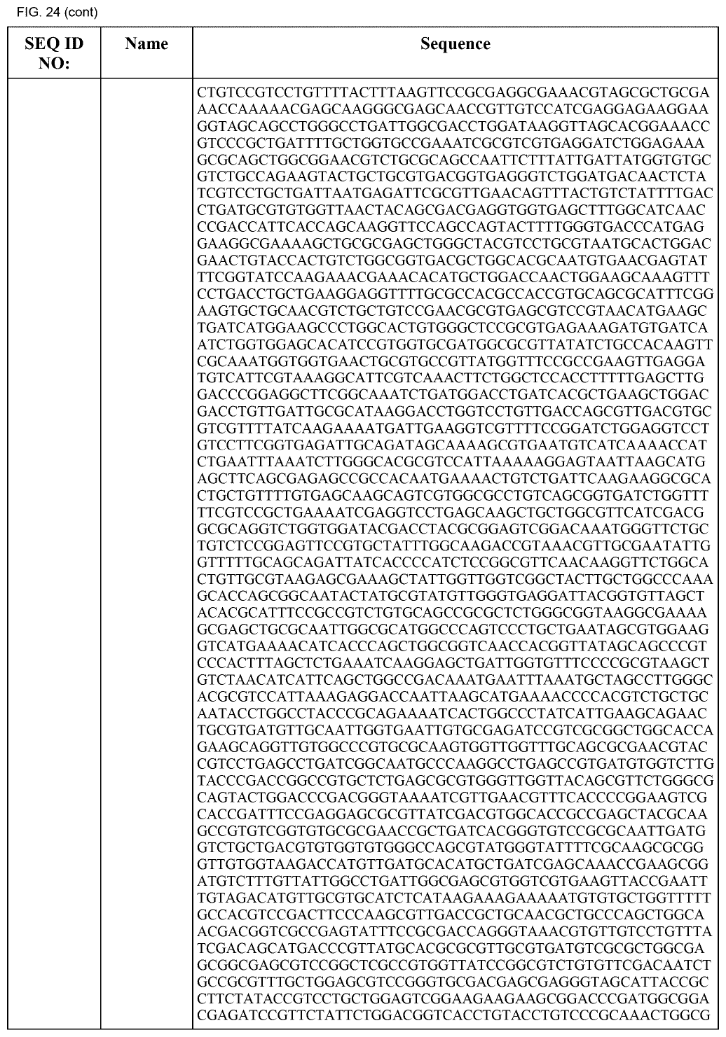

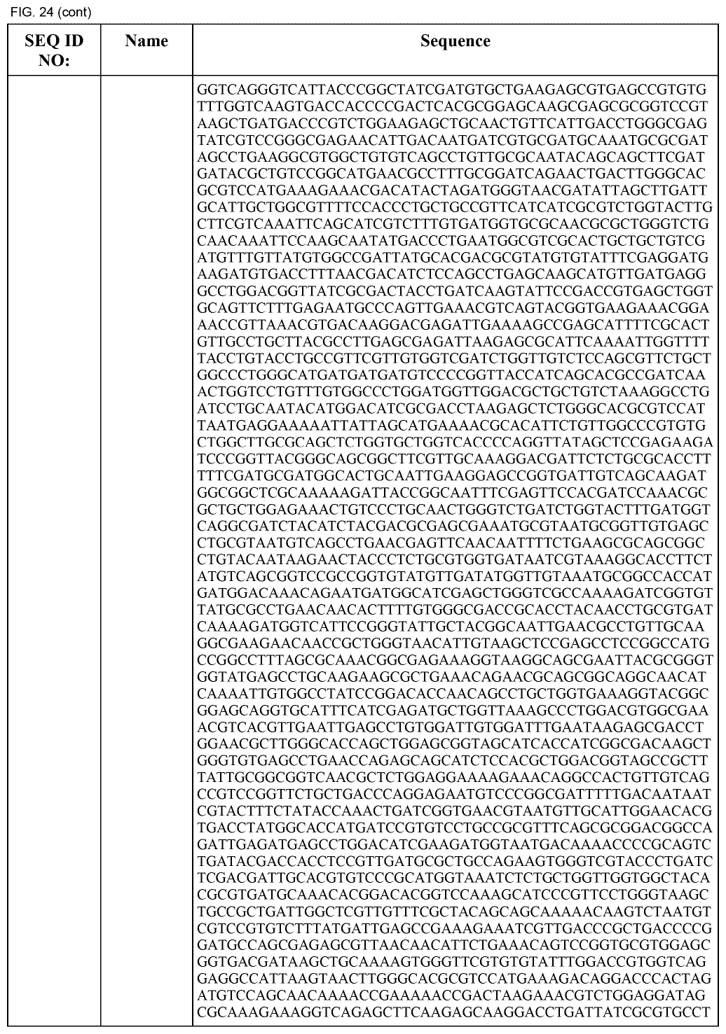

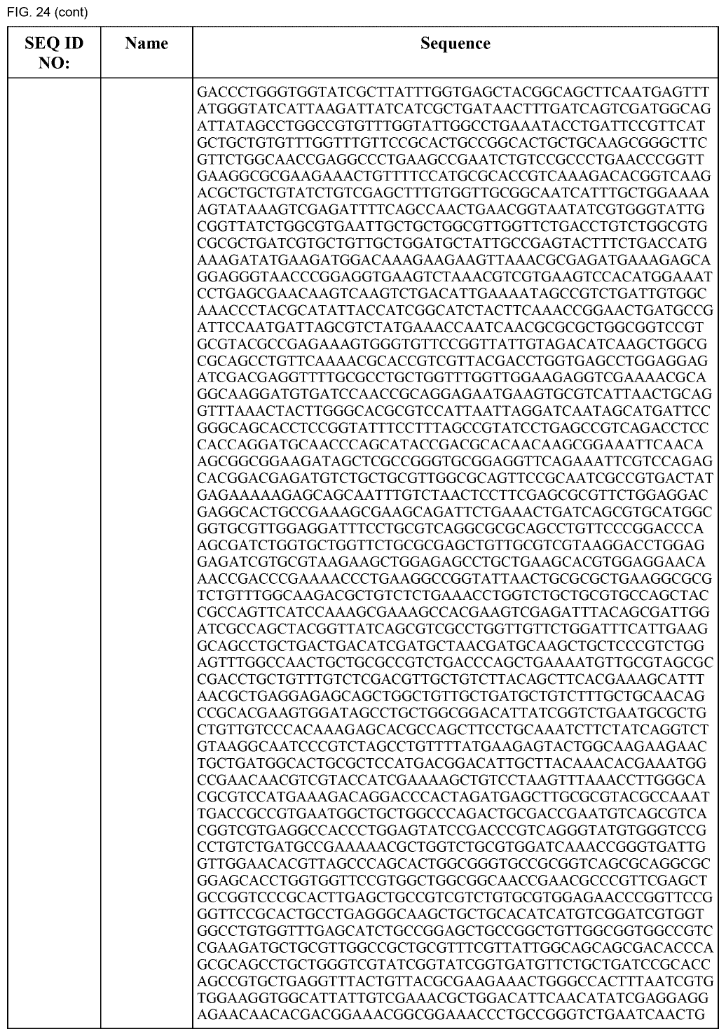

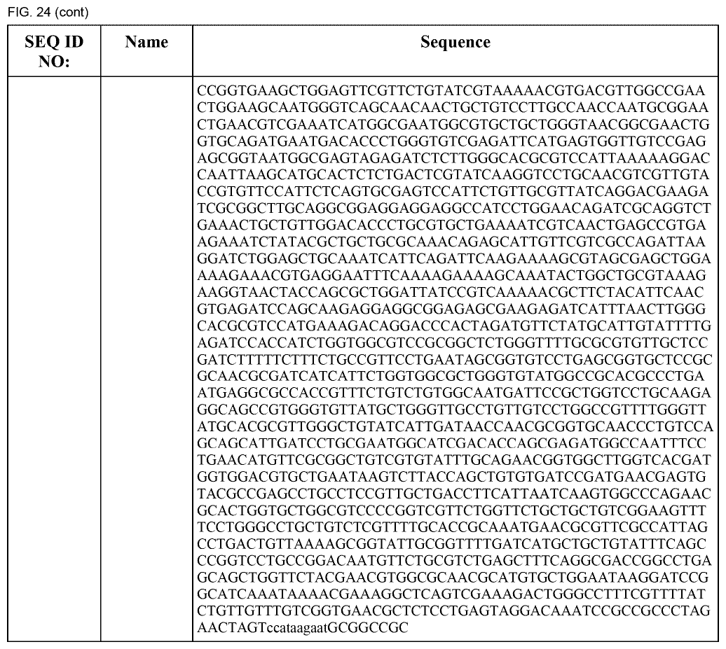

We also generated two operon knockout (prg-org and inv-spa) Salmonella SL1344 cell lines using the method described in Datsenko, Wanner, Proc. Natl. Acad. U.S.A., 2000. FIG. 22A shows a schematic of Δprg-org Salmonella SL1344 knock-out strain. The inv-spa and prg-org operons are boxed. FIG. 23A shows that the Δprg-org knock-out strain does not express the prg-org operon.

The test plasmid (or the control plasmid) and the reporter plasmid were transformed into the appropriate knockout strain. The strains were grown from colony overnight in low-salt media (LB with 5 g/L NaCl) at 37° C. The cultures were subcultured to an OD₂₆₀ of 0.025 in fresh low-salt media and grown for 2 hours. The cultures were diluted 1:10 into high-salt, inducting media (LB with 17 g/L of NaCl) in 50 mL unbaffled flasks and grown for 6-8 hours. 1 mL of each culture was spun down at 3000×g for 5 minutes, then the supernatant filtered through a 0.2 uM filter. This culture was then run on an SDS-PAGE gel and a western blot performed with an anti-FLAG antibody.

FIG. 23B shows that the synthetic refactored prg-org operon in Salmonella Δprg-org cells can be controlled by the addition of IPTG. The level of expression is comparable to that generated from the natural PprgH promoter.

### Example 4: Refactoring Nitrogen Fixation Gene Cluster from Klebsiella oxytoca

Bacterial genes associated with a single trait are often grouped in a contiguous unit of the genome known as a gene cluster. It is difficult to genetically manipulate many gene clusters due to complex, redundant, and integrated host regulation. We have developed a systematic approach to completely specify the genetics of a gene cluster by rebuilding it from the bottom-up using only synthetic, well-characterized parts. This process removes all native regulation, including that which is undiscovered. First, all non-coding DNA, regulatory proteins, and non-essential genes are removed. The codons of essential genes are changed to create a DNA sequence as divergent as possible from the wild-type gene. Recoded genes are computationally scanned to eliminate internal regulation. They are organized into operons and placed under the control of synthetic parts (promoters, ribosome binding sites, and terminators) that are functionally separated by insulator parts. Finally, a controller consisting of genetic sensors and circuits regulates the conditions and dynamics of gene expression. We applied this approach to an agriculturally relevant gene cluster from Klebsiella oxytoca encoding the nitrogen fixation pathway for converting atmospheric N2 to ammonia. The native gene cluster consists of 20 genes in 7 operons and is encoded in 23.5 kb of DNA. We constructed a refactored gene cluster that shares little DNA sequence identity with wild-type and for which the function of every genetic part is defined. This work demonstrates the potential for synthetic biology tools to rewrite the genetics encoding complex biological functions to facilitate access, engineering, and transferability.

### Introduction

Many functions of interest for biotechnology are encoded in gene clusters, including metabolic pathways, nanomachines, nutrient scavenging mechanisms, and energy generators (1). Clusters typically contain internal regulation that is embedded in the global regulatory network of the organism. Promoters and 5′-UTRs are complex and integrate many regulatory inputs (2, 3). Regulation is highly redundant; for example, containing embedded feedforward and feedback loops (4). Regulation can also be internal to genes, including promoters, pause sites, and small RNAs (5, 6). Further, genes often physically overlap and regions of DNA can have multiple functions (7). The redundancy and extent of this regulation makes it difficult to manipulate a gene cluster to break its control by native environmental stimuli, optimize its function, or transfer it between organisms. As a consequence, many clusters are cryptic, meaning that laboratory conditions cannot be identified in which they are active (8).

Gene clusters have been controlled from the top-down by manipulating the native regulation or adding synthetic regulation in an otherwise wild-type background (9). For example, either knocking out a repressor or overexpressing an activator has turned on clusters encoding biosynthetic pathways (10-14). When the cluster is a single operon, it has been shown that a promoter can be inserted upstream to induce expression (15). The entire echinomycin biosynthetic cluster was transferred into E. coli by placing each native gene under the control of a synthetic promoter (16).

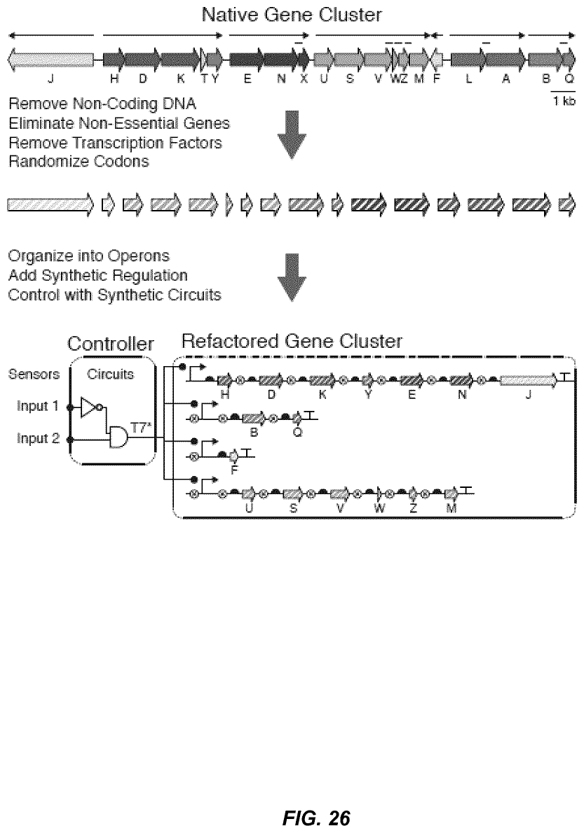

In engineering, one approach to reduce the complexity of a system is to “refactor” it, a term borrowed from software development where the code underlying a program is rewritten to achieve some goal (e.g., stability) without changing functionality (17). This term was first applied to genetics to describe the top-down simplification of a phage genome by redesigning known genetic elements to be individually changeable by standard restriction digest (18). Here, we use it to refer to a comprehensive bottom-up process to systematically eliminate the native regulation of a gene cluster and replace it with synthetic genetic parts and circuits (FIG. 26). The end product is a version of the gene cluster whose DNA sequence has been rewritten, but it encodes the same function. The design process occurs on the computer, and then the resulting DNA sequence is constructed using DNA synthesis (19). The first step of the process is to remove all non-coding DNA, and regulatory genes. Next, each essential gene is recoded by selecting codons that produce a DNA sequence that is as distant as possible from the wild-type sequence. The intent is to introduce mutations throughout the gene to eliminate internal regulation (including that which is undiscovered), such as operators, promoters, mRNA secondary structure, pause sites, methylation sites, and codon regulation. Recoded sequences are further scanned by computational methods to identify putative functional sequences, which are then removed. The recoded genes are organized into artificial operons and the expression levels are controlled by synthetic ribosome binding sites (RBSs), and insulator sequences physically separate the genes. The end result is a refactored gene cluster whose native regulation has been removed and has been organized into a set of discrete, well-characterized genetic parts.

Once the native regulation has been removed, synthetic regulation can be added back to control the dynamics and conditions under which the cluster is expressed. Constructing such regulation has been a major thrust of synthetic biology and involves the design of genetic sensors and circuits and understanding how to connect them to form programs (20). In our design, we genetically separate the sensing/circuitry from the refactored pathway by carrying them on different low copy plasmids (FIG. 26). The plasmid containing the sensors and circuits is referred to as the “controller” and the output of the circuits lead to the expression of an engineered T7 polymerase (T7*). The refactored cluster is under the control of T7 promoters. One advantage of this organization is that T7 polymerase is orthogonal to native transcription and the T7 promoters are tightly off in the absence of the controller. In addition, changing the regulation is simplified to swapping the controller for one that contains different sensors and circuits, so long as the dynamic range of T7* is fixed.

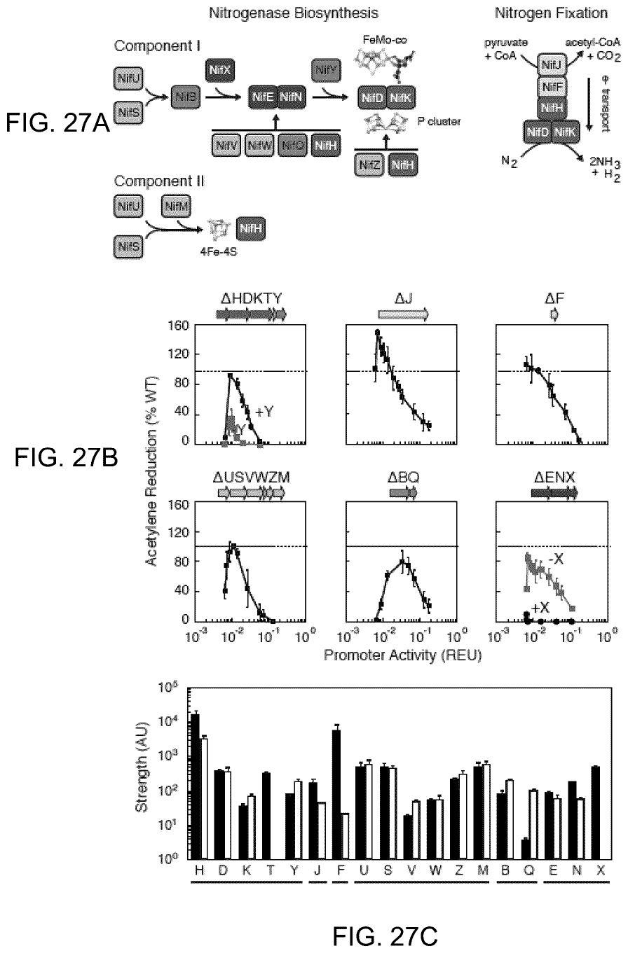

As a demonstration, we have applied this process to refactor the gene cluster encoding nitrogen fixation in Klebsiella oxytoca (21). Nitrogen fixation is the conversion of atmospheric N2 to ammonia (NH3), so that it can enter metabolism (22). Industrial nitrogen fixation through the Haber-Bosch process is used to produce fertilizer. Many microorganisms fix nitrogen and the necessary genes typically occur together in a gene cluster, including the nitrogenase subunits, the metallocluster biosynthetic enzymes and chaperones, e-transport, and regulators (FIG. 27A) (23, 24). The gene cluster from K. oxytoca has been a model system for studying nitrogen fixation and consists of 20 genes encoded in 23.5 kb of DNA (FIG. 26, top) (25). The biosynthesis of nitrogenase is tightly regulated by a two-layer transcriptional cascade in response to fixed nitrogen, oxygen, and temperature (26). The complete cluster has been transferred to E. coli, thus demonstrating that it has all of the genes necessary for nitrogen fixation (27). The encoding of this function is complex, many of the genes overlap, the operons are oriented in opposite directions, and there are many putative hidden regulatory elements, including internal promoters and hairpins (25). The purpose of refactoring is to reorganize the cluster, simplify its regulation, and assign a concrete function to each genetic part.

### Results

### Tolerance of the Native Gene Cluster to Changes in Expression

Prior to refactoring a cluster, a robustness analysis is performed to determine the tolerances of a gene or set of genes to changes in expression level (FIG. 27B). This informs the grouping of genes into operons and the selection of synthetic parts to obtain desired expression levels. In the wild-type background, genes are knocked out and complemented under inducible control. The tolerance is obtained by measuring nitrogenase activity as a function of the activity of the inducible promoter.

Nitrogenase function is notably sensitive to expression changes and each tolerance has a clear optimum (FIG. 27B). The chaperone NifY is required to achieve full activity and broadens the tolerance to changes in expression level. NifT did not have an effect on activity, as observed previously (28), and it it is frequently absent from homologous clusters (29). The genes controlling electron transport (nifJ and nifF) need to be expressed at low levels, and activity falls rapidly as expression increases. The optima for genes participating in the metal cluster biosynthetic pathways vary. The nifUSVWZM operon, which encodes proteins for early Fe—S cluster formation and proteins for component maturation, needs to be expressed at low levels, whereas nifBQ, encoding proteins for FeMo-co core synthesis and molybdenum integration, need to be expressed 10-fold higher. NifEN is tolerant to varied expression levels. However, activity is lost with the inclusion of nifX, which has been characterized as a negative regulator (30). The native cluster also includes the regulatory proteins NifL and NifA, which integrate environmental signals (26). The genes nifT, nifX, and nifLA are not included in the refactored cluster.

### The Complete Refactored Gene Cluster

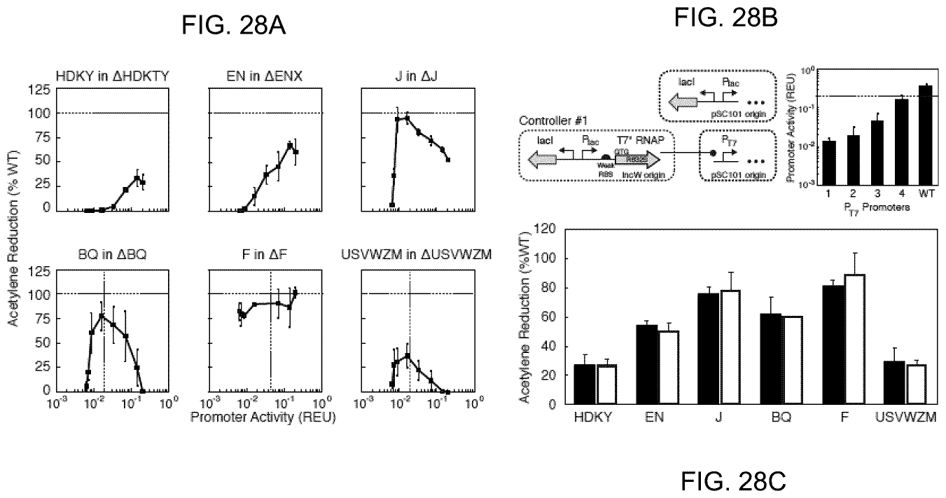

The nitrogenase activities of the refactored operons were measured as a function of the IPTG-inducible P_{tac} promoter (FIG. 28A). Each operon has a different optimum. To combine the operons, the P_{tac} promoters were replaced with T7 promoters that have a strength close to the measured optimum (FIG. 28B and Materials and Methods section). The nitrogenase genes (nifHDK) are highly expressed in Klebsiella under fixing conditions (up to 10% of cell protein) (31), so the strongest promoter was used to control this operon (T7.WT, 0.38 REU) (32). A long operon was built to include the nifEN and nifJ genes, where the lower expression required for nifJ was achieved through transcriptional attenuation. The nifF gene was encoded separately under the control of a medium strength promoter (T7.3, 0.045 REU). Finally, the nijUSVWZM and nifBQ operons were controlled by weak promoters (T7.2, 0.019 REU). Each of the individual refactored operons under the control of a T7 promoter was able to recover the activity observed from the Ptac promoter and corresponding optimal IPTG concentration (FIG. 28C).

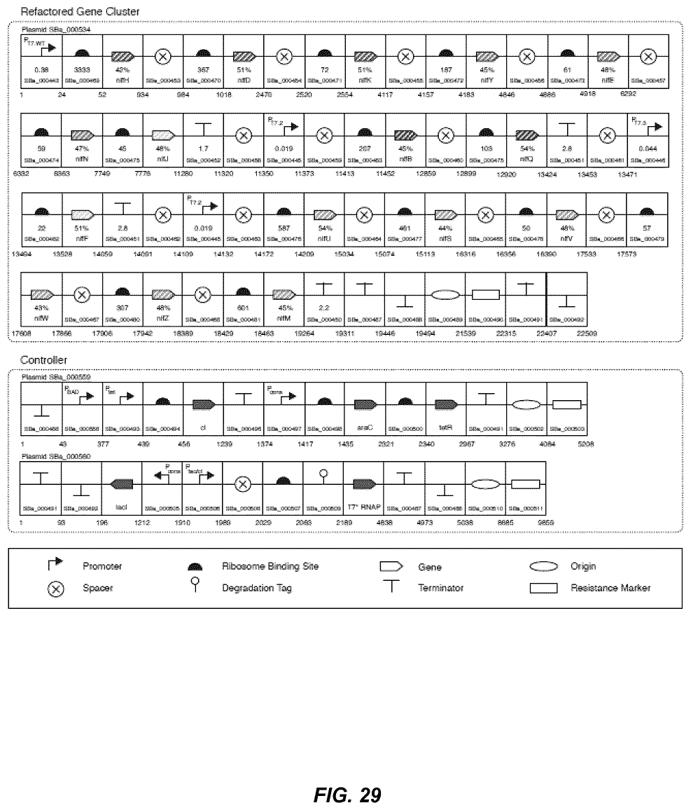

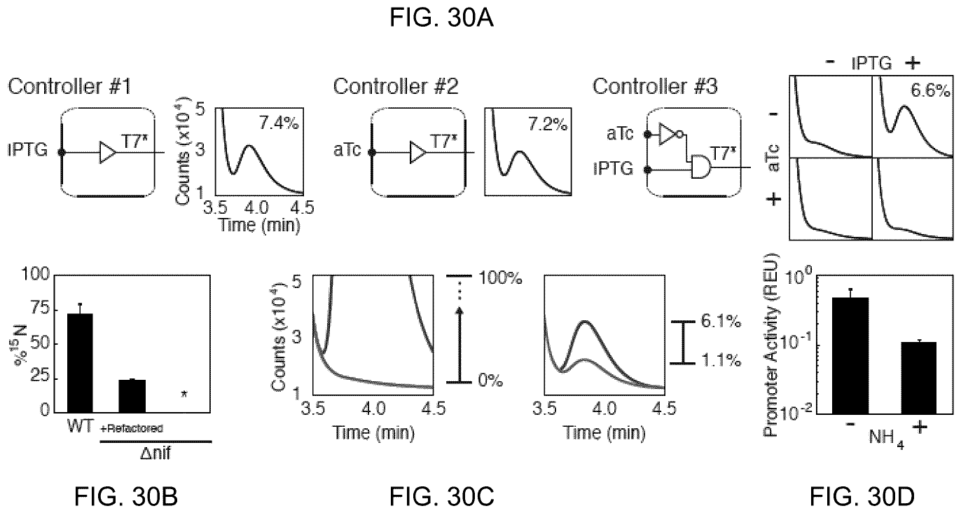

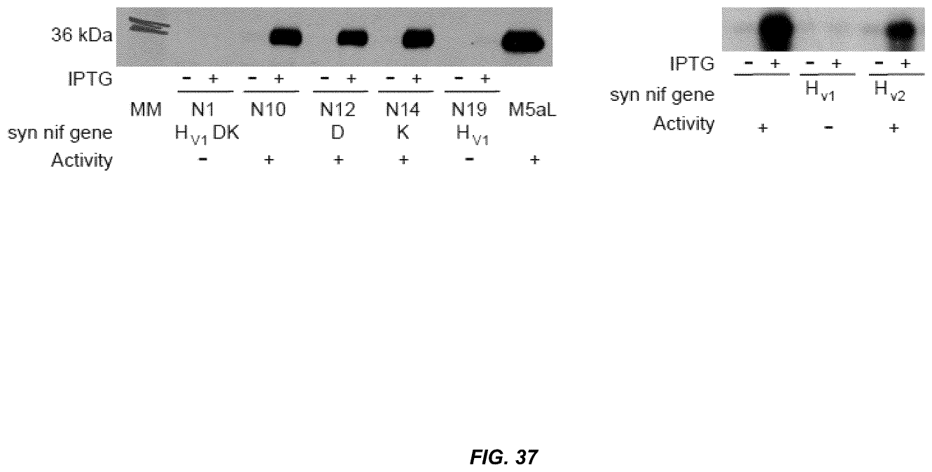

Transitioning the control to T7* and T7 promoters facilitates the assembly of the complete cluster from refactored operons. We first assembled half-clusters using Gibson Assembly (33) and verified their function in strains with the corresponding genes deleted. The first half-cluster consisted of the nifHDKYENJ operon. The second half-cluster was assembled from the nifBQ, nifF, and nfUSVWZM operons. The half-clusters were able to recover 18% 0.7% and 26%±8.4% of wild-type activity, respectively. The full synthetic cluster was assembled from both half-clusters (FIG. 29), and its activity measured in a strain where the full cluster is deleted. The synthetic gene cluster recovers nitrogenase activity at 7.4%±2.4% of the wild-type (FIG. 30A). Strains carrying the synthetic gene cluster utilized ambient N2 as a nitrogen source, growing 3.5-fold slower than the wild-type strain (FIG. 37) and incorporating ¹⁵N-labelled nitrogen into 24%±1.4% of their cellular nitrogen content, as measured by isotope ratio mass spectronomy (IRMS) (FIG. 30B).

The complete refactored cluster consists of 89 genetic parts, including a controller, and the function of each part is defined and characterized. Therefore, the genetics of the refactored system are complete and defined by the schematic in FIG. 29. However, the process of simplification and modularization reduces activity (18). This is an expected outcome of refactoring a highly evolved system.

### Swapping Controllers to Change Regulation

The separation of the controller and the refactored cluster simplifies changing the regulation of the system. This can be achieved by transforming a different controller plasmid, as long as the dynamic range of the T7* RNAP expression is preserved. To demonstrate this, we constructed two additional controllers (FIG. 30A). Controller #2 changes the chemical that induces the system by placing the expression of T7* RNAP under the control of the aTc-inducible Ptet promoter. When induced, Controller #2 produces nitrogenase activity identical to Controller #1 (7.2%±1.7%). The controller can also serve as a platform to encode genetic circuits to control regulatory dynamics or to integrate multiple sensors. To this end, Controller #3 contains two inducible systems (IPTG and aTc) and an ANDN gate (34, 35). In the presence of IPTG and the absence of aTc, nitrogen fixation is 6.6%±1.7% of wild-type activity. These controllers represent the simplicity by which the regulation of the refactored cluster can be changed.

In addition to making it possible to add new regulation, the process of refactoring eliminates the native regulation of the cluster. This is demonstrated through the decoupling of nitrogenase activity from the environmental signals that normally regulate its activity. For example, ammonia is a negative regulator that limits overproduction of fixed nitrogen (26). In the presence of 17.5 mM ammonia, no nitrogenase activity is observed for the wild-type cluster (FIG. 30C). In contrast, the refactored gene cluster maintains activity in the presence of ammonia (1.1%±0.5%). Interestingly, this 7-fold reduction of activity is not due to residual regulation present in the system. Rather, it occurs because the addition of ammonia to the media reduces the output of the controller by 4.5-fold (FIG. 30C). In theory, this could be fixed by increasing the expression level of T7* RNAP, but it speaks to the need to create genetic circuits that are robust to environmental context.

### Discussion

The objective of refactoring is to facilitate the forward engineering of multi-gene systems encoded by complex genetics. Native gene clusters are the product of evolutionary processes; thus, they exhibit high redundancy, efficiency of information coding, and layers of regulation that rely on different biochemical mechanisms (36-38). These characteristics inhibit the quantitative alteration of function by part substitution, because the effect can become embedded in a web of interactions. Here, modularizing the cluster, physically separating and insulating the parts, and simplifying its regulation have guided the selection and analysis of part substitutions. The information gleaned from screening the permutations in a refactored system can be cleanly fed back into the design cycle.

The refactored cluster can also serve as a platform for addressing questions in basic biology. First, it allows for the impact of regulatory interactions to be quantified in isolation. For example, in the natural system, one feedback loop could be embedded in many other regulatory loops. Systematically removing such regulation provides a clean reference system (potentially less active and robust than wild-type) from which improvements can be quantified as a result of adding back regulation. It also serves as a basis for comparison of radically different regulatory programs or organizational principles; for example, to determine the importance of temporal control of gene expression (4, 39) or the need for genes to be encoded with a particular operon structure (40, 41). Second, the process of reconstruction and debugging is a discovery mechanism that is likely to reveal novel genetics and regulatory modes. In this work, the improvement from 0% to 7% revealed only minor changes: misannotations in genes and improper expression levels. However, the debugging process itself is blind to the mechanism—it simply identifies problematic regions of DNA.

One of the immediate applications of refactoring is in the access of gene clusters from genomic sequence information. This could be necessary either because the cluster is silent, meaning that it that cannot be activated in the laboratory, or because the desired cluster is from a metagenomic sample or information database and the physical DNA is unavailable (42). There are have been many elegant methods to activate a gene cluster, including the placement of inducible promoters upstream of the natural operons and the division of the cluster into individual cistrons, which can then be reassembled (43). With advances in DNA synthesis technology, it is possible to construct entire gene clusters with complete control over the identity of every nucleotide in the design. This capability eliminates the reliance on the natural physical DNA for construction and enables the simultaneous redesign of components in the complete system. Fully harnessing this technology will require the marriage of computational methods to select parts and scan designs, characterized part libaries, and methods to reduce their context dependence.

### Material and Methods

### Strains and Media

E. coli strain S17-1 was used for construction and propagation of all plasmids used in Klebsiella oxytoca knockout mutant construction. K. oxytoca strain M5a1 (Paul Ludden, UC Berkeley) and mutants derived from M5a1 were used for nitrogen fixation experiments. Luria-Bertani (LB)-Lennox was used for strain propagation. All assays were carried out in minimal medium containing (per liter) 25 g of Na₂HPO₄, 3 g of KH₂PO₄, 0.25 g of MgSO₄·7H₂O, 1 g of NaC1, 0.1 g of CaCl₂·2H₂O, 2.9 mg of FeCl₃, 0.25 mg of Na₂MO₄·2H₂O, and 20 g of sucrose. Growth media is defined as minimal media supplemented with 6 ml (per liter) of 22% NH₄Ac. Derepression media is defined as minimal media supplemented with 1.5 ml (per liter) of 10% serine. The antibiotics used were 34.4 μg ml⁻¹ Cm, 100 μg ml⁻¹ Spec, 50 μg ml⁻¹ Kan, and/or 100 μg ml−1 Amp.

### Codon Randomization

Initial gene sequences were proposed by DNA2.0 to maximize Hamming distance from the native sequence while seeking an optimal balance between K. oxytoca codon usage and E. coli codon preferences experimentally determined by the company (44). Rare codons (<5% occurrence in K. oxytoca) were avoided, and mRNA structure in the translation initiation region was suppressed. Known sequence motifs, including restriction sites, transposon recognition sites, Shine-Dalgarno sequences and transcriptional terminators, were removed by the DNA2.0 algorithm.

### Elimination of Undesired Regulation

Each synthetic operon was scanned prior to DNA synthesis to identify and remove undesired regulation. Multiple types of regulation were identified using publicly available software. The RBS Calculator was used (Reverse Engineering, 16S RNA: ACCTCCTTA) to identify ribosome binding sites throughout the proposed DNA sequence of the operon (45). The Prokaryotic Promoter Prediction server was used to identify putative σ70 promoter sites (e-value cutoff of 5, sigma.hmm database) (46). The PromScan algorithm was used to identify putative σ54 promoter sites using default options (47). TransTermHP software was used with default parameters to identify terminator sequences in both the forward and reverse directions (48). RBSs greater than 50 AU and all identified promoters and terminators were considered significant.

### Nitrogenase Activity Assay

In vivo nitrogenase activity is determined by acetylene reduction as previously described (49). For Koxytoca whole-cell nitrogenase activity assay, cells harboring the appropriate plasmids were incubated in 5 ml of growth media (supplemented with antibiotics, 30° C., 250 r.p.m.) in 50 ml conical tubes for 14 hours. The cultures were diluted into 2 ml derepression media (supplemented with antibiotics and inducers) to a final OD₆₀₀ of 0.5 in 14 ml bottles, and bottles were sealed with rubber stoppers (Sigma Z564702). Headspace in the bottles was repeatedly evacuated and flushed with N2 past a copper catalyst trap using a vacuum manifold. After incubating the cultures for 5.5 hours at 30° C., 250 r.p.m, headspace was replaced by 1 atmosphere Ar. Acetylene was generated from CaC₂ using a Burris bottle, and 1 ml was injected into each bottle to start the reaction. Cultures were incubated for 1 hour at 30° C., 250 r.p.m before the assay was stopped by injection of 300 μl of 4M NaOH solution into each bottle. To quantify ethylene production, 50 μl of culture headspace was withdrawn through the rubber stopper with a gas tight syringe and manually injected into a HP 5890 gas chromatograph. Nitrogenase activity is reported as a percentage of wild-type activity. Briefly, ethylene production by strains was quantified by integrating area under the peak using HP Chemstation software and dividing ethylene production of experimental strains by the ethylene production of a wild type control included in each assay.

N₂-Dependent Growth and ¹⁵N₂ Incorporation Assay

Nitrogen fixation by synthetic nif cluster in K. oxytoca is further demonstrated by N₂-dependent growth and ¹⁵N₂ incorporation. Cells are diluted as described in the acetylene reduction assay. The headspace of the bottles is replaced by normal N₂ gas or by stable isotope nitrogen, ¹⁵N₂ (¹⁵N atom 99.9%, Icon Isotopes, Cat #: IN 5501). After incubating the cultures for 36 hours at 30° C., 250 r.p.m, N₂-dependent growth of the cells is determined by measuring optical density at 600 nm (OD600). To do the ¹⁵N₂ incorporation assay, the ¹⁵N-enriched cells with corresponding control cultures under normal nitrogen gas are collected by centrifugation, the cell pellets are dried in a laboratory oven at 100° C. for 12 hours. The dried pellets are analysis for ¹⁵N/¹⁴N ratio at the Center for Stable Isotope Biogeochemistry at the University of California, Berkeley using the Finnigan MAT Delta plus Isotope Ratio Mass Spectrometer.

K. oxytoca Knockout Strains

All K. oxytoca mutants are constructed from M5a1 by allele exchange using suicide plasmid pDS132 carrying the corresponding nif gene deletion (pDS132 was graciously provided by the Paul Ludden lab at UC Berkeley as a gift from Dr. Dominique Schneider at Universite Joseph Fourier) (49). We made a slight modification to a previously published protocol (50). Here, a kanamycin resistance cassette was cloned into the suicide plasmid upstream of the left homologous exchange fragment. These operon deletions in nif gene cluster span the promoter and the complete amino acid coding sequences except when specifically designated. All mutants were verified by DNA sequencing of the PCR product of the corresponding gene region to confirm physical DNA deletion and by whole-cell acetylene reduction assay to confirm the lack of nitrogenase activity.

### Promoter Characterization

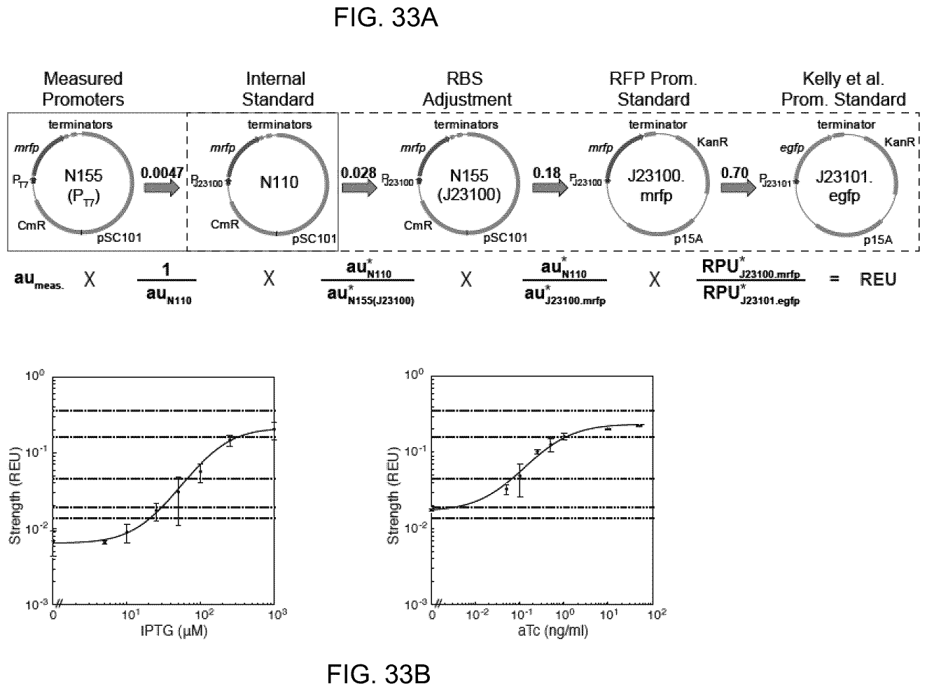

As described in this example, the output of promoters is reported as relative expression units (REU). This is simply a linear factor that is multiplied by the arbitrary units measured by the flow cytometer. The objective of normalizing to REU is to standardize measurements between labs and projects. The linear factor is 1.66×10⁵ and the division by this number back converts to the raw arbitrary units. This number was calculated to be a proxy to the RPU (relative promoter units) reported by Kelly and co-workers (51). Our original standardized measurements were made prior to the Kelly paper and involved a different reference promoter, fluorescent protein (mRFP), RBS, and plasmid backbone. Because of these differences, one cannot calculate RPU as defined by Kelly, et al. Instead, a series of plasmids was made (FIG. 33A) to estimate the relative expression of reporter protein from experimental constructs compared the standard construct in Kelly, et al. Conversion factors between constructs were measured and multiplied to obtain the linear factor above. We renamed the unit to REU (relative expression units) because it is intended to be a simple normalization of fluorescent units (akin to a fluorescent bead) and not a direct measurement of the activity of a promoter (e.g., the polymerase flux).

Cells were grown as in the Acetylene Reduction Assay with two modifications. The initial flush of headspace with N₂ was not performed, and the assay was halted after the 5.5 hour incubation. To halt the assay, 10 μl of cells were transferred from each bottle to a 96-well plate containing phosphate buffered saline supplemented with 2 mg ml⁻¹ kanamycin. Fluorescence data was collected using a BD Biosciences LSRII flow cytometer. Data were gated by forward and side scatter, and each data set consisted of at least 10,000 cells. FlowJo was used to calculate the geometric means of the fluorescence distributions. The autofluorescence value of K. oxytoca cells harboring no plasmid was subtracted from these values to give the values reported in this study. The strengths of T7 promoter mutants were characterized by swapping them in place of the P_{tac} promoter in plasmid N149 (SBa_000516), co-transforming with Controller #1 (plasmid N249), and measuring fluorescence via flow cytometry under 1 mM IPTG induction.

To replace the P_{tac} promoter by a T7 promoter in each synthetic operon, we followed a simple process. First, we identified the IPTG concentration corresponding to the maximal 30 functional activity of each synthetic operon. Second, we translated this IPTG concentration into REU based on characterization of the P_{tac} promoter (FIG. 33B, left). Third, we selected the T7 mutant promoter with the closest strength in REU. For the synthetic nifF operon, we observed broad, robust fixation under the P_{tac} promoter. We found that T7 mut 3 produced inducible functional activity with a maximum at 1 mM IPTG induction of the T7 RNAP. For the synthetic nifJ operon, our method suggests that we use a weak T7 mutant promoter. However, we found that a WT T7 promoter produced inducible activity with a maximum at 1 mM IPTG. We attribute this deviation to a change in RBS strength due to contextual differences between P_{tac} and the T7 promoter.

### Debugging Synthetic Operons

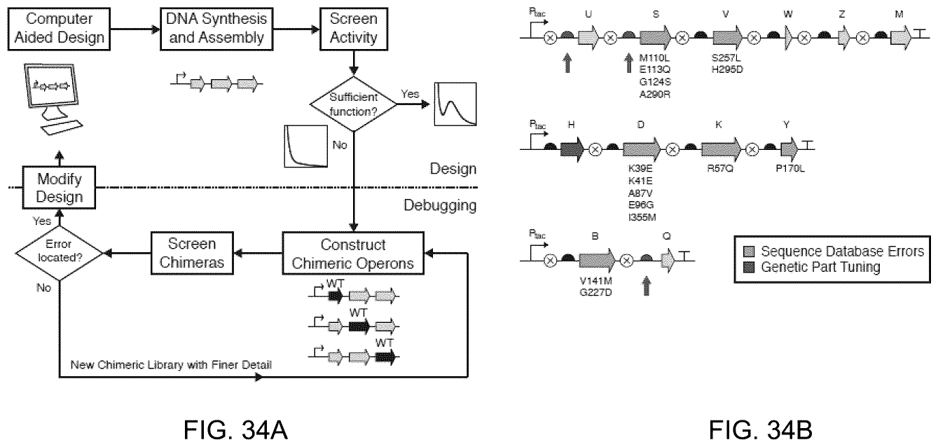

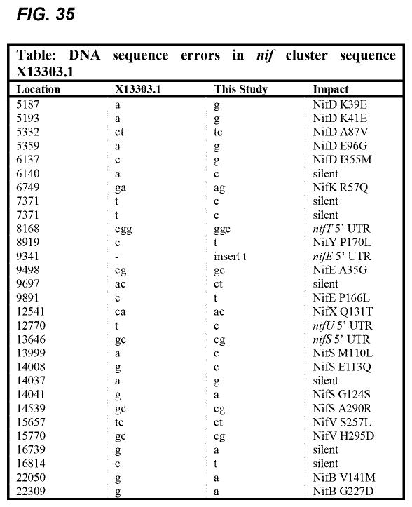

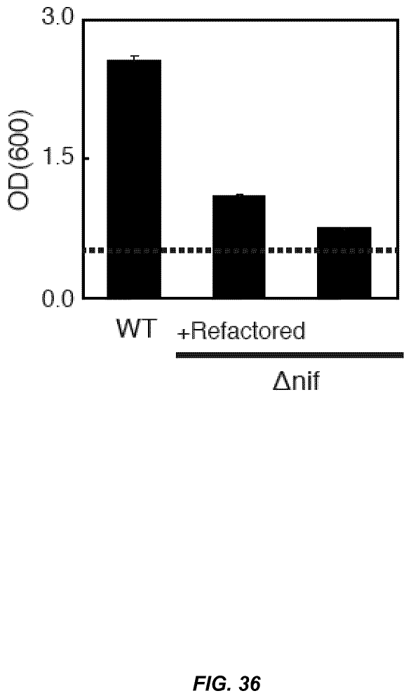

Some of the initial designs for refactored operons showed little or no activity. When this occurs, it is challenging to identify the problem because so many genetic changes have been made simultaneously to the extent that there is almost no DNA identity with the wild-type sequence. To rapidly identify the problem, a debugging method was developed that can be generalized when refactoring different functions (FIG. 34A). Chimeric operons are created by replacing a wild-type region of DNA with its synthetic counterpart. The function of each chimera in this library is assessed to identify which region of synthetic DNA caused a loss of activity. New chimeras are then be constructed with increasingly fine-resolution changes between synthetic and wild-type DNA. This approach “zooms in” on the problematic region of DNA, which can then be fixed. The most common problem is due to errors in the reference DNA sequence (Genbank, X13303.1) (52). Refactored genes were designed using only the amino acid sequence information from the database; thus, they were sensitive to sequencing errors leading to missense mutations that reduced or eliminated activity. Indeed, 18 such mutations were identified and confirmed by carefully resequencing the wild-type cluster (FIG. 35). Fifteen of the 18 mutations occurred in refactored operons that required debugging and were corrected (FIG. 34B). This demonstrates the challenge of reconstituting biological functions using only database information and DNA synthesis (55).

Modifying synthetic RBS strength was also important to debugging. The function of the synthetic nifUSVWZM operon was significantly improved by changing RBSs to match a 1:1 ratio of NifU:NifS. The initial selection of RBSs led to an observed 10:1 ratio in their respective RBS strengths. After debugging, nifU and nifS RBS strength was better balanced (1.25:1) and this improved activity. For one RBS, the measurement method proved to be inaccurate. We found the measured strength of the wild-type nifQ RBS was extremely low (FIG. 27C), and the synthetic nifBQ operon showed low activity when the synthetic nifQ RBS was matched to the measured strength. In contrast, the robustness analysis showed a requirement for high expression level of the nifBQ operon (FIG. 27B). Thus, a strong synthetic RBS near the strength of the nifB RBS was used and significantly improved nfBQ operon activity. In one case, our initial recoded nifH gene did not express well using either wild-type or synthetic regulation (FIG. 37). We designed a new synthetic gene, requiring that it diverge in DNA sequence from both the native and first synthetic DNA sequences and found that the new synthetic gene both expressed well and recovered activity.

### Growth by Nitrogen Fixation

Cells capable of nitrogen fixation should exhibit measurable growth on media that lacks nitrogen by utilizing atmospheric N₂ as a source of nitrogen. Conversely, cells incapable of nitrogen fixation should not grow on nitrogen-free media.

In parallel to the ¹⁵N₂ incorporation assay, we monitored strain growth under nitrogen-limited media conditions and 100% ¹⁵N₂ atmosphere (Methods, N₂-dependent Growth Assay). Cells were grown on derepression media as used in the Nitrogenase Activity Assay. Depression media is not strictly nitrogen-free, containing 1.43 mM serine in order to promote ribosomal RNA production and hasten nitrogenase biosynthesis (54).

Strains containing Controller #1 and the refactored gene cluster grew nearly 30% as much as wild-type strains. In contrast, minimal growth was observed in Δnif strains, consistent with the limited nitrogen available from serine and cell lysis products (55). FIG. 37 illustrates cell growth supported by nitrogen fixation.

Western Blot Assay for Synthetic nifH Expression

The first synthetic nifHDK did not exhibit nitrogenase activity under induction ranging from 0 to 1 mM IPTG, and the nifH gene (synthetic nifHᵥ₁) was identified as a problematic part using the debugging protocol shown in FIG. 34. However there was no mutation found. Western blots were further used to confirm problematic synthetic nifH expression.

A western blot for NifH protein in FIG. 37 (left) showed that wild type nifH expressed well with either synthetic nifD or nifK (construct N10, N12, N14), whereas synthetic nifHᵥ₁ was not expressed regardless of the context of nifDK (construct N₁ and N₁₉). A second synthetic nifH (synthetic nifHᵥ₂) was used to replace synthetic nifHᵥ₁. The western blot in FIG. 37 (right) showed the synthetic nifHᵥ₂ (construct N38) expressed well.

Samples for western blots were prepared by boiling collected K. oxytoca cells in SDS-PAGE loading buffer and run on 12% SDS-Polyacrylamide gels (Lonza Biosciences). Proteins on the gels were transferred to PVDF membranes (BioRad Cat #: 162-0177) using Trans-Blot SD Semi-Dry Transfer Cell (BioRad Cat #: #170-3940). Blocking the membrane and Antibody binding were performed using SNAP i.d. Protein Detection System (Millipore Cat #WBAVDBA). The membranes were blocked by TBST-1% BSA (TBS-Tween20). The anti-NifH and anti-NifDK antibodies (kindly provided by Paul Ludden Lab at UC-Berkeley) were used as the primary antibodies. The anti-NifH antibody was a universal anti-NifH made against a mixture of purified NifH proteins from Azotobacter vinelandii, Clostridium pasteurianum, Rhodospirillum rubrum, and K. oxytoca. The anti-NifDK antibody was made against purified NifDK protein from Azotobacter vinelandii. The anti-NifH and anti-NifDK antibodies were used at 1:500 and 1:2000 respectively. The secondary antibody (Goat anti-Rabbit IgG-HRP, Sigam Cat #: A0545) was used at 1:10,000. Development was done using an enhanced chemiluminescent substrate for HRP (Pierce Cat #: 32209) and captured on film (Kodak: Cat #:178-8207).

### Construction of Plasmids and Parts

Plasmids were designed in silico. Synthetic parts (promoters, RBS, terminators and insulators) were combined with the initial synthetic gene sequences proposed by DNA2.0 in ApE (A Plasmid Editor, http://biologylabs.utah.edu/jorgensen/wayned/ape/) and GeneDesigner (56) to create synthetic operons. Synthetic operons were computationally scanned to eliminate unintended regulation (Methods, “Elimination of Undesired Regulation”), and parts containing such regulation were replaced. This reiterative process continued until the synthetic operons included only designed regulation.

Physical DNA was constructed using standard manipulation techniques. Assembly methods followed published protocols and included BioBrick (57), Megawhop (58), Phusion Site-Directed Mutagenesis or Gibson Assembly methods (59). We found that Gibson Assembly was the most efficient DNA assembly method, except when making small (<10 bp) changes in plasmids under 10 kb in size. We noted assembly failures were infrequent, more common in assemblies above 15 kbp, and linked to the presence of homology within −500 bp of part termini. In these cases, we observed annealing of unexpected parts to create non-intended junctions.

Plasmid pIncW (pSa, SpR) was generated from pEXT21 (pSa, SpR) by deletion of osa, nucl, the Tn21 integrase gene, and ORF18 (60). Plasmid pSB4C5 (pSC101, CmR) was obtained from the Registry of Standard Biological parts and serves as the base vector for wild-type complementation, RBS characterization, and synthetic operons (57). Plasmid N58 (pSC101, CmR) was generated by inserting the P_{tac} cassette (SynBERC Registry, SBa_000561) between the BioBrick prefix and BioBrick suffix of pSB4C5. Plasmid N292 (SBa_000566) was generated by inserting a terminator characterization cassette between the BioBrick prefix and BrioBrick suffix of pSB4C5. The cassette consists of the PT7 promoter, RBS (SBa_000498), GFP, the wild-type T7 terminator, RBS D103 (SBa_000563) from Salis et. al. (13), and mRFP (SBa_000484). Plasmid N149 (SBa_000516) was constructed by inserting the Ptac promoter cassette (SBa_000563), RBS D103 (SBa_000563) from Salis et. al. (13), and mRFP (SBa_000484) between the BioBrick prefix and BioBrick suffix of pSB4C5. Plasmid N505 (SBa_000517) was constructed by inserting the Piet promoter cassette (SBa_000562), RBS D103 (SBa_000563), and mRFP (SBa_000484) between the BioBrick prefix and BioBrick suffix of pSB4C5. Plasmid N110 (SBa_000564) was constructed by inserting a constitutive promoter (SBa_000565), a strong RBS (SBa_000475), and mRFP (SBa_000484) between the BioBrick prefix and BioBrick suffix of pSB4C5. Plasmid N573 (SBa_000559) was constructed by inserting the AmpR resistance marker in pNOR1020 (14).

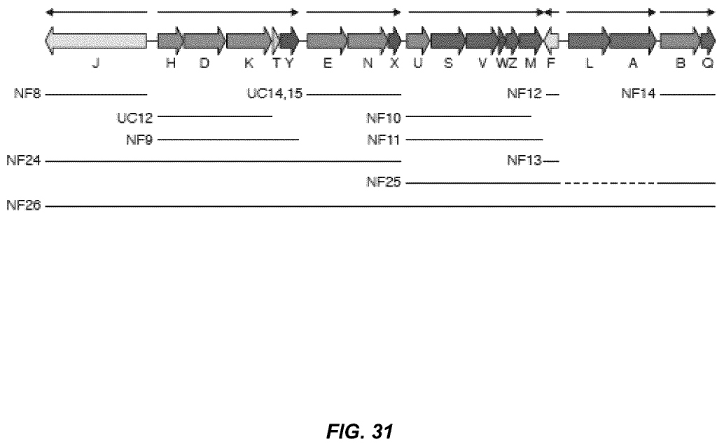

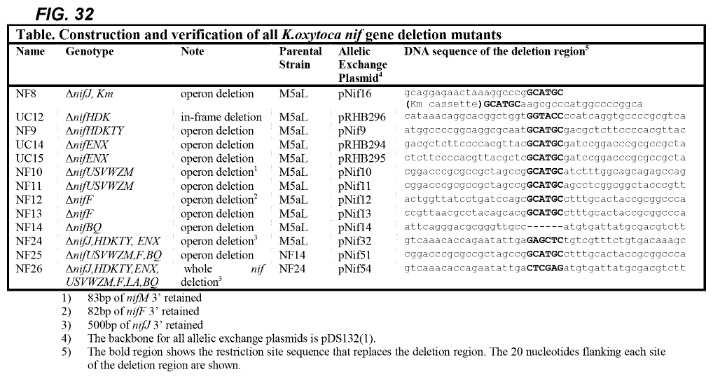

It has been shown that the multicopy expression of some nitrogen fixation genes can eliminate nitrogenase maturation and function (i.e., multicopy inhibition) (63, 64). An additional uncertainty is that the replacement of the native promoter with an inducible promoter could disrupt their function. To examine these effects, we constructed plasmids to complement the activities of the knockout strains (FIG. 31) and tested their activity under inducible control. These plasmids are also the basis for the experiments to quantify the robustness to changes in expression (FIG. 27).

Complementation plasmids were constructed by inserting the DNA encoding each wild-type operon between the Ptac promoter and BioBrick suffix of plasmid N58 (pSC101, CmR). One exception was plasmid Nif18 which was constructed by cloning the nifHDKTY operon into the multi-cloning site of pEXT21 (60). Wild-type operon sequences were defined by published transcription initiation sites (65).

Wild-type RBS characterization vectors were constructed by inserting the region from −60 bp to +90 bp for each native gene and mRFP (SBa_000484) between the Ptac cassette (SBa_000561) and the BioBrick suffix of plasmid N58 (pSC101, CmR). The native gene sequence from +1 bp to +90 bp formed an in-frame fusion with mRFP. In cases where the gene transcript does not extend to −60 bp, a shorter cassette was cloned into N58. RBS strength was characterized using the Promoter Characterization Assay described herein.

Synthetic RBSs of sufficient length to capture the full ribosome footprint (˜35 bp) were generated with the RBS Calculator (61). The strength of each was measured using a synthetic RBS characterization vector. These vectors were constructed similar to the wild-type RBS characterization vectors using −60 bp to +90 bp of the designed synthetic gene. This region includes part of a buffer sequence, the synthetic RBS, and the region from +1 bp to +90 bp of the synthetic gene. If the synthetic and wild-type RBSs differed by more than 3-fold in expression, new RBS sequences were generated and screened. Insulator parts consisting of −50 bp of random DNA precede each synthetic RBS (66).

Synthetic operons were cloned into the pSB4C5 (pSC101, CmR) backbone between the BioBrick prefix and BioBrick suffix.

### Synthetic Part Generation

T7*RNA Polymerase: The T7 RNA polymerase was modified to be non-toxic to both Klebsiella and E. coli at high expression levels. The RNAP was expressed from a low-copy origin (pSa) under control of a weak RBS (SBa_000507, TATCCAAACCAGTAGCTCAATTGGAGTCGTCTAT (SEQ ID NO:341)) and N-terminal degradation tag (SBa_000509, TTGTTTATCAAGCCTGCGGATCTCCGCGAAATTGTGACTTTTCCGCTATTTAGCGATC TTGTTCAGTGTGGCTTTCCTTCACCGGCAGCAGATTACGTTGAACAGCGCATCGATC TGGGTGGC (SEQ ID NO:342)). The start codon was changed from ATG to GTG, and the active site contained a mutation (R632S).

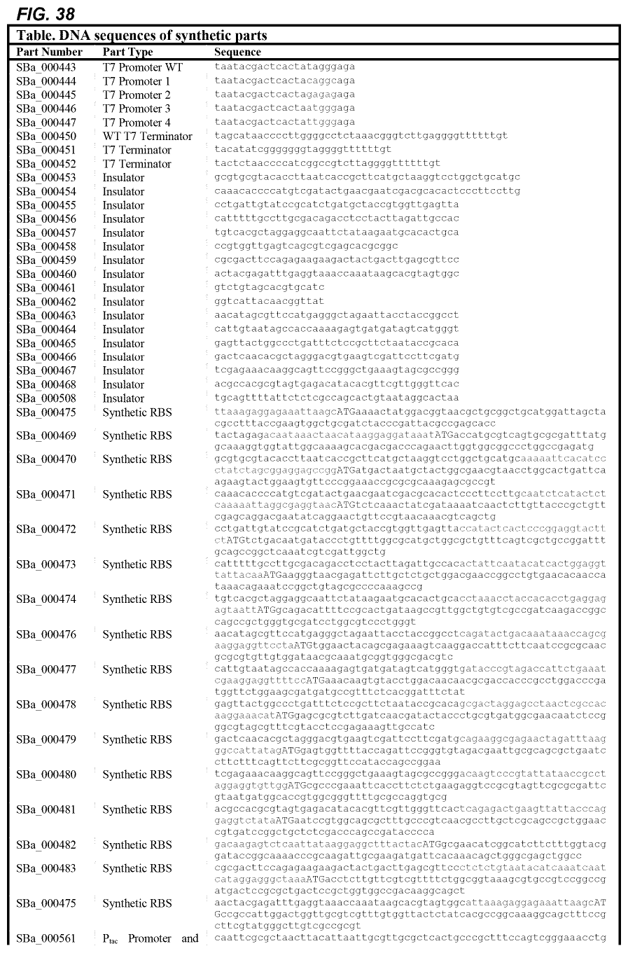

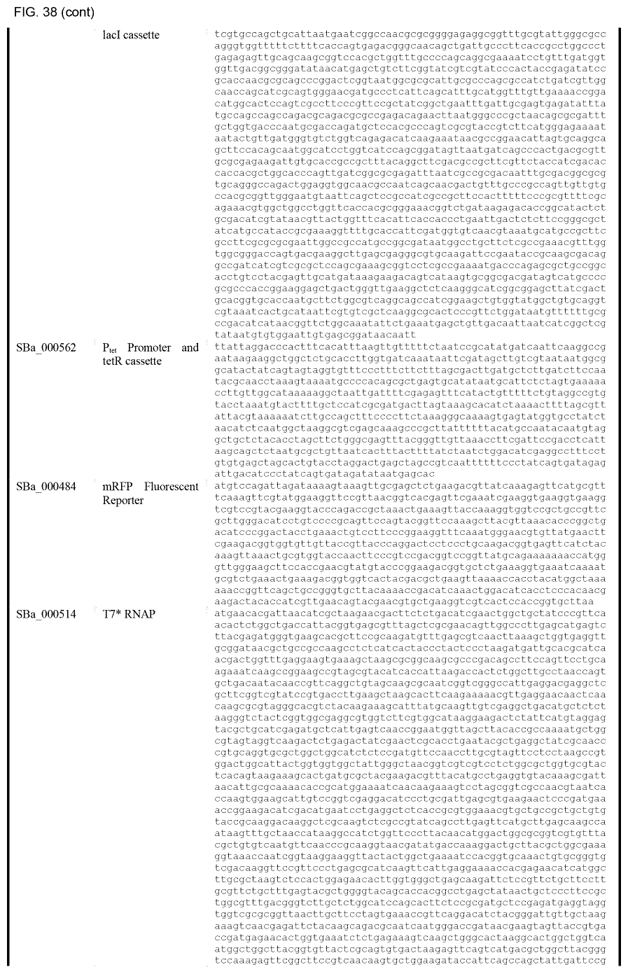

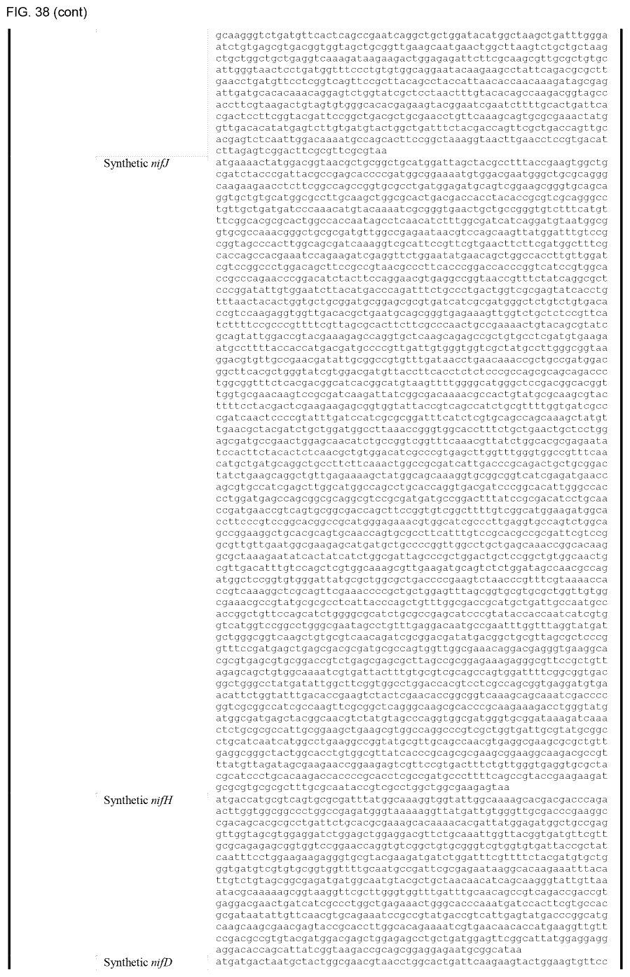

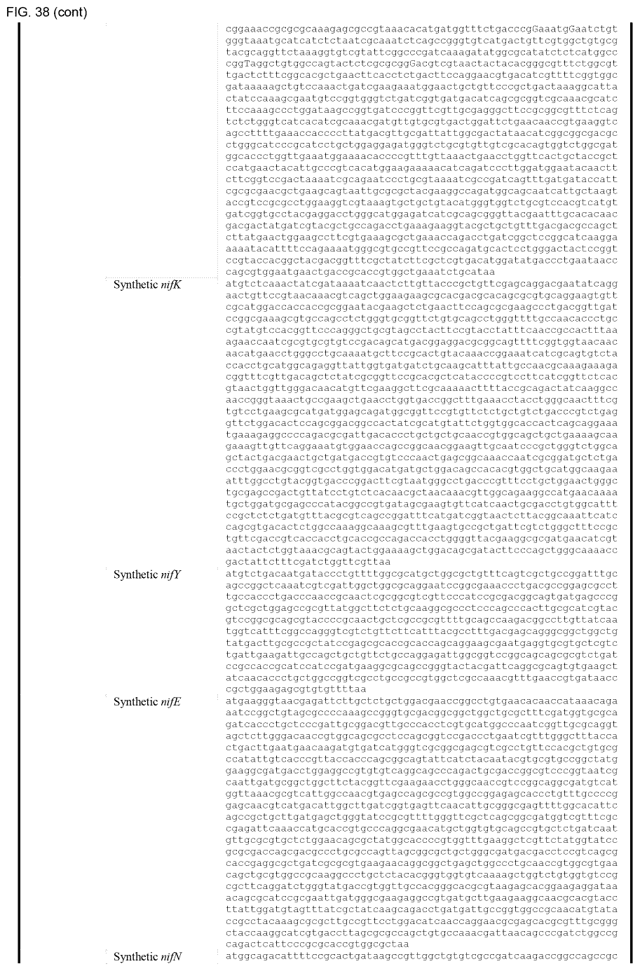

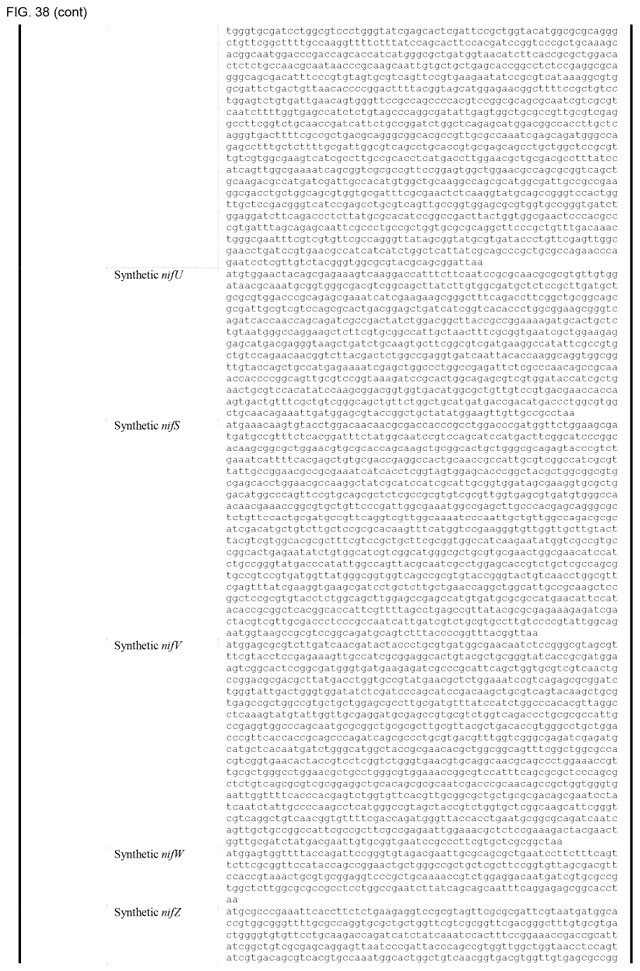

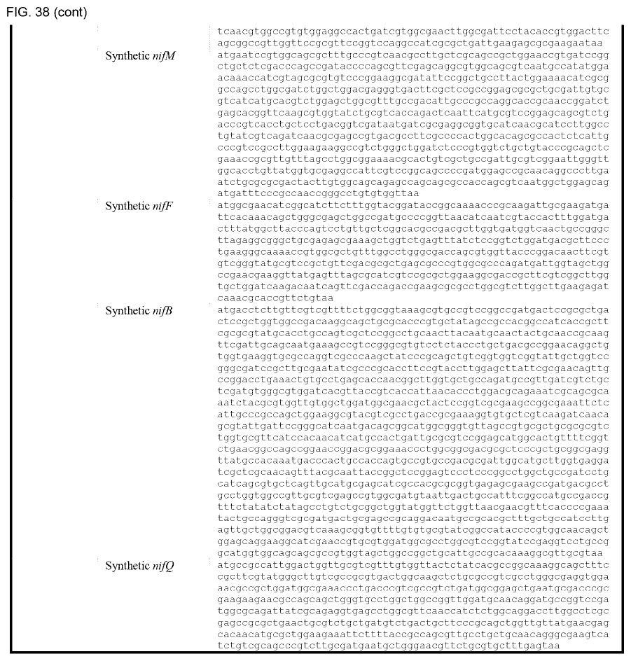

T7 promoters: T7 promoters were generated from a random library. The T7 promoter seed sequence was TAATACGACTCACTANNNNNAGA (SEQ ID NO:156). For the sequences of individual promoters, see FIG. 38.

T7 terminators: T7 terminators were generated from a random library and inserted into the terminator characterization vector N292 (SBa_000566). The T7 terminator seed sequence was TANNNAACCSSWWSSNSSSSTCWWWCGSSSSSSWWSSGTTT (SEQ ID NO:343).

Terminator plasmids were co-transformed with plasmid N249 and characterized (Methods, Fluorescence Characterization) under 1 mM IPTG induction of T7* RNAP. RFP expression was measured for each terminator, and data are reported as the fold reduction in measured fluorescence when compared to a derivative of N292 carrying no terminator. For the sequences of individual terminators, see FIG. 38

Ribosome binding sites: The RBS Calculator was used to generate an RBS that matched the measured strength of the wild-type RBS. In three cases, synthetic RBSs were selected from existing parts (SBa_000475 for nifJ and nifQ, and SBa_000469 for nifH). In cases where the strength of the initial synthetic RBS differed from the WT RBS by more than 3-fold (nifV, nifZ, and nifM), a library of synthetic RBS was constructed by replacing the 15 bp upstream of the start codon with NNNAGGAGGNNNNNN (SEQ ID NO:344). We screened mutants in each library to identify synthetic RBSs within three fold of the WT RBS strength. Ribosome binding site strength is reported in arbitrary fluorescence units measured using the fluorescence characterization assay.

Insulator sequences (spacer sequences): Insulator sequences were generated using the Random DNA Generator using a random GC content of 50% (66).

ANDN Logic: We constructed a genetic circuit encoding the logic A ANDN B and used this circuit to control T7* RNAP in Controller #3. In this circuit, the A ANDN B logic corresponds to the presence or absence of the inducers, IPTG and aTc, such that the cell computes IPTG ANDN aTc. The circuit was constructed by modifying the Ptac promoter in Controller #1 (SBa_000520) to include the cI repressor binding sites OR1 and OR2 to produce plasmid N639 (SBa_000560). Additionally, plasmid pNOR1020 encodes the repressor cI under control of the Ptet promoter (62). We modified pNOR1020 by changing the resistance marker to confer ampicillin resistance to produce N573 (SBa_000559). When N639 and N573 are co-transformed, they produce the logic circuit IPTG ANDN aTc.

Ptac (SBa_000512) sequence (SEQ ID NO: 334):
tattctgaaatgagctgttgacaattaatcatcggctcgtataatgtgt
ggaattgtgagcggataacaatt
Ptac plus OR1 and OR2 (SBa_000506) sequence
(SEQ ID NO: 335):
tattaacaccgtgcgtgttgacagctatacctctggcggttataatgct
agcggaattgtgagcggataacaatt

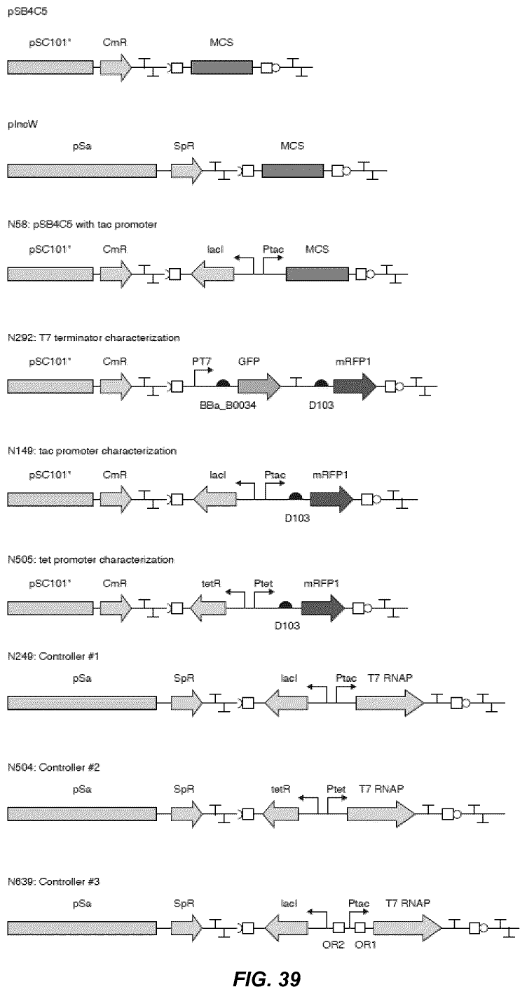

FIG. 39 illustrates maps for key plasmids.

The nif gene cluster in K. oxytoca Ma5L was re-sequenced from PCR fragments. The re-sequenced DNA sequence was compared to the reference sequence from Genbank, X13303.1 (52). Sequence differences are listed in FIG. 35. The nucleotide locations are numbered relative to X13303.1. Amino acid mutations to correct errors in the X13303.1 record are shown (Impact).

### REFERENCES

1. Fischbach M, Voigt, C. A. (2010) Prokaryotic gene clusters: A rich toolbox for synthetic biology. Biotechnol. J. 5:1277-1296.
2. Ishihama A (2010) Prokaryotic genome regulation: multifactor promoters, multitarget regulators and hierarchic networks. FEMS Microbiol Rev 34(5):628-645.
3. Mandal M & Breaker R R (2004) Gene regulation by riboswitches. Nat Rev Mol Cell Biol 5(6):451-463.
4. Temme K, et al. (2008) Induction and relaxation dynamics of the regulatory network controlling the type III secretion system encoded within Salmonella pathogenicity island 1. J Mol Biol 377(1):47-61.
5. Georg J & Hess W R (2011) cis-antisense RNA, another level of gene regulation in bacteria. Microbiol Mol Biol Rev 75(2):286-300.
6. Guell M, Yus E, Lluch-Senar M, & Serrano L (2011) Bacterial transcriptomics: what is beyond the RNA horiz-ome?Nature reviews. Microbiology 9(9):658-669.
7. Johnson Z I & Chisholm S W (2004) Properties of overlapping genes are conserved across microbial genomes. Genome Res 14(11):2268-2272.
8. Zazopoulos E, et al. (2003) A genomics-guided approach for discovering and expressing cryptic metabolic pathways. Nat Biotechnol 21(2):187-190.
9. Medema M H, Breitling R, Bovenberg R, & Takano E (2011) Exploiting plug-and-play synthetic biology for drug discovery and production in microorganisms. Nature reviews. Microbiology 9(2):131-137.
10. Gottelt M, Koi, S., Gomez-Escribano, J. P., Bibb, M., Takano, E. (2010) Deletion of a regulatory gene within the cpk gene cluster reveals novel antibacterial activity in Steptomyces coelicolor A3(2). Microbiology 156:2343-2353.
11. Lombo F, Brana, A. F., Mendez, C., Salas, J. A. (1999) The mithramycin gene cluster of Steptomyces argillaceus contains a positive regulatory gene and two repeated DNA sequences that are located at both ends of the cluster. J. Bacteriol. 181:642-647.
12. Medema M H, Bretiling, R., Takano, E. (2011) Synthetic biology in Steptomyces bacteria. Methods Enzymol 497:485-502.
13. Pickens L B, Tang, Y., Chooi, Y-H. (2011) Metabolic engineering for the production of natural products. Annu. Rev. Chem. Biomol. Eng. 2:1-26.
14. Smanski M J, Peterson, R. M., Rajski, S. R., Shen, B. (2009) Engineered Streptomyces platensis strains that overproduce antibiotics platensimycin and platencin. Antimicrob. Agents Chemother. 53:1299-12304.
15. Biggins J B, Liu, X., Feng, Z., Brady, S.F. (2011) Metabolites from the induced expression of crypic single operons found in the genome of Burkolderia pseudomallei. JACS 133:1638-1641.
16. Watanabe K, Hotta, K., Praseuth, A. P., Koketsu, K., Migita, A., Boddy, C. N., Wang, C. C. C., Oguri, H., Oikawa, H. (2006) Total biosynthesis of antitumor nonribosomal peptides in Escherichia coli. Nature Chemical Biology:1-6.
17. Fowler M & Beck K (1999) Refactoring: improving the design of existing code (Addison-Wesley, Reading, MA) pp xx1, 431 p.
18. Chan L Y, Kosuri S, & Endy D (2005) Refactoring bacteriophage T7. Mol Syst Biol 1:2005 0018.
19. Czar M J, Anderson J C, Bader J S, & Peccoud J (2009) Gene synthesis demystified. Trends Biotechnol 27(2):63-72.
20. Purnick P E & Weiss R (2009) The second wave of synthetic biology: from modules to systems. Nat Rev Mol Cell Biol 10(6):410-422.
21. Stacey G S, Burris R H, & Evans H J (1992) Biological nitrogen fixation (Chapman & Hall, New York) pp xii, 943 p.
22. Burris R H (1991) Nitrogenases. J Biol Chem 266(15):9339-9342.
23. Hu Y, Fay A W, Lee C C, Yoshizawa J, & Ribbe M W (2008) Assembly of nitrogenase MoFe protein. Biochemistry 47(13):3973-3981.
24. Rubio L M & Ludden P W (2005) Maturation of nitrogenase: a biochemical puzzle. J Bacteriol 187(2):405-414.
25. Arnold W, Rump A, Klipp W, Priefer U B, & Puhler A (1988) Nucleotide sequence of a 24,206-base-pair DNA fragment carrying the entire nitrogen fixation gene cluster of Klebsiella pneumoniae. J Mol Biol 203(3):715-738.
26. Dixon R & Kahn D (2004) Genetic regulation of biological nitrogen fixation. Nat Rev Microbiol 2(8):621-631.
27. Dixon R A & Postgate J R (1972) Genetic transfer of nitrogen fixation from Klebsiella pneumoniae to Escherichia coli. Nature 237(5350):102-103.
28. Simon H M, Homer M J, & Roberts G P (1996) Perturbation of nifT expression in Klebsiella pneumoniae has limited effect on nitrogen fixation. J Bacteriol 178(10):2975-2977.
29. Fani R, Gallo R, & Lio P (2000) Molecular evolution of nitrogen fixation: the evolutionary history of the nifD, nifK, nifE, and nifN genes. J Mol Evol 51(1):1-11.
30. Gosink M M, Franklin N M, & Roberts G P (1990) The product of the Klebsiella pneumoniae nifX gene is a negative regulator of the nitrogen fixation (nif) regulon. J Bacteriol 172(3):1441-1447.
31. Orme-Johnson W H (1985) Molecular basis of biological nitrogen fixation. Annu Rev Biophys Biophys Chem 14:419-459.
32. Kelly J R, et al. (2009) Measuring the activity of BioBrick promoters using an in vivo reference standard. J Biol Eng 3:4.
33. Gibson D G, et al. (2009) Enzymatic assembly of DNA molecules up to several hundred kilobases. Nat Methods 6(5):343-345.
34. Yokobayashi Y, Weiss R, & Arnold F H (2002) Directed evolution of a genetic circuit. Proc Natl Acad Sci USA 99(26):16587-16591.
35. Tamsir A, Tabor J J, & Voigt C A (2011) Robust multicellular computing using genetically encoded NOR gates and chemical ‘wires’. Nature 469(7329):212-215.
36. Alon U (2007) An introduction to systems biology: design principles of biological circuits (Chapman & Hall/CRC, Boca Raton, FL) pp xvi, 301 p., 304 p. of plates.
37. Kitano H (2002) Systems biology: a brief overview. Science 295(5560):1662-1664.
38. Palsson B (2006) Systems biology: properties of reconstructed networks (Cambridge University Press, Cambridge; New York) pp xii, 322 p.
39. Kalir S, et al. (2001) Ordering genes in a flagella pathway by analysis of expression kinetics from living bacteria. Science 292(5524):2080-2083.
40. Zaslaver A, Mayo A, Ronen M, & Alon U (2006) Optimal gene partition into operons correlates with gene functional order. Phys Biol 3(3):183-189.
41. Kovacs K, Hurst L D, & Papp B (2009) Stochasticity in protein levels drives colinearity of gene order in metabolic operons of Escherichia coli. PLoS Biol 7(5):e1000115.
42. Wenzel S C & Muller R (2005) Recent developments towards the heterologous expression of complex bacterial natural product biosynthetic pathways. Curr Opin Biotechnol 16(6):594-606.
43. Welch M, et al. (2009) Design parameters to control synthetic gene expression in Escherichia coli. PLoS One 4(9):e7002.
44. Salis H M, Mirsky E A, & Voigt C A (2009) Automated design of synthetic ribosome binding sites to control protein expression. Nat Biotechnol 27(10):946-950.
45. Zomer A L (2011) PPP: Perform Promoter Prediction.
46. Studholme D (2011) PromScan.
47. Kingsford C L, Ayanbule K, & Salzberg S L (2007) Rapid, accurate, computational discovery of Rho-independent transcription terminators illuminates their relationship to DNA uptake. Genome Biol 8(2):R22.
48. Stewart W D, Fitzgerald G P, & Burris R H (1967) In situ studies on nitrogen fixation with the acetylene reduction technique. Science 158(3800):536.
49. Philippe N, Alcaraz J P, Coursange E, Geiselmann J, & Schneider D (2004) Improvement of pCVD442, a suicide plasmid for gene allele exchange in bacteria. Plasmid 51(3):246-255.
50. Zhao D, Curatti L, & Rubio L M (2007) Evidence for nifU and nifS participation in the biosynthesis of the iron-molybdenum cofactor of nitrogenase. J Biol Chem 282(51):37016-37025.
51. Kelly J R, et al. (2009) Measuring the activity of BioBrick promoters using an in vivo reference standard. J Biol Eng 3:4.
52. Arnold W, Rump A, Klipp W, Priefer U B, & Puhler A (1988) Nucleotide sequence of a 24,206-base-pair DNA fragment carrying the entire nitrogen fixation gene cluster of Klebsiella pneumoniae. J Mol Biol 203(3):715-738.
53. Bayer T S, et al. (2009) Synthesis of Methyl Halides from Biomass Using Engineered Microbes. J Am Chem Soc 131(18):6508-6515.
54. Jacob G S, Schaefer J, Garbow J R, & Stejskal E O (1987) Solid-state NMR studies of Klebsiella pneumoniae grown under nitrogen-fixing conditions. J Biol Chem 262(1):254-259.
55. Mason C A & Hamer G (1987) Cryptic Growth in Klebsiella-Pneumoniae. Appl Microbiol Biot 25(6):577-584.
56. Villalobos A, Ness J E, Gustafsson C, Minshull J, & Govindarajan S (2006) Gene Designer: a synthetic biology tool for constructing artificial DNA segments. BMC Bioinformatics 7:285.
57. Shetty R P, Endy D, & Knight T F, Jr. (2008) Engineering BioBrick vectors from BioBrick parts. J Biol Eng 2:5.
58. Miyazaki K (2003) Creating random mutagenesis libraries by megaprimer PCR of whole plasmid (MEGAWHOP). Methods Mol Biol 231:23-28.
59. Gibson D G, et al. (2009) Enzymatic assembly of DNA molecules up to several hundred kilobases. Nat Methods 6(5):343-345.
60. Dykxhoorn D M, St Pierre R, & Linn T (1996) A set of compatible tac promoter expression vectors. Gene 177(1-2):133-136.
61. Salis H M, Mirsky E A, & Voigt C A (2009) Automated design of synthetic ribosome binding sites to control protein expression. Nat Biotechnol 27(10):946-950.
62. Tamsir A, Tabor J J, & Voigt C A (2011) Robust multicellular computing using genetically encoded NOR gates and chemical ‘wires’. Nature 469(7329):212-215.
63. Buck M & Cannon W (1987) Frameshifts close to the Klebsiella pneumoniae nifH promoter prevent multicopy inhibition by hybrid nifH plasmids. Mol Gen Genet 207(2-3):492-498.
64. Riedel G E, Brown S E, & Ausubel F M (1983) Nitrogen fixation by Klebsiella pneumoniae is inhibited by certain multicopy hybrid nif plasmids. J Bacteriol 153(1):45-56.
65. Beynon J, Cannon M, Buchanan-Wollaston V, & Cannon F (1983) The nif promoters of Klebsiella pneumoniae have a characteristic primary structure. Cell 34(2):665-671.
66. Maduro M (2011) Random DNA Generator, faculty.ucr.edu/˜mmaduro/random.htm.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1-81. (canceled)

82. A bacterial nitrogen reduction expression system comprising nucleic acids encoding:
at least one gene cluster comprising a plurality of coding sequences for a set of polypeptides encoded by genes collectively associated with nitrogen fixation within a cell;
a heterologous promoter region that directs expression of the at least one of the coding sequences; and
a transcriptional controller coding sequence that encodes a sequence-specific DNA binding polypeptide that directs expression of the at least one coding sequence of the expression system, wherein the sequence-specific DNA binding polypeptide binds directly or indirectly to the heterologous promoter region.

83. The bacterial nitrogen reduction expression system of claim 82, wherein the gene cluster does not include the nifL gene.

84. The bacterial nitrogen reduction expression system of claim 82, wherein the nifL gene is eliminated from the gene cluster.

85. The bacterial nitrogen reduction expression system of claim 82, wherein the genes collectively associated with nitrogen fixation within a cell are selected from the group consisting of: nifJ, nifH, nifD, nifK, nifY, nifE, nifN, nifU, nifS, nifV, nifW, nifZ, nifM, nifB, and nifQ.

86. The bacterial nitrogen reduction expression system of claim 82, wherein the heterologous promoter of the one or more of the coding sequences causes the one or more coding sequences to be expressed constitutively.

87. The bacterial nitrogen reduction expression system of claim 82, wherein the heterologous promoter of the one or more of the coding sequences causes the one or more coding sequences to be expressed at an expression level which is associated with maximal nitrogenase activity.

88. The bacterial nitrogen reduction expression system of claim 82, wherein the heterologous promoter of the operon causes two or more coding sequences to be expressed at an expression level which is associated with maximal nitrogenase activity.

89. The bacterial nitrogen reduction expression system of claim 82, wherein the heterologous promoter causes each coding sequence of the operon to be expressed at an expression level which is associated with maximal nitrogenase activity.

90. The bacterial nitrogen reduction expression system of claim 82, wherein the sequence-specific DNA binding polypeptide does not regulate the genes collectively associated with nitrogen fixation under native regulation.

91. The bacterial nitrogen reduction expression system of claim 82, wherein the sequence-specific DNA binding polypeptide is a sigma factor.

92. The bacterial nitrogen reduction expression system of claim 91, wherein the sigma factor is a Group 1 sigma factor.

93. A cell comprising the bacterial nitrogen reduction expression system of claim 82.

94. The cell of claim 93, wherein the cell is a prokaryotic cell.

95. The cell of claim 94, wherein the prokaryotic cell is a Klebsiella cell.

96. A cell comprising at least one nif gene cluster comprising a plurality of coding sequences for a set of polypeptides encoded by genes collectively associated with nitrogen fixation within the cell,
wherein the gene cluster comprises at least one heterologous regulatory sequence responsive to a sequence-specific DNA binding polypeptide such that at least one coding sequence is under control of heterologous regulation, and
wherein nifL is omitted from the nif gene cluster.

97. The cell of claim 96, wherein the genes collectively associated with nitrogen fixation within a cell are selected from the group consisting of: nifJ, nifH, nifD, nifK, nifY, nifE, nifN, nifU, nifS, nifV, nifW, nifZ, nifM, nifB, and nifQ.

98. The cell of claim 96, wherein the heterologous regulatory sequence causes the at least one coding sequence to be expressed constitutively.

99. The cell of claim 96, wherein the sequence-specific DNA binding polypeptide does not regulate the nif gene cluster under native regulation.

100. The cell of claim 96, wherein the sequence-specific DNA binding polypeptide is a sigma factor.

101. The cell of claim 100, wherein the sigma factor is a Group 1 sigma factor.

102. The cell of claim 96, wherein the cell is a prokaryotic cell.

103. The cell of claim 102, wherein the prokaryotic cell is a Klebsiella cell.
